(19) United States
(12) Patent Application Publication
(10) Pub. No.: US 2025/0276089 A1
(43) Pub. Date: Sep. 4, 2025

(54) CO-DELIVERY OF INHIBITORY NUCLEIC ACIDS AND GENOME EDITORS FOR TUMOR THERAPY

(71) Applicant: The Board of Regents of The University of Texas System, Austin, TX (US)

(72) Inventors: Di ZHANG, Dallas, TX (US); Daniel SIEGWART, Dallas, TX (US)

(73) Assignee: The Board of Regents of The University of Texas System, Austin, TX (US)

(21) Appl. No.: 18/862,512

(22) PCT Filed: May 3, 2023
(86) PCT No.: PCT/US2023/066555

§ 371 Date: Nov. 1, 2024

Related U.S. Application Data
(60) Provisional application No. 63/337,943, filed on May 3, 2022.

(51) Int. Cl. A61K 48/00; A61K 9/00; A61K 45/06; A61K 47/69; A61P 35/00; C12N 9/22; C12N 15/11; C12N 15/113; C12N 15/88

(52) U.S. Cl. CPC A61K 48/005; A61K 9/0019; A61K 45/06; A61K 47/6911; A61K 48/0075; A61P 35/00; C12N 9/226; C12N 15/111; C12N 15/1137; C12N 15/88; C12N 2310/14; C12N 2310/20; C12Y 207/10002

(57) ABSTRACT

In some aspects, the present disclosure provides compositions comprising an inhibitory polynucleotide and either a guide polynucleotide and/or a polynucleotide that encodes for a nuclease or a nuclease; and a lipid nanoparticle comprising at least one ionizable lipid; wherein the each of the nucleic acids are encapsulated within the lipid nanoparticle, and pharmaceutical compositions thereof. The present disclosure also provides methods employing said compositions and/or pharmaceutical compositions, such as methods of treating diseases or disorders.

This application claims the benefit of priority to U.S. Provisional Application No. 63/337,943, filed on May 3, 2022, the entire contents of which are hereby incorporated by reference.

### STATEMENT REGARDING FEDERALLY SPONSORED RESEARCH OR DEVELOPMENT

This invention was made with government support under Grant Nos. EB025192 and CA269787 awarded by the National Institutes of Health (NIH). The government has certain rights in the invention.

### REFERENCE TO A SEQUENCE LISTING

This application contains a Sequence Listing XML, which has been submitted electronically and is hereby incorporated by reference in its entirety. Said Sequence Listing XML, created on May 3, 2023, is named UTFDP4080WO.xml and is ˜26 kilobytes in size.

### BACKGROUND

### I. Field

The present invention relates generally to the fields of nucleic acid delivery compositions. For example, in certain aspects, it relates to compositions formulated for delivery of a combination of one or more of the following nucleic acids in a lipid nanoparticle comprising at least one ionizable lipid that may be used to treat a disease or disorder.

### II. Description of Related Art

In vivo inactivation or repair of cancer-related genes using the robust and programmable clustered regularly interspaced short palindromic repeat (CRISPR) associated protein (CRISPR/Cas) system (Wang et al, 2017; Jinek et al, 2012; Cong et al, 2013; Mali et al, 2013; Xue et al, 2014; Yin et al, 2014; Maddalo et al, 2014; Long et al, 2016; Miller et al, 2017; Wei et al, 2020) represents an exciting approach for cancer treatment (Huang et al, 2018). However, CRISPR-based therapies for solid tumors face critical obstacles. The first is cancer's limitless replicative potential, a cancer hallmark (Hanahan et al. 2011), that results in an expansive tumor burden whereby editing a small number of cells would not be able to reverse disease symptoms (Huang et al, 2018; Cox et al, 2015). The second is the uniquely stiff and fibrotic stroma of the tumor microenvironment. The physically dense tumor microenvironment thus acts as a barrier to efficient tumor therapy, blocking nanoparticle uptake into tumors, access to enough cells to overcome replicative potential, and immune cell infiltration to tumor tissue (Jiang 2016).

Therefore, there exists a need for therapies that may be used to treat tumors and other indications.

### SUMMARY

In some aspects, the present disclosure provides lipid compositions, specifically lipid nanoparticles, that encapsulate two or more nucleic acids, including an siRNA and at least one of a guide nucleic acid, such as a sgRNA, or a nucleic acid that encodes for a nuclease, such as an mRNA. In particular, the present disclosure relates to compositions such as:

In some aspects, the present disclosure provides compositions comprising:
(A) an interfering polynucleotide;
(B) a second polynucleotide, wherein the second polynucleotide is either:
   (1) a polynucleotide comprising a sequence encoding for a polynucleotide guided nuclease; or
   (2) a guide polynucleotide;
(C) a lipid nanoparticle comprising at least one ionizable lipid.

In some embodiments, the composition comprises a polynucleotide comprising a sequence encoding for a polynucleotide guided nuclease and a guide polynucleotide. In some embodiments, the interfering polynucleotide is an siRNA. In some embodiments, the polynucleotide comprises a sequence encoding for a polynucleotide guided nuclease such as a mRNA. In further embodiments, mRNA encodes for a Cas protein. In still further embodiments, the Cas protein is a Cas9 protein.

In some embodiments, the guide polynucleotide is a polynucleotide configured to complex with at least a portion of a target gene or transcript. In some embodiments, the guide polynucleotide is a polynucleotide that encodes for a polynucleotide that is configured to complex with at least a portion of a target gene or transcript. In some embodiments, the guide polynucleotide comprises from about 10 nucleotides to about 50 nucleotides. In further embodiments, the guide polynucleotide comprises from about 12 nucleotides to about 40 nucleotides. In still further embodiments, the guide polynucleotide comprises from about 15 nucleotides to about 30 nucleotides.

In some embodiments, the interfering polynucleotide comprises from about 10 nucleotides to about 50 nucleotides. In further embodiments, the interfering polynucleotide comprises from about 15 nucleotides to about 40 nucleotides. In still further embodiments, the interfering polynucleotide comprises from about 18 nucleotides to about 30 nucleotides. In yet further embodiments, the interfering polynucleotide comprise about 20 nucleotides to about 24 nucleotides.

In some embodiments, the polynucleotide comprising a sequence encoding for a polynucleotide guided nuclease comprises from about comprises from about 250 nucleotides to about 15,000 nucleotides. In further embodiments, the polynucleotide comprising a sequence encoding for a polynucleotide guided nuclease comprises from about 500 nucleotides to about 5,000 nucleotides. In still further embodiments, the polynucleotide comprising a sequence encoding for a polynucleotide guided nuclease comprises from about 800 nucleotides to about 2,500 nucleotides.

In some embodiments, the composition comprises a weight ratio of the polynucleotide comprising a sequence encoding for a polynucleotide guided nuclease to the guide polynucleotide from about 10:1 to about 1:5. In further embodiments, wherein the weight ratio of a polynucleotide comprising a sequence encoding for a polynucleotide guided nuclease to the guide polynucleotide is from about 8:1 to about 1:2. In still further embodiments, the weight ratio of the polynucleotide comprising a sequence encoding for a polynucleotide guided nuclease to the guide polynucleotide is from about 5:1 to about 1:1. In even further embodiments, the weight ratio of the polynucleotide comprising a sequence encoding for a polynucleotide guided nuclease to the guide polynucleotide is 2:1.

In some embodiments, the composition comprises a weight ratio of the polynucleotide comprising a sequence encoding for a polynucleotide guided nuclease to the inhibitory polynucleotide from about 10:1 to about 1:10. In further embodiments, the weight ratio of the polynucleotide comprising a sequence encoding for a polynucleotide guided nuclease to the inhibitory polynucleotide is from about 5:1 to about 1:5. In still further embodiments, the weight ratio of the polynucleotide comprising a sequence encoding for a polynucleotide guided nuclease to the inhibitory polynucleotide is from about 2:1 to about 1:2. In even further embodiments, the weight ratio of the polynucleotide comprising a sequence encoding for a polynucleotide guided nuclease to the inhibitory polynucleotide is 1:1 or 2:3.

In some embodiments, the composition comprises a weight ratio of the guide polynucleotide to the inhibitory polynucleotide from about 4:1 to about 1:10. In further embodiments, the weight ratio of the guide polynucleotide to the inhibitory polynucleotide is from about 2:1 to about 1:8. In still further embodiments, the weight ratio of the guide polynucleotide to the inhibitory polynucleotide is from about 1:1 to about 1:4. In yet further embodiments, the weight ratio of the guide polynucleotide to the inhibitory polynucleotide is 1:2 or 1:3.

In some embodiments, the ionizable lipid is a cationic lipid. In some embodiments, the ionizable lipid is a dendron or dendrimer.

In some embodiments, the ionizable lipid is a compound of the formula:

Core-Repeating Unit-Terminating Group (D-I)

wherein the core is linked to the repeating unit by removing one or more hydrogen atoms from the core and replacing the atom with the repeating unit and wherein: the core has the formula:

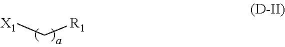

wherein:
X₁ is amino or alkylamino_{(C≤12)}, dialkylamino_{(C≤12)}, heterocycloalkyl_{(C≤12)}, heteroaryl_{(C≤12)}, or a substituted version thereof;
R₁ is amino, hydroxy, or mercapto, or alkylamino_{(C≤12)}, dialkylamino_{(C≤12)}, or a substituted version of either of these groups; and
a is 1, 2, 3, 4, 5, or 6; or
   the core has the formula:

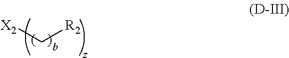

wherein:
X₂ is N(R₅)_{y};
R₅ is hydrogen, alkyl_{(C≤18)}, or substituted alkyl_{(C≤18)}; and
y is 0, 1, or 2, provided that the sum of y and z is 3;
R₂ is amino, hydroxy, or mercapto, or alkylamino_{(C≤12)}, dialkylamino_{(C≤12)}, or a substituted version of either of these groups;
b is 1, 2, 3, 4, 5, or 6; and
z is 1, 2, 3; provided that the sum of z and y is 3; or
   the core has the formula:

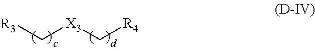

wherein:
X₃ is —NR₆—, wherein R⁶ is hydrogen, alkyl_{(C≤8)}, or substituted alkyl_{(C≤8)}, —O—, or alkylaminodiyl_{(C≤8)}, alkoxydiyl_{(C≤8)}, arenediyl_{(C≤8)}, heteroarenediyl_{(C≤8)}, heterocycloalkanediyl_{(C≤8)}, or a substituted version of any of these groups;
R₃ and R₄ are each independently amino, hydroxy, or mercapto, or alkylamino_{(C≤12)}, dialkylamino_{(C≤12)}, or a substituted version of either of these groups; or a group of the formula: —N(R_{f})_{f}(CH₂CH₂N(R_{c}))ₑR_{d},

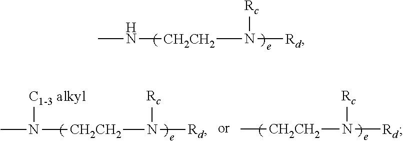

wherein:
e and f are each independently 1, 2, or 3; provided that the sum of e and f is 3;
R_{c}, R_{d}, and R_{f} are each independently hydrogen, alkyl_{(C≤6)}, or substituted alkyl_{(C≤6)};
c and d are each independently 1, 2, 3, 4, 5, or 6; or the core is alkylamine_{(C≤18)}, dialkylamine_{(C≤36)}, heterocycloalkane_{(C≤12)}, or a substituted version of any of these groups;
   wherein the repeating unit comprises a degradable diacyl and a linker;
   the degradable diacyl group has the formula:

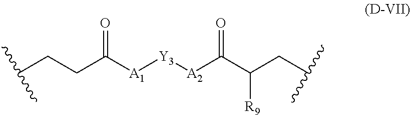

wherein:
A₁ and A₂ are each independently —O—, —S—, or —NRₐ—, wherein:
Rₐ is hydrogen, alkyl_{(C≤6)}, or substituted alkyl_{(C≤6)};
Y₃ is alkanediyl_{(C≤12)}, alkenediyl_{(C≤12)}, arenediyl_{(C≤12)}, or a substituted version of any of these groups; or a group of the formula:

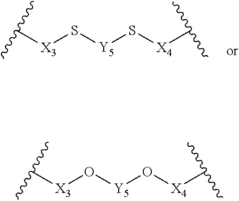

wherein:
   X₃ and X₄ are alkanediyl_{(C≤12)}, alkenediyl_{(C≤12)}, arenediyl_{(C≤12)}, or a substituted version of any of these groups;
   Y₅ is a covalent bond, alkanediyl_{(C≤12)}, alkenediyl_{(C≤12)}, arenediyl_{(C≤12)}, or a substituted version of any of these groups; and
   R₉ is alkyl_{(C≤8)} or substituted alkyl_{(C≤8)};
      the linker group has the formula:

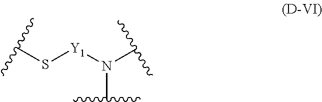

wherein:
Y₁ is alkanediyl_{(C≤12)}, alkenediyl_{(C≤12)}, arenediyl_{(C≤12)}, or a substituted version of any of these groups; and
   wherein when the repeating unit comprises a linker group, then the linker group comprises an independent degradable diacyl group attached to both the nitrogen and the sulfur atoms of the linker group if n is greater than 1, wherein the first group in the repeating unit is a degradable diacyl group, wherein for each linker group, the next repeating unit comprises two degradable diacyl groups attached to the nitrogen atom of the linker group; and wherein n is the number of linker groups present in the repeating unit; and
   the terminating group has the formula:

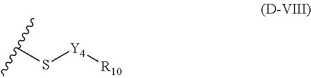

wherein:
Y₄ is alkanediyl_{(C≤24)}, alkenediyl_{(C≤24)}, or a substituted version thereof; R₁₀ is hydrogen, amino, carboxy, hydroxy, or aryl_{(C≤12)}, alkylamino_{(C≤12)}, dialkylamino_{(C≤12)}, N-heterocycloalkyl_{(C≤12)}, —C(O)N(R₁₁)-alkanediyl_{(C≤6)}-heterocycloalkyl_{(C≤12)}, —C(O)-alkyl-amino_{(C≤12)}, —C(O)-dialkylamino_{(C≤12)}, —C(O)—N-heterocyclo-alkyl_{(C≤12)}, wherein:
R₁₁ is hydrogen, alkyl_{(C≤6)}, or substituted alkyl_{(C≤6)};
wherein the final degradable diacyl in the chain is attached to a terminating group;
n is 0, 1, 2, 3, 4, 5, or 6;
   or a pharmaceutically acceptable salt thereof.

In some embodiments, in Formula (D-I), the core is further defined by the formula:

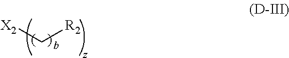

wherein:
X₂ is N(R₅)_{y};
   R₅ is hydrogen or alkyl_{(C≤8)}, or substituted alkyl_{(C≤18)}; and
y is 0, 1, or 2, provided that the sum of y and z is 3;
R₂ is amino, hydroxy, or mercapto, or alkylamino_{(C≤12)}, dialkylamino_{(C≤12)}, or a substituted version of either of these groups;
b is 1, 2, 3, 4, 5, or 6; and
z is 1, 2, 3; provided that the sum of z and y is 3.

In further embodiments, in Formula (D-I), the core is further defined as:

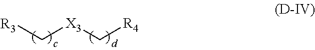

wherein:
X₃ is —NR₆—, wherein R₆ is hydrogen, alkyl(C≤), or substituted alkyl_{(C≤8)}, —O—, or alkylaminodiyl_{(C≤8)}, alkoxydiyl_{(C≤8)}, arenediyl_{(C≤8)}, heteroarenediyl_{(C≤8)}, heterocycloalkanediyl_{(C≤8)}, or a substituted version of any of these groups;
R₃ and R₄ are each independently amino, hydroxy, or mercapto, or alkylamino_{(C≤12)}, dialkylamino_{(C≤12)}, or a substituted version of either of these groups; or a group of the formula: —N(R_{f})_{f}(CH₂CH₂N(R_{c}))ₑR_{d},

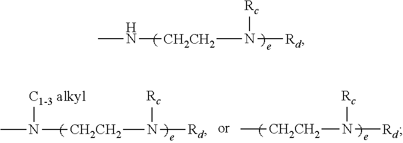

wherein:
e and f are each independently 1, 2, or 3; provided that the sum of e and f is 3;
R_{c}, R_{d}, and R_{f} are each independently hydrogen, alkyl_{(C≤6)}, or substituted alkyl_{(C≤6)};
c and d are each independently 1, 2, 3, 4, 5, or 6.

In even further embodiments, in Formula (D-I), the core is further defined as:

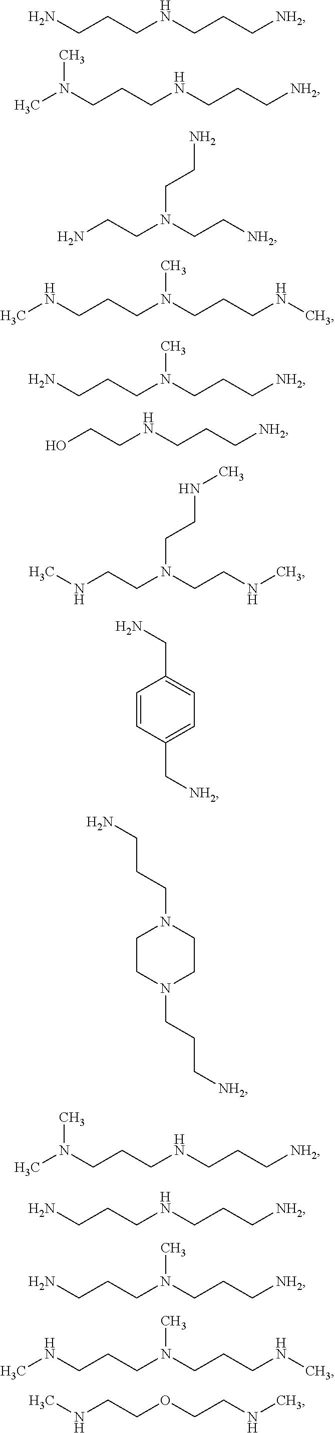

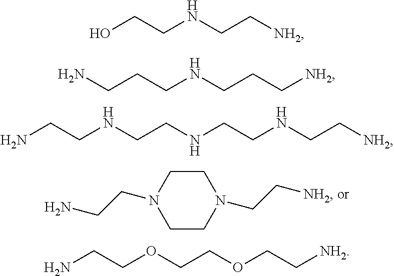

In still further embodiments, in Formula (D-I), the core is further defined as:

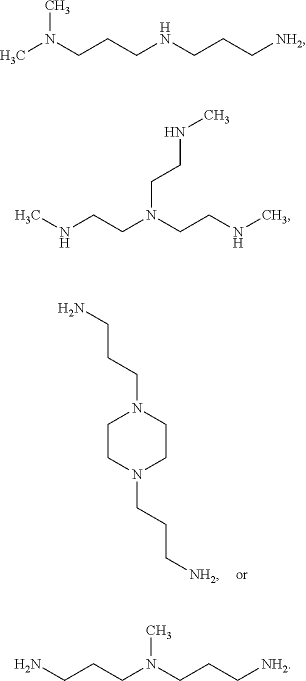

In yet further embodiments, in Formula (D-I), the core is further defined as:

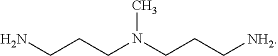

In some embodiments, A₁ and A₂ are O. In some embodiments, Y₃ is alkanediyl_{(C≤12)} or substituted alkanediyl_{(C≤12)}. In some embodiments, Y₁ is alkanediyl_{(C≤12)} or substituted alkanediyl_{(C≤12)}. In some embodiments, the terminating group is further defined as:

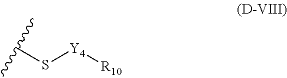

wherein:
Y₄ is alkanediyl_{(C≤18)} or alkenediyl_{(C≤18)}; and R₁₀ is hydrogen.

In further embodiments, the terminating group is further defined as:

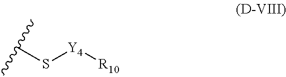

wherein:
Y₄ is alkanediyl_{(C≤18)}; and
R₁₀ is hydrogen.

In some embodiments, the dendrimer or dendron is further defined as:

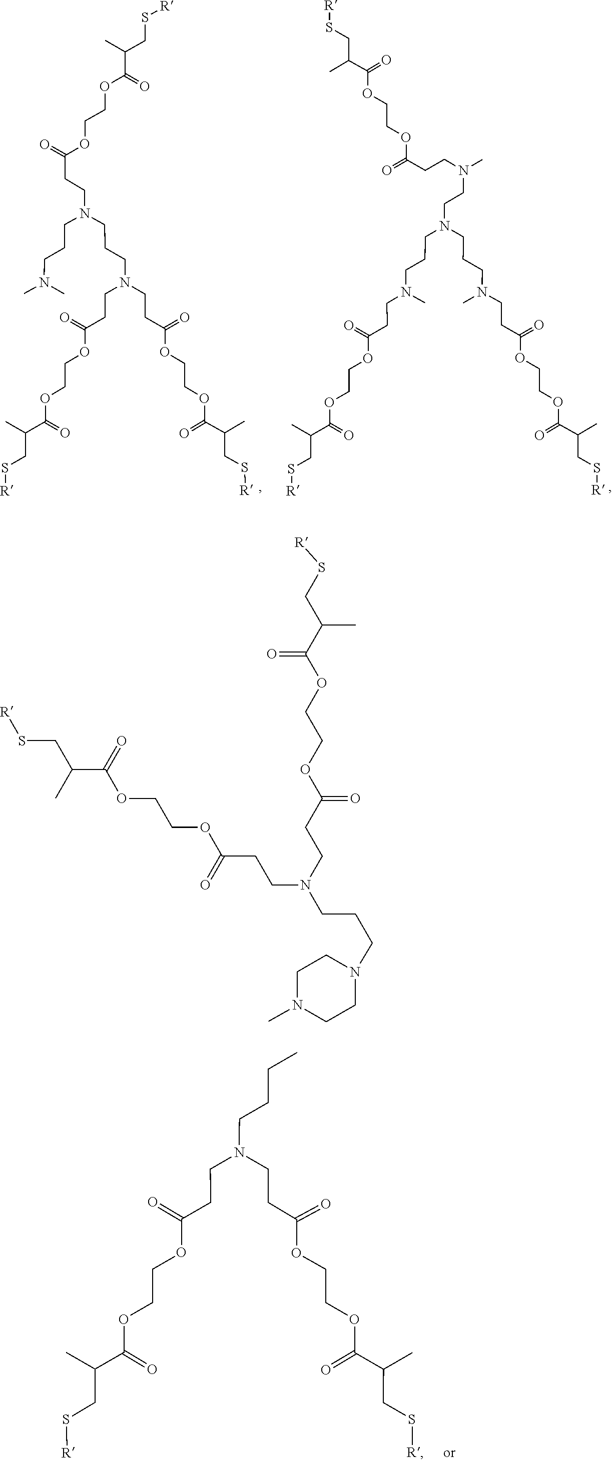

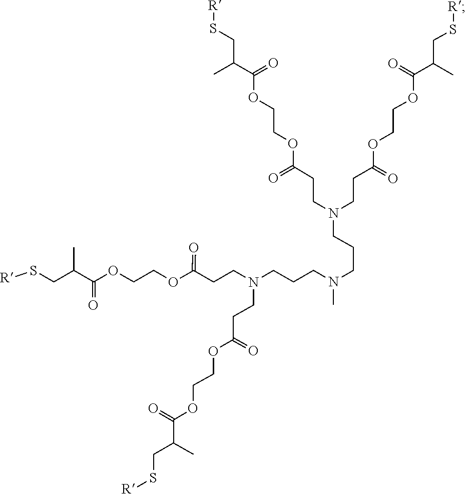

wherein:
R′ is alkyl_{(C≤18)}, alkenyl_{(C≤18)}, or a substituted version thereof.

In further embodiments, the dendrimer or dendron is further defined as:

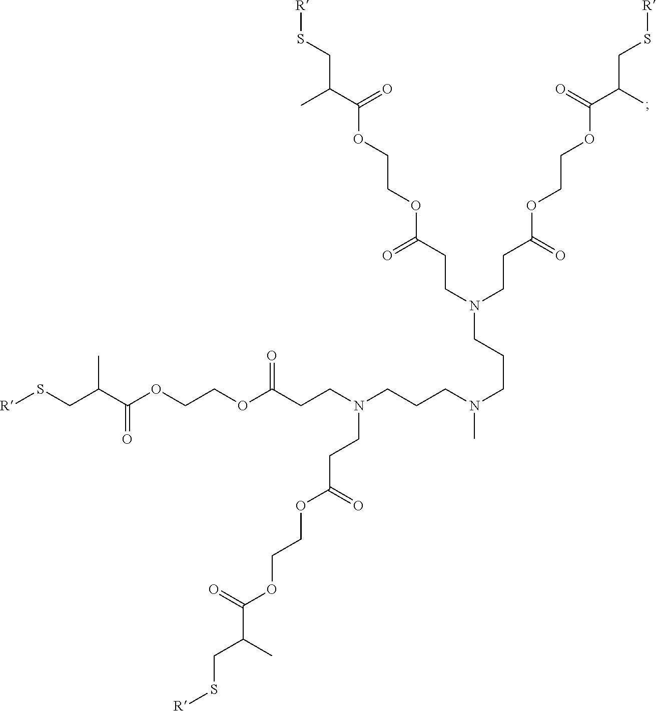

wherein:
R′ is alkyl_{(C≤18)}, alkenyl_{(C≤8)}, or a substituted version thereof.

In still further embodiments, the dendrimer or dendron is further defined as:

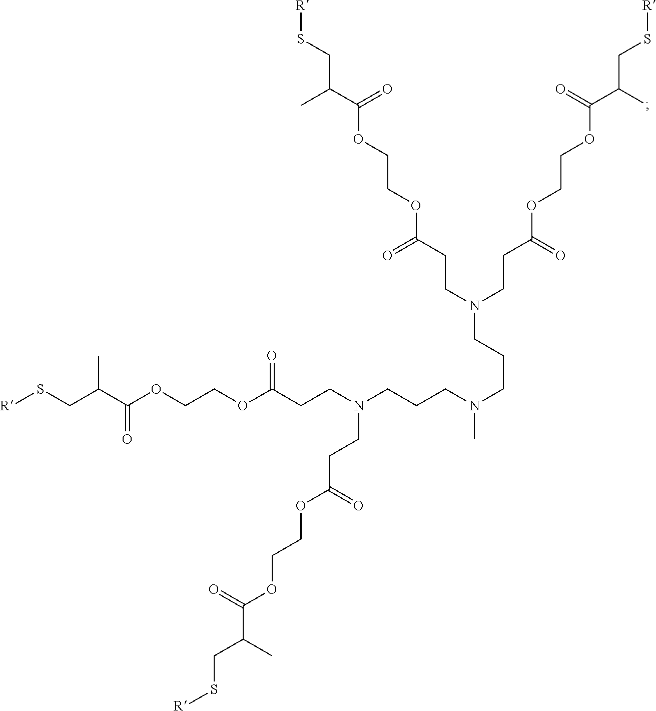

wherein:
R′ is alkyl_{(C6-18)}.

In other embodiments, the ionizable cationic lipid is a dendrimer or dendron of a generation (g) having a structural formula:

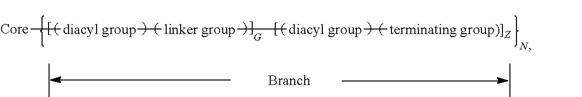

or a pharmaceutically acceptable salt thereof, wherein:
(a) the core comprises a structural formula (X_{Core}):

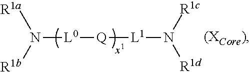

wherein:
Q is independently at each occurrence a covalent bond, —O—, —S—, —NR²—, or —CR^{3a}R_{3b}—;
R² is independently at each occurrence R^{1g} or -L²—NR^{1e}R^{1f},
R^{3a} and R^{3b} are each independently at each occurrence hydrogen or an optionally substituted alkyl;
R^{1a}, R^{1b}, R^{1c}, R^{1d}, R^{1e}, R^{1f}, and R^{1g} (if present) are each independently at each occurrence a point of connection to a branch, hydrogen, or an optionally substituted alkyl;
L⁰, L¹, and L² are each independently at each occurrence selected from a covalent bond, alkanediyl, heteroalkanediyl, [alkanediyl]-[heterocycloalkanediyl]-[alkanediyl], [alkanediyl]-(arenediyl)-[alkanediyl], heterocycloalkyl, and arenediyl; or,
alternatively, part of L¹ form a heterocycloalkyl with one of R^{1c} and R^{1d}; and
x¹ is 0, 1, 2, 3, 4, 5, or 6; and
(b) each branch of the plurality (N) of branches independently comprises a structural formula (X_{Branch}):

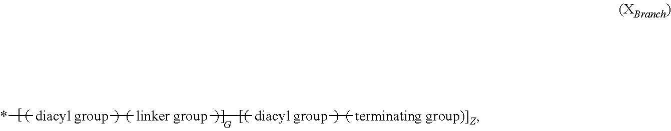

wherein:
* indicates a point of attachment of the branch to the core;
g is 1, 2, 3, or 4;
Z=2^{(g-1)};
G=0, when g=1; or G=Eᵢ₌₀^{g-2}2ⁱ, when g≠1;
(c) each diacyl group independently comprises a structural formula

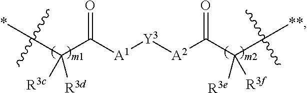

wherein:
indicates a point of attachment of the diacyl group at the proximal end thereof;
* indicates a point of attachment of the diacyl group at the distal end thereof;
Y₃ is independently at each occurrence an optionally substituted alkanediyl, an optionally substituted alkenediyl, or an optionally substituted arenediyl;
A₁ and A₂ are each independently at each occurrence —O—, —S—, or —NR⁴—, wherein:
   R⁴ is hydrogen or optionally substituted alkyl;
m¹ and m² are each independently at each occurrence 1, 2, or 3; and
R^{3c}, R^{3d}, R^{3e}, and R^{3f} are each independently at each occurrence hydrogen or an optionally substituted (e.g., C₁-C₈) alkyl; and
(d) each linker group independently comprises a structural formula

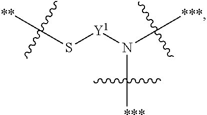

wherein:
   ** indicates a point of attachment of the linker to a proximal diacyl group;
   *** indicates a point of attachment of the linker to a distal diacyl group; and
   Y₁ is independently at each occurrence an optionally substituted alkanediyl, an optionally substituted alkenediyl, or an optionally substituted arenediyl; and
(e) each terminating group is independently selected from optionally substituted alkylthiol and optionally substituted alkenylthiol.

In some embodiments, x¹ is 0, 1, 2, or 3. In some embodiments, R^{1a}, R^{1b}, R^{1c}, R^{1d}, R^{1e}, R^{1f}, and R^{1g} (if present) are each independently at each occurrence a point of connection to a branch as indicated by *, hydrogen, or C₁-C₁₂ alkyl, wherein the alkyl moiety is optionally substituted with one or more substituents each independently selected from —OH, C₄-C₈ heterocycloalkyl, N-(alkyl)-piperidinyl, piperazinyl, N-(alkyl)-piperadizinyl, morpholinyl, N-pyrrolidinyl, pyrrolidinyl, or N-(alkyl)-pyrrolidinyl, aryl, and heteroaryl, or pyridinyl. In further embodiments, R^{1a}, R^{1b}, R^{1c}, R^{1d}, R^{1e}, R^{1f}, and R^{1g} (if present) are each independently at each occurrence a point of connection to a branch as indicated by *, hydrogen, or alkyl, wherein the alkyl moiety is optionally substituted with one substituent-OH. In some embodiments, R^{3a} and R^{3b} are each independently at each occurrence hydrogen.

In some embodiments, the plurality (N) of branches comprises at least 3 branches. In further embodiments, the plurality (N) of branches comprises at least 4 branches. In still further embodiments, the plurality (N) of branches comprises at least 5 branches. In some embodiments, g=1; G=0; and Z=1. In further embodiments, each branch of the plurality of branches comprises a structural formula

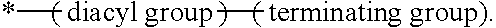

In some embodiments, g=2; G=1; and Z=2. In further embodiments, each branch of the plurality of branches comprises a structural formula

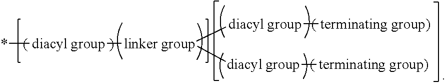

In some embodiments, the core comprises a structural formula:

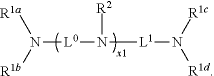

In further embodiments, the core comprises a structural formula:

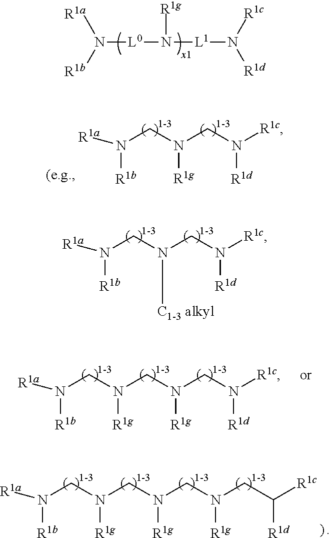

In still further embodiments, the core comprises a structure selected from:

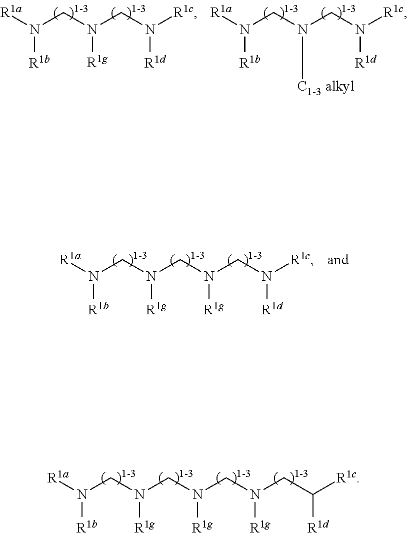

In even further embodiments, the core comprises a structural formula selected from the group consisting of:

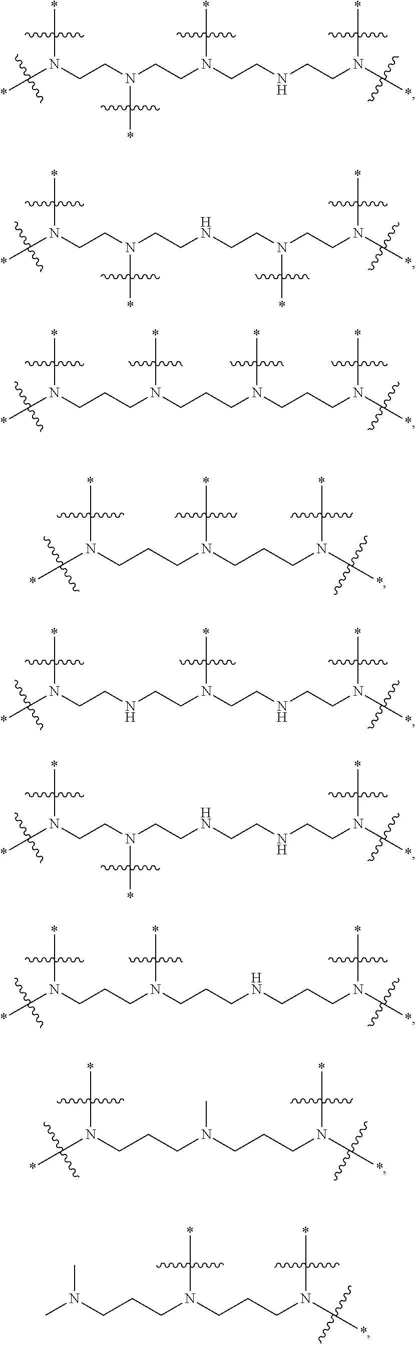

and pharmaceutically acceptable salts thereof, wherein * indicates a point of attachment of the core to a branch of the plurality of branches.

In still further embodiments, the core has the structure

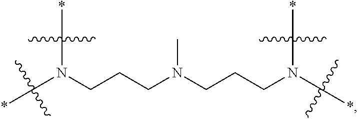

wherein * indicates a point of attachment of the core to a branch of the plurality of branches or H, wherein at least 2 (e.g., at least 3, or at least 4) branches are attached to the core.

In some embodiments, the core further comprises at least 3 branches attached to the core. In further embodiments, the core further comprises at least 4 branches attached to the core.

In some embodiments, the core has the structure

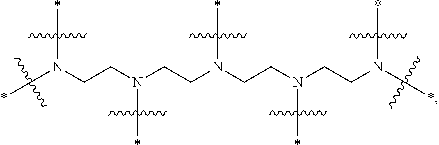

wherein * indicates a point of attachment of the core to a branch of the plurality of branches or H, wherein at least 4 branches are attached to the core. In further embodiments, the core comprises at least 5 branches attached to the core. In still further embodiments, the core comprises at least 6 branches attached to the core. In some embodiments, A¹ is —O— or —NH—. In some embodiments, A₂ is —O— or —NH—. In some embodiments, Y₃ is C₁-C₁₂ alkanediyl.

In some embodiments the diacyl group independently at each occurrence comprises a structural formula

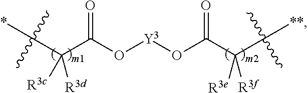

optionally wherein R^{3c}, R^{3d}, R^{3e}, and R^{3f} are each independently at each occurrence hydrogen or C₁-C₃ alkyl. In further embodiments, the diacyl group is further defined as:

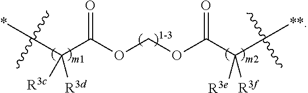

In still further embodiments, the diacyl group is further defined as:

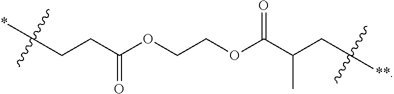

In some embodiments, each terminating group is independently C₁-C₁₈ alkylthiol. In other embodiments, each terminating group is independently C₁-C₁₈ alkenylthiol.

In some embodiments, the composition comprises a molar percentage of the ionizable lipid from about 5 to about 50 of the ionizable lipid relative to the total lipid composition. In further embodiments, the molar percentage of the ionizable lipid is from about 15 to about 40 of the ionizable lipid relative to the total lipid composition. In still further embodiments, the molar percentage of the ionizable lipid is from about 20 to about 30 of the ionizable lipid relative to the total lipid composition.

In some embodiments, the composition further comprises a phospholipid. In further embodiments, the phospholipid comprises one or two long chain alkyl or alkenyl groups, a glycerol or a sphingosine, one or two phosphate groups, and a small organic molecule, wherein the small organic molecule is an amino acid, a sugar, or an amino substituted alkoxy group. In even further embodiments, the phospholipid is 1,2-distearoyl-sn-glycero-3-phosphocholine (DSPC) or 1,2-dioleoyl-sn-glycero-3-phosphoethanolamine (DOPE). In some embodiments the phospholipid is DOPE.

In some embodiments, the composition comprises a molar percentage of the phospholipid from about 5 to about 50 of the phospholipid relative to the total lipid composition. In further embodiments, the molar percentage of the phospholipid is from about 10 to about 40 of the phospholipid relative to the total lipid composition. In still further embodiments, the molar percentage of the phospholipid is from about 20 to about 30 of the phospholipid relative to the total lipid composition.

In some embodiments, the composition further comprises a steroid. In some embodiments, the steroid is cholesterol. In some embodiments, the composition comprises a molar percentage of the steroid from about 10 to about 65 of the steroid relative to the total lipid composition. In further embodiments, the molar percentage of the steroid is from about to about 55 of the steroid relative to the total lipid composition. In some embodiments, the molar percentage of the steroid is from about 30 to about 50 of the steroid relative to the total lipid composition.

In some embodiments, the composition further comprises a polymer-conjugated lipid. In some embodiments, the polymer-conjugated lipid is a PEGylated lipid. In some embodiments, the polymer-conjugated lipid comprises a polyethylene glycol (PEG) component from about 1000 to about 10,000 daltons. In some embodiments, the polymer-conjugated lipid is a PEGylated diacylglycerol. In further embodiments, the polymer-conjugated lipid is further defined by the formula:

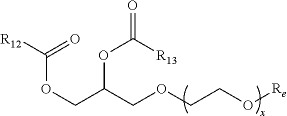

wherein:
R¹² and R¹³ are each independently alkyl_{(C≤24)}, alkenyl_{(C≤24)}, or a substituted version of either of these groups;
Rₑ is hydrogen, alkyl_{(C≤8)}, or substituted alkyl_{(C≤8)}; and
x is 1-250.

In other embodiments, the polymer-conjugated lipid is a PEGylated dimyristoyl-sn-glycerol or a compound of the formula:

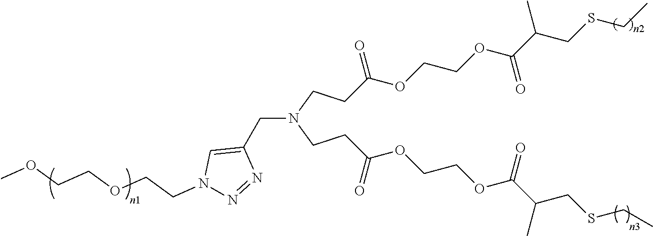

wherein:
n₁ is 5-250; and
n₂ and n₃ are each independently 2-25.

In some embodiments, the composition comprises a molar percentage of the polymer-conjugated lipid from about 0.1 to about 15 of the polymer-conjugated lipid relative to the total lipid composition. In further embodiments, the molar percentage of the polymer-conjugated lipid is from about 0.5 to about 10 of the polymer-conjugated lipid relative to the total lipid composition. In still further embodiments, the molar percentage of the polymer-conjugated lipid is from about 1 to about 6 of the polymer-conjugated lipid relative to the total lipid composition.

In some embodiments, the composition further comprises a second polymer-conjugated lipid. In some embodiments, the second polymer-conjugate lipid is a PEGylated lipid. In some embodiments, the polymer-conjugated lipid comprises a polyethylene glycol (PEG) component from about 1000 to about 10,000 daltons. In some embodiments, the second polymer-conjugated lipid is a PEGylated diacylglycerol. In some embodiments, the PEGylated diacylglycerol further comprises one or more phosphate groups. In further embodiments, the PEGylated diacylglycerol further comprises one or more cell targeting moieties. In some embodiments, the cell targeting moiety is an antibody, a nucleic acid, a protein or peptide, or a small molecule. In some embodiments, the cell targeting moiety is a small molecule. In further embodiments, the small molecule is a vitamin or cofactor, such as folate. In some embodiments, the second polymer-conjugated lipid is DSPE-PEG2000-Folate. In other embodiments, the second polymer-conjugated lipid is DSPE-PEG2000.

In some embodiments, the composition comprises a molar ratio of the ionizable lipid to total polynucleotide components of from about 1:10 to about 100:1. In further embodiments, the composition comprises a molar ratio of the ionizable lipid to total polynucleotide components of from about 1:1 to about 50:1. In even further embodiments, the composition comprises a molar ratio the ionizable lipid to total polynucleotide components from about 5:1 to about 15:1

In some embodiments, the composition comprises 5A2-SC8, cholesterol, DOPE, and DMG-PEG2000. In further embodiments, the composition comprises a molar ratio of 5A2-SC8: DOPE: cholesterol: DMG-PEG2000 of from about 15:15:30:2.

In some embodiments the composition further comprises a pharmaceutically excipient or carrier. In some embodiments, the composition is formulated as a solution.

In some embodiments, the composition is formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctivally, subcutaneously, sublingually, topically, transbuccally, transdermally, vaginally, in crèmes, in lipid compositions, via a catheter, via a lavage, via continuous infusion, via infusion, via inhalation, via injection, via local delivery, or via localized perfusion.

In some embodiments, the composition is formulated for administration via injection or via inhalation. In further embodiments, the composition is formulated for administration via injection. In other embodiments, the composition is formulated for administration via inhalation. In some embodiments, the composition is formulated as a unit dose.

In another aspect, the present disclosure provides methods of treating a disease or disorder comprising administering to a patient a therapeutically effective amount of the compositions disclosed herein.

In some embodiments, the disease or disorder is cancer, such as carcinoma, sarcoma, lymphoma, leukemia, melanoma, mesothelioma, multiple myeloma, or seminoma. In some embodiments, the cancer is of the bladder, blood, bone, brain, breast, central nervous system, cervix, colon, endometrium, esophagus, gall bladder, gastrointestinal tract, genitalia, genitourinary tract, head, kidney, larynx, liver, lung, muscle tissue, neck, oral or nasal mucosa, ovary, pancreas, prostate, skin, spleen, small intestine, large intestine, stomach, testicle, or thyroid. In some embodiments, the cancer is a solid tumor. In some embodiments, the cancer is ovarian cancer or liver cancer. In some embodiments, the inhibitory polynucleotide inhibits a protein overexpressed in the disease or disorder. In some embodiments, the protein is a focal adhesion kinase. In some embodiments, the guide polynucleotide targets PD-L1. In some embodiments, the guide polynucleotide results in reduced expression of PD-L1.

In some embodiments, the method further comprises one or more additional therapeutic modalities. In some embodiments, the additional therapeutic modality is radiation therapy, an additional chemotherapy, surgery, or immunotherapy.

In some embodiments, the method comprises administering the composition: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctivally, subcutaneously, sublingually, topically, transbuccally, transdermally, vaginally, in crèmes, in lipid compositions, via a catheter, via a lavage, via continuous infusion, via infusion, via inhalation, via injection, via local delivery, or via localized perfusion. In further embodiments, the composition is administered via inhalation or via injection.

In some embodiments, the composition is administered locally. In other embodiments, the composition is administered systemically. In some embodiments, the method comprises administering the composition once. In other embodiments the method comprises administering the composition two or more times.

In yet another aspect, the present disclosure provides methods of inhibiting a focal adhesion kinase (FAK) in a patient comprising administering a composition described herein, wherein the inhibitory polynucleotide inhibits FAK.

In some embodiments, the methods result in increased delivery of the nuclease or polynucleotide encoding for the nuclease. In some embodiments, the increased delivery results in improved cell editing compared to a composition without a FAK inhibitory polynucleotide. In some embodiments, the cell editing is gene editing. In other embodiments, the cell editing is gene silencing.

In yet another aspect, the present disclosure provides methods of delivering of a polynucleotide that encodes for a protein comprising contacting the cell with a composition described herein, wherein the inhibitory polynucleotide is a focal adhesion kinase (FAK) inhibitory polynucleotide. In some embodiments, the methods result in improved delivery of the polynucleotide compared to a method without a non-FAK inhibitory polynucleotide.

In yet another aspect, the present disclosure provides methods of editing the genome of a cell that encodes for a protein comprising contacting the cell with a composition described herein, wherein the inhibitory polynucleotide is a focal adhesion kinase (FAK) inhibitory polynucleotide. In some embodiments, the methods result in improved genome editing of the polynucleotide compared to a method without a non-FAK inhibitory polynucleotide.

In yet another aspect, the present disclosure provides methods of silencing the genome of a cell that encodes for a protein comprising contacting the cell with a composition described herein, wherein the inhibitory polynucleotide is a focal adhesion kinase (FAK) inhibitory polynucleotide. In some embodiments, the methods result in improved silencing of the genome compared to a method without a non-FAK inhibitory polynucleotide.

In some embodiments, the methods are carried out in vitro. In other embodiments, the methods are carried out in vivo. In some embodiments, the in vivo method comprises administering to the cell in a patient. In some embodiments, the patient is a mammal such as a human. In some embodiments, the methods are sufficient to treat a disease or disorder.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The invention may be better understood by reference to one of these drawings in combination with the detailed description of specific embodiments presented herein.

FIG. 1A-1M: FAK knockdown enhances LNP-mediated mRNA delivery and CRISPR gene editing. a, Schematic illustration showing triple loading of FAK siRNA, Cas9 mRNA, and sgRNA into 5A2-SC8 LNPs. b, RT-qPCR quantification of time-dependent FAK expression in cells treated with siFAK+CRISPR-PD-L1-LNPs. n=4 biologically independent samples. Error bars represent mean+s.d. c, Representative western blot analysis of FAK expression in IGROV1 cells treated with PBS, siCtrl+CRISPR-PD-L1-LNPs, and siFAK+CRISPR-PD-L1-LNPs for 12 h and 24 h. d, and e, Representative fluorescence microscopy images (d) and quantification of GFP fluorescence intensity (e) of HeLa-GFP cells treated with PBS, siCtrl+CRISPR-GFP-LNPs, and siFAK+CRISPR-GFP-LNPs for 48 h. Scale bar=20 μm. ** P<0.01 determined by two-tailed t-test. n=3 biologically independent samples. Error bars represent mean+s.d . . . f and g, Representative T7E1 assay for siFAK+CRISPR-LNPs-mediated cleavage enhancement of GFP in HeLa-GFP cells (f) and PD-L1 in IGROV1 cells (g). HeLa-GFP cells: total RNA: 2.67 μg/mL, Cas 9 mRNA: sgRNA: siRNA=2:0.25:2). IGROV1 cells: total RNA: 1.25 μg/mL, Cas 9 mRNA: sgRNA: siRNA=2:1:2). h, Representative immunofluorescence of PD-L1 in IGROV1 cells after 48 h treatment with PBS, siCtrl+CRISPR-PD-L1-LNPs, and siFAK+CRISPR-PD-L1-LNPs. (Total RNA: 1.25 μg/mL, Cas 9 mRNA: sgRNA: siRNA=2:1:2). PD-L1 (dark), Nuclei (DAPI, light). Scale bar=10 μm. i, T7E1 mismatch cleavage (top) and quantification of integrated optical density (IOD) (bottom) for siFAK+CRISPR-GFP-LNPs-mediated gene editing enhancement of GFP in HeLa-GFP cell tumor spheroids cultured on the different stiffness substrates (˜100 Pa and ˜300 Pa). The concentration of total RNA for HeLa-GFP tumor spheroids was 2.67 μg/mL, Cas 9 mRNA: sgRNA: siRNA=2:0.25:2. n=3 biologically independent samples. Error bars represent mean±s.d. **** P<0.0001 analyzed two-tailed t-test. Data was analyzed using Image.J. j and k, Representative images (j) and quantification of fluorescence intensity (k) of the penetration of nanoparticles in the IGROV1 tumor spheroids treated with siCtrl+Cy5-mRNA-LNPs and siFAK+Cy5-mRNA-LNPs after 48 h incubation. Scale bar=50 μm. 1 and m, mCherry expression (1) and deep distribution (m) in tumor spheroids treated with PBS, siCtrl+mRNA-LNPs, and siFAK+mRNA-LNPs. Scale bar=100 μm.

FIG. 2A-2D: Characterization of siFAK+CRISPR-LNPs. a, Encapsulation efficiency of total RNA in LNPs with different ratios of 5A2-SC8 to RNA from 40:1 to 8:1 (wt/wt). n=4 biologically independent samples. b and c, The hydrodynamic size (b) and zeta potential (c) of SiFAK+CRISPR-LNPs with the ratios of 5A2-SC8 to RNA from 40:1 to 8:1 (wt/wt). n=3 biologically independent samples. d, Stability of siFAK+CRISPR-LNPs in 5% serum condition at 37° C. was evaluated by measuring particle size changes at various time points up to 72 h. 5A2-SC8: RNA=20:1. n=3 biologically independent samples. Error bars in a-d represent mean±s.d.

FIG. 3A-3F: FAK knockdown enhances mRNA delivery efficacy and protein expression in multiple tumor cell lines. a, Representative fluorescence microscopy images of IGROV1 cells treated with PBS, siCtrl+mRNA-LNPs, and siFAK+mRNA-LNPs, respectively. gray, mCherry expression, light gray, cellular nuclei. Scale bar=20 μm. b, Quantification of time-dependent mCherry expression in IGROV1 cells treated with siCtrl+mRNA-LNPs and siFAK+mRNA-LNPs for 24 h, 48 h, and 72 h. p1=0.7305, p2=0.0033, p3=0.0027, p4=0.0006. ns, no significant difference, ** P<0.01, *** P<0.001 by two-tailed t-test, n=4 biologically independent samples. Error bars represent mean±s.d. c, Quantification of enhancement of mCherry expression with increase of siRNA concentrations from 0.0025 to 0.125 μg/mL, mRNA: 0.25 μg/mL, 5A2-SC8: mRNA=20:1. n=4 biologically independent samples. Error bars represent mean±s.d. d, Representative microscope images of FAK knockdown and mCherry expression in the HeLa-Luc cells after 24 h culturing of siFAK+mRNA-LNPs. mRNA is mCherry mRNA, FAK, mCherry, Nucleus. Scale bar: 10 μm. e, mCherry expression in HepG2, A375, HeLa-Luc cells treated with PBS, siCtrl+mRNA-LNPs, and siFAK+mRNA-LNPs, respectively. p1=0.0002, p2=0.0067, p3=0.0007, ** P<0.01, *** P<0.001 by two-tailed t-test. n=4 biologically independent samples. Error bars represent mean±s.d. f, FAK knockdown enhanced luciferase mRNA delivery in other tumor cells (HepG2, A375, and B16F10) when delivering of siFAK+mRNA-LNPs into cells for 24 h. The luciferase expression was examined through monitoring luciferase the activity in cells using the ONE-Glo+Tox Luciferase Reporter assay kit. Luciferase mRNA was used. p1=0.0003, p2-0.0038, p3=0.0024, ** P<0.01, *** P<0.001 by two-tailed t-test. n=4 biologically independent samples. Error bars represent mean±s.d.

FIG. 4A-4B: The effect of the ratios of siRNA and mRNA in siFAK+mRNA-LNPs on delivery efficiency in HeLa-Luc cells. a, Luciferase knockdown with increasing ratios of mRNA to siRNA from 1:1 to 4:1. n=3 biologically independent samples. Error bars represent mean±s.d. b, Representative microscope images of HeLa-Luc cells with mCherry expression under delivering different ratios of mCherry mRNA to siRNA from 1:1 to 4:1. Cells were treated for 24 h. There was no signal in the HeLa-Luc cells treated with PBS. Scale bar=20 μm.

FIG. 5A-5D: Experiments directly comparing sequential delivery of siRNA and mRNA in separate LNPs versus simultaneous co-delivery of siRNA+mRNA in one LNP formulation in 2D in vitro cell culture. a, Schematic illustration of the experimental design for sequential and simultaneous delivery approaches in a 2-dimensional IGROV1 cell culture model. b, Sequential delivery of siFAK-LNPs followed by mCherry mRNA-LNPs (two separate LNPs) resulted in higher mCherry protein expression than sequential delivery of Control siRNA (siCtrl)-LNPs followed by mCherry mRNA-LNPs (separate LNPs), indicating that FAK silencing enhances mRNA delivery. p1=0.0048, p2=1.87×10⁻⁶, ** P<0.01, **** P<0.0001 by two-tailed t-test. n=5 biologically independent samples. Error bars represent mean±s.d. c, Simultaneous co-delivery of siFAK and mCherry mRNA (siFAK+mRNA-LNPs) (one LNP containing both nucleic acids) resulted in higher mCherry protein expression than siCtrl+mCherry Mrna-LNPs (one LNP), further confirming that FAK silencing enhances mRNA delivery. p1=1.07×10⁻⁶, p2=2.5×10⁻⁶, **** P<0.0001 by two-tailed t-test. n=5 biologically independent samples. Error bars represent mean±s.d. d, Sequential delivery of siFAK and mCherry mRNA in separate LNPs (staged delivery) and simultaneous co-delivery of mCherry mRNA+siFAK in one LNP formulation yielded comparable levels of protein expression (not statistically different) in vitro in this 2-dimensional IGROV1 cell culture model (data normalized to control to show fold enhancement). Thus, without wishing to be bound by any theory, it is believed that sequential delivery and simultaneous co-delivery are both efficacious in 2-dimensional cell culture experiments. The values was calculated from data in b and c. p1=0.5938, p2=0.2334, ns, no significant difference by two-tailed t-test, Error bars represent mean±s.d.

FIG. 6A-6E: Experiments directly comparing sequential delivery of siRNA and CRISPR in separate LNPs versus simultaneous co-delivery of siRNA+mRNA+sgRNA in one LNP formulation in 2D in vitro cell culture. a, Schematic illustration of the experimental design for sequential and simultaneous delivery approaches in a 2-dimensional IGROV1 cell culture model. b, Sequential delivery of siFAK-LNPs followed by CRISPR-PD-L1 (Cas9 mRNA+sgPD-L1)-LNPs (two separate LNPs) and simultaneous co-delivery of siFAK and CRISPR-PD-L1-LNPs (one LNP) resulted in similar levels of PD-L1 gene editing detected by the T7E1 mismatch cleavage assay. Similarly, c, sequential delivery of siFAK-LNPs followed by CRISPR-KRAS (Cas9 mRNA+sgKRAS)-LNPs (two separate LNPs) and simultaneous co-delivery of siFAK and CRISPR-KRAS-LNPs (one LNP) resulted in similar levels of KRAS gene editing detected by the T7E1 mismatch cleavage assay. d and e, Quantification of integrated optical density (IOD) for each treatment group further confirmed that sequential delivery of siFAK and CRISPR in separate LNPs (staged delivery) and simultaneous co-delivery of siFAK+CRISPR in one LNP formulation yielded comparable levels of gene editing in vitro in this 2-dimensional IGROV1 cell culture model. n=3 biologically independent samples, p=0.6387 (d) and p=0.9789 (e), ns, no significant difference by two-tailed t-test. Error bars represent mean±s.d.

FIG. 7A-7C: Experiments directly comparing sequential delivery of siRNA and mRNA in separate LNPs versus simultaneous co-delivery of siRNA+mRNA in one LNP formulation in 3D tumor spheroids. a, Schematic illustration of the experimental design for sequential and simultaneous delivery approaches in a 3-dimensional IGROV1 cell culture model. b, Representative control sequential (siControl-LNPs followed by mCherry mRNA-LNPs) delivery, sequential (siFAK-LNPs followed by mCherry mRNA-LNPs), and simultaneous (co-delivery of siFAK and mCherry mRNA in one LNP) (please note that some data is reproduced from FIG. 1I in order to show side-by-side comparisons) approaches were compared head-to-head. Confocal imaging (b) and (c) mCherry depth quantification show that the simultaneous co-delivery approach resulted in the highest depth penetration into the spheroids leading to mCherry expression across the entire spheroids. Thus, without wishing to be bound by any theory, it is believed that simultaneous co-delivery is more efficacious than sequential delivery in 3-dimensional tumor spheroids. Scale bar=100 μm.

FIG. 8A-8C: Experiments directly comparing in vivo sequential delivery of siRNA and mRNA in separate LNPs versus simultaneous co-delivery of siRNA+mRNA in one LNP formulation in murine tumor xenografts. a, Schematic illustration of the experimental design for sequential and simultaneous delivery approaches in an ID8 xenograft tumor. b, Control sequential (siControl-LNPs followed by mCherry mRNA-LNPs) delivery, sequential (siFAK-LNPs followed by mCherry mRNA-LNPs), and simultaneous (co-delivery of siFAK and mCherry mRNA in one LNP) (please note that some data is reproduced from FIG. 23B in order to show side-by-side comparisons copy from FIG. 23B) approaches were compared head-to-head. Confocal imaging (b) of tumor sections and (c) mCherry quantification from homogenized tumors shows that the simultaneous co-delivery approach resulted in the higher efficacy. Thus, we conclude that simultaneous co-delivery is more efficacious than sequential delivery in ID8 tumors. n=3 biologically independent samples. p1=0.0007, p2=0.0039, ** P<0.01, *** P<0.001 determined by one-way ANOVA with multiple comparison test. Error bars represent mean±s.d. (b). Scale bar=100 μm (c).

FIG. 9A-9E: Experiments directly comparing sequential delivery of siRNA and CRISPR in separate LNPs versus simultaneous co-delivery of siRNA+CRISPR in one LNP formulation in murine tumor xenografts. a, Schematic illustration of the experimental design for simultaneous and sequential delivery approaches in an IGROV1 xenograft tumor. b, Sequential delivery of siFAK-LNPs followed by CRISPR-PD-L1 (Cas9 mRNA+sgPD-L1)-LNPs (two separate LNPs) and simultaneous co-delivery of siFAK and CRISPR-PD-L1-LNPs (one LNP) resulted in different levels of PD-L1 gene editing detected by the T7E1 mismatch cleavage assay. Simultaneous co-delivery was more efficacious. Similarly, c, simultaneous co-delivery of siFAK and CRISPR-KRAS-LNPs (one LNP) was more effective than sequential delivery of siFAK-LNPs followed by CRISPR-KRAS (Cas9 mRNA+sgKRAS)-LNPs (two separate LNPs) for KRAS gene editing detected by the T7E1 mismatch cleavage assay. d and e, Quantification of integrated optical density (IOD) for each treatment group further confirmed that simultaneous co-delivery of siFAK+CRISPR in one LNP formulation was more efficacious than sequential delivery of siFAK and CRISPR in separate LNPs (staged delivery) for gene editing in vivo in this xenograft tumor model. n=3 biologically independent samples. Error bars represent mean±s.d., p=0.0386 (d), p=0.0467 (e), *P<0.05 by two-tailed t-test.

FIG. 10: FAK knockdown enhanced mRNA delivery efficacy (protein expression) in tumor cells treated with an FAK inhibitor. n=4 biologically independent samples. Error bars represent mean±s.d.

FIG. 11: Time-dependent penetration of siFAK+Cy5-mRNA-LNPs into tumor spheroids. Scale bar=50 μm.

FIG. 12: Time-dependent enhancement of mCherry mRNA delivery after FAK knockdown in tumor spheroids cultured on Matrigel matrix. n=4 biologically independent samples, p1=0.0024, p2=0.0065, ** P<0.01 by two-tailed t-test. Error bars represent mean±s.d.

FIG. 13A-13G: FAK-knockdown enhances the endocytosis of siFAK+CRISPR-LNPs through dynamic alteration of the contraction force and cell membrane tension. a and b, Representative confocal images (a) and flow cytometry quantification (b) of time-dependent cellular uptake of siCtrl+cy5-mRNA-LNPs and siFAK+cy5-mRNA-LNPs in IGROV1 cells. LNPs (Cy5), cytoskeleton (phalloidin-iFluor 488), and nucleus (DAPI). Scale bar=20 μm. n=3 biologically independent samples. Error bars represent mean±s.d . . . c, Quantification of the cellular uptake of LNPs when using small molecule inhibitors of various cellular uptake pathways. Cellular uptake was quantified through observing the Cy5-labled-mRNA-LNPs in cells. n=3 biologically independent samples. Error bars represent mean±s.d . . . ns, no significant difference, *** P<0.001 determined by two-tailed t-test. The results were analyzed using Image J. d, Representative confocal images of IGROV1 cells treated with siCtrl+CRISPR-LNPs and siFAK+CRISPR-LNPs at different time points. F-actin, P-myosin II (P-myo.II), Nuclei (DAPI). Scale bar=20 μm. e, Quantification of the distribution of F-actin and P-myo.II on the white arrow lines in the confocal images (d). f, Schematic illustration FAK-knockdown induced reduction of cellular contraction force and membrane tension under siFAK+CRISPR-LNPs treatment. g, Representative membrane invagination and endocytosis regulated by administration of siFAK+cy5-mRNA-LNPs. Scale bar=10 μm.

FIG. 14: Cellular uptake pathway analysis. IGROV1 cells were pre-incubated for 30 min in serum-free medium containing inhibitors (filipin, chlorpromazine, EIPA, and Baf A1) or combinations of inhibitor and siFAK+mRNA-LNPs before delivering with siFAK+Cy5-mRNA-LNPs at a total RNA concentration of 0.75 μg ml-1, mRNA: siRNA=2:1. The cellular uptake was observed through fluorescence of Cy5 used to label mRNA in LNPs. Scale bar=10 μm.

FIG. 15A-15B: FAK knockdown decreased the contraction force of cells treated with siFAK+CRISPR-LNPs. Quantification of time-dependent fluorescence intensity of P-myosin II (P-myo. II) (a) and F-actin (b) from the confocal images using Image J software. n=3 biologically independent samples, p1=0.3156, p2=0.0095, p3=0.013, p4-0.0015 (a), p1=0.6933, p2=0.0132, p3=0.0007, p4=0.0189 (b), ns, no significant difference, *P<0.05, ** P<0.01, *** P<0.001, by two-tailed t-test. Error bars represent mean±s.d.

FIG. 16A-16B: FAK knockdown decreased cell contraction. a and b, Schematic of collagen-based contraction assay (a, top), representative images of cell-gel matrixes (a, down), and quantification of relative decrease of gel-spot diameter over time (b). n=3 biologically independent samples. p1=0.0025, p2=0.0004, p3-0.0006, p4=0.0052, ** P<0.01, *** P<0.001 by two-tailed t-test. Error bars represent mean±s.d.

FIG. 17A-17B: a, The membrane deformation of cells treated with different concentrations of siFAK-LNPs. Scale bar=10 μm. b, The time-dependent membrane deformation regulated by siFAK. The 2-dimensional (2D) images (Top) were single-layer scanned by Confocal microscopy. The 3-dimensional (3D) images were reconstructed by Image J with the view of x, y direction (middle) and x,y, z direction (Down). Scale bar=10 μm.

FIG. 18A-18C: YAP localization and expression in cells treated with siFAK+CRISPR-LNPs. a, Representative confocal immunofluorescence images of YAP localization in the cells. Scale bars, 20 μm. b, Quantification of percentages of cells with nuclear YAP in the cells from 3 biologically independent samples (n=3). p=0.0012, ** P<0.01 analyzed by two-tailed t-test. Error bars represent mean±s.d. c, Representative western blot analysis of YAP expression in the IGROV1 cells treated with PBS and siFAK+CRISPR-LNPs for 24 h.

FIG. 19: FAK knockdown increased the endocytosis of integrin β1 regulated by decreased contraction force in HepG2 cells treated with siFAK+mRNA-LNPs. Representative confocal images of HepG2 cells showed endocytosis enhancement induced by reduction of mechanical properties of cells which is regulated by FAK knockdown by administration of siFAK+mRNA-LNPs compared with the cells treated with siCtrl+mRNA-LNPs. Scale bar=μm.

FIG. 20A-20C: Endocytosis enhancement after knockdown FAK by delivery of siFAK+mRNA-LNPs into IGROV1 cells. a, Representative confocal images of cells treated with siCtrl+mRNA-LNPs and siFAK+mRNA-LNPs for 0, 2 h, 8 h, and 24 h. FAK (light purple), integrin β1 (Int. β1, Greed), nuclei (Blue). Scale bar=10 μm. b, Quantification of time-dependent fluorescence intensity of FAK (top) and integrin β1 (bottom) from the confocal images using Image J software. c, Representative western blot analysis of Integrin β1 in the IGROV1 cells treated with siCtrl+mRNA-LNPs and siFAK+mRNA-LNPs for 24 h.

FIG. 21A-21D: Stiffness of tumor cells regulated by substrates enhanced the gene editing and mRNA expression. a and b, Representative T7E1 mismatch cleavage for siFAK+CRISPR-Luc-LNPs, and siFAK+CRISPR-GFP-LNPs-mediated gene editing enhancement of Luc in the HeLa-Luc (a) and GFP in the HeLa-GFP (b) tumor spheroids cultured on the different stiffness substrates (˜100 Pa and ˜300 Pa) which was modified with same concentration of collagen I (˜30 μg/mL). The concentration of total RNA for HeLa-GFP tumor spheroids was 2.67 μg/mL, Cas 9 mRNA: sgRNA: siRNA=2:0.25:2. c and d, Representative mCherry expression (c) and quantification (b) in the tumor spheroids cultured on the substrates with different stiffness, compared the cells treated with siCtrl+mRNA-LNPs and siFAK+mRNA-LNPs. The concentration of total RNA for IGROV1 tumor sphere is 1.125 μg/mL, mCherry mRNA: siRNA=2:1). n=3 biologically independent samples, p1=0.00009, p2=0.0084, p3=0.0054, ** P<0.01, *** P<0.001, was determined by one-way ANOVA with multiple comparison test. Error bars represent mean±s.d. (b). Scale bar=100 μm (c).

FIG. 22A-22C: a, Scheme outlining the weekly local injection schedule of PBS, empty LNPs, siCtrl+CRISPR-PD-L1-LNPs, siFAK+CRISPR-Ctrl-LNPs, and siFAK+CRISPR-PD-L1-LNPs (50 μL, total RNA, 10 μg, Cas9 mRNA: sgRNA: siRNA=2:1:3). b, Distribution of siFA K+Cy5-mRNA-LNPs in the mice after 6 h and 24 h after injection. c, Representative H&E staining of tumor tissue after 30-day therapy showed less aggressive tumors after treatment with siFAK+CRISPR-PD-L1-LNPs compared to the other groups treated with PBS, empty LNPs, siCtrl+CRISPR-PD-L1-LNPs, siFAK+CRISPR-Ctrl-LNPs. Scale bar=100 μm.

FIG. 23A-23F: siFAK+CRISPR-PD-L1-LNPs targeted tumor stiffness and PD-L1 to inhibit xenograft tumor growth. a and b, Representative confocal images of mCherry expression and distribution at the edge (a) and center (b) of fixed tumor tissues after local administration of PBS, siCtrl+mRNA-LNPs, and siFAK+mRNA-LNPs. Scale bar=50 μm. c and d, Representative 3D construction of immunofluorescence (c) and 3D surface plot of quantification (d) of collagen I and YAP in fixed tumor tissues after 30-day therapy of mice by weekly local injection of PBS, empty LNPs, siCtrl+CRISPR-PD-L1-LNPs, siFAK+CRISPR-Ctrl-LNPs, and siFAK+CRISPR-PD-L1-LNPs. Collagen I, YAP, nuclei. Scale bar=100 μm. e, Bioluminescence imaging of whole animals bearing ID8-Luc xenograft tumors treated with PBS, empty LNPs, siCtrl+CRISPR-PD-L1-LNPs, siFAK+CRISPR-Ctrl-LNPs, and siFAK+CRISPR-PD-L1-LNPs at day 40. n=3 mice per group. f. Excised tumor (left) and tumor size (right) show the in vivo therapeutic efficacy of siFAK+CRISPR-PD-L1-LNPs and the other control groups. Tumor size measurement began on day 10 and continued every 3 days. n=3 mice per group. Error bars represent the mean±s.d., *P<0.05, ** P<0.01, *** P<0.001, was determined by one-way ANOVA with multiple comparison test.

FIG. 24A-24B: a, Representative compressive stress-strain curves of the tumor tissues. b, Compressive modulus values at a range of 25-30% stain of the tumor tissues with different treatments. n=8 mice each group, p1=0.1292, p2=0.000101, p3-0.0001152, p4=0.0026, p5=0.0018, ns, no significant difference, ** P<0.01, *** P<0.001 was determined by one-way ANOVA with multiple comparison test. Error bars represent mean±s.d.

FIG. 25A-25B: a and b, Representative 3D construction of immunofluorescence (a) and 3D surface plot of quantification (b) of integrin βI and P-Myosin II in the tumor after the therapy process. Scale bar=100 μm

FIG. 26A-26D: a, Representative flow cytometry gating strategies for infiltrated immune cells, cytotoxic T cells, macrophages, and regulatory T cells. b, c, and d, Flow cytometric analyses of (b) macrophages, (c) CD8+ T cells, and (d) CD4+ T cells in the tumors from mice bearing ID8-Luc xenograft tumors treated with PBS, empty LNPs, siCtrl+CRISPR-PD-L1-LNPs, siFAK+CRISPR-Ctrl-LNPs, and siFAK+CRISPR-PD-L1-LNPs after 30-day therapy. n=6 mice each group. p1=0.0191, p2=2.99×10⁻⁷, p3=0.000152 in (b), p1=0.1414, p2=0.0029, p3=0.0003 in (c), p1=0.0679, p2=0.0026, p3=0.0005 in (d), ns, no significant difference, *P<0.05, ** P<0.01, *** P<0.001, **** P<0.0001 was determined by one-way ANOVA with multiple comparison test. Error bars represent mean±s.d.

FIG. 27: Average body weights of ID8-Luc tumor-bearing xenograft mice over the course of therapy. Error bars represent mean±s.d. n=3 mice each group.

FIG. 28A-28E: siFAK+CRISPR-PD-L1-LNPs decreased tumor cell adhesion and metastasis in vivo. a and b, Representative microscope images of adhered cell (a) and statistical analysis of adhered cell number (b) cultured on the substrates covered with Matrigel matrix for 30 min and then centrifuge for 5 min under 300 G. Scale bar=100 μm (a top), 20 μm (a down). n=4 biologically independent samples. Error bars represent mean±s.d. (b). c and d, Representative lung metastasis (c) and quantification of metastasis ratios (Mouse with lung metastatic foci to 5 mice per group) (d) of ID8-Luc tumor cells which were pre-treated with PBS, empty LNPs, siCtrl+CRISPR-PD-L1-LNPs, siFAK+CRISPR-Ctrl-LNPs and siFAK+CRISPR-PD-L1-LNPs for 48 h prior to injection. The treated ID8-Luc cells were injected into blood circulation system through tail vein. 30 days after injection, the metastasis foci were observed through bioluminescence imaging of mice and their organs. n=5 mice. p=0.0094, ** P<0.01, was determined by one-way ANOVA with multiple comparison test.

Error bars represent mean±s.d. e, H&E staining of lung tissue section of mice treated with different treatments 30 days after injection. Scale bar=100 μm.

FIG. 29A-29J: siFAK+CRISPR-LNPs enabled enhancement of gene editing though decreasing tumor stiffness in an aggressive, genetically engineered liver cancer model. a, Schematic illustration of administration regimen for systemic therapy in the MYC-driven liver cancer mouse model. b and c, Representative 3D construction of immunofluorescence (b) and 3D surface plot of quantification (c) of collagen I and YAP in the tumor during the therapy process (day 45). Scale bar=100 μm. d and e, Representative T7E1 results (d) and quantification of IOD (e) for siFAK+CRISPR-PD-L1-LNPs-mediated cleavage enhancement of PD-L1 in the tumor compared with that of siCtrl+CRISPR-PD-L1-LNPs. n=3 biologically independent samples. Error bars represent mean±s.d. *** P<0.001 determined by two-tailed t-test. f, Representative PD-L1 expression in liver tissues scanned using confocal imaging and captured under two channels: nuclei (DAPI), PD-L1. Scale bar=100 μm. g, Quantification of PD-L1 expression using Image J. n=4 biologically independent samples. Error bars represent mean±s.d. * P<0.05, *** P<0.001 determined by were determined by one-way ANOVA with multiple comparison test. h and i, Representative T7E1 results (h) and quantification of IOD (i) for siFAK+CRISPR-MYC-LNPs-mediated cleavage enhancement of MYC in the tumor compared with that of siCtrl+CRISPR-MYC-LNPs with sgRNA targeting MYC. n=3 biologically independent samples. Error bars represent mean±s.d. ** P<0.01 determined by two-tailed t-test. j, Representative liver images and H&E staining of liver sections of mice following systemic administration of PBS, siCtrl+CRISPR-MYC-LNPs, and siFAK+CRISPR-MYC-LNPs. 3 mg/kg total RNA administered; Cas9 mRNA (1.0 mg/kg): sgRNA (0.5 mg/kg): siRNA (1.5 mg/kg)=2:1:3 (wt) s. 5A2-SC8: RNA=10:1 (wt). The mice were injected starting on day 21. The liver was examined on day 31. Arrows showed the tumors in the liver. Scale bar=500 μm.

FIG. 30A-30C: Collagen distribution in the tumor of liver of mice during treatment. a, Representative Sirius Red staining performed on liver sections of paraffin-embedded tissue after 45 and 55 days treatments. Scale bar=100 μm. b and c, Representative immunofluorescence (b) and quantification (c) of collagen I in the tumor during the therapy process (day 35 and day 55). Scale bar=100 μm (b). n=3-5 mice per group, p1=0.040426, p2=0.366512, p3=0.001021, p4=0.022479, ns, no significant difference, *P<0.05, ** P<0.01 was determined by one-way ANOVA with multiple comparison test. Error bars represent mean±s.d. (c).

FIG. 31A-31B: a, Representative 3D construction of immunofluorescence of integrin βI and P-Myosin II in the liver tissue after 45-day therapy. Scale bar=100 μm. b, 3D surface plot of quantification P-Myosin II in the liver tissue after 45-day therapy using Image J software.

FIG. 32A-32B: Systemic administration of siFAK+mRNA-LNPs and evaluation the liver accumulation and luciferase expression. a, Biodistribution (Cy5-labeled Luc mRNA) and protein expression (from Luc mRNA) following i.v. injection of siFAK+Cy5-Luc-mRNA-LNPs (time point=6 hours after injection). Total RNA: 0.75 mg/kg. mRNA (0.5 mg/kg): siRNA (0.25 mg/kg)=2:1.

FIG. 33A-33I: Systemic administration of siFAK+CRISPR-PD-L1-LNPs significantly extended survival of mice bearing aggressive, MYC-driven cancer. a, Proposed model for how decreasing ECM stiffness could enhance gene editing to thereby increase immune cells infiltration. b and c, Representative IHC for CD8+ T cells (b) and macrophage cells (c) infiltration in the tumor (day 55). Scale bar=100 μm. d, Quantification of infiltration of macrophage cells and CD8+ T cells from confocal images. Error bars represent mean±s.d. n=3 mice with 2 tissue sections per mouse. ns, no significant difference, ** P<0.01, *** P<0.001, **** P<0.0001 determined by one-way ANOVA with multiple comparison test. e, and f, Representative whole body (e) and liver (f) images of mice treated with PBS, empty LNPs, siCtrl+CRISPR-PD-L1-LNPs, siFAK+CRISPR-Ctrl-LNPs, and siFAK+CRISPR-PD-L1-LNPs (55 days). g, H&E staining of liver sections of mice treated with PBS, empty LNPs, siCtrl+CRISPR-PD-L1-LNPs, siFAK+CRISPR-Ctrl-LNPs, and siFAK+CRISPR-PD-L1-LNPs at day 35 (14 days after treatment initiation). The arrows in the images indicate tumors. Scale bar=500 μm. h and i, Abdominal circumference measurements (therapy began on day 21) (h) and survival curves (i) of mice show the in vivo therapeutic efficacy of siFAK+CRISPR-PD-L1-LNPs as compared to the other control groups (PBS, empty LNPs, siCtrl+CRISPR-PD-L1-LNPs, and siFAK+CRISPR-Ctrl-LNPs, (n=6 mice each group). Error bars represent mean±s.d. (h) 3 mg/kg total RNA injection; Cas9 mRNA (1.0 mg/kg): sgRNA (PD-L1, 0.5 mg/kg); siRNA (1.5 mg/kg)=2:1:3 (wt). 5A2-SC8: RNA=10:1 (wt).

FIG. 34: Enhancement of immune cells in tumor microenvironment with siFAK+CRISPR-PD-L1-LNPs treatment. Representative IHC for CD8+ T cells and macrophages infiltration in the tumor during the therapeutic regimen (day 35). The mice were administered i.v. with PBS, empty LNPs, siCtrl+CRISPR-LNPs, siFAK+CRISPR-Ctrl-LNPs, and siFAK+CRISPR-PD-L1-LNPs for inhibiting tumor growth through silencing of FAK and gene editing of PD-L1. Injection total RNA, 3 mg/ml, Cas9 mRNA (1.0 mg/kg): sgRNA (PD-L1, 0.5 mg/kg): siRNA (1.5 mg/kg)=2:1:3. Dendrimer: RNA=10:1. Top, CD8+ T cells, and Liver tissue in the brightfield. Bottom, Macrophages (F4/80), nucleus. Scale bar=100 μm.

FIG. 35A-35B: a and b, Representative H&E staining of liver sections of paraffin-embedded tissue with start therapy point (at day 21) (a), after 45 and 55 days (b) treatments with PBS, empty LNPs, siCtrl+CRISPR-PD-L1-LNPs, siFAK+CRISPR-Ctrl-LNPs, and siFAK+CRISPR-PD-L1-LNPs and the liver images of mice after 55-day treatment. Scale bar=100 μm.

FIG. 36A-36H: Inhibition of tumor growth through enhancing the gene editing of KRAS by FAK knockdown/inhibition. a and b, Relative cell viability of A549 cells after 96 h treatments with PBS, empty LNPs, siCtrl+CRISPR-KRAS-LNPs, siFAK+CRISPR-Ctrl-LNPs, siFAK+CRISPR-KRAS-LNPs (a) and PBS, CRISPR-KRAS-LNPs, small molecule of FAK inhibitor (SM, PF573228), SM+CRISPR-KRAS-LNPs. n=3 biologically independent samples. p1=0.8869, p2=0.000081, p3=0.0009, P4=0.0085 (a), p1=1.6×10⁻⁶, p2=1.89×10⁻⁷, p3=1.56×10⁻⁷, p4=0.0009 (b), ns, no significant difference, ** P<0.01, *** P<0.001, **** P<0.0001 was determined by one-way ANOVA with multiple comparison test. Error bars represent mean±s.d. c, T7E1 mismatch cleavage for siFAK+CRISPR-KRAS-LNPs-mediated gene editing enhancement of KRAS in the A549 cells. d and e, Time-dependent tumor volume (d) and excised tumor images (e) show the in vivo therapeutic efficacy of siFAK+CRISPR-KRAS-LNPs and the other control groups. n=4 mice per group, p1=3.45×10⁻⁵, p2=0.0116, *P<0.05, **** P<0.0001 was determined by one-way ANOVA with multiple comparison test. Error bars represent mean±s.d. f, T7E1 result for siFAK+CRISPR-KRAS-LNPs-mediated cleavage enhancement of KRAS in the tumor compared with that of siCtrl+CRISPR-KRAS-LNPs. g and h, Time-dependent tumor volume (g) and excised tumor images (h) show the in vivo therapeutic efficacy of SM+CRISPR-KRAS-LNPs (n=4 mice) and SM. n=4 mice per group, p=0.021886, *P<0.05 was determined by two-tailed t-test. Error bars represent mean±s.d.

FIG. 37A-37D: Cytotoxicity of nanoparticles to IGROV1 (a, n=3 biologically independent samples) and HepG2 cells (b, n=3 biologically independent samples), and time-dependent cell viability after treated with small molecules of FAK inhibitor (c, n=5 biologically independent samples) and with different concentrations of LNPs (d, n=5 biologically independent samples). Error bars represent mean±s.d. in a-d.

FIG. 38A-38C: Toxicity and off-target analysis of siFAK+CRISPR-LNPs. a, The toxicity of nanoparticles (BUN, AST and ALT) after 24 h systematic administration. n=3 mice. Error bars represent mean #s.d. b, In vivo toxicity evaluation through various organ histological (Heart, Spleen, Lung, Kidney) examination of mice after 55-day treated with PBS, siCtrl+CRISPR-PD-L1-LNPs, siFAK+CRISPR-Ctrl-LNPs, and siFAK+CRISPR-PD-L1-LNPs. Scale bar=100 μm. c, Representative T7E1 assay demonstrated there was no gene editing cutting at the top 8 predicted potential off-target sites after 55-day administration of siCtrl+CRISPR-PD-L1-LNPs and siFAK+CRISPR-PD-L1-LNPs.

### DESCRIPTION OF ILLUSTRATIVE EMBODIMENTS

In some aspects, the present disclosure provides lipid nanoparticle compositions for use in the delivery of one or more of each of the following polynucleotides or nucleic acids, such as an inhibitory polynucleotide and one or more guide or nuclease encoding polynucleotide and a lipid nanoparticle comprising at least one ionizable lipid; wherein the each of the polynucleotides are encapsulated within the lipid nanoparticle. These compositions may be used to treat diseases and disorders for which the polynucleotides would be useful, such as diseases or disorders associated with a mutation in one or more genes.

In some aspects, the present disclosure provides a multiplexed nanoparticle siRNA+Cas9 mRNA+sgRNA that may be used to treat a tumor. The lipid nanoparticle may decrease tumor mechanics and ECM stiffness, increase nanoparticle endocytosis and tissue penetration, and reduce the therapeutic modification threshold to allow gene editing therapy to provide significant survival benefit in genetically engineered mice harboring aggressive tumors.

Among properties of the tumor microenvironment, increased stiffness results from abundant extracellular matrix (ECM) that enhance intrinsic mechanical properties (Mohammadi et al, 2018). Cell-induced deformation is a particular process that ECM undergoes in tumors. Cancer and stromal cells can exert considerable actomyosin-generated forces on the ECM, which contribute to increased ECM stiffness (Humphrey et al, 2014; Lampi et al, 2018). These “inside-out” transmitted tensile forces are primarily mediated by integrin-dependent adhesions of attached cells, in a process involving focal adhesion kinase (FAK) activation (Seong et al, 2013). Therefore, targeting FAK in tumor tissue with the compounds and compositions provided herein can modulate the mechanical properties of tumor cells, as well as stromal cells and the tumor ECM. In addition, inhibition of FAK activity regulates the tumor immunoenvironment leading to elevated CD8+ cytotoxic T cells infiltration (Jiang et al, 2016; Serrels et al, 2015). However, infiltrated T cells will be inhibited by PD-L1 overexpression on tumor cells, which acts as an inhibitor of T cell responses through sending a critical “don't find me” signal to the immune system (Casey et al, 2016). This genetic alteration of PD-L1 in cancer cells represents an immune checkpoint blockade of cancer immunotherapy (Topalian et al. 2012). Taken together, these features of the tumor microenvironment, stiff ECM and PD-L1 overexpression, present an opportunity for CRISPR-mediated disruption of PD-L1 expression in solid tumors for efficient cancer therapy if the aforementioned challenges can be overcome. Details regarding the above aspects are elaborated upon below.

### A. CRISPR Systems

Gene editing is a technology that allows for the modification of target genes within living cells. Recently, harnessing the bacterial immune system of CRISPR to perform on demand gene editing revolutionized the way scientists approach genomic editing. The Cas9 protein of the CRISPR system, which is an RNA guided DNA endonuclease, can be engineered to target new sites with relative ease by altering its guide RNA sequence. This discovery has made sequence specific gene editing functionally effective.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes.

The CRISPR system can induce double stranded breaks (DSBs) at the target site, followed by disruptions as discussed herein. In other embodiments, Cas9 variants, deemed “nickases,” are used to nick a single strand at the target site. Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor (e.g., KRAB) or activator, to affect gene expression. Alternatively, a CRISPR system with a catalytically inactivate Cas9 further comprises a transcriptional repressor or activator fused to a ribosomal binding protein.

In some aspects, a Cas nuclease and gRNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. The target site may be selected based on its location immediately 5′ of a protospacer adjacent motif (PAM) sequence, such as typically NGG, or NAG. In this respect, the gRNA is targeted to the desired sequence by modifying the first 20, 19, 18, 17, 16, 15, 14, 14, 12, 11, or 10 nucleotides of the guide RNA to correspond to the target DNA sequence. In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The target sequence may comprise any polynucleotide, such as DNA or RNA polynucleotides. The target sequence may be located in the nucleus or cytoplasm of the cell, such as within an organelle of the cell. Generally, a sequence or template that may be used for recombination into the targeted locus comprising the target sequences is referred to as an “editing template” or “editing polynucleotide” or “editing sequence.” In some aspects, an exogenous template polynucleotide may be referred to as a DNA template. In some aspects, the recombination is homologous recombination.

Typically, in the context of an endogenous CRISPR system, formation of the CRISPR complex (comprising the guide sequence hybridized to the target sequence and complexed with one or more Cas proteins) results in cleavage of one or both strands in or near (e.g. within 1, 2, 3, 4, 5, 6, 7, 8, q_{, 10, 20, 50}, or more base pairs from) the target sequence. The tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. The tracr sequence has sufficient complementarity to a tracr mate sequence to hybridize and participate in formation of the CRISPR complex, such as at least 50%, 60%, 70%, 80%, 90%, 95% or 99% of sequence complementarity along the length of the tracr mate sequence when optimally aligned.

The elements of the CRISPR system can be introduced into a cell such that expression of the elements of the CRISPR system direct formation of the CRISPR complex at one or more target sites. Components can be delivered to cells as proteins and/or RNA. For example, a Cas enzyme can be delivered as an mRNA encoding the Cas enzyme, the guide RNA can be delivered as an sgRNA, and the DNA template for HDR can be delivered as a DNA.

Non-limiting examples of Cas proteins include Cas1, Cas1B, Cas2, Cas3, Cas4, Cas5, Cas6. Cas7, Cas8, Cas9 (also known as Csn1 and Csx12). Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx 16, CsaX, Csx3, Csx1, Csx 15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2.

The CRISPR enzyme can be Cas9 (e.g., from S. pyogenes or S. pneumonia). The CRISPR enzyme can direct cleavage of one or both strands at the location of a target sequence, such as within the target sequence and/or within the complement of the target sequence. The vector can encode a CRISPR enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce HDR.

In general, a guide sequence is any polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), Clustal W, Clustal X, BLAT, Novoalign (Novocraft Technologies, ELAND (Illumina, San Diego, Calif.), SOAP (available at soap.genomics.org.cn), and Maq (available at maq.sourceforge.net).

The CRISPR enzyme may be part of a fusion protein comprising one or more heterologous protein domains. A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. Additional domains that may form part of a fusion protein comprising a CRISPR enzyme are described in US20110059502, incorporated herein by reference.

### B. Inhibitory Polynucleotides

“RNA interference (RNAi)” is the process of sequence-specific, post-transcriptional gene silencing initiated by siRNA. During RNAi, siRNA induces degradation of target mRNA with consequent sequence-specific inhibition of gene expression.

An “inhibitory polynucleotide,” “inhibitory RNA,” “RNAi,” “small interfering RNA” or “short interfering RNA” or “siRNA” molecule, “short hairpin RNA” or “shRNA” molecule, or “miRNA” is an RNA duplex of nucleotides that is targeted to a nucleic acid sequence of interest. As used herein, the term “siRNA” is a generic term that encompasses the subset of shRNAs and miRNAs. An “RNA duplex” refers to the structure formed by the complementary pairing between two regions of an RNA molecule. siRNA is “targeted” to a gene in that the nucleotide sequence of the duplex portion of the siRNA is complementary to a nucleotide sequence of the targeted gene. In certain embodiments, the siRNAs are targeted to the sequence encoding huntingtin. In some embodiments, the length of the duplex of siRNAs is less than 30 base pairs. In some embodiments, the duplex can be 29, 28, 27, 26, 25, 24, 23, 22, 21, 20, 19, 18, 17, 16, 15, 14, 13, 12, 11 or 10 base pairs in length. In some embodiments, the length of the duplex is 19 to 25 base pairs in length. In certain embodiment, the length of the duplex is 19 or 21 base pairs in length. The RNA duplex portion of the siRNA can be part of a hairpin structure. In addition to the duplex portion, the hairpin structure may contain a loop portion positioned between the two sequences that form the duplex. The loop can vary in length. In some embodiments the loop is 5, 6, 7, 8, q_{, 10, 11, 12, 13, 14, 15, 16, 17, 18, 19, 20, 21, 22, 23, 24} or 25 nucleotides in length. In certain embodiments, the loop is 18 nucleotides in length. The hairpin structure can also contain 3′ and/or 5′ overhang portions. In some embodiments, the overhang is a 3′ and/or a 5′ overhang 0, 1, 2, 3, 4 or 5 nucleotides in length.

shRNAs are comprised of stem-loop structures which are designed to contain a 5′ flanking region, siRNA region segments, a loop region, a 3′ siRNA region and a 3′ flanking region. Most RNAi expression strategies have utilized short-hairpin RNAs (shRNAs) driven by strong polIII-based promoters. Many shRNAs have demonstrated effective knock down of the target sequences in vitro as well as in vivo, however, some shRNAs which demonstrated effective knock down of the target gene were also found to have toxicity in vivo.

miRNAs are small cellular RNAs (˜22 nt) that are processed from precursor stem loop transcripts. Known miRNA stem loops can be modified to contain RNAi sequences specific for genes of interest. miRNA molecules can be preferable over shRNA molecules because miRNAs are endogenously expressed. Therefore, miRNA molecules are unlikely to induce dsRNA-responsive interferon pathways, they are processed more efficiently than shRNAs, and they have been shown to silence 80% more effectively.

A recently discovered alternative approach is the use of artificial miRNAs (pri-miRNA scaffolds shuttling siRNA sequences) as RNAi vectors. Artificial miRNAs more naturally resemble endogenous RNAi substrates and are more amenable to Pol-II transcription (e.g., allowing tissue-specific expression of RNAi) and polycistronic strategies (e.g., allowing delivery of multiple siRNA sequences). See U.S. Pat. No. 10,093,927, which is incorporated by reference.

The transcriptional unit of a “shRNA” is comprised of sense and antisense sequences connected by a loop of unpaired nucleotides. shRNAs are exported from the nucleus by

Exportin-5, and once in the cytoplasm, are processed by Dicer to generate functional siRNAs. “miRNAs” stem-loops are comprised of sense and antisense sequences connected by a loop of unpaired nucleotides typically expressed as part of larger primary transcripts (pri-miRNAs), which are excised by the Drosha-DGCR⁸ complex generating intermediates known as pre-miRNAs, which are subsequently exported from the nucleus by Exportin-5, and once in the cytoplasm, are processed by Dicer to generate functional siRNAs. “Artificial miRNA” or an “artificial miRNA shuttle vector”, as used herein interchangeably, refers to a primary miRNA transcript that has had a region of the duplex stem loop (at least about 9-20 nucleotides) which is excised via Drosha and Dicer processing replaced with the siRNA sequences for the target gene while retaining the structural elements within the stem loop necessary for effective Drosha processing. The term “artificial” arises from the fact the flanking sequences (˜35 nucleotides upstream and ˜40 nucleotides downstream) arise from restriction enzyme sites within the multiple cloning site of the siRNA. As used herein the term “miRNA” encompasses both the naturally occurring miRNA sequences as well as artificially generated miRNA shuttle vectors.

The siRNA can be encoded by a nucleic acid sequence, and the nucleic acid sequence can also include a promoter. The nucleic acid sequence can also include a polyadenylation signal. In some embodiments, the polyadenylation signal is a synthetic minimal polyadenylation signal or a sequence of six Ts.

In designing RNAi there are several factors that need to be considered, such as the nature of the siRNA, the durability of the silencing effect, and the choice of delivery system. To produce an RNAi effect, the siRNA that is introduced into the organism will typically contain exonic sequences. Furthermore, the RNAi process is homology dependent, so the sequences must be carefully selected so as to maximize gene specificity, while minimizing the possibility of cross-interference between homologous, but not gene-specific sequences. Preferably the siRNA exhibits greater than 80%, 85%, 90%, 95%, 98%, or even 100% identity between the sequence of the siRNA and the gene to be inhibited. Sequences less than about 80% identical to the target gene are substantially less effective. Thus, the greater homology between the siRNA and the gene to be inhibited, the less likely expression of unrelated genes will be affected.

In addition, the size of the siRNA is an important consideration. In some embodiments, the present invention relates to siRNA molecules that include at least about 19-25 nucleotides and are able to modulate gene expression. In the context of the present invention, the siRNA is preferably less than 500, 200, 100, 50, or 25 nucleotides in length. More preferably, the siRNA is from about 19 nucleotides to about 25 nucleotides in length.

A siRNA target generally means a polynucleotide comprising a region that encodes a polypeptide, or a polynucleotide region that regulates replication, transcription, or translation or other processes important to expression of the polypeptide, or a polynucleotide comprising both a region that encodes a polypeptide and a region operably linked thereto that regulates expression. Any gene being expressed in a cell can be targeted. Preferably, a target gene is one involved in or associated with the progression of cellular activities important to disease or of particular interest as a research object.

### C. Ionizable Lipids

In some aspects of the present disclosure, composition containing compounds containing lipophilic and cationic components, wherein the cationic component is ionizable, are provided. In some embodiments, these cationic ionizable lipids are dendrimers, which are a polymer exhibiting regular dendritic branching, formed by the sequential or generational addition of branched layers to or from a core and are characterized by a core, at least one interior branched layer, and a surface branched layer. (See Petar R. Dvornic and Donald A. Tomalia in Chem. in Britain, 641-645, August 1994.) In other embodiments, the term “dendrimer” as used herein is intended to include, but is not limited to, a molecular architecture with an interior core, interior layers (or “generations”) of repeating units regularly attached to this initiator core, and an exterior surface of terminal groups attached to the outermost generation. A “dendron” is a species of dendrimer having branches emanating from a focal point which is or can be joined to a core, either directly or through a linking moiety to form a larger dendrimer. In some embodiments, the dendrimer structures have radiating repeating groups from a central core which doubles with each repeating unit for each branch. In some embodiments, the dendrimers described herein may be described as a small molecule, medium-sized molecules, lipids, or lipid-like material. These terms may be used to described compounds described herein which have a dendron like appearance (e.g., molecules which radiate from a single focal point).

While dendrimers are polymers, dendrimers may be preferable to traditional polymers because they have a controllable structure, a single molecular weight, numerous and controllable surface functionalities, and traditionally adopt a globular conformation after reaching a specific generation. Dendrimers can be prepared by sequentially reactions of each repeating unit to produce monodisperse, tree-like and/or generational structure polymeric structures. Individual dendrimers consist of a central core molecule, with a dendritic wedge attached to one or more functional sites on that central core. The dendrimeric surface layer can have a variety of functional groups disposed thereon including anionic, cationic, hydrophilic, or lipophilic groups, according to the assembly monomers used during the preparation. In some embodiments, the ionizable cationic lipid is a dendrimer or dendron further defined by the formula:

Core-Repeating Unit-Terminating Group (D-I)

wherein the core is linked to the repeating unit by removing one or more hydrogen atoms from the core and replacing the atom with the repeating unit and wherein:
the core has the formula:

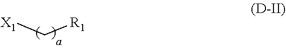

wherein:
   X₁ is amino or alkylamino_{(C≤12)}, dialkylamino_{(C≤12)}, heterocycloalkyl_{(C≤12)}, heteroaryl_{(C≤12)}, or a substituted version thereof;
   R¹ is amino, hydroxy, or mercapto, or alkylamino_{(C≤12)}, dialkylamino_{(C≤12)}, or a substituted version of either of these groups; and
   a is 1, 2, 3, 4, 5, or 6; or
the core has the formula:

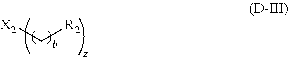

wherein:
   X₂ is N(R₅)_{y};
   R₅ is hydrogen, alkyl_{(C≤18)}, or substituted alkyl_{(C≤18)}; and
   y is 0, 1, or 2, provided that the sum of y and z is 3;
   R₂ is amino, hydroxy, or mercapto, or alkylamino_{(C≤12)}, dialkylamino_{(C=12)}, or a substituted version of either of these groups;
   b is 1, 2, 3, 4, 5, or 6; and
   z is 1, 2, 3; provided that the sum of z and y is 3; or
the core has the formula:

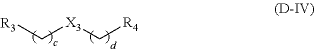

wherein:
   X₃ is —NR₆—, wherein R₆ is hydrogen, alkyl_{(C≤8)}, or substituted alkyl_{(C≤8)}, —O—, or alkylaminodiyl_{(C≤8)}, alkoxydiyl_{(C≤8)}, arenediyl_{(C≤8)}, heteroarenediyl_{(C≤8)}, heterocycloalkanediyl_{(C≤8)}, or a substituted version of any of these groups;
   R₃ and R₄ are each independently amino, hydroxy, or mercapto, or alkylamino_{(C≤12)}, dialkylamino_{(C≤12)}, or a substituted version of either of these groups; or a group of the formula: —N(R_{f})_{f}(CH₂CH₂N(R_{c}))ₑR_{d},

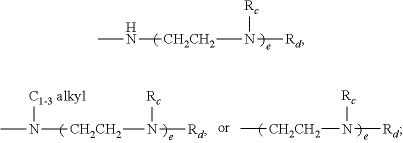

wherein:
   e and f are each independently 1, 2, or 3; provided that the sum of e and f is 3;
   R_{c}, R_{d}, and R_{f} are each independently hydrogen, alkyl_{(C≤6)}, or substituted alkyl_{(C≤6)};
c and d are each independently 1, 2, 3, 4, 5, or 6; or
the core is alkylamine_{(C≤18)}, dialkylamine_{(C≤36)}, heterocycloalkane_{(C≤12)}, or a substituted version of any of these groups;
wherein the repeating unit comprises a degradable diacyl and a linker;
   the degradable diacyl group has the formula:

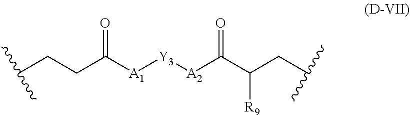

wherein:
   A₁ and A₂ are each independently —O—, —S—, or —NRₐ—, wherein:
   Rₐ is hydrogen, alkyl_{(C≤6)}, or substituted alkyl_{(C≤6)};
   Y₃ is alkanediyl_{(C≤12)}, alkenediyl_{(C≤12)}, arenediyl_{(C≤12)}, or a substituted version of any of these groups; or a group of the formula:

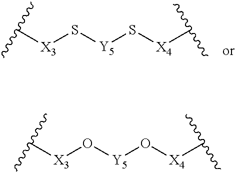

wherein:
   X₃ and X₄ are alkanediyl_{(C≤12)}, alkenediyl_{(C≤12)}, arenediyl_{(C≤12)}, or a substituted version of any of these groups;
   Y₅ is a covalent bond, alkanediyl_{(C≤12)}, alkenediyl_{(C≤12)}, arenediyl_{(C≤12)}, or a substituted version of any of these groups; and
   R₉ is alkyl_{(C≤8)} or substituted alkyl_{(C≤8)};
the linker group has the formula:

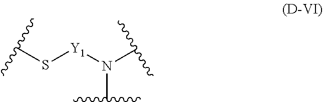

wherein:
   Y₁ is alkanediyl_{(C≤12)}, alkenediyl_{(C≤12)}, arenediyl_{(C≤12)}, or a substituted version of any of these groups; and
wherein when the repeating unit comprises a linker group, then the linker group comprises an independent degradable diacyl group attached to both the nitrogen and the sulfur atoms of the linker group if n is greater than 1, wherein the first group in the repeating unit is a degradable diacyl group, wherein for each linker group, the next repeating unit comprises two degradable diacyl groups attached to the nitrogen atom of the linker group; and wherein n is the number of linker groups present in the repeating unit; and
the terminating group has the formula:

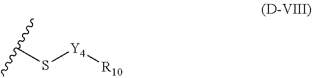

wherein:
   Y₄ is alkanediyl_{(C≤18)} or an alkanediyl_{(C≤18)} wherein one or more of the hydrogen atoms on the alkanediyl_{(C≤18)} has been replaced with —OH, —F, -Cl, —Br, —I, —SH, —OCH₃, —OCH₂CH₃, —SCH3, or —OC(O)CH₃;
   R₁₀ is hydrogen, carboxy, hydroxy, or
   aryl_{(C≤12)}, alkylamino (<12), dialkylamino (<12), N-heterocycloalkyl_{(C≤12)}, —C(O)N(R₁₁)-alkanediyl_{(C≤6)}-heterocycloalkyl_{(C≤12)}, —C(O)-alkyl-amino_{(C≤12)}, —C(O)-dialkylamino_{(C≤12)}, —C(O)—N-heterocyclo-alkyl_{(C≤12)}, wherein:
   R₁₁ is hydrogen, alkyl_{(C≤6)}, or substituted alkyl_{(C≤6)};
   wherein the final degradable diacyl in the chain is attached to a terminating group;
   n is 0, 1, 2, 3, 4, 5, or 6;
or a pharmaceutically acceptable salt thereof.

In some embodiments, the terminating group is further defined by the formula:

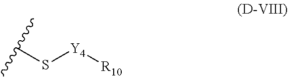

wherein:
Y₄ is alkanediyl_{(C≤18)}; and
R₁₀ is hydrogen.

In some embodiments, A₁ and A₂ are each independently —O— or —NRₐ—.

In some embodiments of the dendrimer or dendron of formula (D-I), the core is further defined by the formula:

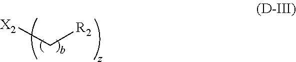

wherein:
X₂ is N(R₅)_{y};
   R₅ is hydrogen or alkyl_{(C≤8)}, or substituted alkyl_{(C≤18)}; and
   y is 0, 1, or 2, provided that the sum of y and z is 3;
R₂ is amino, hydroxy, or mercapto, or alkylamino_{(C≤12)}, dialkylamino_{(C≤12)}, or a substituted version of either of these groups;
b is 1, 2, 3, 4, 5, or 6; and
z is 1, 2, 3; provided that the sum of z and y is 3.

In some embodiments of the dendrimer or dendron of formula (D-I), the core is further defined by the formula:

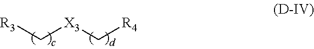

wherein:
X₃ is —NR₆—, wherein R₆ is hydrogen, alkyl_{(C≤8)}, or substituted alkyl_{(C≤8)}, —O—, or alkylaminodiyl_{(C≤8)}, alkoxydiyl_{(C≤8)}, arenediyl_{(C≤8)}, heteroarenediyl_{(C≤8)}, heterocycloalkanediyl_{(C≤8)}, or a substituted version of any of these groups;
R₃ and R₄ are each independently amino, hydroxy, or mercapto, or alkylamino_{(C≤12)}, dialkylamino_{(C≤12)}, or a substituted version of either of these groups; or a group of the formula: —N(R_{f}) ((CH₂CH₂N(R_{c}))ₑR_{d},

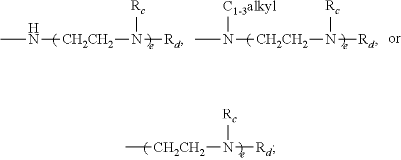

wherein:
   e and f are each independently 1, 2, or 3; provided that the sum of e and f is 3;
   R_{c}, R_{d}, and R_{f} are each independently hydrogen, alkyl_{(C≤8)}, or substituted alkyl_{(C≤6)};
c and d are each independently 1, 2, 3, 4, 5, or 6.

In some embodiments of the dendrimer or dendron of formula (1), the terminating group is represented by the formula:

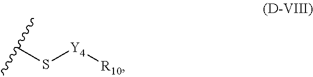

wherein:
Y₄ is alkanediyl_{(C≤18)}; and
R₁₀ is hydrogen.

In some embodiments of the dendrimer or dendron of formula (D-I), the core is further defined as:

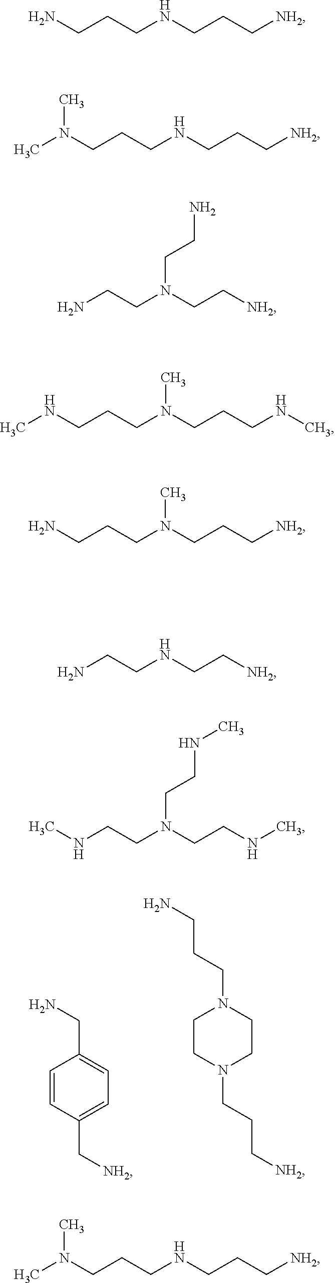

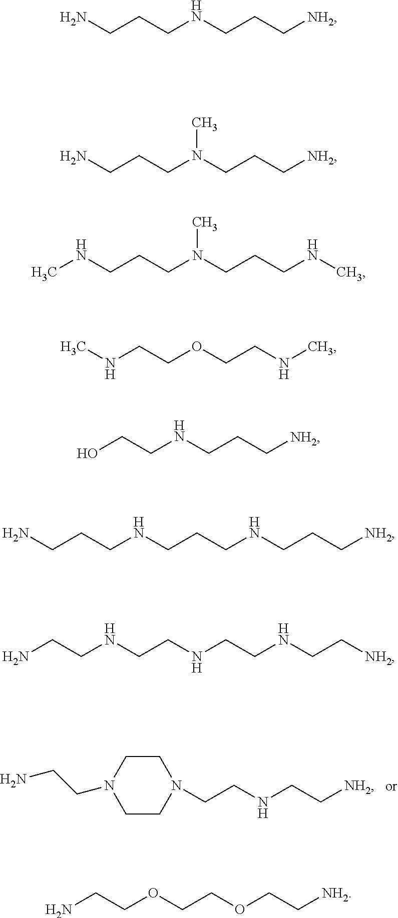

In some embodiments of the dendrimer or dendron of formula (D-I), the degradable diacyl is further defined as:

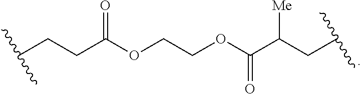 [SMILES: *CCC(=O)OCCOC(=O)C(C)C*]

In some embodiments of the dendrimer or dendron of formula (D-I), the linker is further defined as,

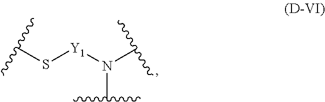

wherein Y₁ is alkanediyl_{(C≤8)} or substituted alkanediyl_{(C≤8)}. 1. In some embodiments of the dendrimer or dendron of formula (D-I), the dendrimer or dendron is selected from the group consisting of: 10

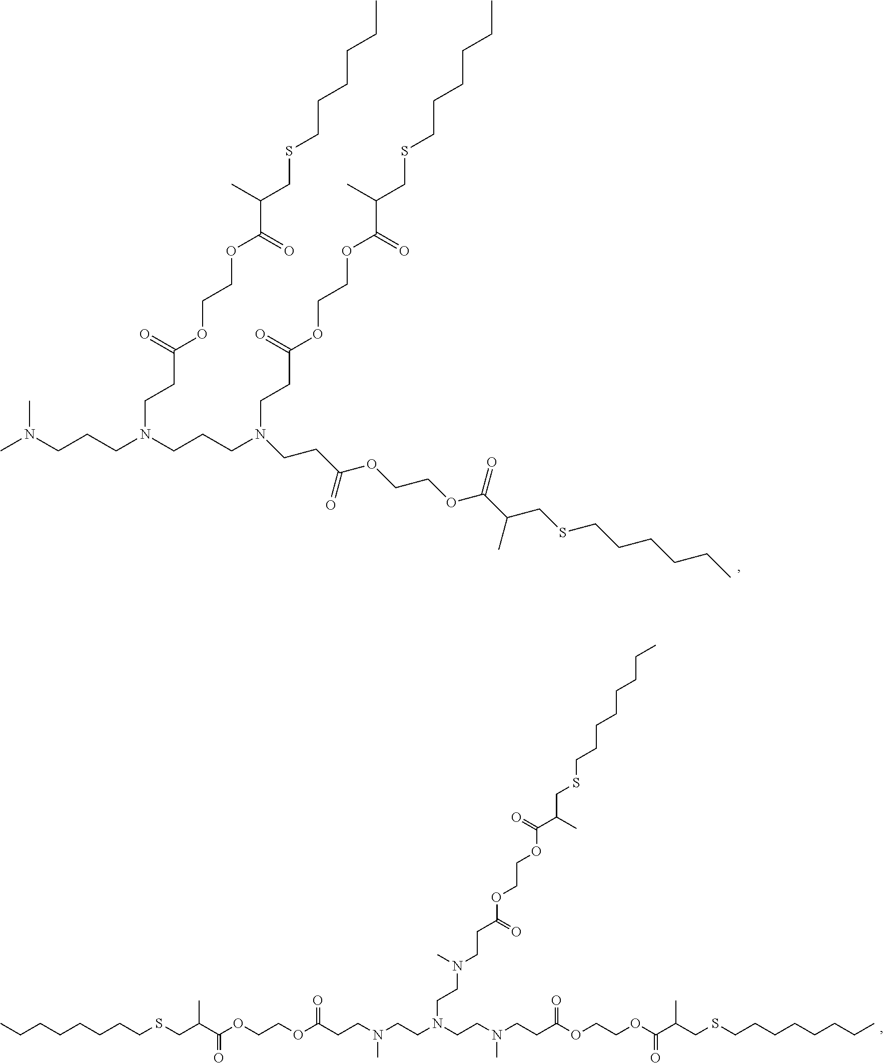

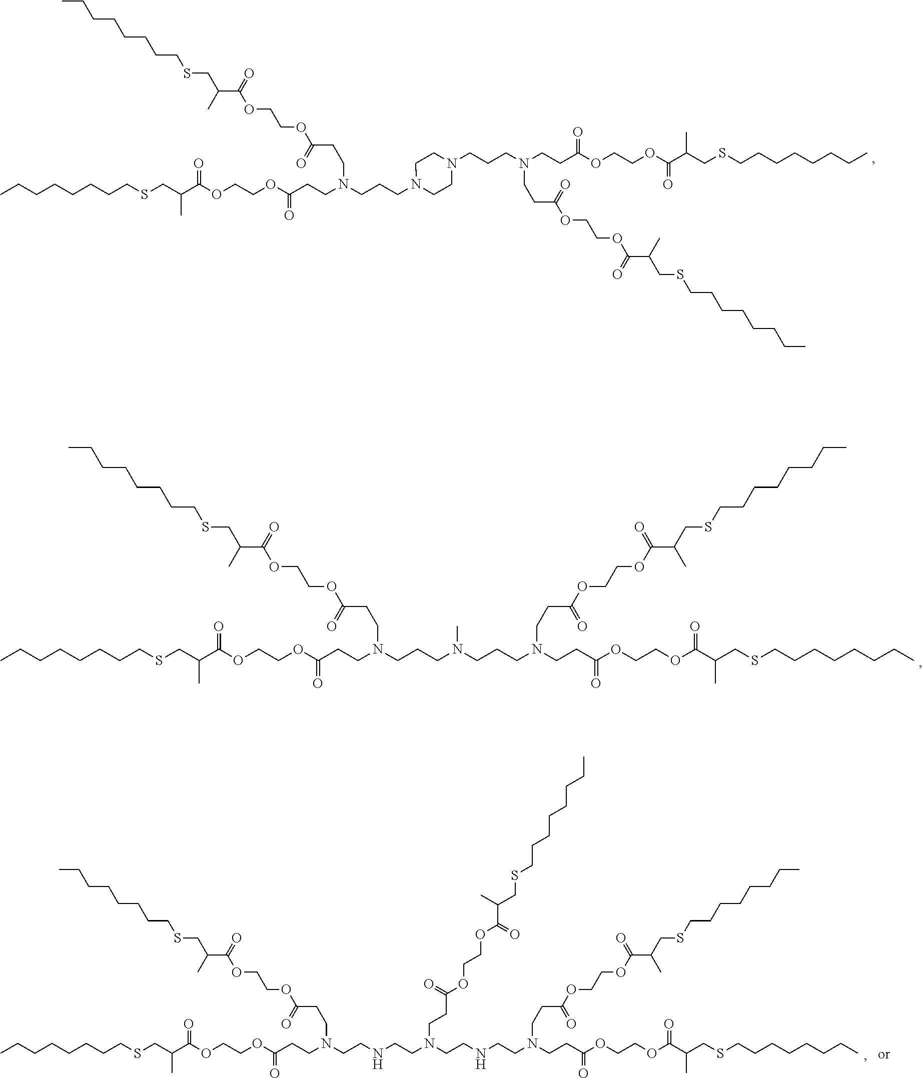

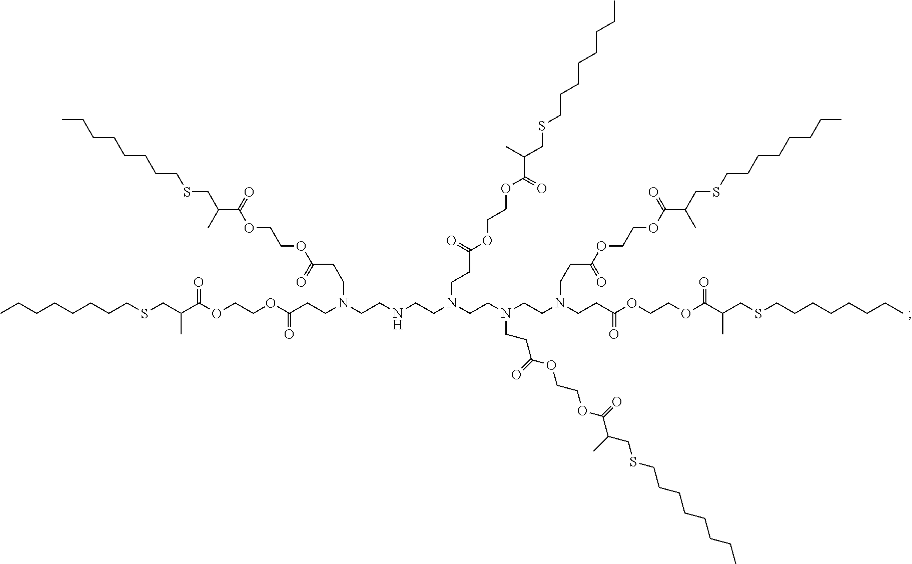 [SMILES: CCCCCCCCSCC(C)C(=O)OCCOC(=O)CCN(CCNCCN(CCC(=O)OCCOC(=O)C(C)CSCCCCCCCC)CCN(CCC(=O)OCCOC(=O)C(C)CSCCCCCCCC)CCN(CCC(=O)OCCOC(=O)C(C)CSCCCCCCCC)CCC(=O)OCCOC(=O)C(C)CSCCCCCCCC)CCC(=O)OCCOC(=O)C(C)CSCCCCCCCC]

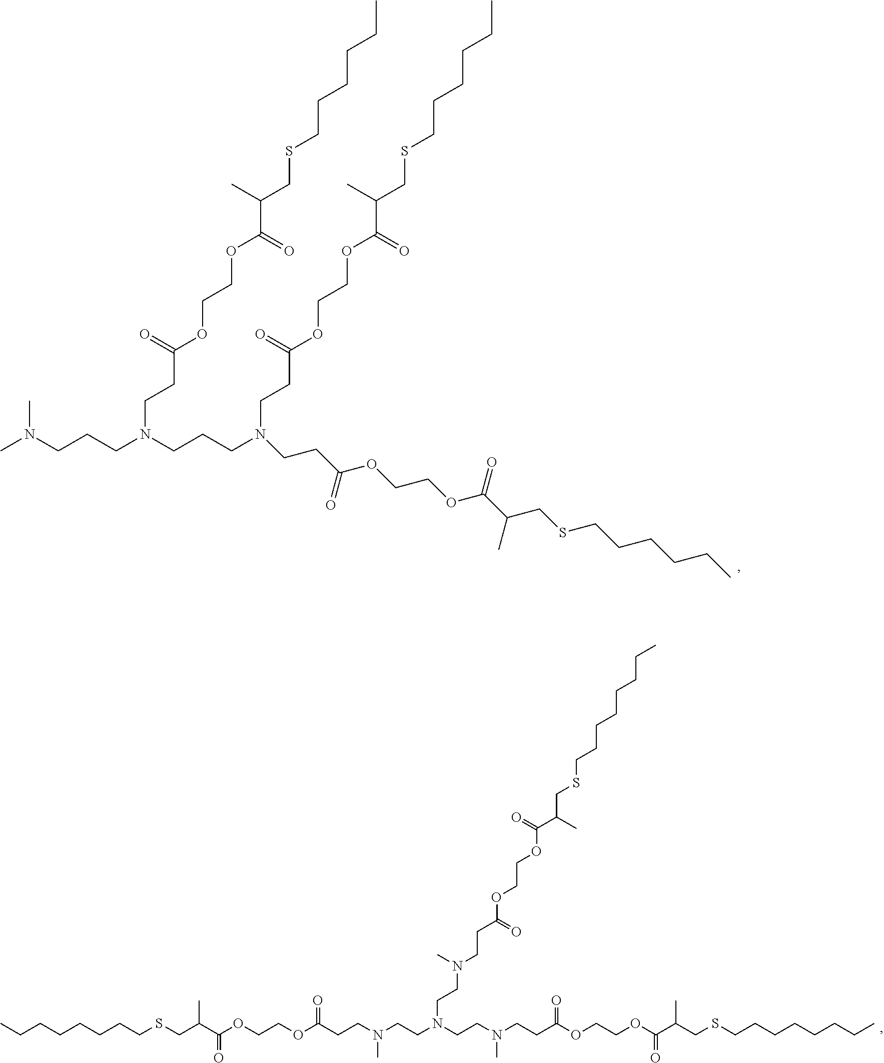

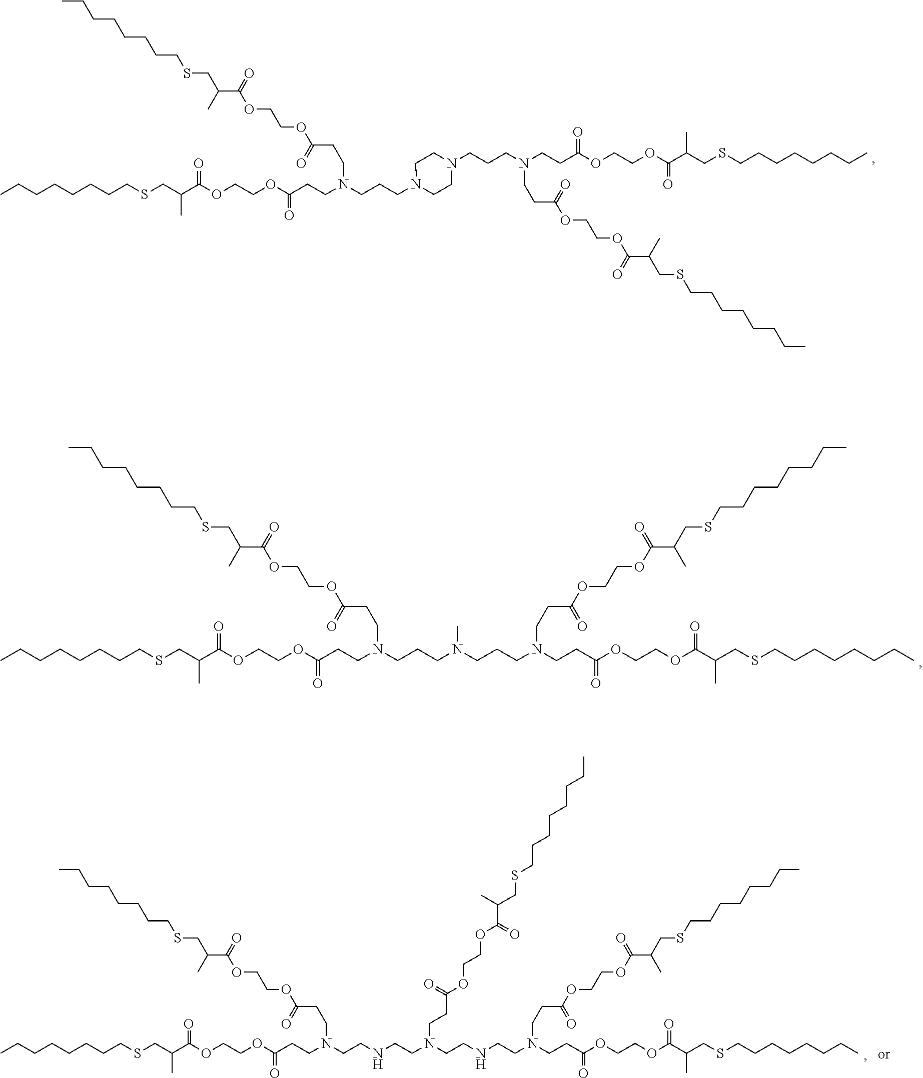

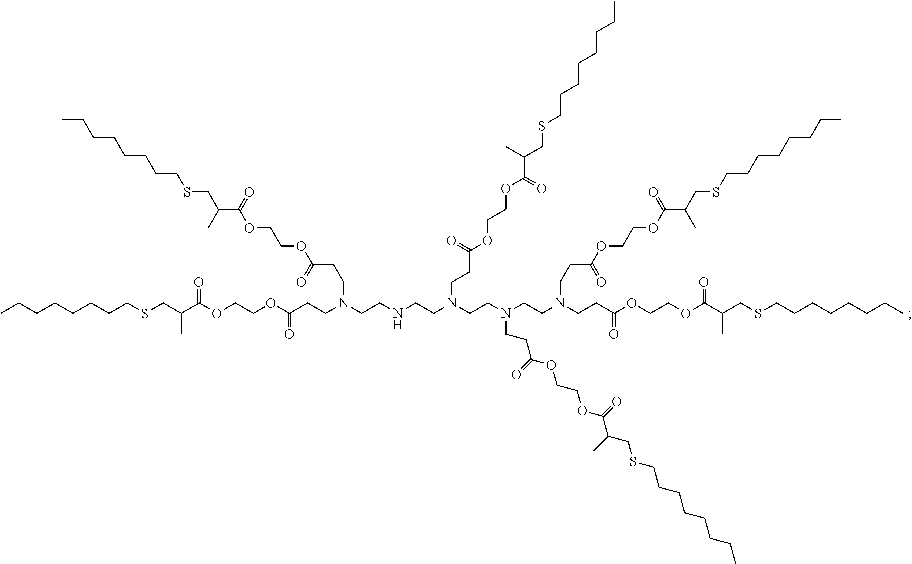 [SMILES: CCCCCCCCSCC(C)C(=O)OCCOC(=O)CCN(CCNCCN(CCC(=O)OCCOC(=O)C(C)CSCCCCCCCC)CCN(CCC(=O)OCCOC(=O)C(C)CSCCCCCCCC)CCN(CCC(=O)OCCOC(=O)C(C)CSCCCCCCCC)CCC(=O)OCCOC(=O)C(C)CSCCCCCCCC)CCC(=O)OCCOC(=O)C(C)CSCCCCCCCC]

and pharmaceutically acceptable salts thereof.

### B. Dendrimers or dendrons of Formula (X)

A. In some embodiments of the lipid composition, the ionizable cationic lipid is a dendrimer or dendron of the formula Core—(Branch)_{N}. In some embodiments, the ionizable cationic lipid is a dendrimer or dendron of the formula

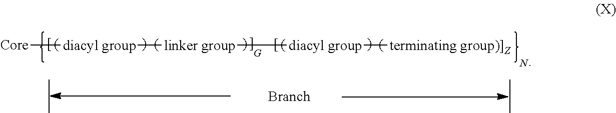

B. In some embodiments of the lipid composition, the ionizable cationic lipid is a dendrimer or dendron of a generation (g) having a structural formula:

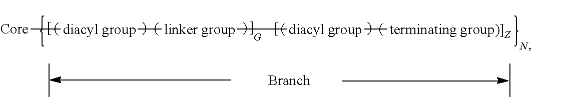

or a pharmaceutically acceptable salt thereof, wherein:
(a) the core comprises a structural formula (X_{Core}):

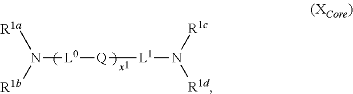

wherein:
   Q is independently at each occurrence a covalent bond, —O—, —S—, —NR²—, or —CR^{3a}R^{3b}—;
   R² is independently at each occurrence R^{1g} or -L²—NR^{1e}R^{1f};
   R^{3a} and R^{3b} are each independently at each occurrence hydrogen or an optionally substituted (e.g., C₁-C₆, such as C₁-C₃) alkyl;
   R^{1a}, R¹, R^{1c}, R^{1d}, R^{1e}, R^{1f}, and R^{1g} (if present) are each independently at each occurrence a point of connection to a branch, hydrogen, or an optionally substituted (e.g., C₁-C₁₂) alkyl;
   L⁰, L¹, and L² are each independently at each occurrence selected from a covalent bond, alkylene, heteroalkylene, [alkylene]-[heterocycloalkyl]-[alkylene], [alkylene]-(arylene)-[alkylene], heterocycloalkyl, and arylene; or,
   alternatively, part of L¹ form a (e.g., C₄-C₆) heterocycloalkyl (e.g., containing one or two nitrogen atoms and, optionally, an additional heteroatom selected from oxygen and sulfur) with one of R^{1c} and R^{1d}; and
   x¹ is 0, 1, 2, 3, 4, 5, or 6; and
(b) each branch of the plurality (N) of branches independently comprises a structural formula (X_{Branch}):

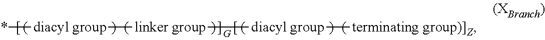

wherein:
   * indicates a point of attachment of the branch to the core;
   g is 1, 2, 3, or 4;
   Z=2^{(g-1)};
G=0, when g=1; or G=Σᵢ₌₀^{i=g-2}2ⁱ, when g≠1;
   (c) each diacyl group independently comprises a structural formula

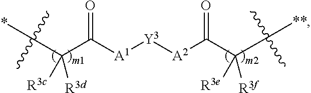

wherein:
*indicates a point of attachment of the diacyl group at the proximal end thereof;
** indicates a point of attachment of the diacyl group at the distal end thereof;
Y₃ is independently at each occurrence an optionally substituted (e.g., C₁-C₁₂); alkylene, an optionally substituted (e.g., C₁-C₁₂) alkenylene, or an optionally substituted (e.g., C₁-C₁₂) arenylene;
A₁ and A₂ are each independently at each occurrence —O—, —S—, or —NR⁴—, wherein:
   R⁴ is hydrogen or optionally substituted (e.g., C₁-C₆) alkyl;
m¹ and m² are each independently at each occurrence 1, 2, or 3; and
R^{3c}, R^{3d}, R^{3e}, and R^{3f} are each independently at each occurrence hydrogen or an optionally substituted (e.g., C₁-C₈) alkyl; and
(d) each linker group independently comprises a structural formula

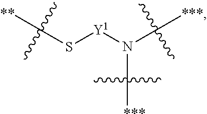

wherein:
   ** indicates a point of attachment of the linker to a proximal diacyl group;
   *** indicates a point of attachment of the linker to a distal diacyl group; and
   Y₁ is independently at each occurrence an optionally substituted (e.g., C₁-C₁₂) alkylene, an optionally substituted (e.g., C₁-C₁₂) alkenylene, or an optionally substituted (e.g., C₁-C₁₂) arenylene; and
(e) each terminating group is independently selected from optionally substituted (e.g., C₁-C₁₈, such as C₄-C₁₈) alkylthiol, and optionally substituted (e.g., C₁-C₁₈, such as C₄-C₁₈) alkenylthiol.

In some embodiments of X_{Core}, Q is independently at each occurrence a covalent bond, —O—, —S—, —NR²—, or —CR^{3a}R^{3b}. In some embodiments of X_{Core} Q is independently at each occurrence a covalent bond. In some embodiments of X_{Core} Q is independently at each occurrence an—O—. In some embodiments of X_{Core} Q is independently at each occurrence a-S—. In some embodiments of X_{Core} Q is independently at each occurrence a—NR² and R² is independently at each occurrence R^{1g} or -L²—NR^{1e}R^{1f}. In some embodiments of X_{Core} Q is independently at each occurrence a-CR^{3a}R^{3b}R^{3a}, and R^{3a} and R^{3b} are each independently at each occurrence hydrogen or an optionally substituted alkyl (e.g., C₁-C₆, such as C₁-C₃). In some embodiments of X_{Core}, R^{1a}, R^{1b}, R^{1c}, R^{1d}, R^{1e}, R^{1f}, and R^{1g} (if present) are each independently at each occurrence a point of connection to a branch, hydrogen, or an optionally substituted alkyl. In some embodiments of X_{Core}, R^{1a}, R^{1b}, R^{1e}, R^{1d}, R^{1e}, R^{1f}, and R^{1g} (if present) are each independently at each occurrence a point of connection to a branch, hydrogen. In some embodiments of X_{Core}, R^{1a}, R^{1b}, R^{1c}, R^{1d}, R^{1e}, R^{1f}, and R^{1g} (if present) are each independently at each occurrence a point of connection to a branch an optionally substituted alkyl (e.g., C₁-C₁₂).

In some embodiments of X_{Core}, L⁰, L¹, and L² are each independently at each occurrence selected from a covalent bond, alkylene, heteroalkylene, [alkylene]-[heterocycloalkyl]-[alkylene], [alkylene]-(arylene)-[alkylene], heterocycloalkyl, and arylene; or, alternatively, part of L′ form a heterocycloalkyl (e.g., C₄-C₆ and containing one or two nitrogen atoms and, optionally, an additional heteroatom selected from oxygen and sulfur) with one of R^{1e} and R^{1d}. In some embodiments of X_{Core}, L⁰, L¹, and L′ are each independently at each occurrence can be a covalent bond. In some embodiments of X_{Core}, L⁰, L¹, and L² are each independently at each occurrence can be a hydrogen. In some embodiments of X_{Core}, L⁰, L¹, and L² are each independently at each occurrence can be an alkylene (e.g., C₁-C₁₂, such as C₁-C₆ or C₁-C₃). In some embodiments of X_{Core}, L⁰, L¹, and L′ are each independently at each occurrence can be a heteroalkylene (e.g., C₁-C₁₂, such as C₁-C₈ or C₁-C₆). In some embodiments of X_{Core}, L⁰, L¹, and L² are each independently at each occurrence can be a heteroalkylene (e.g., C₂-C₈ alkyleneoxide, such as oligo (ethyleneoxide)). In some embodiments of X_{Core}, L⁰, L¹, and L² are each independently at each occurrence can be a [alkylene]-[heterocycloalkyl]-[alkylene][(e.g., C₁-C₆) alkylene]-[(e.g., C₄-C₆) heterocycloalkyl]-[(e.g., C₁-C₆) alkylene]. In some embodiments of X_{Core}, L⁰, L¹, and L′ are each independently at each occurrence can be a [alkylene]-(arylene)-[alkylene][(e.g., C₁-C₆) alkylene]-(arylene)-[(e.g., C₁-C₆) alkylene]. In some embodiments of X_{Core}, L⁰, L′, and L′ are each independently at each occurrence can be a [alkylene]-(arylene)-[alkylene] (e.g., [(e.g., C₁-C₆) alkylene]-phenylene-[(e.g., C₁-C₆) alkylene]). In some embodiments of X_{Core}, L⁰, L¹, and L² are each independently at each occurrence can be a heterocycloalkyl (e.g., C₄-C₆heterocycloalkyl). In some embodiments of X_{Core}, L⁰, L¹, and L² are each independently at each occurrence can be an arylene (e.g., phenylene). In some embodiments of X_{Core}, part of L¹ form a heterocycloalkyl with one of R^{1c} and R^{1d}. In some embodiments of X_{Core}, part of L′ form a heterocycloalkyl (e.g., C₄-C₆ heterocycloalkyl) with one of R^{1c} and R^{1d} and the heterocycloalkyl can contain one or two nitrogen atoms and, optionally, an additional heteroatom selected from oxygen and sulfur.

In some embodiments of X_{Core}, L⁰, L¹, and L′ are each independently at each occurrence selected from a covalent bond, C₁-C₆ alkylene (e.g., C₁-C₃ alkylene), C₂-C₁₂ (e.g., C₂-C₈) alkyleneoxide (e.g., oligo (ethyleneoxide), such as —(CH₂CH₂O)₁₋₄—(CH₂CH₂)—), [(C₁-C₄) alkylene]-[(C₄-C₆) heterocycloalkyl]-[(C₁-C₄) alkylene]

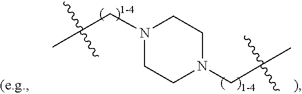

and [(C₁-C₄) alkylene]-phenylene-[(C₁-C₄) alkylene]

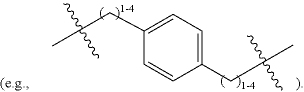

In some embodiments of X_{Core}, L⁰, L¹, and L² are each independently at each occurrence selected from C₁-C₆ alkylene (e.g., C₁-C₃ alkylene), —(C₁-C₃ alkylene-O)₁₋₄-(C₁-C₃ alkylene), —(C₁-C₃ alkylene)-phenylene-(C₁-C₃ alkylene)-, and —(C₁-C₃ alkylene)-piperazinyl-(C₁-C₃ alkylene)-. In some embodiments of X_{Core}. L⁰, L¹, and L′ are each independently at each occurrence C₁-C₆ alkylene (e.g., C₁-C₃ alkylene). In some embodiments, L⁰, L¹, and L² are each independently at each occurrence C₂-C₁₂ (e.g., C₂-C₈) alkyleneoxide (e.g., —(C₁-C₃ alkylene-O)₁₋₄-(C₁-C₃ alkylene)). In some embodiments of X_{Core}, L⁰, L′, and L² are each independently at each occurrence selected from [(C₁-C₄) alkylene]-[(C₄-C₆) heterocycloalkyl]-[(C₁-C₄) alkylene] (e.g., (C₁-C₃ alkylene)-phenylene-(C₁-C₃ alkylene)-) and [(C₁-C₄) alkylene]-[(C₄-C₆) heterocycloalkyl]-[(C₁-C₄) alkylene] (e.g., —(C₁-C₃ alkylene)-piperazinyl-(C₁-C₃ alkylene)-).

In some embodiments of X_{Core}. X₁ is 0, 1, 2, 3, 4, 5, or 6. In some embodiments of X_{Core}. x¹ is 0. In some embodiments of X_{Core}, X¹ is 1. In some embodiments of X_{Core}, X¹ is 2. In some embodiments of X_{Core}. X¹ is 0. In some embodiments of X_{Core}. x¹ is 1. In some embodiments of X_{Core}. x¹ is 2. In some embodiments of X_{Core}. X¹ is 3. In some embodiments of X_{Core} X¹ is 4. In some embodiments of X_{Core} X¹ is 5. In some embodiments of X_{Core}. X¹ is 6.

2. In some embodiments of X_{Core}, the core comprises a structural formula:

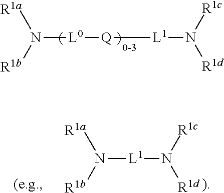

In some embodiments of X_{Core}, the core comprises a structural formula:

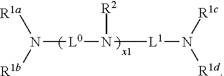

In some embodiments of X_{Core}, the core comprises a structural formula:

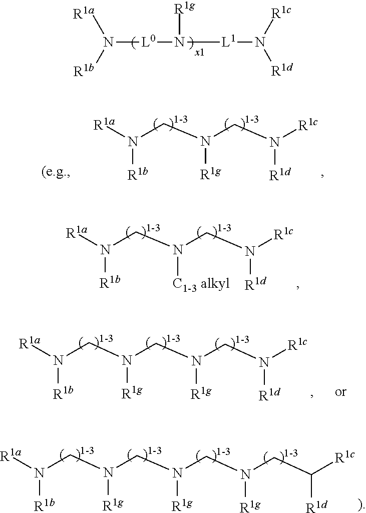

In some embodiments of X_{Core}, the core comprises a structural formula:

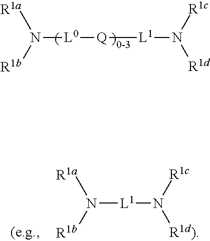

In some embodiments of X_{Core}, the core comprises a structural formula:

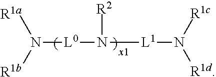

In some embodiments of X_{Core}. the core comprises a structural formula:

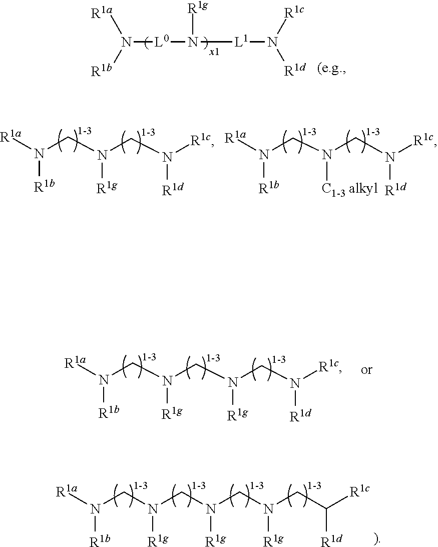

In some embodiments of X_{Core}, the core comprises a structural formula:

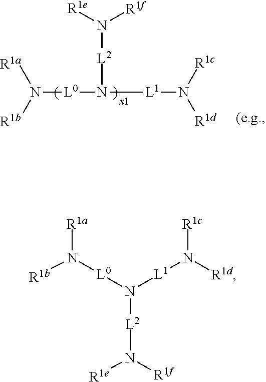

such as

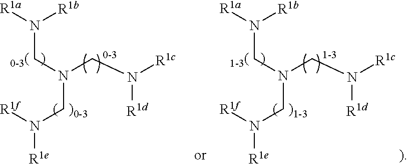

In some embodiments of X_{Core}, the core comprises a structural formula:

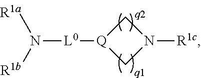

wherein Q′ is —NR²— or -CR³aR3b-; q1 and q2 are each independently 1 or 2. In some embodiments of X_{Core}, the core comprises a structural formula:

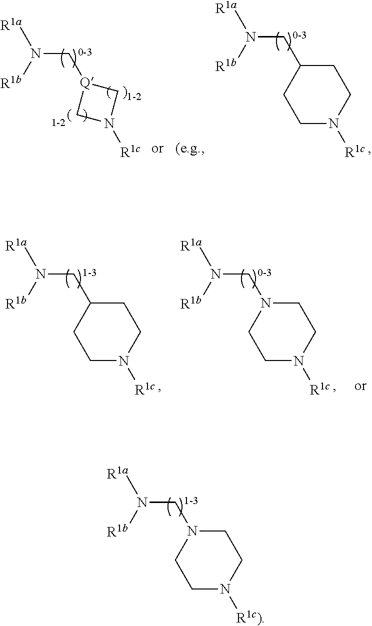

In some embodiments of X_{Core}, the core comprises a structural formula

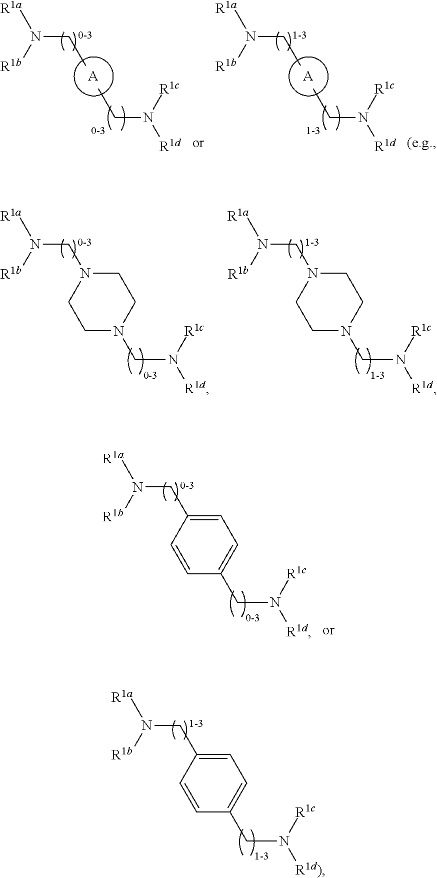

wherein ring A is an optionally substituted aryl or an optionally substituted (e.g., C₃-C₁₂, such as C₃-C₅) heteroaryl. In some embodiments of X_{Core}, the core comprises has a structural formula

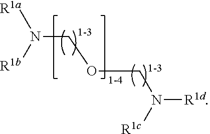

In some embodiments of X_{Core}, the core comprises a structural formula set forth in Table A and pharmaceutically acceptable salts thereof, wherein * indicates a point of attachment of the core to a branch of the plurality of branches. In some embodiments, the example cores of Table. A are not limited to the stereoisomers (i.e., enantiomers, diastereomers) listed.

TABLE A
Example core structures

| ID # | Structure |
|---|---|
| 1A1 | 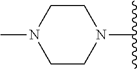 [SMILES: *N1CCN(C)CC1] |
| 1A2-1 | 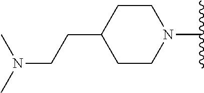 [SMILES: *N1CCC(CCN(C)C)CC1] |
| 1A2-2 | 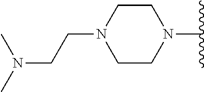 [SMILES: *N1CCN(CCN(C)C)CC1] |
| 1A3-1 | 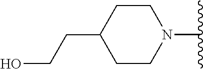 [SMILES: *N1CCC(CCO)CC1] |
| 1A3-2 | 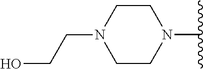 [SMILES: *N1CCN(CCO)CC1] |
| 1A4 | 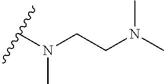 [SMILES: *N(C)CCN(C)C] |
| 1A5-1 | 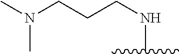 [SMILES: *NCCCN(C)C] |
| 1A5-2 | 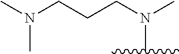 |
| 2A1-1 | 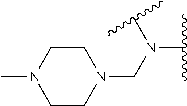 [SMILES: *N(*)CN1CCN(C)CC1] |
| 2A1-2 | 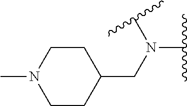 [SMILES: *N(*)CC1CCN(C)CC1] |
| 2A2-1 | 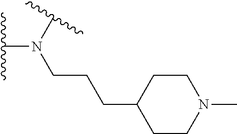 [SMILES: *N(*)CCCC1CCN(C)CC1] |
| 2A2-2 | 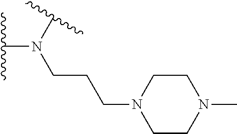 [SMILES: *N(*)CCCN1CCN(C)CC1] |
| 2A3 | 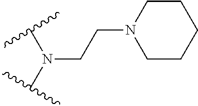 [SMILES: *N(*)CCN1CCCCC1] |
| 2A4 | 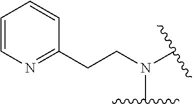 [SMILES: *N(*)CCc1ccccn1] |
| 2A5 | 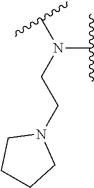 [SMILES: *N(*)CCN1CCCC1] |
| 2A6 | 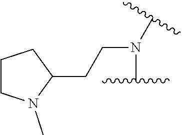 [SMILES: *N(*)CCC1CCCN1C] |
| 2A7-1 | 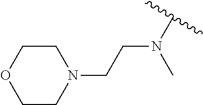 [SMILES: *N(C)CCN1CCOCC1] |
| 2A7-2 | 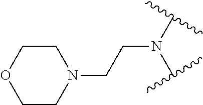 [SMILES: *N(*)CCN1CCOCC1] |
| 2A8 | 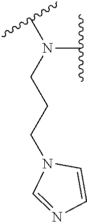 [SMILES: *N(*)CCCn1ccnc1] |
| 2A9 | 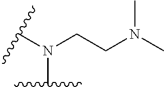 [SMILES: *N(*)CCN(C)C] |
| 2A9V | 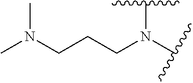 [SMILES: *N(*)CCCN(C)C] |
| 2A10 | 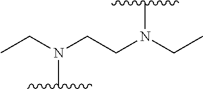 [SMILES: *N(CC)CCN(*)CC] |
| 2A11 | 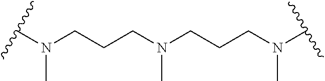 [SMILES: *N(C)CCCN(C)CCCN(*)C] |
| 2A12 | 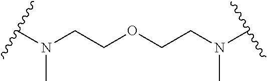 [SMILES: *N(C)CCOCCN(*)C] |
| 3A1 | 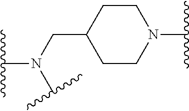 [SMILES: *N(*)CC1CCN(*)CC1] |
| 3A2 | 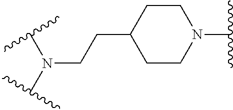 [SMILES: *N(*)CCC1CCN(*)CC1] |
| 3A3 | 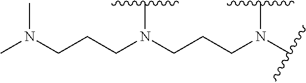 [SMILES: *N(*)CCCN(*)CCCN(C)C] |
| 3A4 | 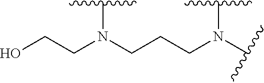 [SMILES: *N(*)CCCN(*)CCO] |
| 3A5 | 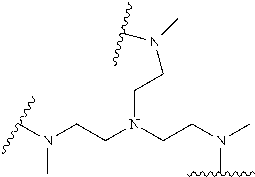 [SMILES: *N(C)CCN(CCN(*)C)CCN(*)C] |
| 3A6 | 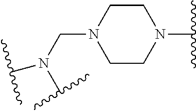 [SMILES: *N(*)CN1CCN(*)CC1] |
| 3A7 | 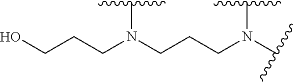 [SMILES: *N(*)CCCN(*)CCCO] |
| 4A1 | 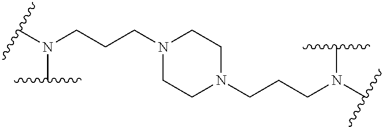 [SMILES: *N(*)CCCN1CCN(CCCN(*)*)CC1] |
| 4A2 | 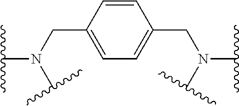 [SMILES: *N(*)Cc1ccc(CN(*)*)cc1] |
| 4A3 | 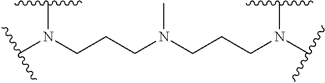 [SMILES: *N(*)CCCN(C)CCCN(*)*] |
| 4A4 | 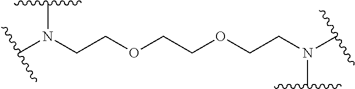 [SMILES: *N(*)CCOCCOCCN(*)*] |
| 5A1 | 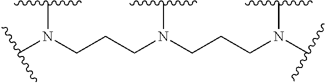 [SMILES: *N(*)CCCN(*)CCCN(*)*] |
| 5A2-1 (5-arm) | 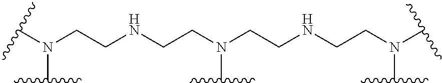 [SMILES: *N(*)CCNCCN(*)CCNCCN(*)*] |
| 5A2-2 (5-arm) | 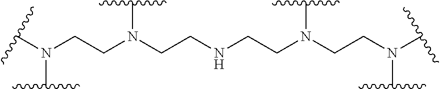 [SMILES: *N(*)CCN(*)CCNCCN(*)CCN(*)*] |
| 5A2-3 (5-arm) | 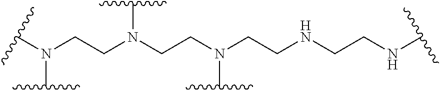 [SMILES: *NCCNCCN(*)CCN(*)CCN(*)*] |
| 5A2-4 (5-arm) | 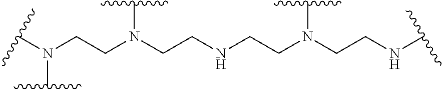 [SMILES: *NCCN(*)CCNCCN(*)CCN(*)*] |
| 5A3-1 (5-arm) | 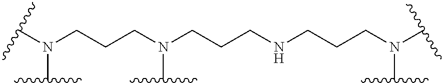 [SMILES: *N(*)CCCNCCCN(*)CCCN(*)*] |
| 5A4-1 (5-arm) | 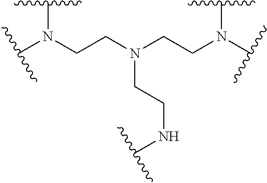 [SMILES: *NCCN(CCN(*)*)CCN(*)*] |
| 5A5 | 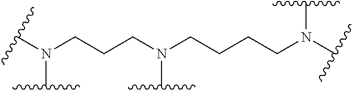 [SMILES: *N(*)CCCCN(*)CCCN(*)*] |
| 5A6 | 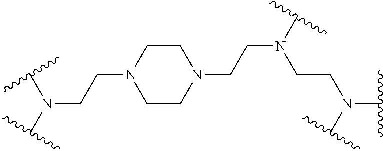 [SMILES: *N(*)CCN(*)CCN1CCN(CCN(*)*)CC1] |
| 5A2-4 (6-arm) | 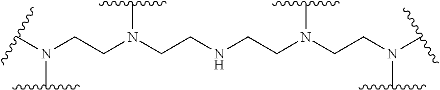 [SMILES: *N(*)CCN(*)CCNCCN(*)CCN(*)*] |
| 5A2-5 (6 arm) | 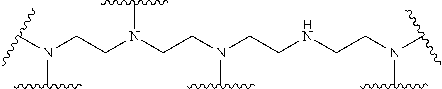 [SMILES: *N(*)CCNCCN(*)CCN(*)CCN(*)*] |
| 5A2-6 (6 arm) | 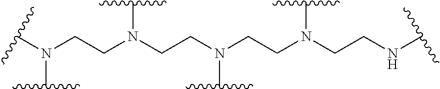 [SMILES: *NCCN(*)CCN(*)CCN(*)CCN(*)*] |
| 6A2 | 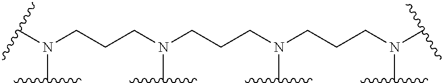 [SMILES: *N(*)CCCN(*)CCCN(*)CCCN(*)*] |
| 5A3-2 (6 arm) | 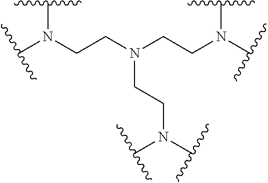 [SMILES: *N(*)CCN(CCN(*)*)CCN(*)*] |
| 5A4-2 (6 arm) | 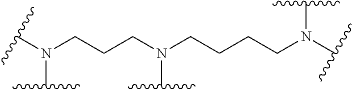 [SMILES: *N(*)CCCCN(*)CCCN(*)*] |
| 6A4 | 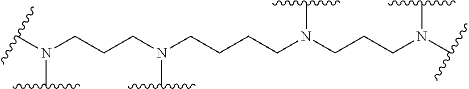 [SMILES: *N(*)CCCN(*)CCCCN(*)CCCN(*)*] |
| 1H1 | 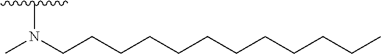 |
| 1H2 | 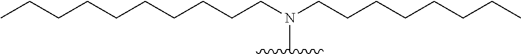 [SMILES: *N(CCCCCCCC)CCCCCCCCCC] |
| 1H3 | 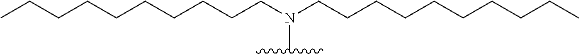 [SMILES: *N(CCCCCCCCCC)CCCCCCCCCC] |
| 2H1 | 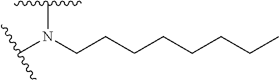 [SMILES: *N(*)CCCCCCCC] |
| 2H2 | 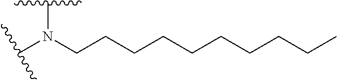 [SMILES: *N(*)CCCCCCCCCC] |
| 2H3 | 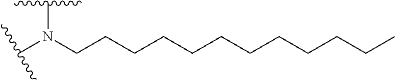 [SMILES: *N(*)CCCCCCCCCCCC] |
| 2H4 | 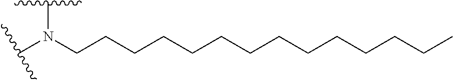 [SMILES: *N(*)CCCCCCCCCCCCCC] |
| 2H5 | 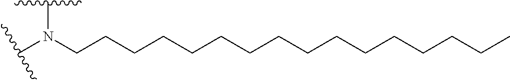 [SMILES: *N(*)CCCCCCCCCCCCCCCC] |
| 2H6 | 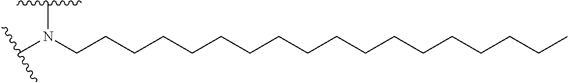 [SMILES: *N(*)CCCCCCCCCCCCCCCCCC] |

In some embodiments of X_{Core}, the core comprises a structural formula selected from the group consisting of:

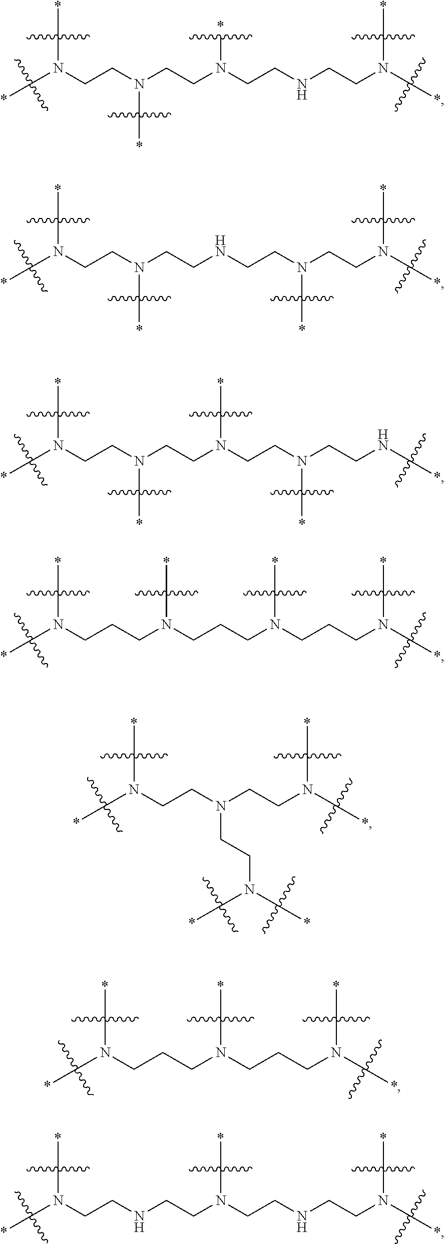

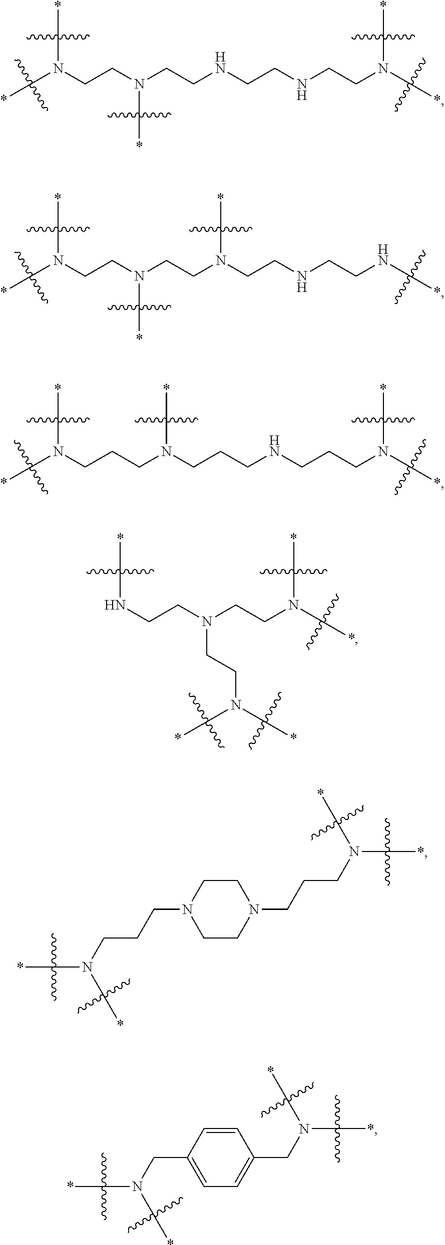

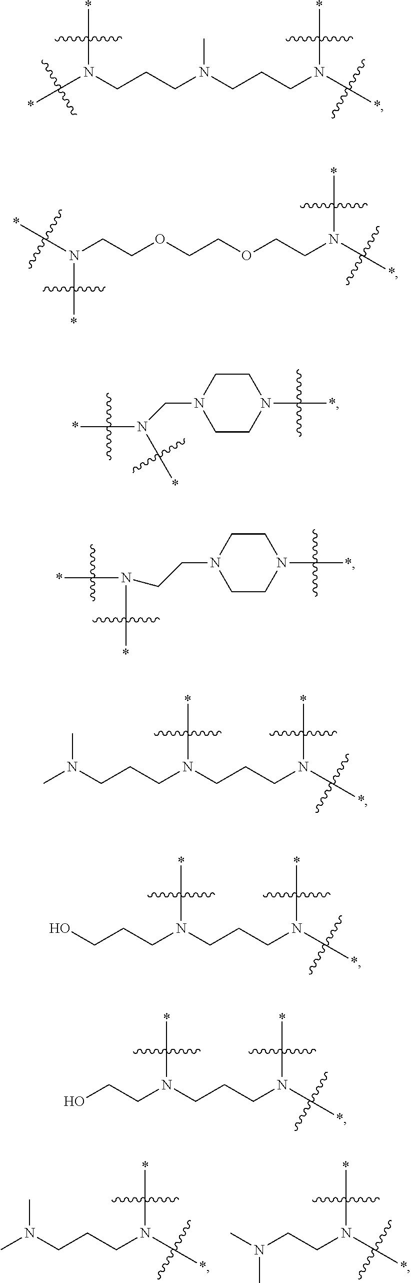

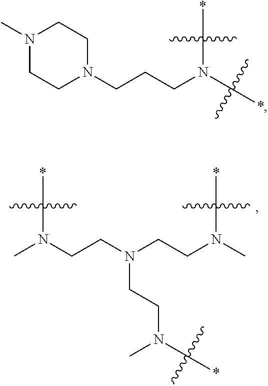

and pharmaceutically acceptable salts thereof, wherein * indicates a point of attachment of the core to a branch of the plurality of branches or H. In some embodiments, wherein * indicates a point of attachment of the core to a branch of the plurality of branches.

In some embodiments of X_{Core}, the core has the structure

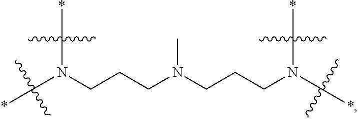

wherein * indicates a point of attachment of the core to a branch of the plurality of branches or H. In some embodiments, at least 2 branches are attached to the core. In some embodiments, at least 3 branches are attached to the core. In some embodiments, at least 4 branches are attached to the core.

In some embodiments of X_{Core}, the core has the structure

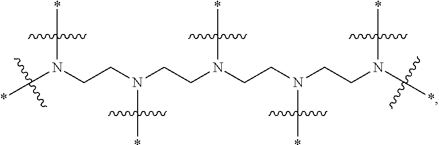

wherein * indicates a point of attachment of the core to a branch of the plurality of branches or H. In some embodiments, at least 4 branches are attached to the core. In some embodiments, at least 5 branches are attached to the core. In some embodiments, at least 6 branches are attached to the core.

In some embodiments, the plurality (N) of branches comprises at least 3 branches, at least 4 branches, at least 5 branches. In some embodiments, the plurality (N) of branches comprises at least 3 branches. In some embodiments, the plurality (N) of branches comprises at least 4 branches. In some embodiments, the plurality (N) of branches comprises at least 5 branches.

In some embodiments of X_{Branch}, g is 1, 2, 3, or 4. In some embodiments of X_{Branch}, g is 1. In some embodiments of X_{Branch}, g is 2. In some embodiments of X_{Branch}, g is 3. In some embodiments of X_{Branch}, g is 4.

In some embodiments of X_{Branch}, Z=2(g−1) and when g=1, G=0. In some embodiments of X_{Branch}, Z=2(g−1) and GΣᵢ₌₀^{i=g-2}2ⁱ, when g≠1.

In some embodiments of X_{Branch}, g=1, G=0, Z=1, and each branch of the plurality of branches comprises a structural formula each branch of the plurality of branches comprises a structural formula

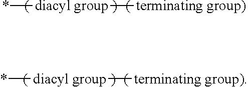

In some embodiments of X_{Branch}, g=2, G=1, Z=2, and each branch of the plurality of branches comprises a structural formula

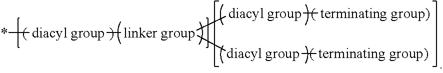

In some embodiments of X_{Branch}, g=3, G=3, Z=4, and each branch of the plurality of branches comprises a structural formula

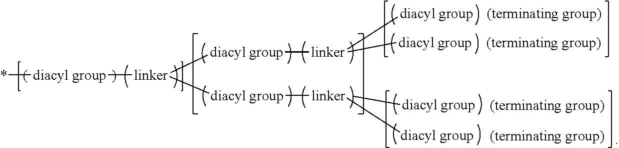

In some embodiments of X_{Branch}, g=4, G=7, Z=8, and each branch of the plurality of branches comprises a structural formula

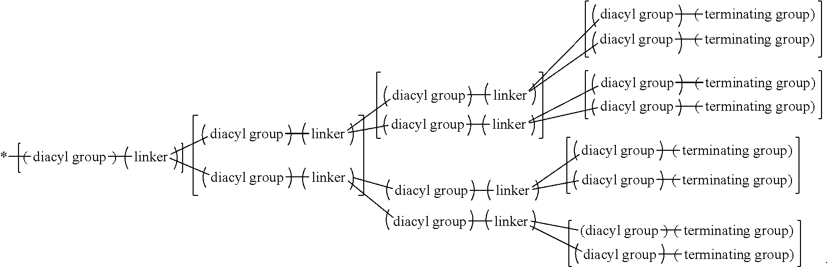

In some embodiments, the dendrimers or dendrons described herein with a generation (g)=1 has the structure:

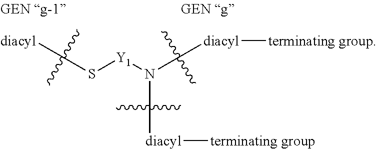

In some embodiments, the dendrimers or dendrons described herein with a generation (g)=1 has the structure:

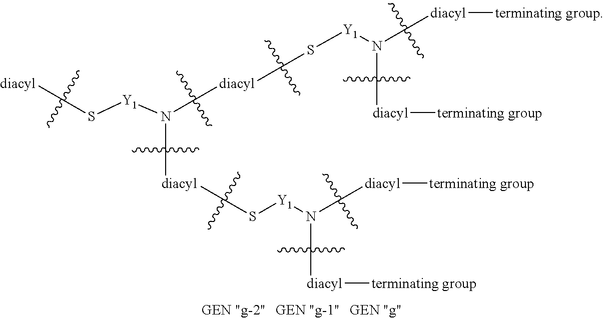

An example formulation of the dendrimers or dendrons described herein for generations 1-4 is shown in Table B. The number of diacyl groups, linker groups, and terminating groups can be calculated based on g.

TABLE B
Formulation of Dendrimer or Dendron
Groups Based on Generation (g)

| | g = 1 | g = 2 | g = 3 | g = 4 | |
|---|---|---|---|---|---|
| # of | 1 | 1 + 2 = 3 | 1 + 2 + | 1 + 2 + 2² + | 1 + 2 + . . . + |
| diacyl grp | | | 2² = 7 | 2³ = 15 | 2^{g-1} |
| # of | 0 | 1 | 1 + 2 | 1 + 2 + 2² | 1 + 2 + . . . + |
| linker grp | | | | | 2^{g-2} |
| # of | 1 | 2 | 2² | 2³ | 2^{(g-1)} |
| terminating | | | | | |
| grp | | | | | |

In some embodiments, the diacyl group independently comprises a structural formula

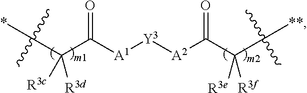

* indicates a point of attachment of the diacyl group at the proximal end thereof, and ** indicates a point of attachment of the diacyl group at the distal end thereof.

In some embodiments of the diacyl group of X_{Branch}, Y₃ is independently at each occurrence an optionally substituted; alkylene, an optionally substituted alkenylene, or an optionally substituted arenylene. In some embodiments of the diacyl group of X_{Branch}, Y₃ is independently at each occurrence an optionally substituted alkylene (e.g., C₁-C₁₂). In some embodiments of the diacyl group of X_{Branch}, Y₃ is independently at each occurrence an optionally substituted alkenylene (e.g., C₁-C₁₂). In some embodiments of the diacyl group of X_{Branch}, Y₃ is independently at each occurrence an optionally substituted arenylene (e.g., C₁-C₁₂).

In some embodiments of the diacyl group of X_{Branch}, A₁ and A₂ are each independently at each occurrence —O—, —S—, or —NR⁴—. In some embodiments of the diacyl group of X_{Branch}, A and A₂ are each independently at each occurrence —O—. In some embodiments of the diacyl group of X_{Branch}, A₁ and A₂ are each independently at each occurrence-S—. In some embodiments of the diacyl group of X_{Branch}, A₁ and A₂ are each independently at each occurrence—NR⁴— and R⁴ is hydrogen or optionally substituted alkyl (e.g., C₁-C₆). In some embodiments of the diacyl group of X_{Branch}, m¹ and m² are each independently at each occurrence 1, 2, or 3. In some embodiments of the diacyl group of X_{Branch}, m¹ and m² are each independently at each occurrence 1. In some embodiments of the diacyl group of X_{Branch}, m¹ and m² are each independently at each occurrence 2. In some embodiments of the diacyl group of X_{Branch}, m¹ and m² are each independently at each occurrence 3. In some embodiments of the diacyl group of X_{Branch}, R^{3c}, R^{3d}, R^{3e}, and R^{3f} are each independently at each occurrence hydrogen or an optionally substituted alkyl. In some embodiments of the diacyl group of X_{Branch}, R^{3c}, R^{3d}, R³, and R^{3f} are each independently at each occurrence hydrogen. In some embodiments of the diacyl group of X_{Branch}, R^{3c}, R^{3d}, R^{3e}, and R^{3f} are each independently at each occurrence an optionally substituted (e.g., C₁-C₈) alkyl.

In some embodiments of the diacyl group, A₁ is —O— or —NH—. In some embodiments of the diacyl group, A₁ is —O—. In some embodiments of the diacyl group, A₂ is —O— or —NH—. In some embodiments of the diacyl group, A₂ is —O—. In some embodiments of the diacyl group, Y₃ is C₁-C₁₂ (e.g., C₁-C₆, such as C₁-C₃) alkylene.

In some embodiments of the diacyl group, the diacyl group independently at each occurrence comprises a structural formula

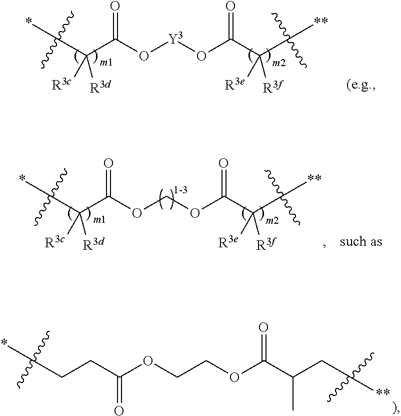

and optionally R^{3c}. R^{3d}R^{3e}, and R^{3f} are each independently at each occurrence hydrogen or C₁-C₃ alkyl.

In some embodiments, linker group independently comprises a structural formula

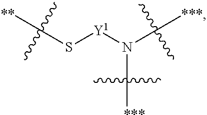

** indicates a point of attachment of the linker to a proximal diacyl group, and indicates a point of attachment of the linker to a distal diacyl group.

In some embodiments of the linker group of X_{Branch} if present, Y₁ is independently at each occurrence an optionally substituted alkylene, an optionally substituted alkenylene, or an optionally substituted arenylene. In some embodiments of the linker group of X_{Branch} if present, Y₁ is independently at each occurrence an optionally substituted alkylene (e.g., C₁-C₁₂). In some embodiments of the linker group of X_{Branch} if present, Y₁ is independently at each occurrence an optionally substituted alkenylene (e.g., C₁-C₁₂). In some embodiments of the linker group of X_{Branch} if present, Y₁ is independently at each occurrence an optionally substituted arenylene (e.g., C₁-C₁₂).

In some embodiments of the terminating group of X_{Branch}, each terminating group is independently selected from optionally substituted alkylthiol and optionally substituted alkenylthiol. In some embodiments of the terminating group of X_{Branch}, each terminating group is an optionally substituted alkylthiol (e.g., C₁-C₁₈, such as C₄-C₁₈). In some embodiments of the terminating group of X_{Branch}, each terminating group is optionally substituted alkenylthiol (e.g., C₁-C₁₈, such as C₄-C₁₈).

In some embodiments of the terminating group of X_{Branch}, each terminating group is independently C₁-C₁₈ alkenylthiol or C₁-C₁₈ alkylthiol, and the alkyl or alkenyl moiety is optionally substituted with one or more substituents each independently selected from halogen, C₆-C₁₂ aryl, C₁-C₁₂ alkylamino, C₄-C₆ N-heterocycloalkyl, —OH, —C(O)OH, —C(O)N(C₁-C₃ alkyl)-(C₁-C₆ alkylene)-(C₁-C₁₂ alkylamino), —C(O)N(C₁-C₃ alkyl)-(C₁-C₆ alkylene)-(C₄-C₆ N-heterocycloalkyl), —C(O)—(C₁-C₁₂ alkylamino), and —C(O)—(C₄-C₆ N-heterocycloalkyl), and the C₄-C₆ N-heterocycloalkyl moiety of any of the preceding substituents is optionally substituted with C₁-C₃ alkyl or C₁-C₃ hydroxyalkyl.

In some embodiments of the terminating group of X_{Branch}, each terminating group is independently C₁-C₁₈ (e.g., C₄-C₁₈) alkenylthiol or C₁-C₁₈ (e.g., C₄-C₁₈) alkylthiol, wherein the alkyl or alkenyl moiety is optionally substituted with one or more substituents each independently selected from halogen, C₆-C₁₂ aryl (e.g., phenyl), C₁-C₁₂ (e.g., C₁-C₈) alkylamino (e.g., C₁-C₆ mono-alkylamino (such as —NHCH₂CH₂CH₂CH₃) or C₁-C₈ di-alkylamino (such as

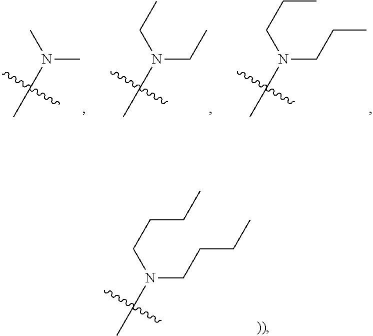

C₄-C₆ N-heterocycloalkyl (e.g., N-pyrrolidinyl

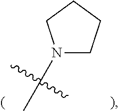 [SMILES: *N1CCCC1]

### N-piperidinyl

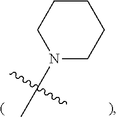 [SMILES: *N1CCCCC1]

### N-azepanyl

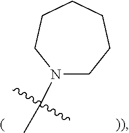 [SMILES: *N1CCCCCC1]

—OH, —C(O)OH, —C(O)N(C₁-C₃ alkyl)-(C₁-C₆ alkylene)-(C₁-C₁₂ alkylamino (e.g., mono- or di-alkylamino))

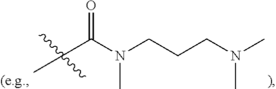 [SMILES: *C(=O)N(C)CCCN(C)C]

—C(O)N(C₁-C₃ alkyl)-(C₁-C₆ alkylene)-(C₄-C₆ N-heterocycloalkyl)

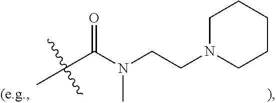 [SMILES: *C(=O)N(C)CCN1CCCCC1]

—C(O)—(C₁-C₁₂ alkylamino (e.g., mono- or di-alkylamino)), and —C(O)—(C₄-C₆ N-heterocycloalkyl)

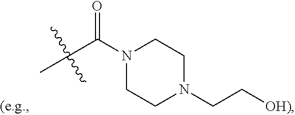

wherein the C₄-C₆ N-heterocycloalkyl moiety of any of the preceding substituents is optionally substituted with C₁-C₃ alkyl or C₁-C₃ hydroxyalkyl. In some embodiments of the terminating group of X_{Branch}, each terminating group is independently C₁-C₁₈ (e.g., C₄-C₁₈) alkylthiol, wherein the alkyl moiety is optionally substituted with one substituent-OH. In some embodiments of the terminating group of X_{Branch}, each terminating group is independently C₁-C₁₈ (e.g., C₄-C₁₈) alkylthiol, wherein the alkyl moiety is optionally substituted with one substituent selected from C₁-C₁₂ (e.g., C₁-C₈) alkylamino (e.g., C₁-C₆ mono-alkylamino (such as —NHCH₂CH₂CH₂CH₃) or C₁-C₈ di-alkylamino (such as

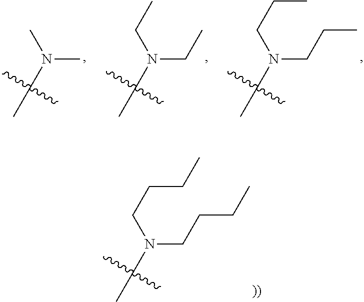

and C₄-C₆ N-heterocycloalkyl (e.g., N-pyrrolidinyl

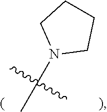 [SMILES: *N1CCCC1]

### N-piperidinyl

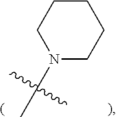 [SMILES: *N1CCCCC1]

### N-azepanyl

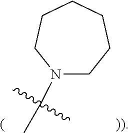 [SMILES: *N1CCCCCC1]

In some embodiments of the terminating group of X_{Branch}, each terminating group is independently C₁-C₁₈ (e.g., C₄-C₁₈) alkenylthiol or C₁-C₁₈ (e.g., C₄-C₁₈) alkylthiol. In some embodiments of the terminating group of X_{Branch}, each terminating group is independently C₁-C₁₈ (e.g., C₄-C₁₈) alkylthiol.

In some embodiments of the terminating group of X_{Branch}, each terminating group is independently a structural set forth in Table C. In some embodiments, the dendrimers or dendrons described herein can comprise a terminating group or pharmaceutically acceptable salt, or thereof selected in Table C. In some embodiments, the example terminating group of Table C are not limiting of the stereoisomers (i.e., enantiomers, diastereomers) listed.

TABLE C
Example terminating group/peripheries structures

| ID # | Structure |
|---|---|
| SC1 | 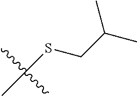 [SMILES: *SCC(C)C] |
| SC2 | 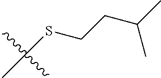 [SMILES: *SCCC(C)C] |
| SC3 | 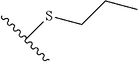 [SMILES: *SCCC] |
| SC4 | 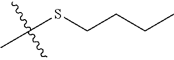 [SMILES: *SCCCC] |
| SC5 | 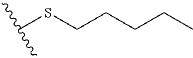 [SMILES: *SCCCCC] |
| SC6 | 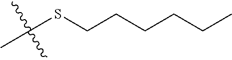 [SMILES: *SCCCCCC] |
| SC7 | 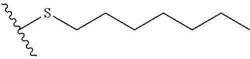 [SMILES: *SCCCCCCC] |
| SC8 | 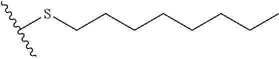 [SMILES: *SCCCCCCCC] |
| SC9 | 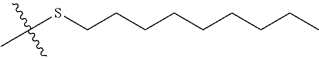 [SMILES: *SCCCCCCCCC] |
| SC10 | 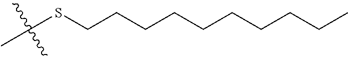 [SMILES: *SCCCCCCCCCC] |
| SC11 | 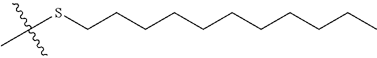 [SMILES: *SCCCCCCCCCCC] |
| SC12 | 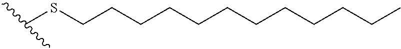 [SMILES: *SCCCCCCCCCCCC] |
| SC14 | 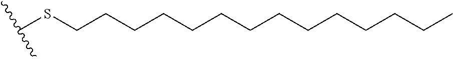 [SMILES: *SCCCCCCCCCCCCCC] |
| SC16 | 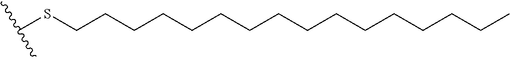 [SMILES: *SCCCCCCCCCCCCCCCC] |
| SC18 | 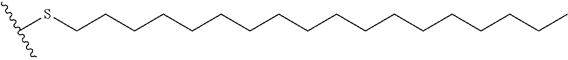 [SMILES: *SCCCCCCCCCCCCCCCCCC] |
| SC19 | 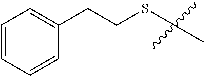 [SMILES: *SCCc1ccccc1] |
| SO1 | 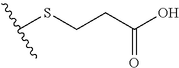 [SMILES: *SCCC(=O)O] |
| SO2 | 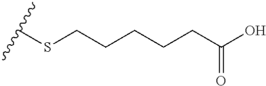 [SMILES: *SCCCCCC(=O)O] |
| SO3 | 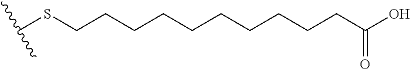 [SMILES: *SCCCCCCCCCCC(=O)O] |
| SO4 | 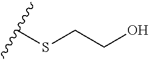 [SMILES: *SCCO] |
| SO5 | 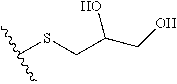 [SMILES: *SCC(O)CO] |
| SO6 | 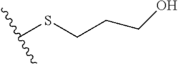 [SMILES: *SCCCO] |
| SO7 | 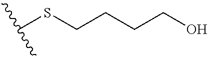 [SMILES: *SCCCCO] |
| SO8 | 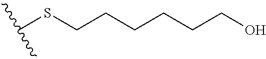 [SMILES: *SCCCCCCO] |
| SO9 | 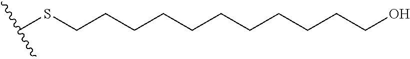 [SMILES: *SCCCCCCCCCCCO] |
| SN1 | 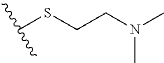 [SMILES: *SCCN(C)C] |
| SN2 | 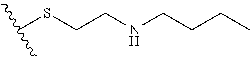 [SMILES: *SCCNCCCC] |
| SN3 | 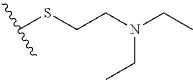 [SMILES: *SCCN(CC)CC] |
| SN4 | 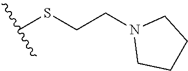 [SMILES: *SCCN1CCCC1] |
| SN5 | 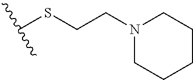 [SMILES: *SCCN1CCCCC1] |
| SN6 | 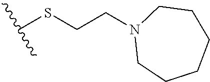 [SMILES: *SCCN1CCCCCC1] |
| SN7 | 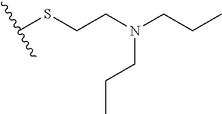 [SMILES: *SCCN(CCC)CCC] |
| SN8 | 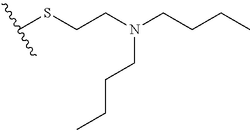 [SMILES: *SCCN(CCCC)CCCC] |
| SN9 | 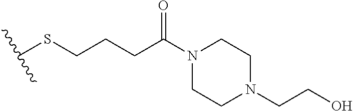 [SMILES: *SCCCC(=O)N1CCN(CCO)CC1] |
| SN10 | 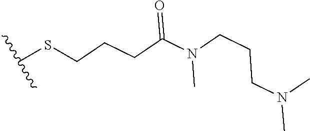 [SMILES: *SCCCC(=O)N(C)CCCN(C)C] |
| SN11 | 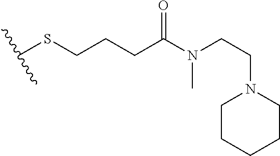 [SMILES: *SCCCC(=O)N(C)CCN1CCCCC1] |

In some embodiments, the dendrimer or dendron of Formula (X) is selected from those set forth in Table D and pharmaceutically acceptable salts thereof.

TABLE D
Example Ionizable Cationic Lipo-dendrimers

| ID # | Structure |
|---|---|
| 2A2- SC14 | 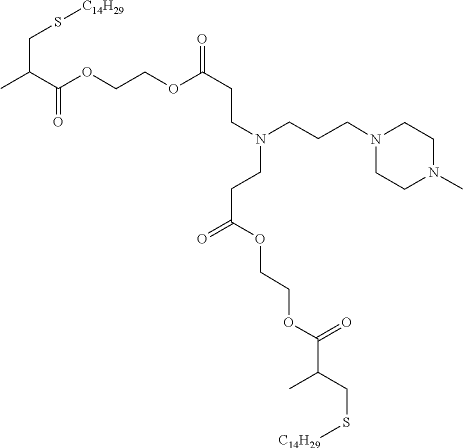 |
| 2A6- SC14 | 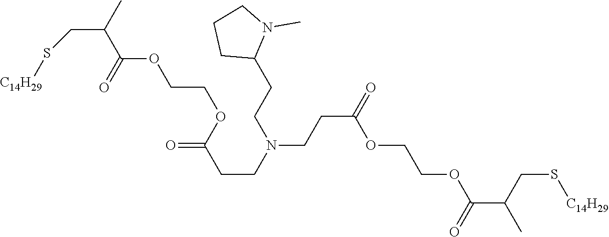 |
| 2A9- SC14 | 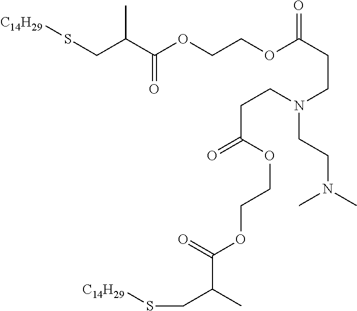 |
| 3A3- SC10 | 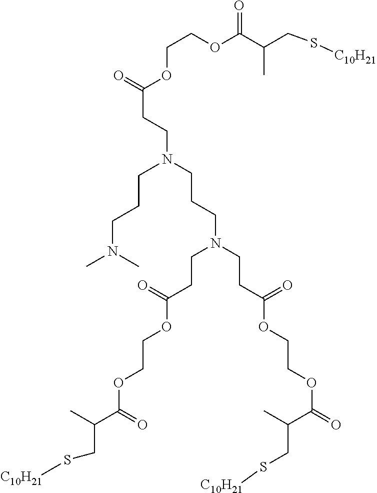 |
| 3A3- SC14 | 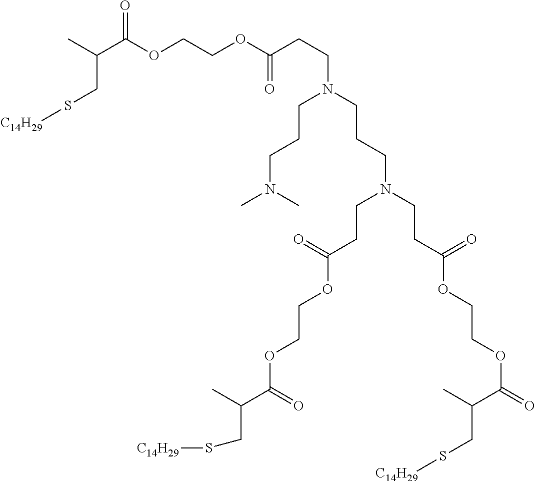 |
| 3A5- SC10 | 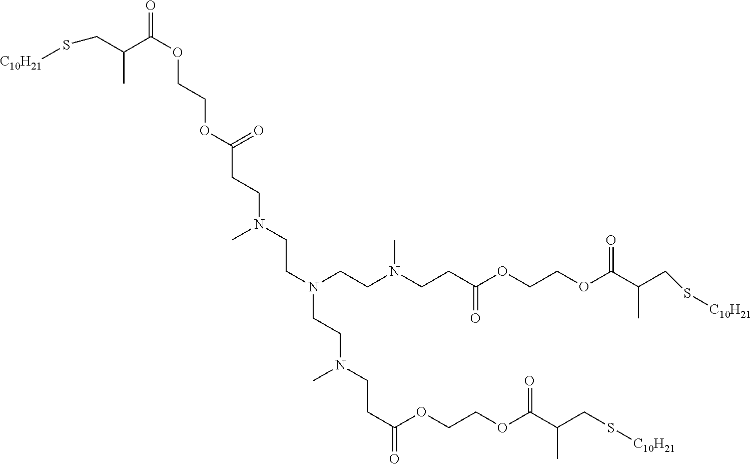 |
| 3A5- SC14 | 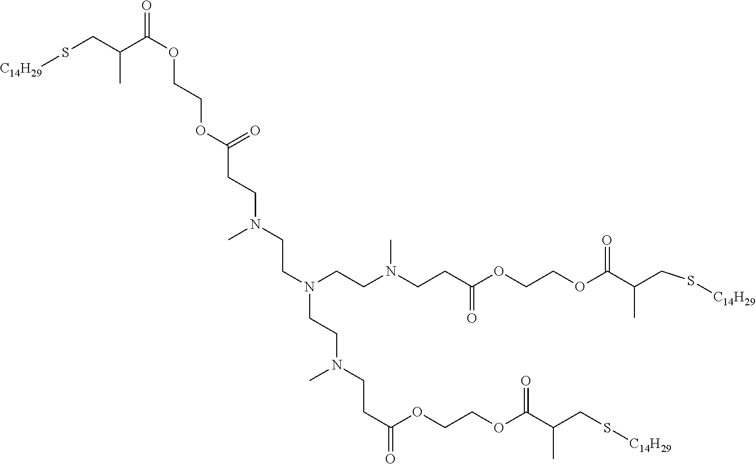 |
| 4A1- SC12 | 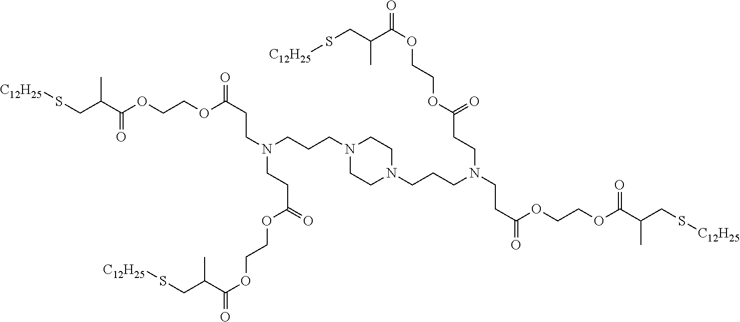 |
| 4A3- SC12 | 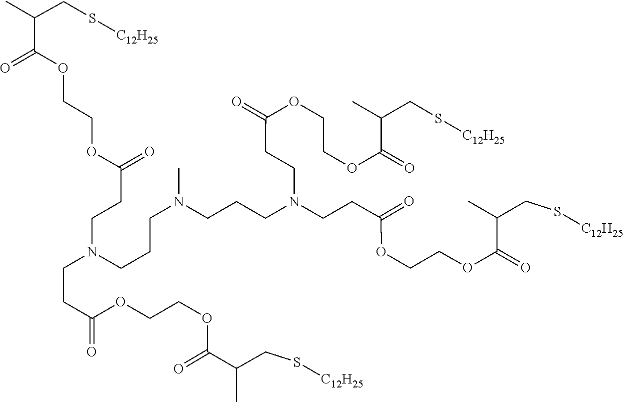 |
| 5A1- SC12 | 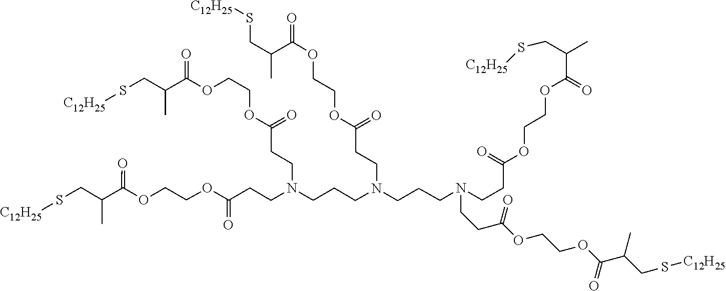 |
| 5A1- SC8 | 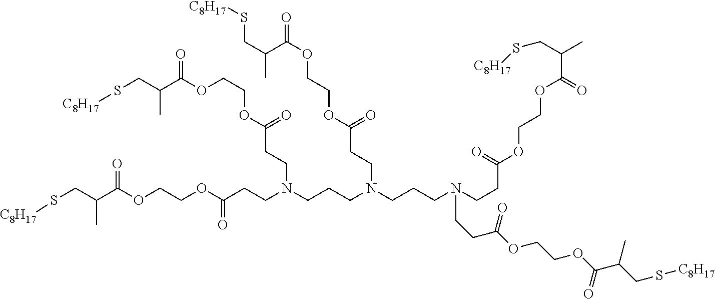 |
| 5A2- 2- SC12 (5-arm) | 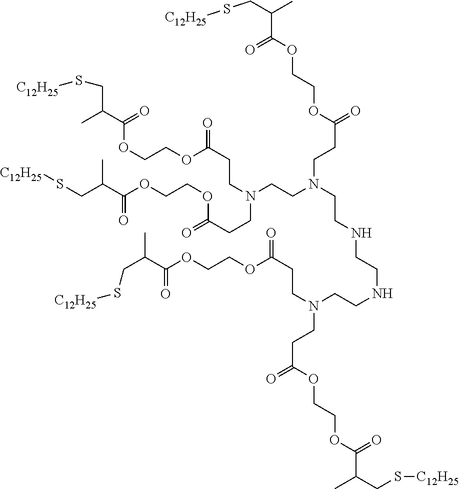 |
| 5A3- 1- SC12 (5-arm) | 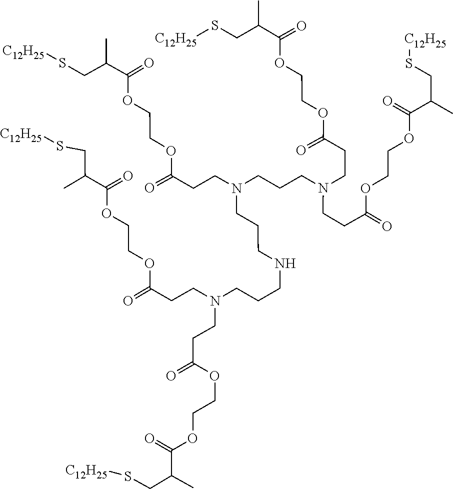 |
| 5A3- 1- SC8 (5-arm) | 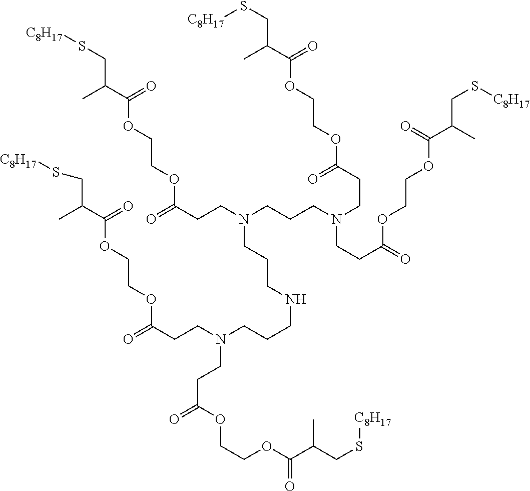 |
| 5A4- 1- SC12 (5-arm) | 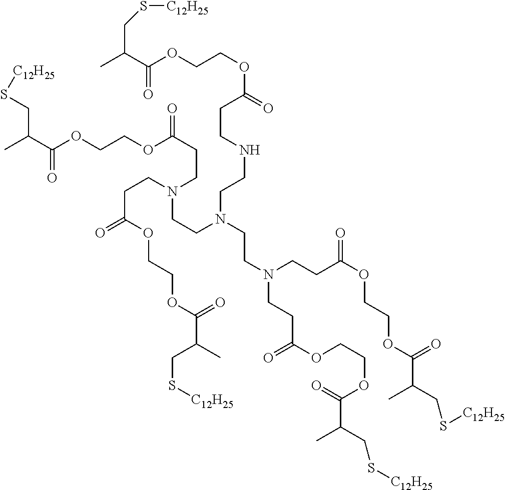 |
| 5A4- 1- SC8 (5-arm) | 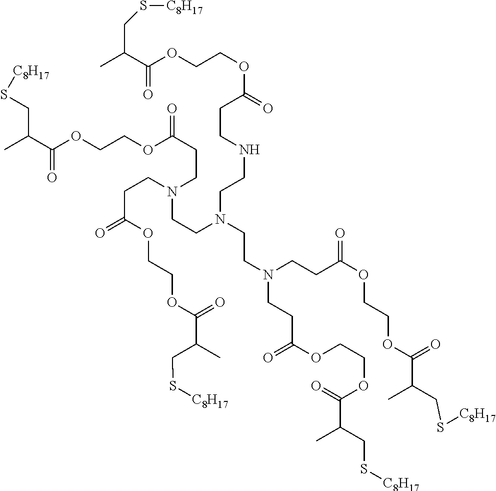 |
| 5A5- SC8 | 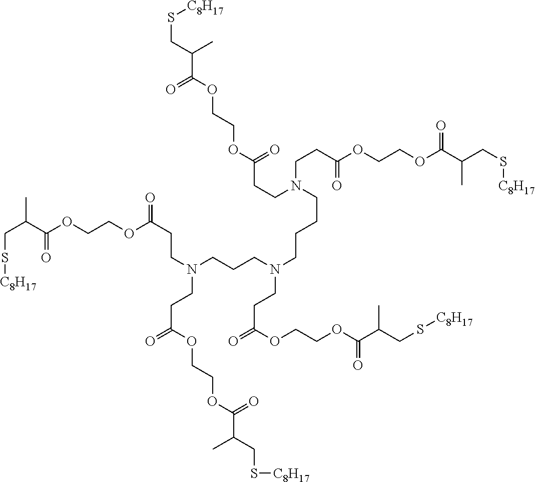 |
| 5A5- SC12 | 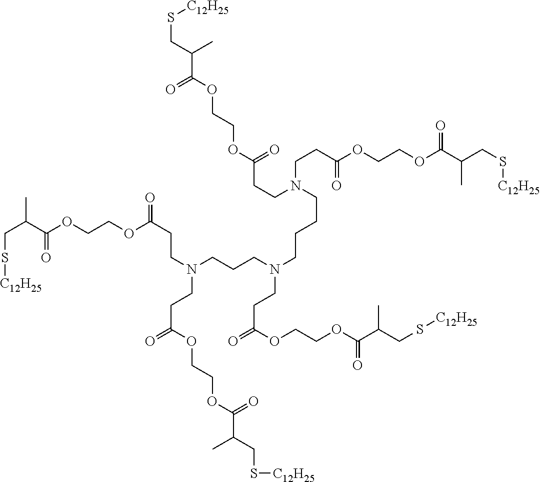 |
| 5A2- 4- SC12 (6-arm) | 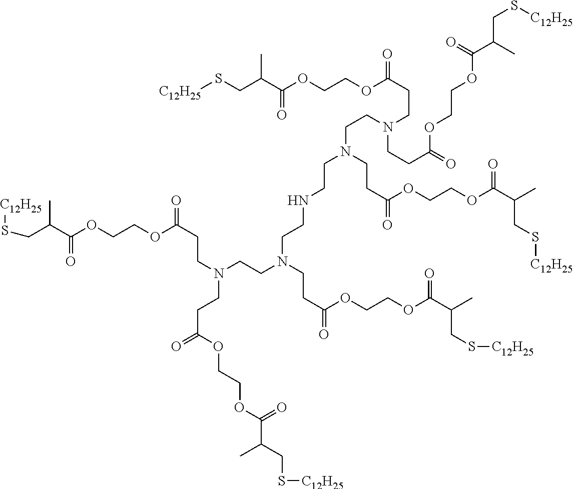 |
| 5A2- 4- SC10 (6-arm) | 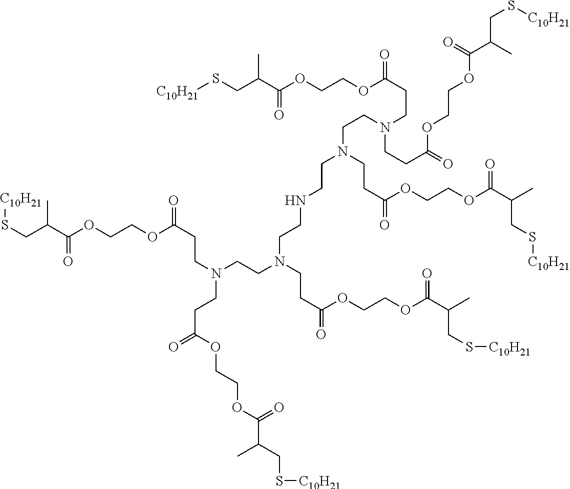 |
| 5A3- 2-- SC8 (6-arm) | 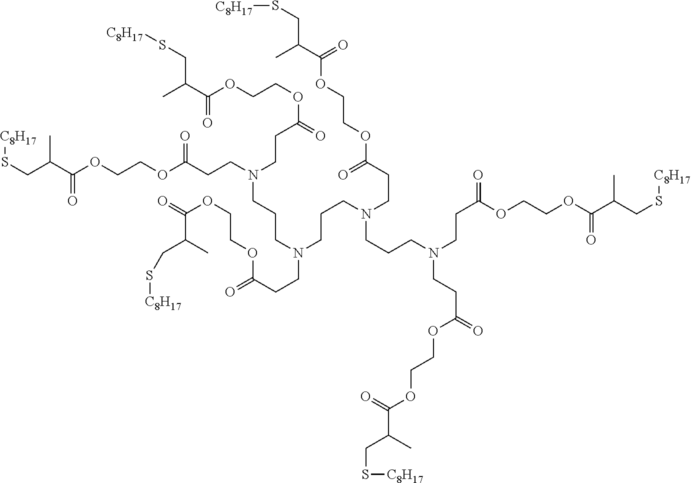 |
| 5A3- 2- SC12 (6-arm) | 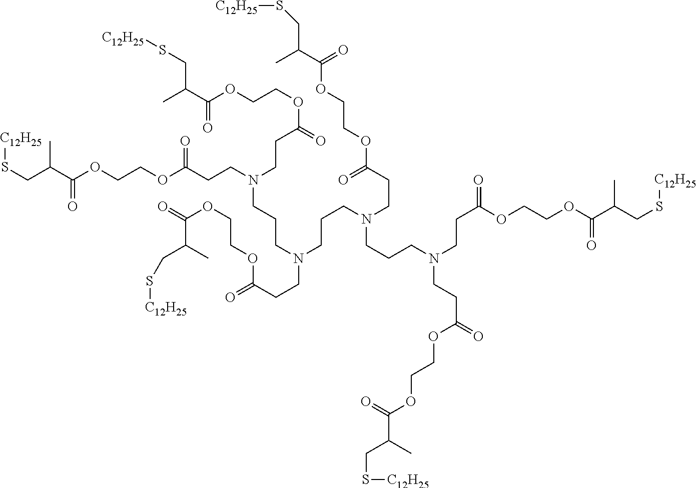 |
| 5A4- 2- SC8 (6-arm) | 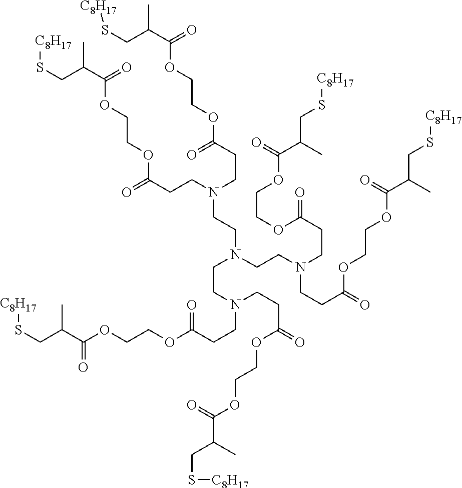 |
| 5A4- 2- SC12 (6-arm) | 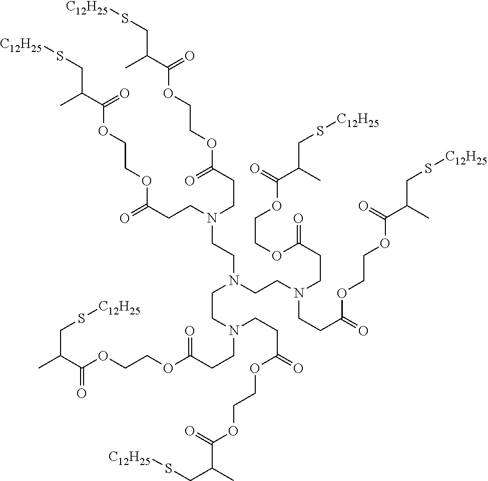 |
| 6A4- SC8 | 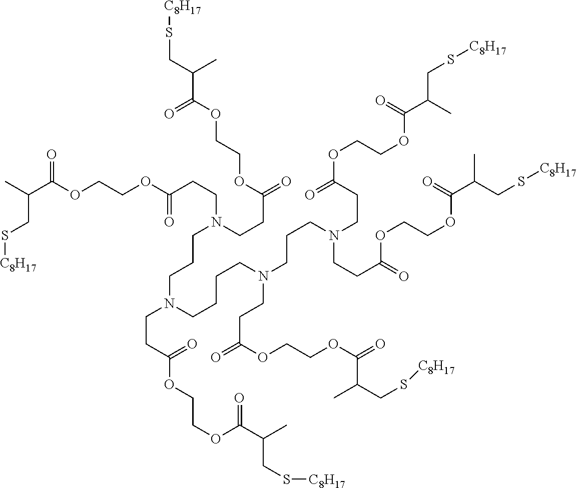 |
| 6A4- SC12 | 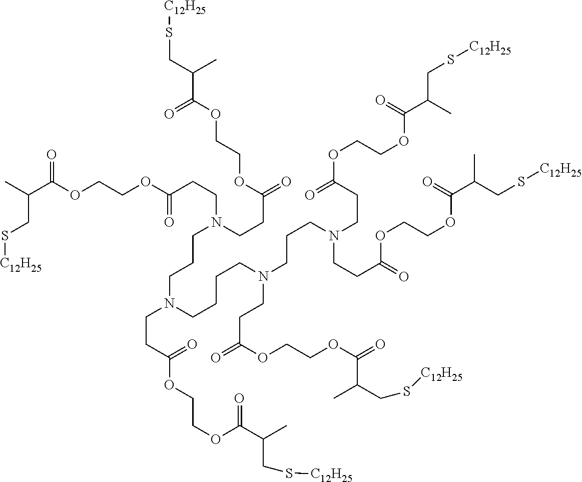 |
| 2A2- g2- SC12 | 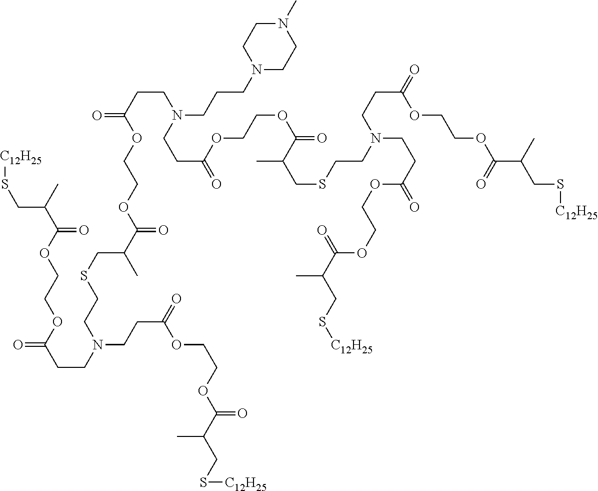 |
| 2A2- g2- SC8 | 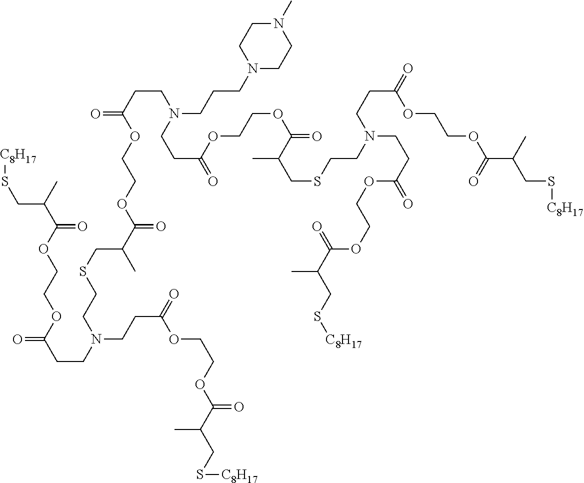 |
| 2A11- g2- SC12 | 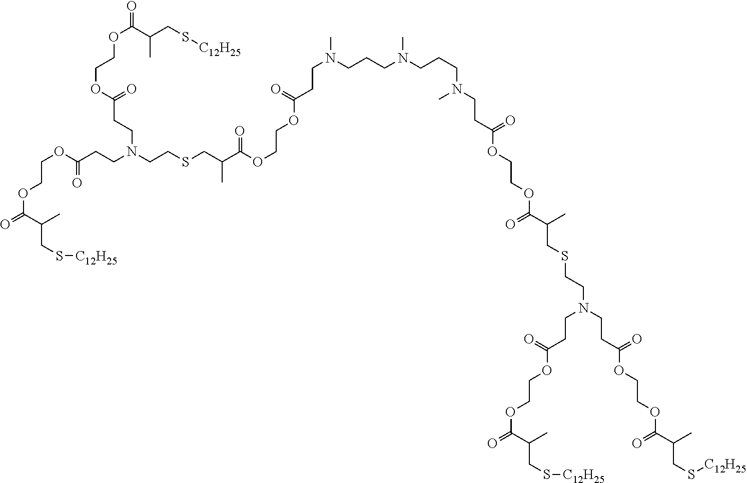 |
| 2A11- g2- SC8 | 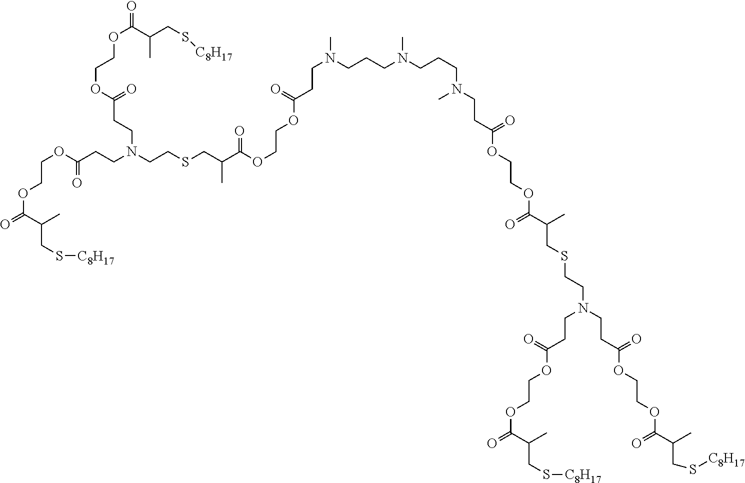 |
| 3A3- g2- SC12 | 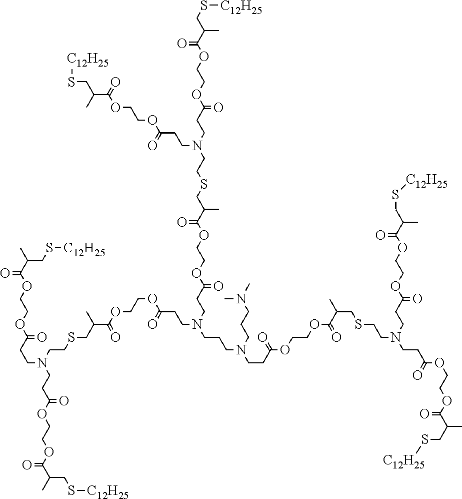 |
| 3A3- g2- SC8 | 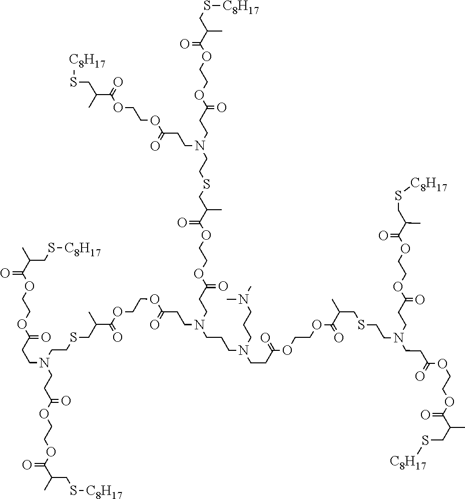 |
| 3A5- g2- SC12 | 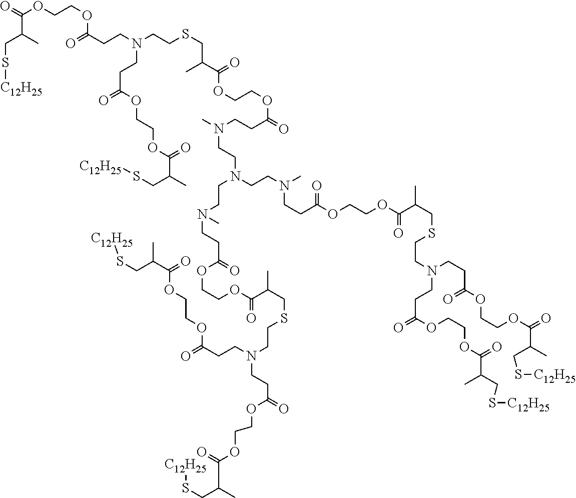 |
| 2A11- g3- SC12 | 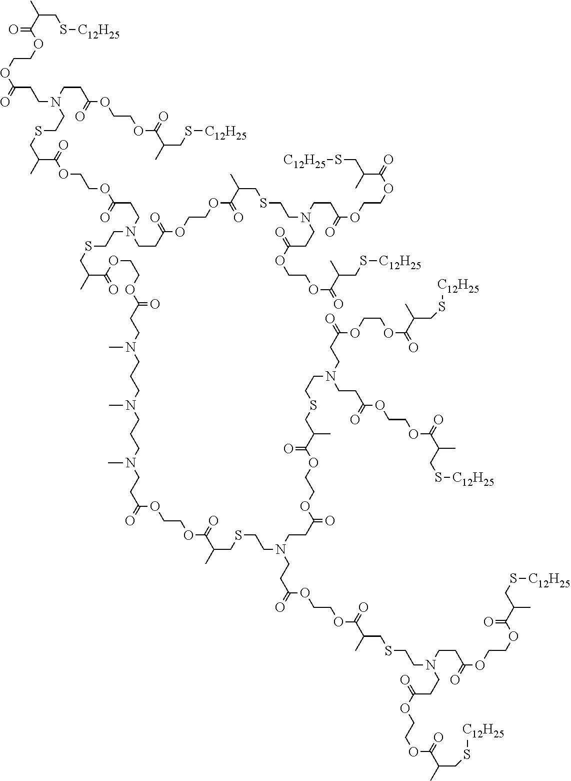 |
| 2A11- g3- SC8 | 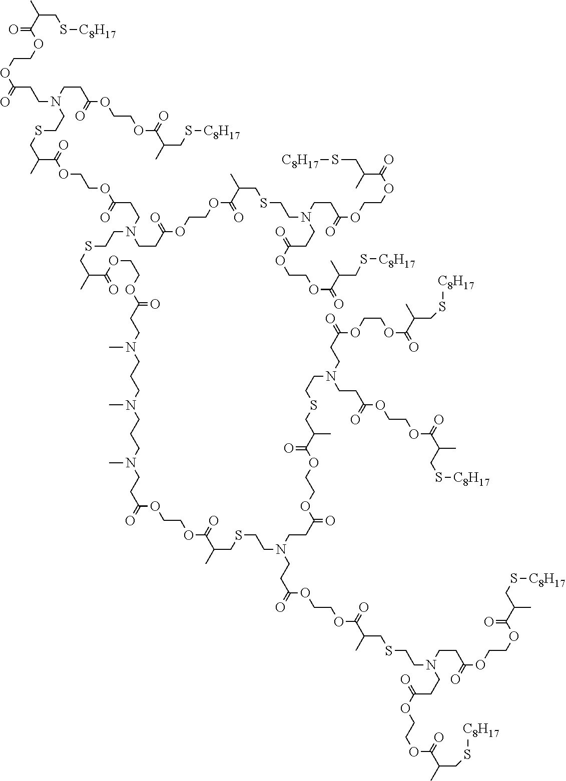 |
| 1A2- g4- SC12 | 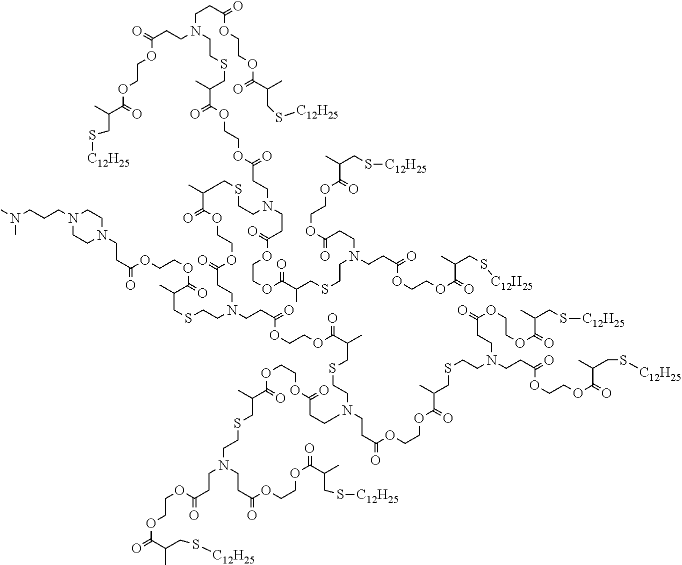 |
| 4A1- g2- SC12 | 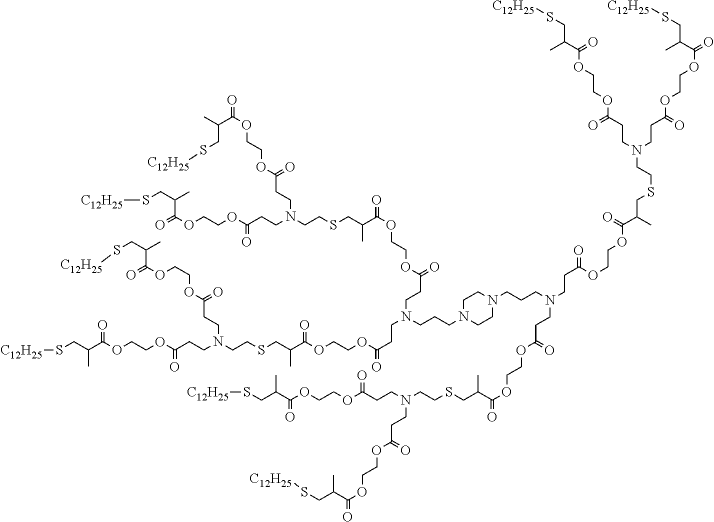 |
| 1A2- g4- SC8 | 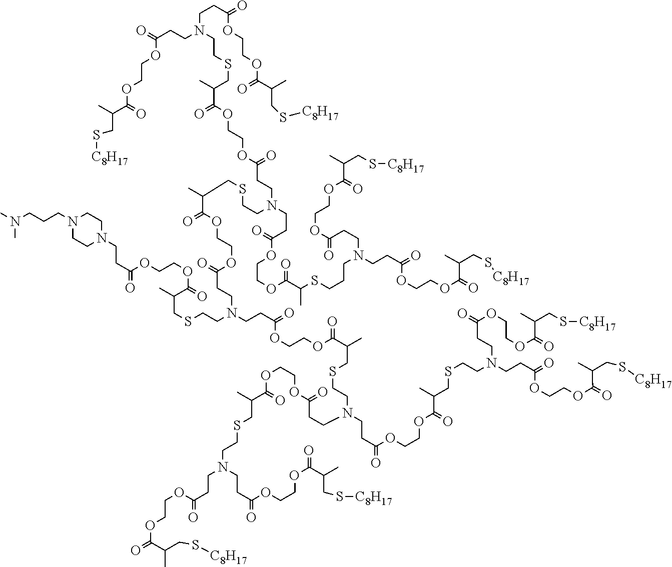 |
| 4A1- g2- SC8 | 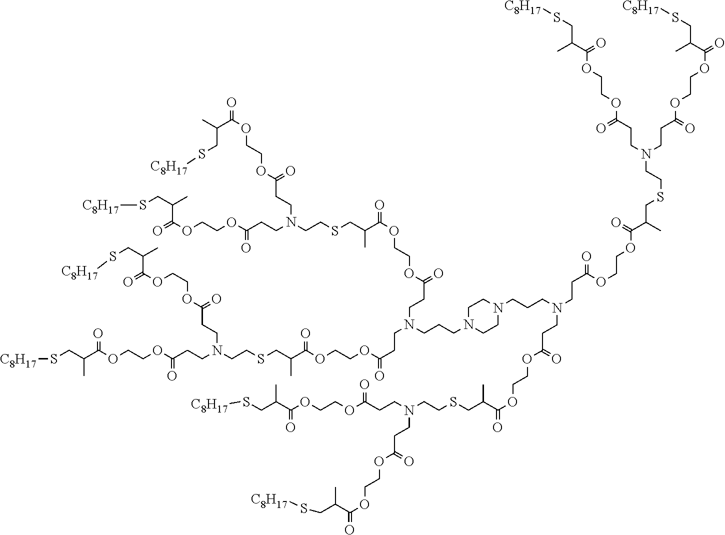 |
| 4A3- g2- SC12 | 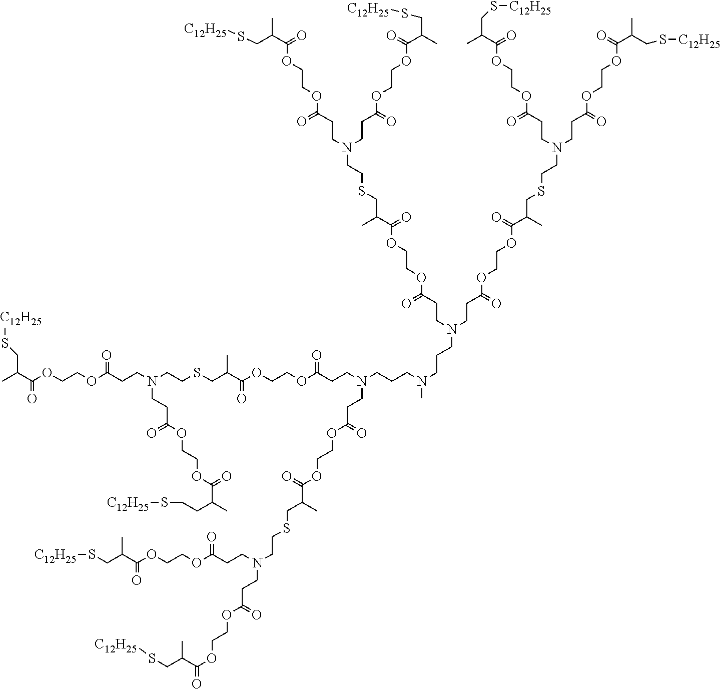 |
| 4A3- g2- SC8 | 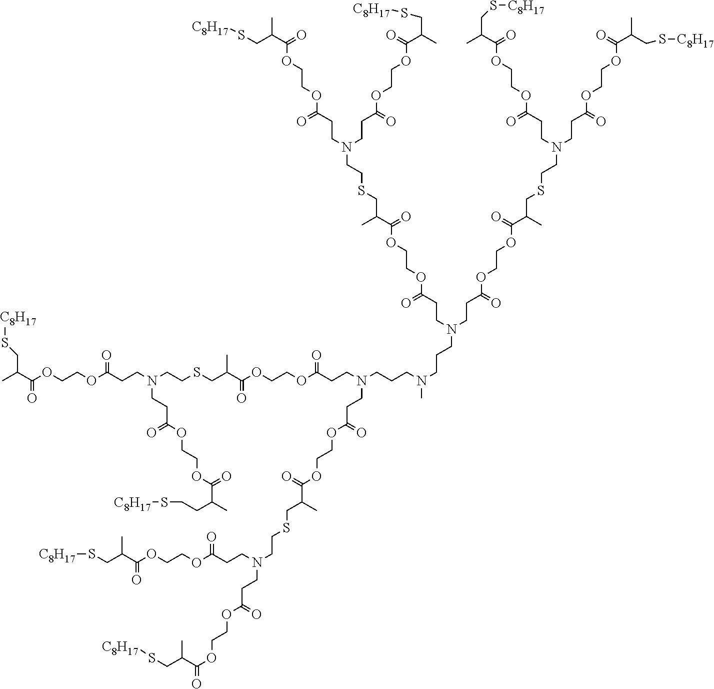 |
| 1A2- g3- SC12 | 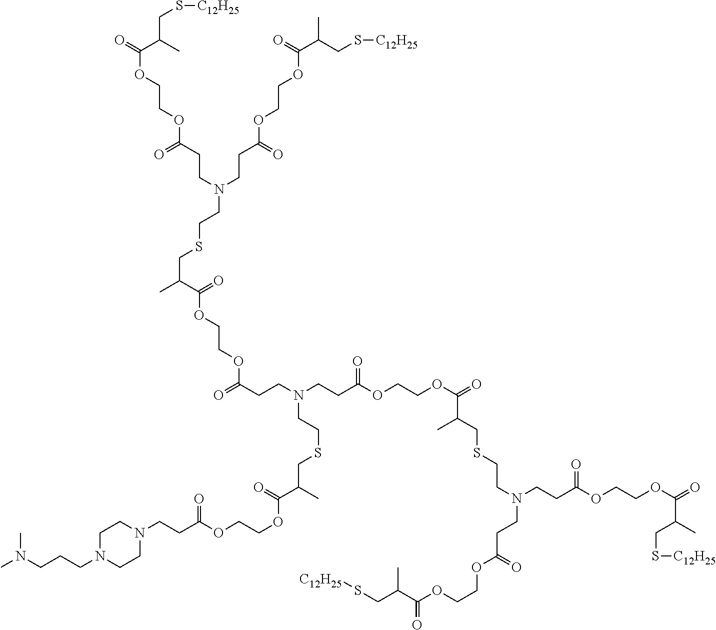 |
| 1A2- g3- SC8 | 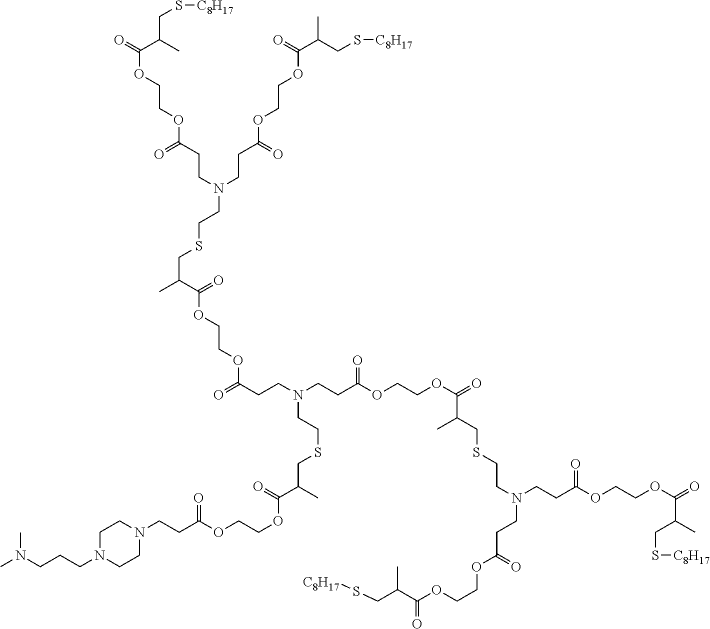 |
| 2A2- g3- SC12 | 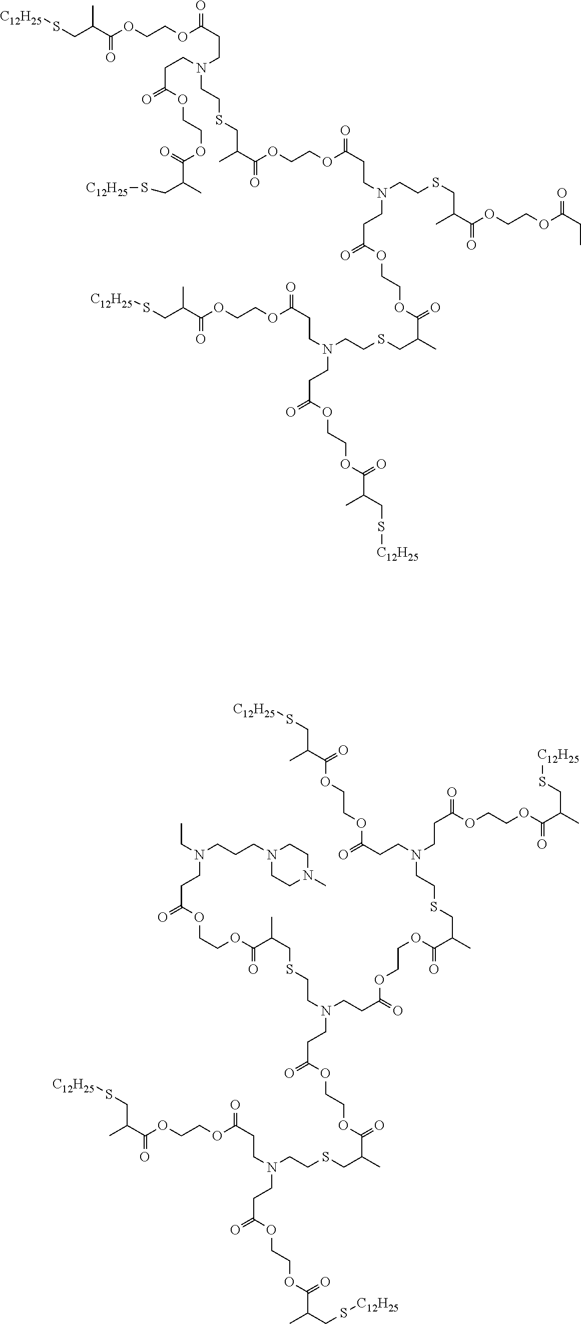 |
| 2A2- g3- SC8 | 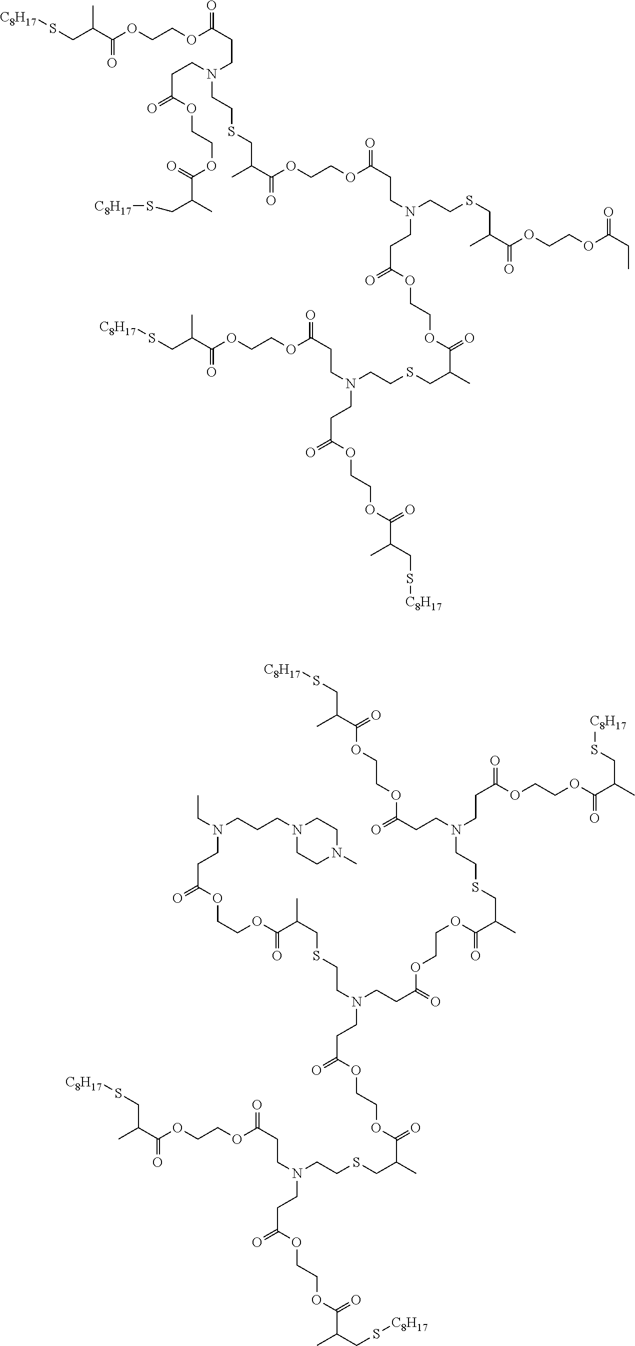 |
| 5A2- 4- SC8 (6-arm) | 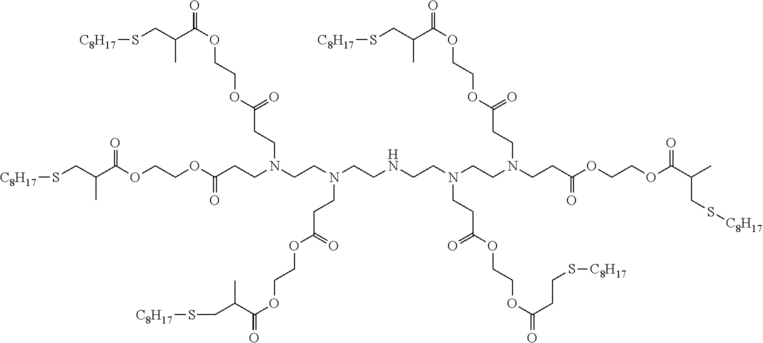 |
| 5A- 5- SC8 (6 arm) | 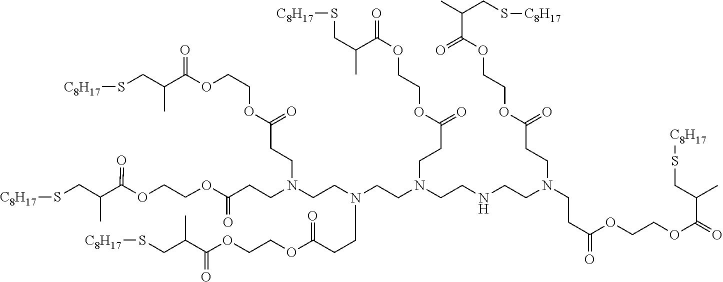 |
| 5A2- 6- SC8 (6 arm) | 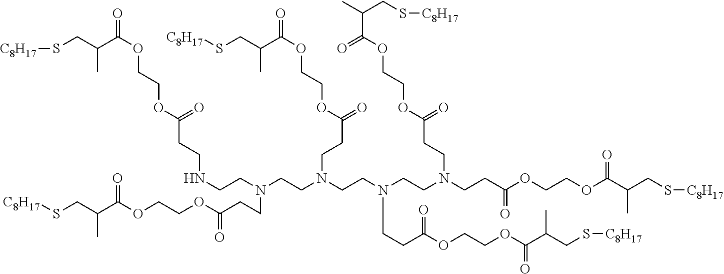 |
| 5A2- 1- SC8 (5-arm) | 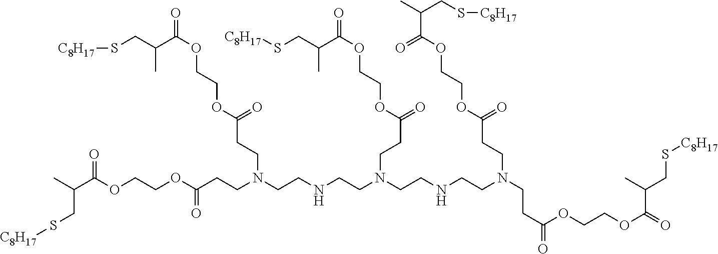 |
| 5A2- 2- SC8 | 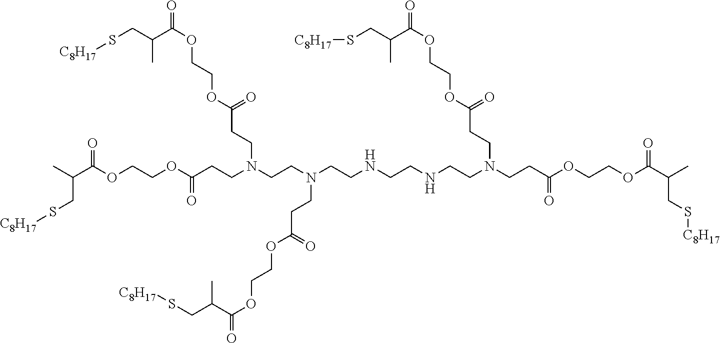 |
| 4A1- SC5 | 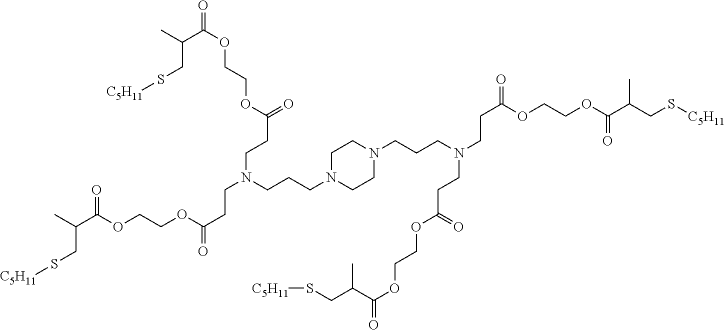 |
| 4A1- SC8 | 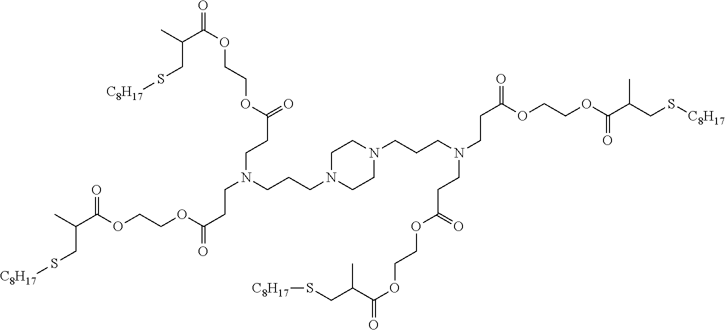 |
| 4A3- SC6 | 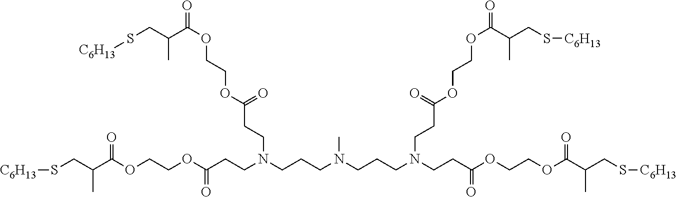 |
| 4A3- SC7 | 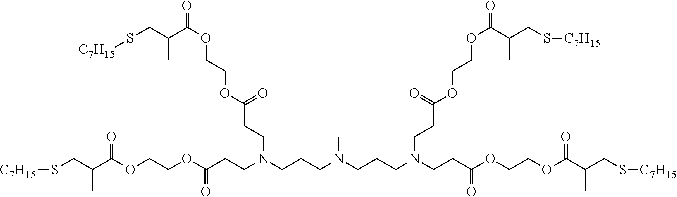 |
| 4A3- SC8 | 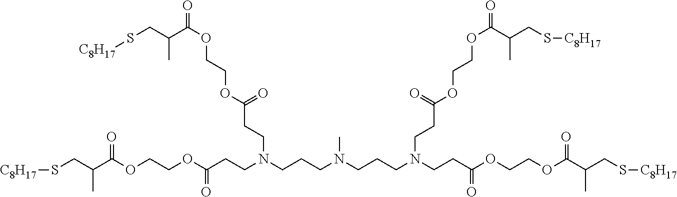 |
| 5A4- 2- SC5 (6 arm) | 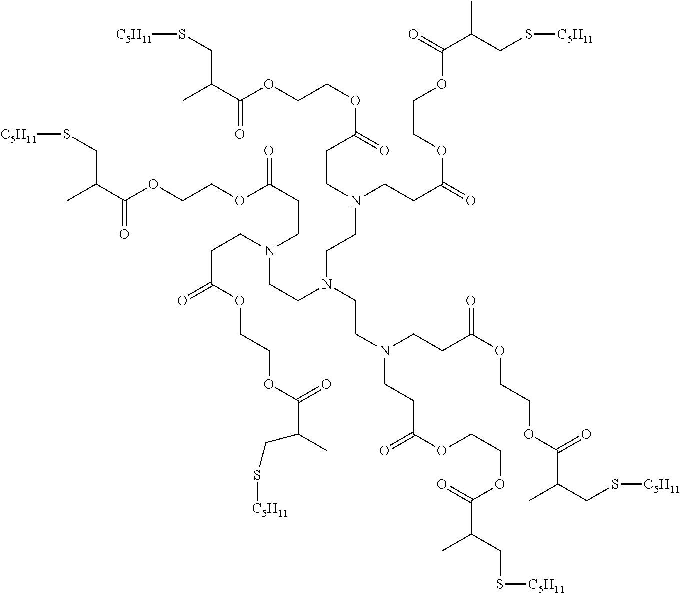 |
| 5A4- 2- SC6 (6 arm) | 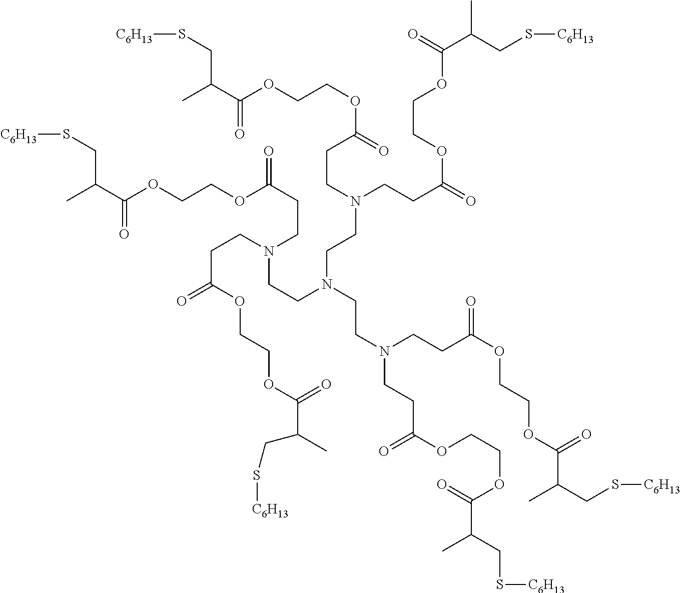 |
| 5A2- 4- SC8 (5-arm) | 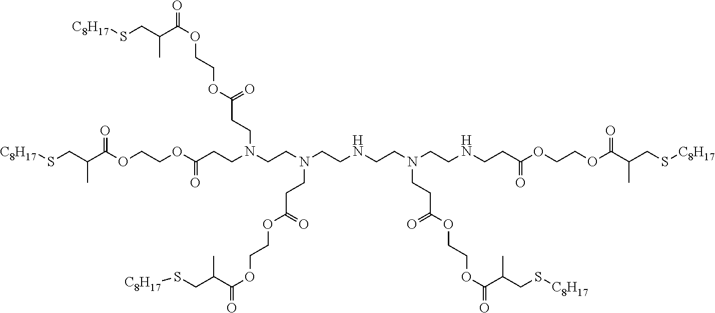 |
| 3A5- g2- SC8 | 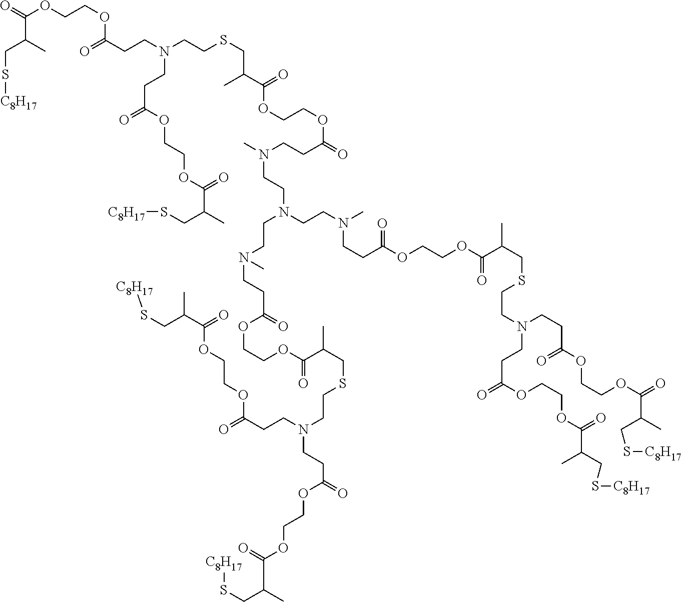 |

C. Modifying the functional groups and/or the chemical properties of the core, repeating units, and the surface or terminating groups, their physical properties can be modulated. Some properties which can be varied include, but are not limited to, solubility, toxicity, immunogenicity and bioattachment capability. Dendrimers are often described by their generation or number of repeating units in the branches. A dendrimer consisting of only the core molecule is referred to as Generation 0, while each consecutive repeating unit along all branches is Generation 1, Generation 2, and so on until the terminating or surface group. In some embodiments, half generations are possible resulting from only the first condensation reaction with the amine and not the second condensation reaction with the thiol.

Preparation of dendrimers requires a level of synthetic control achieved through series of stepwise reactions comprising building the dendrimer by each consecutive group. Dendrimer synthesis can be of the convergent or divergent type. During divergent dendrimer synthesis, the molecule is assembled from the core to the periphery in a stepwise process involving attaching one generation to the previous and then changing functional groups for the next stage of reaction. Functional group transformation is necessary to prevent uncontrolled polymerization. Such polymerization would lead to a highly branched molecule that is not monodisperse and is otherwise known as a hyperbranched polymer. Due to steric effects, continuing to react dendrimer repeat units leads to a sphere shaped or globular molecule, until steric overcrowding prevents complete reaction at a specific generation and destroys the molecule's monodispersity. Thus, in some embodiments, the dendrimers of G1-G10 generation are specifically contemplated. In some embodiments, the dendrimers comprise 1, 2, 3, 4, 5, 6, 7, 8, 9, or 10 repeating units, or any range derivable therein. In some embodiments, the dendrimers used herein are G0, G1, G2, or G3. However, the number of possible generations (such as 11, 12, 13, 14, 15, 20, or 25) may be increased by reducing the spacing units in the branching polymer.

Additionally, dendrimers have two major chemical environments: the environment created by the specific surface groups on the termination generation and the interior of the dendritic structure which due to the higher order structure can be shielded from the bulk media and the surface groups. Because of these different chemical environments, dendrimers have found numerous different potential uses including in therapeutic applications.

In some aspects, the dendrimers that may be used in the present compositions are assembled using the differential reactivity of the acrylate and methacrylate groups with amines and thiols. The dendrimers may include secondary or tertiary amines and thioethers formed by the reaction of an acrylate group with a primary or secondary amine and a methacrylate with a mercapto group. Additionally, the repeating units of the dendrimers may contain groups which are degradable under physiological conditions. In some embodiments, these repeating units may contain one or more germinal diethers, esters, amides, or disulfides groups. In some embodiments, the core molecule is a monoamine which allows dendritic polymerization in only one direction. In other embodiments, the core molecule is a polyamine with multiple different dendritic branches which each may comprise one or more repeating units. The dendrimer may be formed by removing one or more hydrogen atoms from this core. In some embodiments, these hydrogen atoms are on a heteroatom such as a nitrogen atom. In some embodiments, the terminating group is a lipophilic groups such as a long chain alkyl or alkenyl group. In other embodiments, the terminating group is a long chain haloalkyl or haloalkenyl group. In other embodiments, the terminating group is an aliphatic or aromatic group containing an ionizable group such as an amine (—NH₂) or a carboxylic acid (—CO₂H). In still other embodiments, the terminating group is an aliphatic or aromatic group containing one or more hydrogen bond donors such as a hydroxide group, an amide group, or an ester.

In some embodiments, the compositions may further comprise a molar ratio of the ionizable lipids to the total lipid composition from about 15 to about 60. In some embodiments, the molar ratio is from about 10, 15, 20, 25, 30, 35, 40, 45, 50, 55, to about 60 or any range derivable therein. In some embodiments, the molar ratio is from about 30 to about 45.

The cationic ionizable lipids of the present disclosure may contain one or more asymmetrically-substituted carbon or nitrogen atoms, and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Cationic ionizable lipids may occur as racemates and racemic mixtures, single enantiomers, diastereomeric mixtures and individual diastereomers. In some embodiments, a single diastereomer is obtained. The chiral centers of the cationic ionizable lipids of the present disclosure can have the S or the R configuration. Furthermore, it is contemplated that one or more of the cationic ionizable lipids may be present as constitutional isomers. In some embodiments, the compounds have the same formula but different connectivity to the nitrogen atoms of the core. Without wishing to be bound by any theory, it is believed that such cationic ionizable lipids exist because the starting monomers react first with the primary amines and then statistically with any secondary amines present. Thus, the constitutional isomers may present the fully reacted primary amines and then a mixture of reacted secondary amines.

Chemical formulas used to represent cationic ionizable lipids of the present disclosure will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given formula, and regardless of which one is most prevalent, all tautomers of a given chemical formula are intended.

The cationic ionizable lipids of the present disclosure may also have the advantage that they may be more efficacious than, be less toxic than, be longer acting than, be more potent than, produce fewer side effects than, be more easily absorbed than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

In addition, atoms making up the cationic ionizable lipids of the present disclosure are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

It should be recognized that the particular anion or cation forming a part of any salt form of a cationic ionizable lipids provided herein is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

### D. Additional Lipids in the Lipid Nanoparticles

In some aspects of the present disclosure, compositions containing one or more lipids are mixed with the cationic ionizable lipids to create a composition. In some embodiments, the polymers are mixed with 1, 2, 3, 4, or 5 different types of lipids. It is contemplated that the cationic ionizable lipids can be mixed with multiple different lipids of a single type. In some embodiments, the cationic ionizable lipids compositions comprise at least a steroid or a steroid derivative, a PEG lipid, and a phospholipid.

### Steroids and Steroid Derivatives

In some aspects of the present disclosure, the cationic ionizable lipids are mixed with one or more steroid or a steroid derivative to create a composition. In some embodiments, the steroid or steroid derivative comprises any steroid or steroid derivative. As used herein, in some embodiments, the term “steroid” is a class of compounds with a four ring 17 carbon cyclic structure which can further comprises one or more substitutions including alkyl groups, alkoxy groups, hydroxy groups, oxo groups, acyl groups, or a double bond between two or more carbon atoms. In one aspect, the ring structure of a steroid comprises three fused cyclohexyl rings and a fused cyclopentyl ring as shown in the formula below:

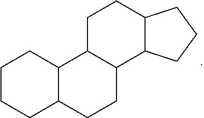 [SMILES: C1CCC2C(C1)CCC1C3CCCC3CCC21]

In some embodiments, a steroid derivative comprises the ring structure above with one or more non-alkyl substitutions. In some embodiments, the steroid or steroid derivative is a sterol wherein the formula is further defined as:

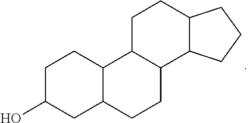 [SMILES: OC1CCC2C(CCC3C4CCCC4CCC23)C1]

In some embodiments of the present disclosure, the steroid or steroid derivative is a cholestane or cholestane derivative. In a cholestane, the ring structure is further defined by the formula:

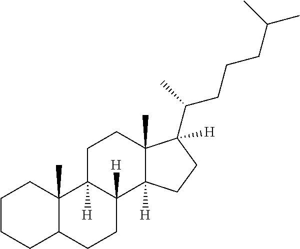

As described above, a cholestane derivative includes one or more non-alkyl substitution of the above ring system. In some embodiments, the cholestane or cholestane derivative is a cholestene or cholestene derivative or a sterol or a sterol derivative. In other embodiments, the cholestane or cholestane derivative is both a cholestere and a sterol or a derivative thereof.

In some embodiments, the compositions may further comprise a molar ratio of the steroid to the total lipid composition from about 10 to about 60. In some embodiments, the molar ratio is from about 10, 15, 20, 25, 30, 35, 40, 45, 50, 55, to about 60 or any range derivable therein. In some embodiments, the molar ratio is from about 25 to about 50 such as 30.

### Polymer Conjugated Lipids

In some aspects of the present disclosure, the polymers are mixed with one or more polymer conjugated lipid such as PEGylated lipids (or PEG lipid) to create a dendrimer composition. In some embodiments, the present disclosure comprises using any lipid to which a PEG group has been attached. In some embodiments, the PEG lipid is a diglyceride which also comprises a PEG chain attached to the glycerol group. In other embodiments, the PEG lipid is a compound which contains one or more C6-C24 long chain alkyl or alkenyl group or a C6-C24 fatty acid group attached to a linker group with a PEG chain. Some non-limiting examples of a PEG lipid includes a PEG modified phosphatidylethanolamine and phosphatidic acid, a PEG ceramide conjugated, PEG modified dialkylamines and PEG modified 1,2-diacyloxypropan-3-amines, PEG modified diacylglycerols and dialkylglycerols. In some embodiments, PEG modified diastearoylphosphatidylethanolamine or PEG modified dimyristoyl-sn-glycerol. In some embodiments, the PEG modification is measured by the molecular weight of PEG component of the lipid. In some embodiments, the PEG modification has a molecular weight from about 100 to about 15,000. In some embodiments, the molecular weight is from about 200 to about 500, from about 400 to about 5,000, from about 500 to about 3,000, or from about 1,200 to about 3,000. The molecular weight of the PEG modification is from about 100, 200, 400, 500, 600, 800, 1,000, 1,250, 1,500, 1,750, 2,000, 2,250, 2,500, 2,750, 3,000, 3,500, 4,000, 4,500, 5,000, 6,000, 7,000, 8,000, q_{,000, 10,000, 12,500}, to about 15,000. Some non-limiting examples of lipids that may be used in the present invention are taught by U.S. Pat. No. 5,820,873, WO 2010/141069, or U.S. Pat. No. 8,450,298, which is incorporated herein by reference.

In another aspect, the PEG lipid has the formula:

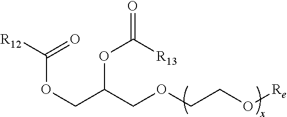

wherein: R₁₂ and R₁₃ are each independently alkyl_{(C≤24)}, alkenyl_{(C≤24)}, or a substituted version of either of these groups; Rₑ is hydrogen, alkyl_{(C≤8)}, or substituted alkyl_{(C≤8)}; and x is 1-250. In some embodiments, Rₑ is alkyl_{(C≤8)} such as methyl. R₁₂ and R₁₃ are each independently alkyl_{(C≤4-20)}. In some embodiments, x is 5-250. In one embodiment, x is 5-125 or x is 100-250. In some embodiments, the PEG lipid is 1,2-dimyristoyl-sn-glycerol, methoxypolyethylene glycol.

In another aspect, the PEG lipid has the formula:

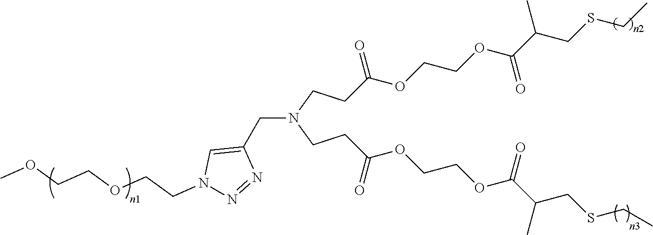

wherein: n₁ is an integer between 1 and 100 and n₂ and n₃ are each independently selected from an integer between 1 and 29. In some embodiments, n₁ is 5, 10, 15, 20, 25, 30, 31, 32, 33, 34, 35, 36, 37, 38, 39, 40, 41, 42, 43, 44, 45, 46, 47, 48, 49, 50, 55, 60, 65, 70, 75, 80, 85, q₀, q₅, or 100, or any range derivable therein. In some embodiments, n₁ is from about 30 to about 50. In some embodiments, n₂ is from 5 to 23. In some embodiments, n₂ is 11 to about 17. In some embodiments, n₃ is from 5 to 23. In some embodiments, n₃ is 11 to about 17.

In some embodiments, the compositions may further comprise a molar ratio of the PEG lipid to the ionizable total lipid composition from about 0.25, 0.5, 0.75, 1, 1.5, 2, 2.5, 3, 4, 5, 6, 8, 10, 12, to about 12.5 or any range derivable therein. In some embodiments, the molar ratio is from about 1 to about 6.

### Phospholipids

In some aspects of the present disclosure, the polymers are mixed with one or more phospholipids to create a composition. In some embodiments, any lipid which also comprises a phosphate group. In some embodiments, the phospholipid is a structure which contains one or two long chain C6-C24 alkyl or alkenyl groups, a glycerol or a sphingosine, one or two phosphate groups, and, optionally, a small organic molecule. In some embodiments, the small organic molecule is an amino acid, a sugar, or an amino substituted alkoxy group, such as choline or ethanolamine. In some embodiments, the phospholipid is a phosphatidylcholine. In some embodiments, the phospholipid is distearoylphosphatidylcholine or dioleoylphosphatidylethanolamine.

In some embodiments, the compositions may further comprise a molar ratio of the phospholipid to the total lipid composition from about 5 to about 50. In some embodiments, the molar ratio is from about 5, 10, 15, 20, 25, 30, 35, 40, 45, to about 50 or any range derivable therein. In some embodiments, the molar ratio is from about 20 to about 40.

### E. Nucleic Acids and Nucleic Acid Based Therapeutic Agents

Nucleic acids

In some aspects of the present disclosure, the dendrimer compositions comprise one or more nucleic acids. In some embodiments, the dendrimer composition comprises one or more nucleic acids present in a weight ratio to the ionizable lipid from about 5:1 to about 1:100. In some embodiments, the weight ratio of nucleic acid to dendrimer is from about 5:1, 2.5:1, 1:1, 1:5, 1:10, 1:15, 1:20, 1:25, 1:30, 1:35, 1:40, 1:45, 1:50, 1:60, 1:70, 1:80, 1:90, or 1:100, or any range derivable therein. In addition, it should be clear that the present disclosure is not limited to the specific nucleic acids disclosed herein. The present invention is not limited in scope to any particular source, sequence, or type of nucleic acid, however, as one of ordinary skill in the art could readily identify related homologs in various other sources of the nucleic acid including nucleic acids from non-human species (e.g., mouse, rat, rabbit, dog, monkey, gibbon, chimp, ape, baboon, cow, pig, horse, sheep, cat and other species). It is contemplated that the nucleic acid used in the present disclosure can comprises a sequence based upon a naturally occurring sequence. Allowing for the degeneracy of the genetic code, sequences that have at least about 50%, usually at least about 60%, more usually about 70%, most usually about 80%, preferably at least about 90% and most preferably about 95% of nucleotides that are identical to the nucleotide sequence of the naturally occurring sequence can encode the same protein as the naturally occurring sequence. In another embodiment, the nucleic acid is a complementary sequence to a naturally occurring sequence, or complementary to at least 80%, 90%. 98%, 98% and 99%. Longer polynucleotides encoding 250, 500, 1000, 1212, 1500, 2000, 2500, 3000 or longer are contemplated herein.

The nucleic acid used herein may be derived from genomic DNA, i.e., cloned directly from the genome of a particular organism. In preferred embodiments, however, the nucleic acid would comprise complementary DNA (cDNA). Also contemplated is a cDNA plus a natural intron or an intron derived from another gene; such engineered molecules are sometime referred to as “mini-genes.” At a minimum, these and other nucleic acids of the present invention may be used as molecular weight standards in, for example, gel electrophoresis.

The term “cDNA” is intended to refer to DNA prepared using messenger RNA (mRNA) as template. The advantage of using a cDNA, as opposed to genomic DNA or DNA polymerized from a genomic, non- or partially processed RNA template, is that the cDNA primarily contains coding sequences of the corresponding protein. There may be times when the full or partial genomic sequence is preferred, such as where the non-coding regions are required for optimal expression or where non-coding regions such as introns are to be targeted in an antisense strategy.

In some embodiments, the nucleic acid comprises one or more antisense segments which targets a desired HDR site in a gene or gene product. Antisense methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G: C) and adenine paired with either thymine (A: T) in the case of DNA, or adenine paired with uracil (A: U) in the case of RNA. Inclusion of less common bases such as inosine, 5-methylcytosine, 6-methyladenine, hypoxanthine and others in hybridizing sequences does not interfere with pairing.

In some embodiments, the nucleic acid comprises one or more antisense segments which targets a desired HDR site in a gene or gene product. Antisense methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G: C) and adenine paired with either thymine (A: T) in the case of DNA, or adenine paired with uracil (A: U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNA's, may be employed to target a gene editing event within a host cell, either in vitro or in vivo, such as within a host animal, including a human subject.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozyme; see below) could be designed. These molecules, though having less than 50% homology, would bind to target sequences under appropriate conditions.

In some embodiments, the polynucleotide comprising a sequence encoding for a polynucleotide-guided nuclease such as an mRNA comprises from about 250 to about 15,000 nucleotides, from about 500 to about 5,000 nucleotides, from about 800 to about 2,500 nucleotides, or from about 100, 150, 200, 250, 300, 350, 400, 450, 500, 550, 600, 650, 700, 750, 800, 850, 900, 1,000, 2,000, 3,000, 4,000, 5,000, 6,000, 7,000, 8,000, 9,000, 10,000, 11,000, 12,000, 13.000, 14,000, to about 15,000 nucleotides, or any range derivable therein. In some embodiments, the guide polynucleotide, particularly a polynucleotide which has been configured to complex with at least a portion of a target gene or transcript or a polynucleotide with a sequence that encodes for such a guide polynucleotide such as a sgRNA comprises from about 25 to about 500 nucleotides, from about 50 to about 300 nucleotides, from about 80 to about 200 nucleotides or from about 10, 15, 20, 25, 30, 35, 40, 45, 50, 60, 65, 70, 75, 80, 85, 90, 95, 100, 150, 200, 250, 300, 350, 400, 450, to about 500 nucleotides, or any range derivable therein. In some embodiments, the donor polynucleotide, particularly a polynucleotide configured to repair a modified target gene or transcript such as a DNA comprises from about 25 to about 2,500 nucleotides, from about 25 to about 500 nucleotides, from about 50 to about 300 nucleotides, from about 80 to about 200 nucleotides or from about 10, 15, 20, 25, 30, 35, 40, 45, 50, 60, 65, 70, 75, 80, 85, 90, 95, 100, 150, 200, 250, 300, 350, 400, 450, to about 500 nucleotides, or any range derivable therein.

In some embodiments, the composition comprises a weight ratio of the polynucleotide comprising a sequence encoding for a polynucleotide-guided nuclease such as an mRNA to the guide polynucleotide, particularly a polynucleotide which has been configured to complex with at least a portion of a target gene or transcript or a polynucleotide with a sequence that encodes for such a guide polynucleotide such as a sgRNA from about 10:1 to about 1:5, from about 5:1 to about 1:3, from about 3:1 to about 1:2, or from about 10:1, q_{:1, 8:1, 7:1, 6:1, 5:1, 4:1, 3:1, 2:1, 1:1, 1:2, 1:3, 1:4}, to about 1:5, or any range derivable therein. In some embodiments, the composition comprises a weight ratio of the polynucleotide comprising a sequence encoding for a polynucleotide-guided nuclease such as an mRNA to the interfering polynucleotide, particularly a polynucleotide configured to interfere with the expression of a target gene or transcript such as a DNA, from about 10:1 to about 1:10, from about 5:1 to about 1:5, from about 2:1 to about 1:2, or from about 10:1, q_{:1, 8:1, 7:1, 6:1, 5:1, 4:1, 3:1, 2:1, 1:1, 1:2, 1:3, 1:4, 1:5, 1:6, 1:7, 1:8, 1:9}, to about 1:10, or any range derivable therein. In some embodiments, the composition comprises a weight ratio of the polynucleotide comprising a sequence encoding for a polynucleotide-guided nuclease such as an mRNA to the inhibitory polynucleotide of about 2:3. In some embodiments, the composition comprises a weight ratio of the guide polynucleotide, particularly a polynucleotide which has been configured to complex with at least a portion of a target gene or transcript or a polynucleotide with a sequence that encodes for such a guide polynucleotide such as a sgRNA to the interfering polynucleotide, particularly a polynucleotide configured to interfere with the expression of a target gene or transcript such as DNA, from about 4:1 to about 1:10, from about 2:1 to about 1:8, from about 1:1 to about 1:4, or from about 4:1, 3:1, 2:1, 2:3, 1:1, 1:2, 1:3, 1:4, 1:5, 1:6, 1:7, 1:8, 1:9, to about 1:10, or any range derivable therein.

In some embodiments, the composition comprises a molar ratio of lipid components to nucleic acid components of from about 1,000:1 to about 5,000:1, from about 2,000:1 to about 4,000:1, or from about 1,000:1, 1,500:1, 2,000:1, 2,500:1, 3,000:1, 3,500:1, 4,000:1, 4,500:1, to about 1,500:1, or any range derivable therein. In some embodiments, the composition comprises an N: P ratio of from about 1:1 to about 20:1, from about 2:1 to about 10:1, from about 4:1 to about 8:1, or from about 1:1, 2:1, 3:1, 4:1, 5:1, 6:1, 7:1, 8:1, q_{:1, 10:1, 11:1, 12:1, 13:1, 14:1, 15:1, 16:1, 17:1, 18:1, 19:1}, to about 20:1, or any range derivable therein.

### Modified Nucleobases

In some embodiments, the nucleic acids of the present disclosure comprise one or more modified nucleosides comprising a modified sugar moiety. Such compounds comprising one or more sugar-modified nucleosides may have desirable properties, such as enhanced nuclease stability or increased binding affinity with a target nucleic acid relative to an oligonucleotide comprising only nucleosides comprising naturally occurring sugar moieties. In some embodiments, modified sugar moieties are substituted sugar moieties. In some embodiments, modified sugar moieties are sugar surrogates. Such sugar surrogates may comprise one or more substitutions corresponding to those of substituted sugar moieties.

In some embodiments, modified sugar moieties are substituted sugar moieties comprising one or more non-bridging sugar substituent, including but not limited to substituents at the 2′ and/or 5′ positions. Examples of sugar substituents suitable for the 2′-position, include, but are not limited to: 2′-F, 2′—OCH₃ (“OMe” or “O-methyl”), and 2′-O(CH₂)₂OCH₃ (“MOE”). In certain embodiments, sugar substituents at the 2′ position is selected from allyl, amino, azido, thio, O-allyl, O—C₁-C₁₀ alkyl, O—C₁-C₁₀ substituted alkyl; OCF₃, O(CH₂)₂SCH3, O(CH₂)₂—O—N(Rₘ)(Rₙ), and O—CH₂—C(═O)—N(Rₘ)(Rₙ), where each Rₘ and Rₙ is, independently, H or substituted or unsubstituted C₁-C₁₀ alkyl. Examples of sugar substituents at the 5′-position, include, but are not limited to: 5′-methyl (R or S); 5′-vinyl, and 5′-methoxy. In some embodiments, substituted sugars comprise more than one non-bridging sugar substituent, for example, T-F-5′-methyl sugar moieties (see, e.g., PCT International Application WO 2008/101157, for additional 5′,2′-b is substituted sugar moieties and nucleosides).

Nucleosides comprising 2′-substituted sugar moieties are referred to as 2′-substituted nucleosides. In some embodiments, a 2′-substituted nucleoside comprises a 2′-substituent group selected from halo, allyl, amino, azido, SH, CN, OCN, CF₃, OCF₃, O, S, or N(Rₘ)-alkyl; O, S, or N(Rₘ)-alkenyl; O, S or N(Rₘ)-alkynyl; O-alkylenyl-O-alkyl, alkynyl, alkaryl, aralkyl, O-alkaryl, O-aralkyl, O(CH₂)₂SCH3, O(CH₂)₂—O—N(Rₘ)(Rₙ) or O—CH₂—C(═O)—N(Rₘ)(Rₙ), where each Rₘ and Rₙ is, independently, H, an amino protecting group or substituted or unsubstituted C₁-C₁₀ alkyl. These 2′-substituent groups can be further substituted with one or more substituent groups independently selected from hydroxyl, amino, alkoxy, carboxy, benzyl, phenyl, nitro (NO₂), thiol, thioalkoxy (S-alkyl), halogen, alkyl, aryl, alkenyl and alkynyl.

In some embodiments, a 2′-substituted nucleoside comprises a 2′-substituent group selected from F, NH₂, N₃, OCF₃, O—CH₃, O(CH₂)₃NH₂, CH₂—CH═CH₂, O—CH₂—CH═CH₂, OCH₂CH₂OCH₃, O(CH₂)₂SCH3, O—(CH₂)₂—O—N(Rₘ)(Rₙ), O(CH₂)₂₀ (CH₂)₂N(CH₃)₂, and N-substituted acetamide (O—CH₂—C(═O)—N(Rₘ)(Rₙ) where each Rₘ and Rₙ is, independently, H, an amino protecting group or substituted or unsubstituted C₁-C₁₀ alkyl.

In some embodiments, a 2′-substituted nucleoside comprises a sugar moiety comprising a 2′-substituent group selected from F, OCF₃, O—CH₃, OCH, CH₂OCH₃, O(CH₂): SCH3, O(CH₂)₂—O—N(CH₃)₂, —O(CH₂)₂O(CH₂)₂N(CH₃)₂, and O—CH₂—C(═O)—N(H) CH₃.

In some embodiments, a 2′-substituted nucleoside comprises a sugar moiety comprising a 2′-substituent group selected from F, O—CH₃, and OCH₂CH₂OCH₃.

Certain modified sugar moieties comprise a bridging sugar substituent that forms a second ring resulting in a bicyclic sugar moiety. In some such embodiments, the bicyclic sugar moiety comprises a bridge between the 4′ and the 2′ furanose ring atoms. Examples of such 4′ to 2′ sugar substituents, include, but are not limited to: —[C(R^{a})(R^{b}) In—-, —[C(R^{a})(R^{b})]ₙ—O—-, —C(RₐR_{b})—N(R)—O—- or, —C(RₐR_{b})—O—N(R)—-; 4′-CH₂-2′, 4′—(CH₂)₂-2′, 4′—(CH₂)—O-2′ (LNA); 4′-(CH₂)—S-2′; 4′—(CH₂)₂—O-2′ (ENA); 4′-CH (CH₃)—O-2′ (cEt) and 4′-CH (CH₂OCH₃)—O-2′, and analogs thereof (see, e.g., U.S. Pat. No. 7,399,845); 4′-C(CH₃)(CH₃)—O-2′ and analogs thereof, (see, e.g., WO 2009/006478); 4′-CH₂—N(OCH₃)-2′ and analogs thereof (see, e.g., WO2008/150729); 4′-CH₂—O—N(CH₃)-2′ (see, e.g., US2004/0171570, published Sep. 2, 2004); 4′-CH₂—O—N(R)-2′, and 4′-CH₂—N(R)—O-2′-, wherein each R is, independently, H, a protecting group, or C₁-C₁₂ alkyl; 4′-CH₂—N(R)—O-2′, wherein R is H, C₁-C₁₂ alkyl, or a protecting group (see, U.S. Pat. No. 7,427,672); 4′-CH₂—C(H)(CH₃)-2′ (see, e.g., Chattopadhyaya et al., J. Org. Chem., 2009, 74, 118-134); and 4′-CH₂—C(═CH₂)-2′ and analogs thereof (see, PCT International Application WO 2008/154401).

In some embodiments, such 4′ to 2′ bridges independently comprise from 1 to 4 linked groups independently selected from —[C(R^{a})(R^{b})]ₙ—-, —C(R^{a})═C(R^{b})—-, —C(R^{a})═N—-, —C(═NR^{a})—-, —C(═O)—-, —C(═S)—-, —O—-, —Si (R^{a}) 2--, —S(═O)ₓ—-, and —N(R^{a})—-; wherein:
x is 0, 1, or 2;
n is 1, 2, 3, or 4;
each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl(C(═O)—H), substituted acyl, CN, sulfonyl (S(═O)₂-J₁), or sulfoxyl (S(═O)-J₁); and
each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl(C(═O)—H), substituted acyl, a heterocycle radical, a substituted heterocycle radical, C₁-C₁₂ aminoalkyl, substituted C₁-C₁₂ aminoalkyl, or a protecting group.

Nucleosides comprising bicyclic sugar moieties are referred to as bicyclic nucleosides or BNAs. Bicyclic nucleosides include, but are not limited to, (A) a-L-Methyleneoxy (4′-CH₂—O-2′) BNA, (B) B-D-Methyleneoxy (4′-CH₂—O-2′) BNA (also referred to as locked nucleic acid or LNA), (C) Ethyleneoxy (4′-(CH₂)₂—O-2′) BNA, (D) Aminooxy (4′-CH₂—O—N(R)-2′) BNA, (E) Oxyamino (4′-CH₂—N(R)—O-2′) BNA, (F) Methyl(methyleneoxy) (4′-CH (CH₃)—O-2′) BNA (also referred to as constrained ethyl or cEt), (G) methylene-thio (4′-CH₂—S-2′) BNA, (H) methylene-amino (4′-CH₂—N(R)-2′) BNA, (I) methyl carbocyclic (4′-CH₂—CH (CH₃)-2′) BNA, (J) propylene carbocyclic (4′-(CH₂)₃-2′) BNA, and (K) Methoxy (ethyleneoxy) (4′-CH (CH₂OMe)-O-2′) BNA (also referred to as constrained MOE or cMOE).

Additional bicyclic sugar moieties are known in the art, for example: Singh et al., Chem. Commun., 1998, 4, 455-456; Koshkin et al., Tetrahedron, 1998, 54, 3607-3630; Wahlestedt et al., Proc. Natl. Acad. Sci. U.S.A., 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 129 (26) 8362-8379 (Jal. 4, 2007); Elayadi et al., Curr. Opinion Invens. Drugs, 2001, 2, 5561; Braasch et al., Chem. Biol., 2001, 8, 1-7; Orum et al., Curr. Opinion Mol. Ther., 2001, 3, 239-243; U.S. Pat. Nos. 7,053,207, 6,268,490, 6,770,748, 6,794,499, 7,034,133, 6,525,191, 6,670,461, and 7,399,845; WO 2004/106356, WO 1994/14226, WO 2005/021570, and WO 2007/134181; U.S. Patent Publication Nos. US 2004/0171570, US 2007/0287831, and US 2008/0039618; U.S. Ser. Nos. 12/129,154, 60/989,574, 61/026,995, 61/026,998, 61/056,564, 61/086,231, 61/097,787, and 61/099,844; and PCT International Applications Nos. PCT/US2008/064591, PCT/US2008/066154, and PCT/US2008/068922. In some embodiments, bicyclic sugar moieties and nucleosides incorporating such bicyclic sugar moieties are further defined by isomeric configuration. For example, a nucleoside comprising a 4′-2′ methylene-oxy bridge, may be in the a-L configuration or in the B-D configuration. Previously, a-L-methyleneoxy (4′-CH₂—O-2′) bicyclic nucleosides have been incorporated into antisense oligonucleotides that showed antisense activity (Frieden et al., Nucleic Acids Research, 2003, 21, 6365-6372).

In some embodiments, substituted sugar moieties comprise one or more non-bridging sugar substituent and one or more bridging sugar substituent (e.g., 5′-substituted and 4′-2′ bridged sugars; PCT International Application WO 2007/134181, wherein LNA is substituted with, for example, a 5′-methyl or a 5′-vinyl group).

In some embodiments, modified sugar moieties are sugar surrogates. In some such embodiments, the oxygen atom of the naturally occurring sugar is substituted, e.g., with a sulfer, carbon or nitrogen atom. In some such embodiments, such modified sugar moiety also comprises bridging and/or non-bridging substituents as described above. For example, certain sugar surrogates comprise a 4′-sulfur atom and a substitution at the 2′-position (see, e.g., published U.S. Patent Application US 2005/0130923) and/or the 5′ position. By way of additional example, carbocyclic bicyclic nucleosides having a 4′-2′ bridge have been described (see, e.g., Freier et al., Nucleic Acids Research, 1997, 25 (22), 4429-4443 and Albaek et al., J. Org. Chem., 2006, 71, 7731-7740).

In some embodiments, sugar surrogates comprise rings having other than 5-atoms. For example, in some embodiments, a sugar surrogate comprises a six-membered tetrahydropyran. Such tetrahydropyrans may be further modified or substituted. Nucleosides comprising such modified tetrahydropyrans include, but are not limited to, hexitol nucleic acid (HNA), anitol nucleic acid (ANA), manitol nucleic acid (MNA) (see Leumann, C J. Bioorg. & Med. Chem. (2002) 10:841-854), and fluoro HNA (F-HNA).

In some embodiments, the modified THP nucleosides of Formula VII are provided wherein q₁, q₂, q₃, q₄, q₅, q₆ and q₇ are each H. In certain embodiments, at least one of q₁, q₂, q₃, q₄, q₅, q₆ and q₇ is other than H. In some embodiments, at least one of q₁, q₂, q₃, q₄, q₅, q₆ and q₇ is methyl. In some embodiments, THP nucleosides of Formula VII are provided wherein one of R₁ and R₂ is F. In certain embodiments, R₁ is fluoro and R₂ is H, R₁ is methoxy and R₂ is H, and R₁ is methoxyethoxy and R₂ is H.

Many other bicyclo and tricyclo sugar surrogate ring systems are also known in the art that can be used to modify nucleosides for incorporation into antisense compounds (see, e.g., review article: Leumann, J. C, Bioorganic & Medicinal Chemistry, 2002, 10, 841-854).

Combinations of modifications are also provided without limitation, such as 2′-F-5′-methyl substituted nucleosides (see PCT International Application WO 2008/101157 for other disclosed 5′,2′-b is substituted nucleosides) and replacement of the ribosyl ring oxygen atom with S and further substitution at the 2′-position (see U.S. Patent Publication US 2005/0130923) or alternatively 5′-substitution of a bicyclic nucleic acid (see PCT International Application WO 2007/134181 wherein a 4′-CH₂—O-2′ bicyclic nucleoside is further substituted at the 5′ position with a 5′-methyl or a 5′-vinyl group). The synthesis and preparation of carbocyclic bicyclic nucleosides along with their oligomerization and biochemical studies have also been described (see, e.g., Srivastava et al., 2007).

In some embodiments, the present invention provides oligonucleotides comprising modified nucleosides. Those modified nucleotides may include modified sugars, modified nucleobases, and/or modified linkages. The specific modifications are selected such that the resulting oligonucleotides possess desirable characteristics. In some embodiments, oligonucleotides comprise one or more RNA-like nucleosides. In some embodiments, oligonucleotides comprise one or more DNA-like nucleotides.

In some embodiments, nucleosides of the present invention comprise one or more unmodified nucleobases. In certain embodiments, nucleosides of the present invention comprise one or more modified nucleobases.

In some embodiments, modified nucleobases are selected from: universal bases, hydrophobic bases, promiscuous bases, size-expanded bases, and fluorinated bases as defined herein. 5-substituted pyrimidines, 6-azapyrimidines and N-2, N-6 and O-6 substituted purines, including 2-aminopropyladenine, 5-propynyluracil; 5-propynylcytosine; 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines. 5-halo particularly 5-bromo, 5-trifluoromethyl and other 5-substituted uracils and cytosines, 7-methylguanine and 7-methyladenine, 2—F-adenine, 2-amino-adenine, 8-azaguanine and 8-azaadenine, 7-deazaguanine and 7-deazaadenine, 3-deazaguanine and 3-deazaadenine, universal bases, hydrophobic bases, promiscuous bases, size-expanded bases, and fluorinated bases as defined herein. Further modified nucleobases include tricyclic pyrimidines such as phenoxazine cytidine ([5,4-b][1,4]benzoxazin-2 (3H)-one), phenothiazine cytidine (1H-pyrimido[5,4-b][1,4]benzothiazin-2 (3H)-one), G-clamps such as a substituted phenoxazine cytidine (e.g., 9-(2-aminoethoxy)-H-pyrimido[5,4-13][1,4]benzoxazin-2 (3H)-one), carbazole cytidine (2H-pyrimido[4,5-b]indol-2-one), pyridoindole cytidine (H-pyrido[3′,2′: 4.5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, Kroschwitz, J. I., Ed., John Wiley & Sons, 1990, 858-859; those disclosed by Englisch et al., 1991; and those disclosed by Sanghvi, Y. S., 1993.

Representative United States Patents that teach the preparation of certain of the above noted modified nucleobases as well as other modified nucleobases include without limitation,

U.S. Pat. Nos. 3,687,808; 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121; 5,596,091; 5,614,617; 5,645,985; 5,681,941; 5,750,692; 5,763,588; 5,830,653 and 6,005,096, each of which is herein incorporated by reference in its entirety.

In some embodiments, the present invention provides oligonucleotides comprising linked nucleosides. In such embodiments, nucleosides may be linked together using any internucleoside linkage. The two main classes of internucleoside linking groups are defined by the presence or absence of a phosphorus atom. Representative phosphorus containing internucleoside linkages include, but are not limited to, phosphodiesters (P═O), phosphotriesters, methylphosphonates, phosphoramidate, and phosphorothioates (P═S). Representative non-phosphorus containing internucleoside linking groups include, but are not limited to, methylenemethylimino (—CH₂—N(CH₃)—O—CH₂—-), thiodiester (—O—C(O)—S—), thionocarbamate (—O—C(O)(NH)—S—-); siloxane (—O—Si (H)₂—O—-); and N,N′-dimethylhydrazine (—CH₂—N(CH₃)—N(CH₃)—-). Modified linkages, compared to natural phosphodiester linkages, can be used to alter, typically increase, nuclease resistance of the oligonucleotide. In some embodiments, internucleoside linkages having a chiral atom can be prepared as a racemic mixture, or as separate enantiomers. Representative chiral linkages include, but are not limited to, alkylphosphonates and phosphorothioates. Methods of preparation of phosphorous-containing and non-phosphorous-containing internucleoside linkages are well known to those skilled in the art.

The oligonucleotides described herein contain one or more asymmetric centers and thus give rise to enantiomers, diastereomers, and other stereoisomeric configurations that may be defined, in terms of absolute stereochemistry, as (R) or(S), a or β such as for sugar anomers, or as (D) or (L) such as for amino acids etc. Included in the antisense compounds provided herein are all such possible isomers, as well as their racemic and optically pure forms.

Neutral internucleoside linkages include without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

Additional modifications may also be made at other positions on the oligonucleotide, particularly the 3′ position of the sugar on the 3′ terminal nucleotide and the 5′ position of 5′ terminal nucleotide. For example, one additional modification of the ligand conjugated oligonucleotides of the present invention involves chemically linking to the oligonucleotide one or more additional non-ligand moieties or conjugates which enhance the activity, cellular distribution or cellular uptake of the oligonucleotide. Such moieties include but are not limited to lipid moieties such as a cholesterol moiety (Letsinger et al., 1989), cholic acid (Manoharan et al., 1994), a thioether, e.g., hexyl-5-tritylthiol (Manoharan et al., 1992; Manoharan et al., 1993), a thiocholesterol (Oberhauser et al., 1992), an aliphatic chain, e.g., dodecandiol or undecyl residues (Saison-Behmoaras et al., 1991; Kabanov et al., 1990; Svinarchuk et al., 1993), a phospholipid, e.g., di-hexadecyl-rac-glycerol or triethylammonium 1,2-di-O-hexadecyl-rac-glycero-3-H-phosphonate (Manoharan et al., 1995; Shea et al., 1990), a polyamine or a polyethylene glycol chain (Manoharan et al., 1995), or adamantane acetic acid (Manoharan et al., 1995), a palmityl moiety (Mishra et al., 1995), or an octadecylamine or hexylamino-carbonyl-oxycholesterol moiety (Crooke et al., 1996).

Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779:4,789,737; 4,824.941; 4,835,263; 4.876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

### F. Cancer and Hyperproliferative Diseases

While hyperproliferative diseases can be associated with any disease which causes a cell to begin to reproduce uncontrollably, the prototypical example is cancer. One of the key elements of cancer is that the cell's normal apoptotic cycle is interrupted and thus agents that interrupt the growth of the cells are important as therapeutic agents for treating these diseases. In some embodiments, the target gene or transcript with which the guide polynucleotide may form a complex may be found in a human cell, such as a cancer cell. In some embodiments, the compounds of the disclosure may interfere with gene expression in a human cell, such as an cancer cell. The methods described in the present disclosure contemplate interference with gene expression of either or both a healthy cell or a cancerous cell. In this disclosure, the cell membrane disrupting compounds described herein may be used to lead to decreased cell counts and as such can potentially be used to treat a variety of types of cancer lines. In some aspects, it is anticipated that the compounds and compositions described herein may be used to treat virtually any malignancy.

Cancer cells that may be treated with the compounds or compositions of the present disclosure include but are not limited to cells from the skin, bladder, blood, bone, bone marrow, brain, breast, colon, esophagus, gastrointestine, gum, head, kidney, liver, lung, nasopharynx, neck, ovary, prostate, skin, stomach, pancreas, testis, tongue, cervix, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; Mullerian mixed tumor; nephroblastoma; hepatoblastoma; carcinosarcoma; mesenchymoma, malignant; Brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. In certain aspects, the tumor may comprise an osteosarcoma, angiosarcoma, rhabdosarcoma, leiomyosarcoma, Ewing sarcoma, glioblastoma, neuroblastoma, or leukemia.

### G. Methods of Treatment

Disclosed herein includes methods for treating a subject having or suspected of having a disease or disorder, such as a genetic disease or disorder or a disease or disorder associated with a mutation to one or more genes, the method comprising administering to the subject a composition comprising one or more of each of the following nucleic acids: a polynucleotide comprising a sequence encoding for a polynucleotide-guided nuclease such as an mRNA; a guide polynucleotide, particularly a polynucleotide which has been configured to complex with at least a portion of a target gene or transcript or a polynucleotide with a sequence that encodes for such a guide polynucleotide such as a sgRNA; and the interfering polynucleotide, particularly a polynucleotide configured to interfere with the expression of a target gene or transcript such as a DNA; and a lipid nanoparticle comprising at least one ionizable lipid; wherein the each of the nucleic acids are encapsulated within the lipid nanoparticle. The subject may be a mammal. The subject may be a non-human species (e.g., mouse, rat, rabbit, dog, monkey, gibbon, chimp, ape, baboon, cow, pig, horse, sheep, cat and other species). The subject may be a human. The subject may be determined to exhibit a mutation in a gene. In some embodiments, the administering comprises systemic (e.g., intravenous) administration. In some embodiments, the subject is selected from the group consisting of mouse, rat, monkey, and human. In some embodiments, the subject is a human. The present disclosure provides methods of using the compositions in conjunction with other therapeutic modalities such as surgery, chemotherapy, radiotherapy, or immunotherapy.

### Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammalI and calicheamicin omegaII); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine. thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine: arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DFMO); retinoids, such as retinoic acid; capecitabine; carboplatin, procarbazine, plicomycin, gemcitabien, navelbine, farnesyl-protein transferase inhibitors, transplatinum, and pharmaceutically acceptable salts, acids, or derivatives of any of the above.

### Radiotherapy

Other factors that cause DNA damage and have been used extensively include what are commonly known as y-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely and depend on the half-life of the isotope, the strength and type of radiation emitted, and the uptake by the neoplastic cells.

### Immunotherapy

The skilled artisan will understand that immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include B-cell maturation antigen, CD20, carcinoembryonic antigen, tyrosinase (p97), gp68, GPRC5D, TAG-72, HMFG, Sialyl Lewis Antigen, MucA, MucB, PLAP, laminin receptor, erb B, and p155. An alternative aspect of immunotherapy is to combine anticancer effects with immune-stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons α, β, ε, ω, and κ, IL-1, GM-CSF, and TNF (Bukowski et al., 1998; Davidson et al., 1998; Hellstrand et al., 1998); gene therapy, e.g., TNF. IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

In some aspects, a combination described herein includes an agent that decreases tumor immunosuppression, such as a chemokine (C—X—C motif) receptor 2 (CXCR²) inhibitor. In some embodiments, the CXCR² inhibitor is danirixin (CAS Registry Number: 954126-98-8). Danirixin is also known as GSK1325756 or 1-(4-chloro-2-hydroxy-3-piperidin-3-ylsulfonylphenyl)-3-(3-fluoro-2-methylphenyl) urea. Danirixin is disclosed, e.g., in Miller et al. Eur J Drug Metab Pharmacokinet (2014) 39:173-181; and Miller et al. BMC Pharmacology and Toxicology (2015), 16:18. In some embodiments, the CXCR² inhibitor is reparixin (CAS Registry Number: 266359-83-5). Reparixin is also known as repertaxin or (2R)-2-[4-(2-methylpropyl)phenyl]-N-methylsulfonylpropanamide. Reparixin is a non-competitive allosteric inhibitor of CXCR^{1/2}. Reparixin is disclosed, e.g., in Zarbock et al. British Journal of Pharmacology (2008), 1-8. In some embodiments, the CXCR² inhibitor is navarixin. Navarixin is also known as MK-7123, SCH527123, PS291822, or 2-hydroxy-N,N-dimethyl-3-[[2-[[(1R)-1-(5-methylfuran-2-yl) propyl]amino]-3,4-dioxocyclobuten-1-yl]amino]benzamide Navarixin is disclosed, e.g., in Ning et al. Mol Cancer Ther. 2012; 11 (C6): 1353-64. In some embodiments, the CXCR² inhibitor is AZD5069, also known as N-[2-[[(2,3-difluoropheny) methyl]thio]-6- {[(1R,2S)-2,3-dihydroxy-1-methylpropyl]oxy}-4-pyrimidinyl]-1-azetidinesulfonamide. In some embodiments, the CXCR² inhibitor is an anti-CXCR² antibody, such as those disclosed in WO2020/028479.

In some aspects, a combination described herein includes an agent that activates dendritic cells, such as, for example, a TLR agonist. A “TLR agonist” as defined herein is any molecule which activates a toll-like receptor as described in Bauer et al., 2001, Proc. Natl. Acad. Sci. USA 98:9237-9242. A TLR agonist may be a small molecule, a recombinant protein, an antibody or antibody fragment, a nucleic acid, or a protein. In certain embodiments, the TLR agonist is recombinant, a natural ligand, an immunostimulatory nucleotide sequence, a small molecule, a purified bacterial extract or an inactivated bacteria preparation.

Several agonists of TLR derived from microbes have been described, such as lipopolysaccharides, peptidoglycans, flagellin and lipoteichoic acid (Aderem et al., 2000, Nature 406:782-787; Akira et al., 2001, Nat. Immunol. 2:675-680) Some of these ligands can activate different dendritic cell subsets, that express distinct patterns of TLRs (Kadowaki et al., 2001, J. Exp. Med. 194:863-869). Therefore, a TLR agonist could be any preparation of a microbial agent that possesses TLR agonist properties. Certain types of untranslated DNA have been shown to stimulate immune responses by activating TLRs. In particular, immunostimulatory oligonucleotides containing CpG motifs have been widely disclosed and reported to activate lymphocytes (see, U.S. Pat. No. 6,194,388). A “CpG motif” as used herein is defined as an unmethylated cytosine-guanine (CpG) dinucleotide. Immunostimulatory oligonucleotides which contain CpG motifs can also be used as TLR agonists according to the methods of the present invention. The immunostimulatory nucleotide sequence may be stabilized by structure modification such as phosphorothioate modification or may be encapsulated in cationic liposomes to improve in vivo pharmacokinetics and tumor targeting.

In some embodiments, the immunotherapy may be an immune checkpoint inhibitor. The present disclosure may also provide compositions that inhibit an immune checkpoint Immune checkpoints either turn up a signal (e.g., co-stimulatory molecules) or turn down a signal. Immune checkpoints either turn up a signal (e.g., co-stimulatory molecules) or turn down a signal. Immune checkpoint proteins that may be targeted by immune checkpoint blockade include adenosine A₂A receptor (A₂AR), B7-H3 (also known as CD276), B and T lymphocyte attenuator (BTLA), CCL5, CD27, CD38, CD8A, CMKLR1, cytotoxic T-lymphocyte-associated protein 4 (CTLA-4, also known as CD152), CXCL9, CXCR⁵, glucocorticoid-induced tumour necrosis factor receptor-related protein (GITR), HLA-DRB1, ICOS (also known as CD278), HLA-DQA1, HLA-E, indoleamine 2,3-dioxygenase 1 (IDO1), killer-cell immunoglobulin (KIR), lymphocyte activation gene-3 (LAG-3, also known as CD223), Mer tyrosine kinase (MerTK), NKG7, OX40 (also known as CD134), programmed death 1 (PD-1), programmed death-ligand 1 (PD-L1, also known as CD274), PDCDILG2, PSMB10, STATI, T cell immunoreceptor with Ig and ITIM domains (TIGIT), T-cell immunoglobulin domain and mucin domain 3 (TIM-3), and V-domain Ig suppressor of T cell activation (VISTA, also known as C10orf54). In particular, the immune checkpoint inhibitors target the PD-1 axis and/or CTLA-4.

The immune checkpoint inhibitors may be drugs, such as small molecules, recombinant forms of ligand or receptors, or antibodies, such as human antibodies (e.g., International Patent Publication WO2015/016718; Pardoll, Nat Rev Cancer, 12 (4): 252-264, 2012; both incorporated herein by reference). Known inhibitors of the immune checkpoint proteins or analogs thereof may be used, in particular chimeric, humanized, or human forms of antibodies may be used. As the skilled person will know, alternative and/or equivalent names may be in use for certain antibodies mentioned in the present disclosure. Such alternative and/or equivalent names are interchangeable in the context of the present disclosure. For example, it is known that lambrolizumab is also known under the alternative and equivalent names MK-3475 and pembrolizumab.

In some embodiments, a PD-1 binding antagonist is a molecule that inhibits the binding of PD-1 to its ligand binding partners. In a specific aspect, the PD-1 ligand binding partners are PD-L1 and/or PD-L2. In another embodiment, a PD-L1 binding antagonist is a molecule that inhibits the binding of PD-L1 to its binding partners. In a specific aspect, PD-L1 binding partners are PD-1 and/or B7-1. In another embodiment, a PD-L2 binding antagonist is a molecule that inhibits the binding of PD-L2 to its binding partners. In a specific aspect, a PD-L2 binding partner is PD-1. The antagonist may be an antibody, an antigen-binding fragment thereof, an immunoadhesin, a fusion protein, or an oligopeptide. Exemplary antibodies are described in U.S. Pat. Nos. 8,735,553, 8,354,509, and 8,008,449, all of which are incorporated herein by reference. Other PD-1 axis antagonists for use in the methods provided herein are known in the art, such as described in U.S. Patent Application Publication Nos. 2014/0294898, 2014/022021, and 2011/0008369, all of which are incorporated herein by reference.

In some embodiments, a PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence)). In some embodiments, the PD-1 binding antagonist is AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PD-L2-Fc fusion soluble receptor described in WO2010/027827 and WO2011/066342.

Another immune checkpoint protein that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA-4 is similar to the T-cell co-stimulatory protein, CD28, and both molecules bind to CD80 and CD86, also called B7-1 and B7-2 respectively, on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen-binding fragment thereof, an immunoadhesin, a fusion protein, or oligopeptide. Anti-human-CTLA-4 antibodies (or VH and/or VL domains derived therefrom) suitable for use in the present methods can be generated using methods well known in the art. Alternatively, art-recognized anti-CTLA-4 antibodies can be used. For example, the anti-CTLA-4 antibodies disclosed in U.S. Pat. No. 8,119,129; PCT Publn. Nos. WO 01/14424, WO 98/42752, WO 00/37504 (CP675,206, also known as tremelimumab; formerly ticilimumab); U.S. Pat. No. 6,207,156; Hurwitz et al. (1998) Proc Natl Acad Sci USA, 95 (17): 10067-10071; Camacho et al. (2004) J Clin Oncology, 22 (145): Abstract No. 2505 (antibody CP-675206); and Mokyr et al. (1998) Cancer Res, 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen-binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2, and CDR3 domains of the VL region of ipilimumab. In another embodiment, the antibody competes for binding with and/or binds to the same epitope on CTLA-4 as the above-mentioned antibodies. In another embodiment, the antibody has an at least about 90% variable region amino acid sequence identity with the above-mentioned antibodies (e.g., at least about 90%, 95%, or 99% variable region identity with ipilimumab). Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

Another immune checkpoint protein that can be targeted in the methods provided herein is lymphocyte-activation gene 3 (LAG-3), also known as CD223. The complete protein sequence of human LAG-3 has the Genbank accession number NP-002277. LAG-3 is found on the surface of activated T cells, natural killer cells, B cells, and plasmacytoid dendritic cells. LAG-3 acts as an “off” switch when bound to MHC class II on the surface of antigen-presenting cells. Inhibition of LAG-3 both activates effector T cells and inhibitor regulatory T cells. In some embodiments, the immune checkpoint inhibitor is an anti-LAG-3 antibody (e.g., a human antibody, a humanized antibody, or a chimeric antibody), an antigen-binding fragment thereof, an immunoadhesin, a fusion protein, or oligopeptide. Anti-human-LAG-3 antibodies (or VH and/or VL domains derived therefrom) suitable for use in the present methods can be generated using methods well known in the art. Alternatively, art-recognized anti-LAG-3 antibodies can be used. An exemplary anti-LAG-3 antibody is relatlimab (also known as BMS-986016) or antigen-binding fragments and variants thereof (see, e.g., WO 2015/116539). Other exemplary anti-LAG-3 antibodies include TSR-033 (see, e.g., WO 2018/201096), MK-4280, and REGN3767. MGD013 is an anti-LAG-3/PD-1 bispecific antibody described in WO 2017/019846. FS118 is an anti-LAG-3/PD-L1 bispecific antibody described in WO 2017/220569.

Another immune checkpoint protein that can be targeted in the methods provided herein is V-domain Ig suppressor of T cell activation (VISTA), also known as C10orf54. The complete protein sequence of human VISTA has the Genbank accession number NP_071436. VISTA is found on white blood cells and inhibits T cell effector function. In some embodiments, the immune checkpoint inhibitor is an anti-VISTA3 antibody (e.g., a human antibody, a humanized antibody, or a chimeric antibody), an antigen-binding fragment thereof, an immunoadhesin, a fusion protein, or oligopeptide. Anti-human-VISTA antibodies (or VH and/or VL domains derived therefrom) suitable for use in the present methods can be generated using methods well known in the art. Alternatively, art-recognized anti-VISTA antibodies can be used. An exemplary anti-VISTA antibody is JNJ-61610588 (also known as onvatilimab) (see, e.g., WO 2015/097536, WO 2016/207717, WO 2017/137830, WO 2017/175058). VISTA can also be inhibited with the small molecule CA-170, which selectively targets both PD-L1 and VISTA (see, e.g., WO 2015/033299, WO 2015/033301).

Another immune checkpoint protein that can be targeted in the methods provided herein is indoleamine 2,3-dioxygenase (IDO). The complete protein sequence of human IDO has Genbank accession number NP_002155. In some embodiments, the immune checkpoint inhibitor is a small molecule IDO inhibitor. Exemplary small molecules include BMS-986205, epacadostat (INCB24360), and navoximod (GDC-0919).

Another immune checkpoint protein that can be targeted in the methods provided herein is CD38. The complete protein sequence of human CD38 has Genbank accession number NP_001766. In some embodiments, the immune checkpoint inhibitor is an anti-CD38 antibody (e.g., a human antibody, a humanized antibody, or a chimeric antibody), an antigen-binding fragment thereof, an immunoadhesin, a fusion protein, or oligopeptide. Anti-human-CD38 antibodies (or VH and/or VL domains derived therefrom) suitable for use in the present methods can be generated using methods well known in the art. Alternatively, art-recognized anti-CD38 antibodies can be used. An exemplary anti-CD38 antibody is daratumumab (see, e.g., U.S. Pat. No. 7,829,673).

Another immune checkpoint protein that can be targeted in the methods provided herein is ICOS, also known as CD278. The complete protein sequence of human ICOS has Genbank accession number NP_036224. In some embodiments, the immune checkpoint inhibitor is an anti-ICOS antibody (e.g., a human antibody, a humanized antibody, or a chimeric antibody), an antigen-binding fragment thereof, an immunoadhesin, a fusion protein, or oligopeptide.

Anti-human-ICOS antibodies (or VH and/or VL domains derived therefrom) suitable for use in the present methods can be generated using methods well known in the art. Alternatively, art-recognized anti-ICOS antibodies can be used. Exemplary anti-ICOS antibodies include JTX-2011 (see, e.g., WO 2016/154177, WO 2018/187191) and GSK3359609 (see, e.g., WO 2016/059602).

Another immune checkpoint protein that can be targeted in the methods provided herein is T cell immunoreceptor with Ig and ITIM domains (TIGIT). The complete protein sequence of human TIGIT has Genbank accession number NP_776160. In some embodiments, the immune checkpoint inhibitor is an anti-TIGIT antibody (e.g., a human antibody, a humanized antibody, or a chimeric antibody), an antigen-binding fragment thereof, an immunoadhesin, a fusion protein, or oligopeptide. Anti-human-TIGIT antibodies (or VH and/or VL domains derived therefrom) suitable for use in the present methods can be generated using methods well known in the art. Alternatively, art-recognized anti-TIGIT antibodies can be used. An exemplary anti-TIGIT antibody is MK-7684 (see, e.g., WO 2017/030823, WO 2016/028656).

Another immune checkpoint protein that can be targeted in the methods provided herein is OX40, also known as CD134. The complete protein sequence of human OX40 has Genbank accession number NP_003318. In some embodiments, the immune checkpoint inhibitor is an anti-OX40 antibody (e.g., a human antibody, a humanized antibody, or a chimeric antibody), an antigen-binding fragment thereof, an immunoadhesin, a fusion protein, or oligopeptide. Anti-human-OX40 antibodies (or VH and/or VL domains derived therefrom) suitable for use in the present methods can be generated using methods well known in the art. Alternatively, art-recognized anti-OX40 antibodies can be used. An exemplary anti-OX40 antibody is PF-04518600 (see, e.g., WO 2017/130076). ATOR-1015 is a bispecific antibody targeting CTLA4 and OX40 (see, e.g., WO 2017/182672, WO 2018/091740, WO 2018/202649, WO 2018/002339).

Another immune checkpoint protein that can be targeted in the methods provided herein is glucocorticoid-induced tumour necrosis factor receptor-related protein (GITR), also known as TNFRSF18 and AITR. The complete protein sequence of human GITR has Genbank accession number NP_004186. In some embodiments, the immune checkpoint inhibitor is an anti-GITR antibody (e.g., a human antibody, a humanized antibody, or a chimeric antibody), an antigen-binding fragment thereof, an immunoadhesin, a fusion protein, or oligopeptide. Anti-human-GITR antibodies (or VH and/or VL domains derived therefrom) suitable for use in the present methods can be generated using methods well known in the art. Alternatively, art-recognized anti-GITR antibodies can be used. An exemplary anti-GITR antibody is TRX518 (see, e.g., WO 2006/105021).

In some embodiment, the immune therapy could be adoptive immunotherapy, which involves the transfer of autologous antigen-specific T cells generated ex vivo. The T cells used for adoptive immunotherapy can be generated either by expansion of antigen-specific T cells or redirection of T cells through genetic engineering (Park, Rosenberg et al. 2011). Isolation and transfer of tumor-specific T cells has been shown to be successful in treating melanoma. Novel specificities in T cells have been successfully generated through the genetic transfer of transgenic T cell receptors or chimeric antigen receptors (CARs) (Jena, Dotti et al. 2010). CARs are synthetic receptors consisting of a targeting moiety that is associated with one or more signaling domains in a single fusion molecule. In general, the binding moiety of a CAR consists of an antigen-binding domain of a single-chain antibody (scFv), comprising the light and variable fragments of a monoclonal antibody joined by a flexible linker. Binding moieties based on receptor or ligand domains have also been used successfully. The signaling domains for first-generation CARs are derived from the cytoplasmic region of the CD3zeta or the Fc receptor gamma chains. CARs have successfully allowed T cells to be redirected against antigens expressed at the surface of tumor cells from various malignancies including lymphomas and solid tumors (Jena, Dotti et al. 2010).

In one embodiment, the present application provides for a combination therapy for the treatment of cancer wherein the combination therapy comprises adoptive T cell therapy and a checkpoint inhibitor. In one aspect, the adoptive T cell therapy comprises autologous and/or allogenic T-cells. In another aspect, the autologous and/or allogenic T-cells are targeted against tumor antigens.

### D. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, q_{, 10, 11}, or 12 months. These treatments may be of varying dosages as well.

### Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy. The present compositions may perform one or more of these functions and then combined with another agent to enhance the activity of the present compositions.

### H. Kits

The present disclosure also provides kits. Any of the components disclosed herein may be combined in the form of a kit. In some embodiments, the kits comprise a composition as described above or in the claims.

The kits will generally include at least one vial, test tube, flask, bottle, syringe or other container, into which a component may be placed, and preferably, suitably aliquoted. Where there is more than one component in the kit, the kit also will generally contain a second, third or other additional containers into which the additional components may be separately placed. However, various combinations of components may be comprised in a container. In some embodiments, all of the lipid nanoparticle components are combined in a single container. In other embodiments, some or all of the lipid nanoparticle components are provided in separate containers.

The kits of the present invention also will typically include packaging for containing the various containers in close confinement for commercial sale. Such packaging may include cardboard or injection or blow molded plastic packaging into which the desired containers are retained. A kit may also include instructions for employing the kit components. Instructions may include variations that can be implemented.

### I. Chemical Definitions

When used in the context of a chemical group; “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanate” means —N═C═O; “azido” means —N₃; in a monovalent context “phosphate” means —OP(O)(OH)₂ or a deprotonated form thereof; in a divalent context “phosphate” means —OP(O)(OH)O—or a deprotonated form thereof; “mercapto” means —SH; and “thio” means ═S; “sulfonyl” means —S(O)₂—; “hydroxysulfonyl” means —S(O)₂OH; “sulfonamide” means —S(O)₂NH₂; and “sulfinyl” means —S(O)—.

In the context of chemical formulas, the symbol “—” means a single bond, “═” means a double bond, and “≡” means triple bond. The symbol “- - - -” represents an optional bond, which if present is either single or double. The symbol “ [symbol] ” represents a single bond or a double bond. Thus, for example, the formula

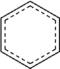

includes

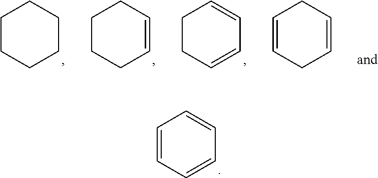

And it is understood that no one such ring atom forms part of more than one double bond. Furthermore, it is noted that the covalent bond symbol “-”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.,

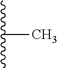

for methyl) indicates a point or attachment of the group. It is noted that the point of attachment is typically only identified in this manner for larger groups in order to assist the reader in unambiguously identifying a point of attachment. The symbol “ [symbol] ” “means a single bond where the group attached to the thick end of the wedge is “out of the page.” The symbol “ [symbol] ” “means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a group “R” is depicted as a “floating group” on a ring system, for example, in the formula:

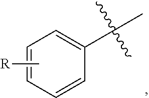

then R may replace any hydrogen atom attached to any of the ring atoms, including a depicted, implied, or expressly defined hydrogen, so long as a stable structure is formed. When a group “R” is depicted as a “floating group” on a fused ring system, as for example in the formula:

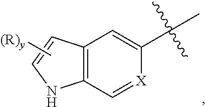

then R may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals-CH—), so long as a stable structure is formed. In the example depicted, R may reside on either the 5-membered or the 6-membered ring of the fused ring system. In the formula above, the subscript letter “y” immediately following the group “R” enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows; “Cn” defines the exact number (n) of carbon atoms in the group/class. “C≤n” defines the maximum number (n) of carbon atoms that can be in the group/class, with the minimum number as small as possible for the group/class in question, e.g., it is understood that the minimum number of carbon atoms in the group “alkenyl_{(C≤8)}” or the class “alkene_{(C≤8)}” is two. Compare with “alkoxy_{(C≤10)}”, which designates alkoxy groups having from 1 to 10 carbon atoms. “Cn-n” “′ defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C5 olefin”, “C5-olefin”. “olefin (C5)”, and “olefincs” are all synonymous.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” when used without the “substituted” modifier signifies that the compound or chemical group so modified is an acyclic or cyclic, but non-aromatic hydrocarbon compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” when used to modify a compound or a chemical group atom means the compound or chemical group contains a planar unsaturated ring of atoms that is stabilized by an interaction of the bonds forming the ring.

The term “alkyl” when used without the “substituted” modifier refers to a monovalent saturated aliphatic group with a carbon atom as the point of attachment, a linear or branched acyclic structure, and no atoms other than carbon and hydrogen. The groups-CH₃ (Me), —CH₂CH₃ (Et), —CH₂CH₂CH₃ (n-Pr or propyl), —CH (CH₃)₂ (i-Pr, ⁱPr or isopropyl), —CH₃CH₂CH₂CH₃ (n-Bu), —CH (CH₃) CH₂CH₃ (sec-butyl), —CH₂CH (CH₃)₂ (isobutyl), —C(CH₃)₃ (tert-butyl, t-butyl, t-Bu or ^{t}Bu), and —CH₂C (CH₃)₃ (neo-pentyl) are non-limiting examples of alkyl groups. The term “alkanediyl” when used without the “substituted” modifier refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups-CH₂-(methylene), —CH₃CH₂—, —CH₂C (CH₃)₂CH₂—, and —CH₂CH₂CH₂—are non-limiting examples of alkanediyl groups. An “alkane” refers to the class of compounds having the formula H—R, wherein R is alkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups-CH₂F, —CF₃, and —CH₂CF₃ are non-limiting examples of fluoroalkyl groups.

The term “cycloalkyl” when used without the “substituted” modifier refers to a monovalent saturated aliphatic group with a carbon atom as the point of attachment, said carbon atom forming part of one or more non-aromatic ring structures, no carbon-carbon double or triple bonds, and no atoms other than carbon and hydrogen. Non-limiting examples include: —CH (CH₂)₂ (cyclopropyl), cyclobutyl, cyclopentyl, or cyclohexyl(Cy). The term “cycloalkanediyl” when used without the “substituted” modifier refers to a divalent saturated aliphatic group with two carbon atoms as points of attachment, no carbon-carbon double or triple bonds, and no atoms other than carbon and hydrogen. The group

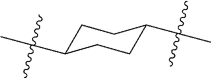 [SMILES: *C1CCC(*)CC1]

is a non-limiting example of cycloalkanediyl group. A “cycloalkane” refers to the class of compounds having the formula H—R, wherein R is cycloalkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The term “alkenyl” when used without the “substituted” modifier refers to an monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH—CH₂. The term “alkenediyl” when used without the “substituted” modifier refers to a divalent unsaturated aliphatic group, with two carbon atoms as points of attachment, a linear or branched, a linear or branched acyclic structure, at least one nonaromatic carbon-carbon double bond, no carbon-carbon triple bonds, and no atoms other than carbon and hydrogen. The groups-CH═CH—, —CH═C (CH₃) CH₂—, —CH═CHCH₂—, and —CH₂CH—CHCH₂—are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H—R, wherein R is alkenyl as this term is defined above. Similarly the terms “terminal alkene” and “α-olefin” are synonymous and refer to an alkene having just one carbon-carbon double bond, wherein that bond is part of a vinyl group at an end of the molecule. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)—NH₂. The groups-CH═CHF, —CH═CHCI and —CH═CHBr are non-limiting examples of substituted alkenyl groups.

The term “alkynyl” when used without the “substituted” modifier refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups-C≡CH, —C≡CCH₃, and —CH₂C≡CCH₃ are non-limiting examples of alkynyl groups. An “alkyne” refers to the class of compounds having the formula H—R, wherein R is alkynyl. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aryl” when used without the “substituted” modifier refers to a monovalent unsaturated aromatic group with an aromatic carbon atom as the point of attachment, said carbon atom forming part of a one or more six-membered aromatic ring structure, wherein the ring atoms are all carbon, and wherein the group consists of no atoms other than carbon and hydrogen. If more than one ring is present, the rings may be fused or unfused. As used herein, the term does not preclude the presence of one or more alkyl or aralkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl. The term “arenediyl” when used without the “substituted” modifier refers to a divalent aromatic group with two aromatic carbon atoms as points of attachment, said carbon atoms forming part of one or more six-membered aromatic ring structure(s) wherein the ring atoms are all carbon, and wherein the monovalent group consists of no atoms other than carbon and hydrogen. As used herein, the term does not preclude the presence of one or more alkyl, aryl or aralkyl groups (carbon number limitation permitting) attached to the first aromatic ring or any additional aromatic ring present. If more than one ring is present, the rings may be fused or unfused. Unfused rings may be connected via one or more of the following: a covalent bond, alkanediyl, or alkenediyl groups (carbon number limitation permitting). Non-limiting examples of arenediyl groups include:

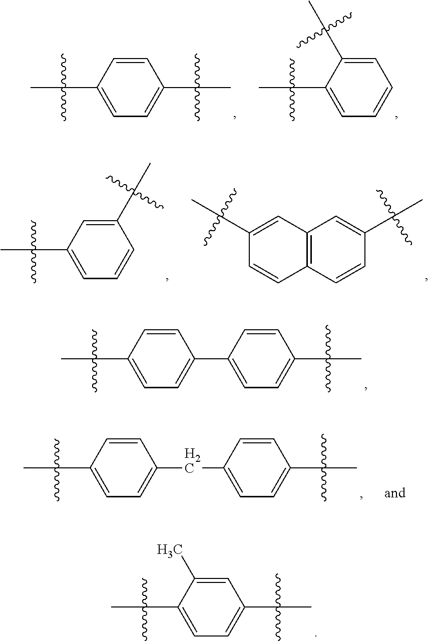

An “arene” refers to the class of compounds having the formula H—R, wherein R is aryl as that term is defined above. Benzene and toluene are non-limiting examples of arenes. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aralkyl” when used without the “substituted” modifier refers to the monovalent group-alkanediyl-aryl, in which the terms alkanediyl and aryl are each used in a manner consistent with the definitions provided above. Non-limiting examples are: phenylmethyl (benzyl, Bn) and 2-phenyl-ethyl. When the term aralkyl is used with the “substituted” modifier one or more hydrogen atom from the alkanediyl and/or the aryl group has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. Non-limiting examples of substituted aralkyls are: (3-chlorophenyl)-methyl, and 2-chloro-2-phenyl-eth-1-yl.

The term “heteroaryl” when used without the “substituted” modifier refers to a monovalent aromatic group with an aromatic carbon atom or nitrogen atom as the point of attachment, said carbon atom or nitrogen atom forming part of one or more aromatic ring structures wherein at least one of the ring atoms is nitrogen, oxygen or sulfur, and wherein the heteroaryl group consists of no atoms other than carbon, hydrogen, aromatic nitrogen, aromatic oxygen and aromatic sulfur. Heteroaryl rings may contain 1, 2, 3, or 4 ring atoms selected from are nitrogen, oxygen, and sulfur. If more than one ring is present, the rings may be fused or unfused. As used herein, the term does not preclude the presence of one or more alkyl, aryl, and/or aralkyl groups (carbon number limitation permitting) attached to the aromatic ring or aromatic ring system. Non-limiting examples of heteroaryl groups include furanyl, imidazolyl, indolyl, indazolyl (Im), isoxazolyl, methylpyridinyl, oxazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. The term “heteroarenediyl” when used without the “substituted” modifier refers to an divalent aromatic group, with two aromatic carbon atoms, two aromatic nitrogen atoms, or one aromatic carbon atom and one aromatic nitrogen atom as the two points of attachment, said atoms forming part of one or more aromatic ring structure(s) wherein at least one of the ring atoms is nitrogen, oxygen or sulfur, and wherein the divalent group consists of no atoms other than carbon, hydrogen, aromatic nitrogen, aromatic oxygen and aromatic sulfur. If more than one ring is present, the rings may be fused or unfused. Unfused rings may be connected via one or more of the following: a covalent bond, alkanediyl, or alkenediyl groups (carbon number limitation permitting). As used herein, the term does not preclude the presence of one or more alkyl, aryl, and/or aralkyl groups (carbon number limitation permitting) attached to the aromatic ring or aromatic ring system. Non-limiting examples of heteroarenediyl groups include:

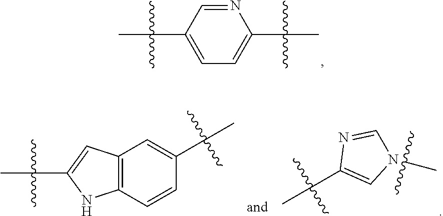

A “heteroarene” refers to the class of compounds having the formula H—R, wherein R is heteroaryl. Pyridine and quinoline are non-limiting examples of heteroarenes. When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH—CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “heterocycloalkyl” when used without the “substituted” modifier refers to a monovalent non-aromatic group with a carbon atom or nitrogen atom as the point of attachment, said carbon atom or nitrogen atom forming part of one or more non-aromatic ring structures wherein at least one of the ring atoms is nitrogen, oxygen or sulfur, and wherein the heterocycloalkyl group consists of no atoms other than carbon, hydrogen, nitrogen, oxygen and sulfur. Heterocycloalkyl rings may contain 1, 2, 3, or 4 ring atoms selected from nitrogen, oxygen, or sulfur. If more than one ring is present, the rings may be fused or unfused. As used herein, the term does not preclude the presence of one or more alkyl groups (carbon number limitation permitting) attached to the ring or ring system. Also, the term does not preclude the presence of one or more double bonds in the ring or ring system, provided that the resulting group remains non-aromatic. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, pyranyl, oxiranyl, and oxetanyl. The term “N-heterocycloalkyl” refers to a heterocycloalkyl group with a nitrogen atom as the point of attachment. N-pyrrolidinyl is an example of such a group. The term “heterocycloalkanediyl” when used without the “substituted” modifier refers to an divalent cyclic group, with two carbon atoms, two nitrogen atoms, or one carbon atom and one nitrogen atom as the two points of attachment, said atoms forming part of one or more ring structure(s) wherein at least one of the ring atoms is nitrogen, oxygen or sulfur, and wherein the divalent group consists of no atoms other than carbon, hydrogen, nitrogen, oxygen and sulfur. If more than one ring is present, the rings may be fused or unfused. Unfused rings may be connected via one or more of the following: a covalent bond, alkanediyl, or alkenediyl groups (carbon number limitation permitting). As used herein, the term does not preclude the presence of one or more alkyl groups (carbon number limitation permitting) attached to the ring or ring system. Also, the term does not preclude the presence of one or more double bonds in the ring or ring system, provided that the resulting group remains non-aromatic. Non-limiting examples of heterocycloalkanediyl groups include:

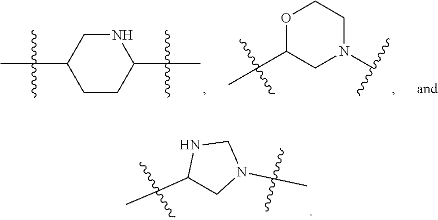 [SMILES: *C1CCC(*)NC1 ; *C1CN(*)CCO1 ; *C1CN(*)CN1]

When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “acyl” when used without the “substituted” modifier refers to the group —C(O)R, in which R is a hydrogen, alkyl, cycloalkyl, alkenyl, aryl, aralkyl or heteroaryl, as those terms are defined above. The groups, —CHO, —C(O)CH₃ (acetyl, Ac), —C(O)CH₂CH₃, —C(O)CH₂CH₂CH₃, —C(O)CH (CH₃)₂, —C(O)CH (CH₂)₂, —C(O)C₆H₅, —C(O)C₆H₄CH₃, —C(O)CH₂C₆H₅, —C(O)(imidazolyl) are non-limiting examples of acyl groups. A “thioacyl” is defined in an analogous manner, except that the oxygen atom of the group —C(O)R has been replaced with a sulfur atom, —C(S) R. The term “aldehyde” corresponds to an alkane, as defined above, wherein at least one of the hydrogen atoms has been replaced with a-CHO group. When any of these terms are used with the “substituted” modifier one or more hydrogen atom (including a hydrogen atom directly attached to the carbon atom of the carbonyl or thiocarbonyl group, if any) has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups, —C(O)CH: CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups.

The term “alkoxy” when used without the “substituted” modifier refers to the group —OR, in which R is an alkyl, as that term is defined above. Non-limiting examples include: —OCH₃ (methoxy), —OCH₂CH₃ (ethoxy), —OCH₂CH₂CH₃, —OCH (CH₃)₂ (isopropoxy), —OC (CH₃)₃ (tert-butoxy), —OCH (CH₂)₂, —O-cyclopentyl, and —O-cyclohexyl. The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkoxydiyl” refers to the divalent group —O-alkanediyl-, —O-alkanediyl —O—, or-alkanediyl-O-alkanediyl-. The term “alkylthio” and “acylthio” when used without the “substituted” modifier refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃): , —C(O)NH₂, —C(O)NHCH₃, —C(O)N(CH₃)₂, —OC(O)CH₃, —NHC(O)CH₃, —S(O)₂OH, or —S(O)₂NH₂.

The term “alkylamino” when used without the “substituted” modifier refers to the group —NHR, in which R is an alkyl, as that term is defined above. Non-limiting examples include: —NHCH₃ and —NHCH₂CH₃. The term “dialkylamino” when used without the “substituted” modifier refers to the group —NRR′, in which R and R′ can be the same or different alkyl groups, or R and R′ can be taken together to represent an alkanediyl. Non-limiting examples of dialkylamino groups include: —N(CH₃)₂ and —N(CH₃)(CH₂CH₃) . The terms “cycloalkylamino”, “alkenylamino”, “alkynylamino”, “arylamino”, “aralkylamino”, “heteroaryl lamino”, “heterocycloalkylamino”, “alkoxyamino”, and “alkylsulfonylamino” when used without the “substituted” modifier, refers to groups, defined as —NHR, in which R is cycloalkyl, alkenyl, alkynyl, aryl, aralkyl, heteroaryl, heterocycloalkyl, alkoxy, and alkylsulfonyl, respectively. A non-limiting example of an arylamino group is —NHC₆H₅. The term “alkylaminodiyl” refers to the divalent group —NH-alkanediyl-, —NH-alkanediyl —NH—, or -alkanediyl-NH-alkanediyl-. The term “amido” (acylamino), when used without the “substituted” modifier, refers to the group —NHR, in which R is acyl, as that term is defined above. A non-limiting example of an amido group is —NHC(O)CH₃. The term “alkylimino” when used without the “substituted” modifier refers to the divalent group ═NR, in which R is an alkyl, as that term is defined above. When any of these terms is used with the “substituted” modifier one or more hydrogen atom attached to a carbon atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups —NHC(O)OCH₃ and —NHC(O)NHCH₃ are non-limiting examples of substituted amido groups.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects.

As used in this application, the term “average molecular weight” refers to the relationship between the number of moles of each polymer species and the molar mass of that species. In particular, each polymer molecule may have different levels of polymerization and thus a different molar mass. The average molecular weight can be used to represent the molecular weight of a plurality of polymer molecules. Average molecular weight is typically synonymous with average molar mass. In particular, there are three major types of average molecular weight: number average molar mass, weight (mass) average molar mass, and Z-average molar mass. In the context of this application, unless otherwise specified, the average molecular weight represents either the number average molar mass or weight average molar mass of the formula. In some embodiments, the average molecular weight is the number average molar mass. In some embodiments, the average molecular weight may be used to describe a PEG component present in a lipid.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to a subject or patient for treating a disease, is sufficient to effect such treatment for the disease.

As used herein, the term “IC50” refers to an inhibitory dose which is 50% of the maximum response obtained. This quantitative measure indicates how much of a particular drug or other substance (inhibitor) is needed to inhibit a given biological, biochemical or chemical process (or component of a process, i.e., an enzyme, cell, cell receptor or microorganism) by half.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat. guinea pig, or transgenic species thereof. In certain embodiments, the patient or subject is a primate. Non-limiting examples of human subjects are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds of the present invention which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis (3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

The term “pharmaceutically acceptable carrier,” as used herein means a pharmaceutically-acceptable material, composition or vehicle, such as a liquid or solid filler, diluent, excipient, solvent or encapsulating material, involved in carrying or transporting a chemical agent.

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

A “repeat unit” is the simplest structural entity of certain materials, for example, frameworks and/or polymers, whether organic, inorganic or metal- organic. In the case of a polymer chain, repeat units are linked together successively along the chain, like the beads of a necklace. For example, in polyethylene, -[—CH₂CH₂-]ₙ-, the repeat unit is —CH₂CH₂—. The subscript “n” denotes the degree of polymerization, that is, the number of repeat units linked together. When the value for “n” is left undefined or where “n” is absent, it simply designates repetition of the formula within the brackets as well as the polymeric nature of the material. The concept of a repeat unit applies equally to where the connectivity between the repeat units extends three dimensionally, such as in metal organic frameworks, modified polymers, thermosetting polymers, etc. Within the context of the dendrimer, the repeating unit may also be described as the branching unit, interior layers, or generations. Similarly, the terminating group may also be described as the surface group.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer.

In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2″, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

The above definitions supersede any conflicting definition in any reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

### J. Examples

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1: SiFAK+CRISPR-LNPs Enhance Gene Editing In Vitro Via Modulation of Tumor Tensile Force

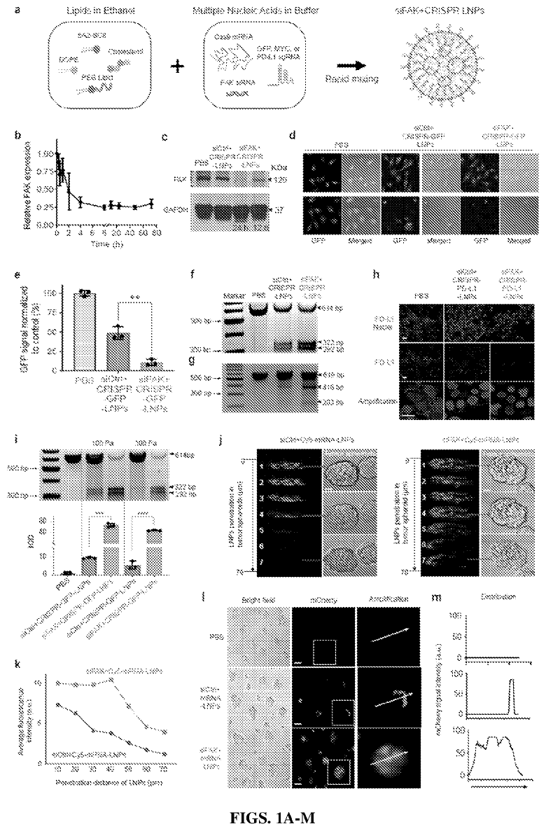

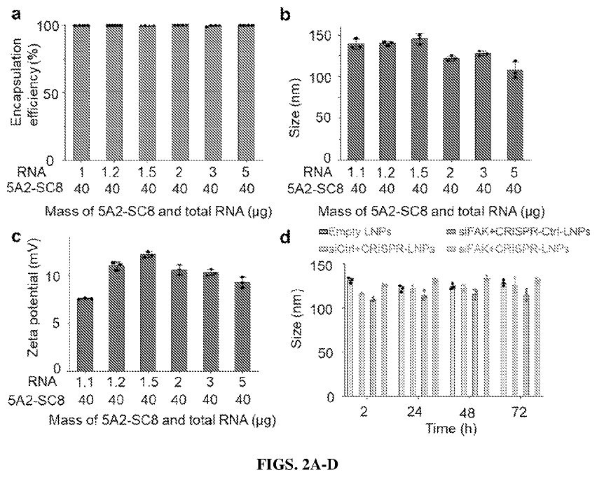

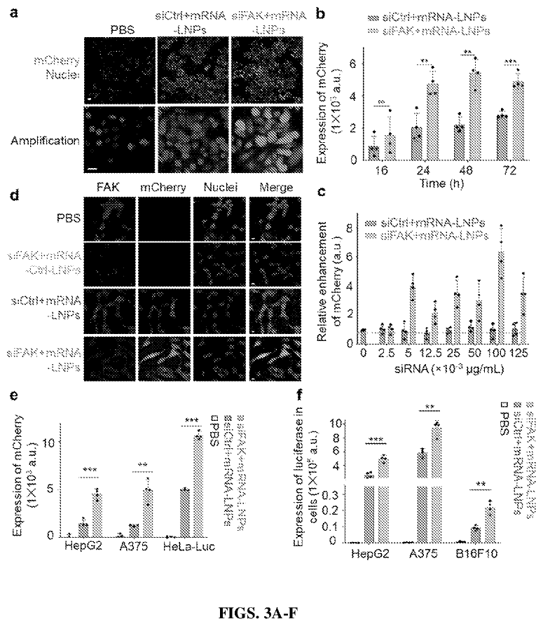

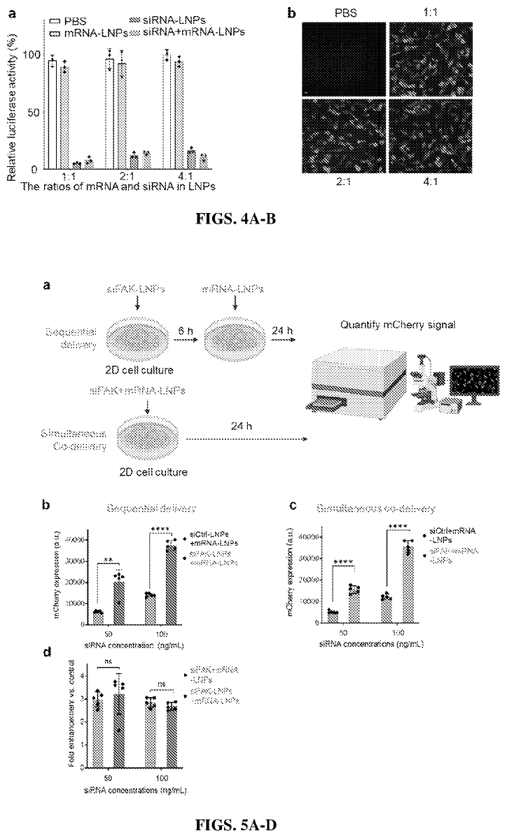

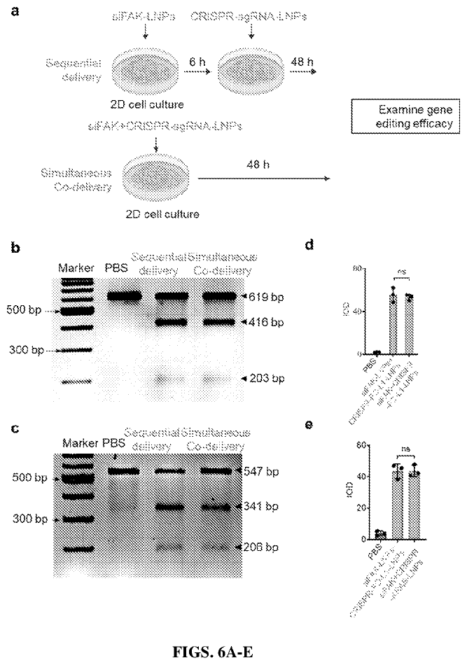

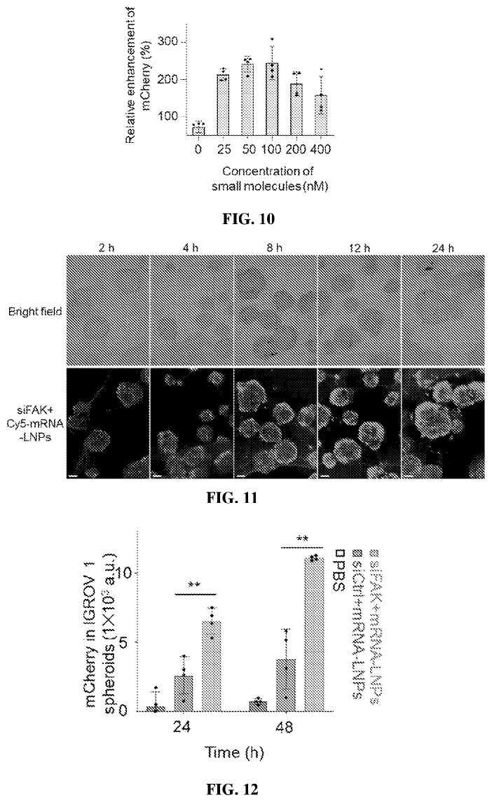

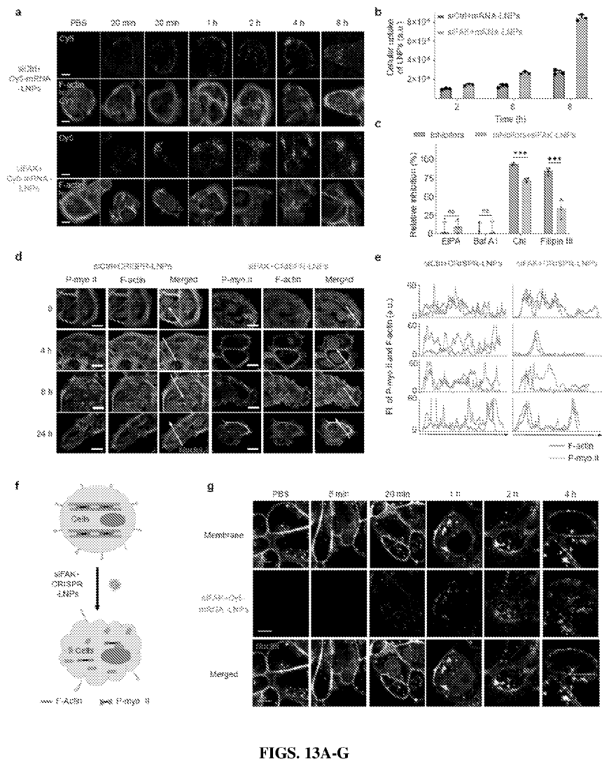

To examine the hypothesis that FAK silencing would improve CRISPR gene editing, a nanoparticle system capable of delivering three types of nucleic acids (FAK siRNA (siFAK), Cas9 mRNA, and sgRNA) in one nanoparticle was developed. Self-assembled lipid nanoparticles (siFAK+CRISPR-LNPs) (FIG. 1A) were constructed that utilized 5A2-SC8 (see Example 7) as the ionizable amino lipid dendrimer to bind negatively charged RNAs and facilitate endosomal escape after cellular uptake due to charge acquisition at endosomal pH. 5A2-SC8 was selected based on prior work delivering siRNA and mRNA to the liver (Zhou et al, 2016; Cheng et al, 2018; Cheng et al, 2020). To further develop LNPs for co-encapsulation of three nucleic acids of different physiochemical properties (Miller et al, 2017; Cheng et al, 2020; Ball et al, 2018; Patel et al, 2020; Abumanhal-Masarweh et al, 2019; Wei et al, 2020; Liu et al, 2021; Lee et al, 2021), the mass ratio of 5A2-SC8: RNA and LNP molecular composition with respect to other lipids (5A2-SC8: cholesterol: DOPE: DMG-PEG2000: DSPE-PEG2000=15:30:15:2:1 (mol)) was optimized. This led to targeted LNPs capable of triple RNA loading with high RNA encapsulation efficiency (FIG. 2A). Optimized LNPs were ˜ 120 nm in size and ˜ 10 mV in average surface charge, which can remain stable in serum-containing medium (5% w/v) (FIG. 2B, FIG. 2C) without obvious changes in size over 72 h incubation (FIG. 2D). It is noted that CRISPR was deployed using Cas9 mRNA because this approach results in transient Cas9 expression, which can minimize off-target effects without risk of integration, as compared to viral or pDNA delivery approaches (Wang et al, 2017). siFAK+CRISPR-LNPs were then delivered specifically targeting GFP into human HeLa cells stably expressing green fluorescent protein (HeLa-GFP) and the efficacy of gene silencing and gene editing was assessed. siRNA-mediated gene silencing successfully inhibited FAK expression (FIG. 1B and FIG. 1C). Maximal knockdown (˜80%) was quantified from 4 h to 72 h post-administration of siFAK+CRISPR-LNPs (FIG. 1B-1C). GFP expression was inhibited through administration of all LNPs containing Cas9 mRNA and sgGFP (siFAK+CRISPR-GFP-LNPs). GFP expression was reduced more in Hel a-GFP cells treated with siFAK+CRISPR-GFP-LNPs compared to cells treated with siCtrl+CRISPR-GFP-LNPs (in which luciferase siRNA served as control) (FIG. 1D and FIG. 1E). T7 endonuclease 1 (T7E1) mutation detection assay results revealed that the frequency of indels in GFP DNA were higher in cells treated with siFAK+CRISPR-GFP-LNPs than in cells treated with siCtrl+CRISPR-GFP-LNPs (FIG. 1F). These data indicated that multiplexed LNPs successfully delivered all three types of nucleic acids to human cancer cell lines to achieve highly efficient gene silencing and genome editing. An intriguing phenomenon was also revealed where FAK knockdown significantly enhanced gene editing of GFP. The results were confirmed through administration of mCherry mRNA and luciferase mRNA in multiple tumor cell types (FIG. 3A-FIG. 3F). The enhancement of mRNA delivery and protein expression was time- and siRNA-concentration-dependent irrespective of the ratio of loaded cargoes (FIG. 3B, FIG. 3C, FIG. 4A-FIG. 4B). Based on the higher delivery efficacy of simultaneous co-delivery of siRNA and mRNA in vivo than sequential delivery using multiple LNPs (FIGS. 5-9), the simultaneous delivery approach was utilized for further studies. Next, to confirm that delivery enhancement was regulated by FAK knockdown, FAK was inactivated using a small molecule inhibitor in cells co-administered with siCtrl+mRNA-LNPs. Consistent with the siFAK co-delivery results, mRNA delivery efficacy and protein expression were also enhanced in cells treated with an FAK inhibitor drug (FIG. 10). Furthermore, siFAK+CRISPR-LNPs were delivered to human ovarian cancer cells (IGROV1) specifically targeting PD-L1 (siFAK+CRISPR-PD-L1-LNPs) to examine the gene editing efficiency. Insertions and deletions (indels) at the PD-L1 locus were also significantly increased with FAK knockdown (FIG. 1G), leading to distinctly reduced PD-L1 protein expression (FIG. 1H). Thus, it was concluded that suppression of FAK improves nanoparticle-based CRISPR gene editing efficacy in multiple tumor cell lines. Next, delivery in 3D multicellular spheroids that have been shown to recapitulate critical in vivo physiologic tumor parameters (Mehta et al, 2012) was examined. siFAK+CRISPR-GFP-LNPs were able to achieve genome editing in spheroids, where DNA cleavage was 7-fold higher than siCtrl+CRISPR-GFP-LNPs (FIG. 1I). Reporter mCherry mRNA and Cy5-labelled mRNA were also delivered to examine both efficacy and spatial LNP delivery. siFAK+mRNA-LNPs penetrated throughout the entire spheroid within 4 hours as tracked by Cy5 fluorescence (FIG. 1J-K and FIG. 11), which led to mCherry expression following mRNA translation throughout the entire spheroid (FIG. 1L and FIG. 1M). In contrast, siCtrl+mRNA-LNPs were unable to penetrate the center of the spheroids and only delivered mRNA to the periphery (FIG. 1J-1M and FIG. 12). These results suggested, without being bound by theory, that FAK knockdown can overcome the physical barriers of tumor spheroids and distinctly increase RNA delivery.

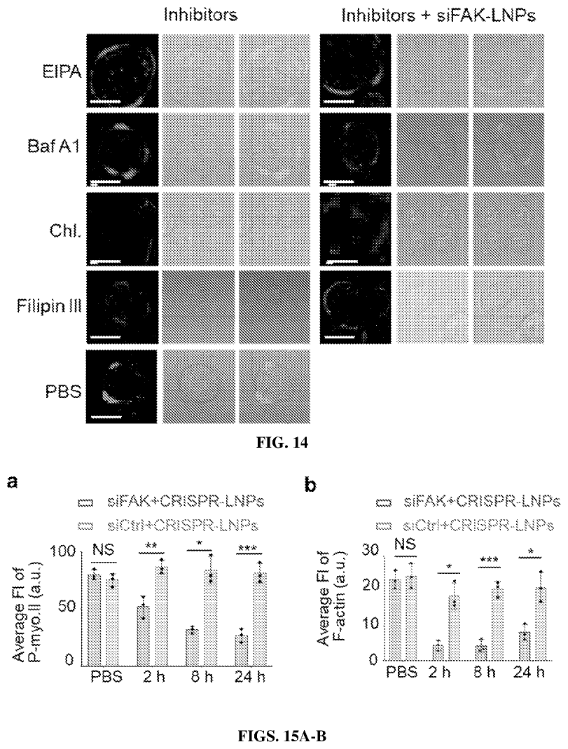

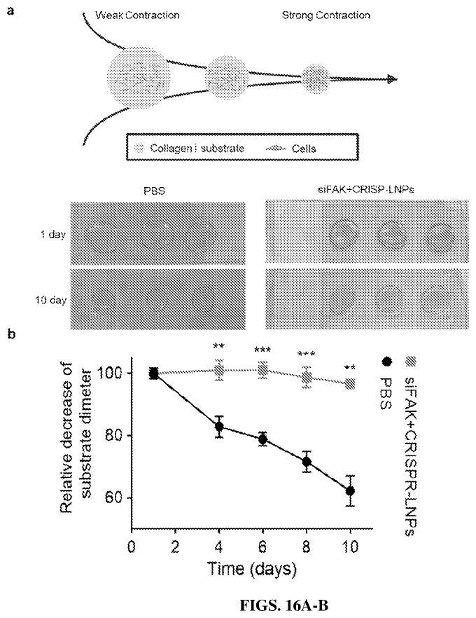

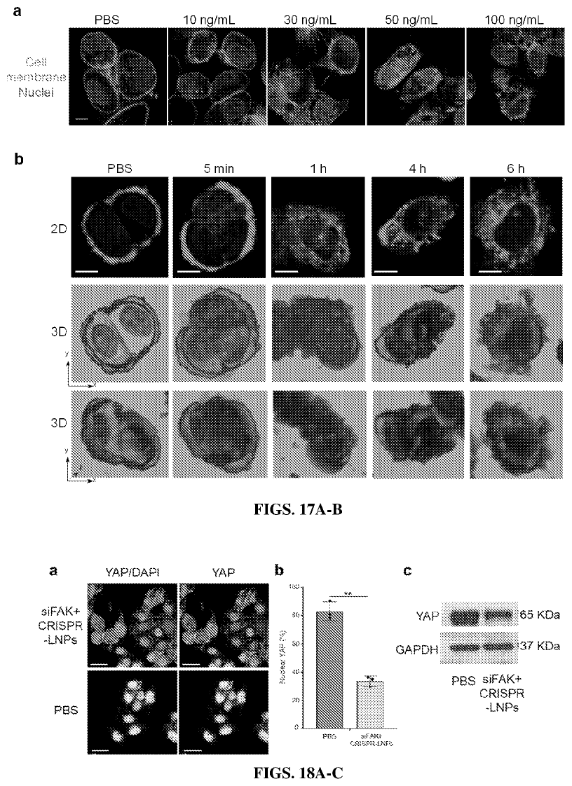

To investigate the mechanism for how FAK knockdown enhances gene editing efficacy of siFAK+CRISPR-LNPs, the cellular uptake of LNPs was quantified using confocal microscopy and flow cytometry. Comparing Cy5-labeled siCtrl+mRNA-LNPs and Cy5-labeled siFAK+mRNA-LNPs treated cells, higher cellular uptake was observed in the siFAK group (FIG. 13A-13B). Next, various small molecules were used to inhibit distinct cellular uptake pathways. The results indicated that FAK silencing increased cellular uptake of LNPs mainly through modulating clathrin- and caveolae-dependent endocytosis pathways (FIG. 13C and FIG. 14). Membrane invagination and endocytosis are the major steps for these two pathways, involving membrane tension regulated by tensile forces generated by actin filament (F-actin) and the actomyosin network. With this insight, the dynamic changes of F-actin/stress fibers and the actomyosin network distribution (FIG. 13D-13E and FIG. 15) were examined. Compared with the strong stress fibers and rich actomyosin network in cells treated with siCtrl+CRISPR-LNPs, both stress fibers and actomyosin network were decreased, indicating that the contraction force of cells treated with siFAK+CRISPR-LNPs was distinctly decreased. The decreased contraction was also examined using a collagen-based contraction assay (Laklai et al 2016) (FIG. 16). Interestingly, in cells treated with siFAK+CRISPR-LNPs, the actomyosin network majorly accumulated at the peripheral area of cells with distinct decrease of stress fibers and F-actin alignment (FIG. 14D-14E), which might induce membrane invagination, which can also decrease the membrane tension because membranes flatten at high tension and invaginate at low tension (Kaksonen et al, 2018) (Echarri et al, 2015). The cell membrane was further stained and it was noticed that the membrane was deformed and endocytosed after administration of siFAK+CRISPR-LNPs, which was time-, and siFAK-concentration dependent (FIG. 14F-14G and FIG. 17A-17B). In addition, decreased Yes-associated protein (YAP) expression and localization of YAP to the nucleus further showed that cellular contraction was decreased by FAK knockdown (Dupont et al. 2011) (FIG. 18A-18C).

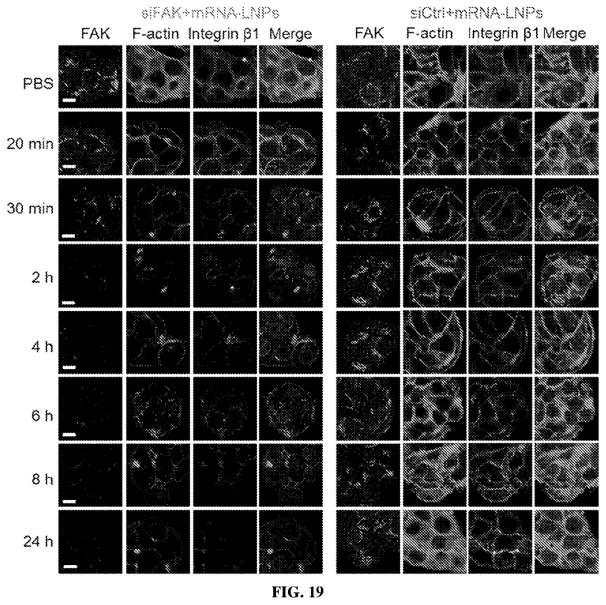

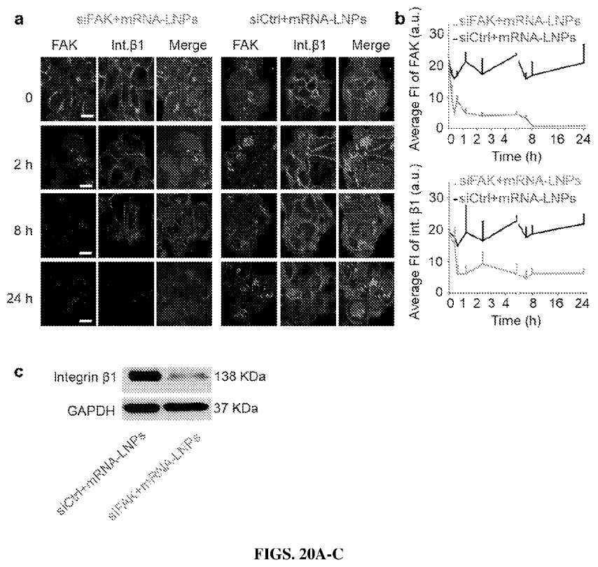

Combined with the endocytosis of cell membrane-protein integrin β1 in IGROV1 and HepG2 cells treated with siFAK+CRISPR-LNPs (FIG. 19 and FIG. 20A-20C), siFAK+CRISPR-LNPs were determined to reduce the contraction force to enhance the cellular endocytosis and penetration of nanoparticles.

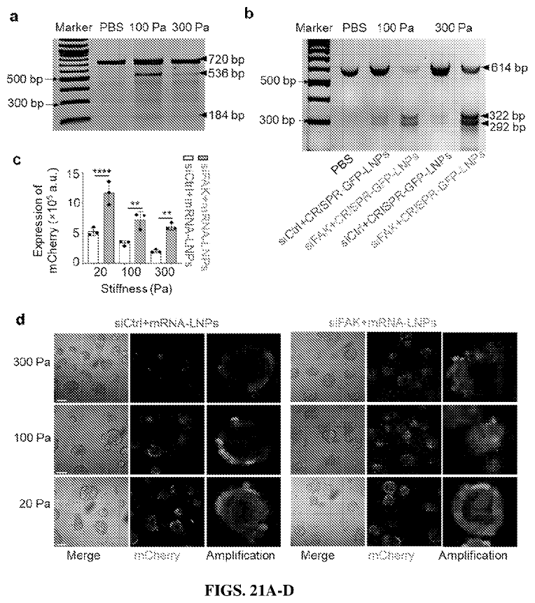

To further demonstrate that decreasing mechanical properties of cells could enhance gene editing, cell stiffness was modulated by controlling the substrate stiffness using different concentrations of matrigel matrix (˜20 mg/mL, ˜300 Pa; and ˜10 mg/mL, ˜100 Pa) (Kraning-Rush et al, 2012; Chaudhuri et al, 2014). mRNA delivery and gene editing efficacy were significantly enhanced through decreasing the mechanical properties of the tumor tissue regulated by soft substrates through administration of reporter mRNA, siFAK+CRISPR-LNPs targeting luciferase in HeLa-Luc, and targeting GFP in HeLa-GFP tumor spheroids (FIG. 1I, FIG. 21A-21D). Notably, a >12-fold enhancement of CRISPR/Cas-mediated gene editing in the stiffer tumor spheroids was quantified following administration of siFAK+CRISPR-GFP-LNPs (FIG. 11), indicating that FAK knockdown enhances gene editing in complex tumor models.

### Example 2: SiFAK+CRISPR-PD-L1-LNPs Inhibit Xenograft Tumor Growth In Vivo

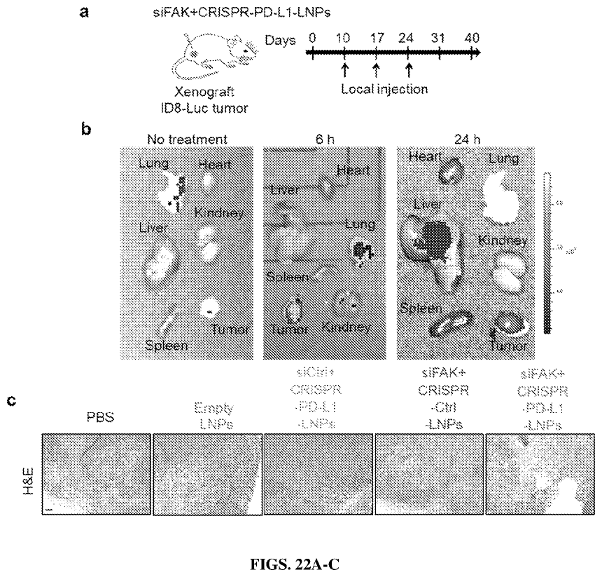

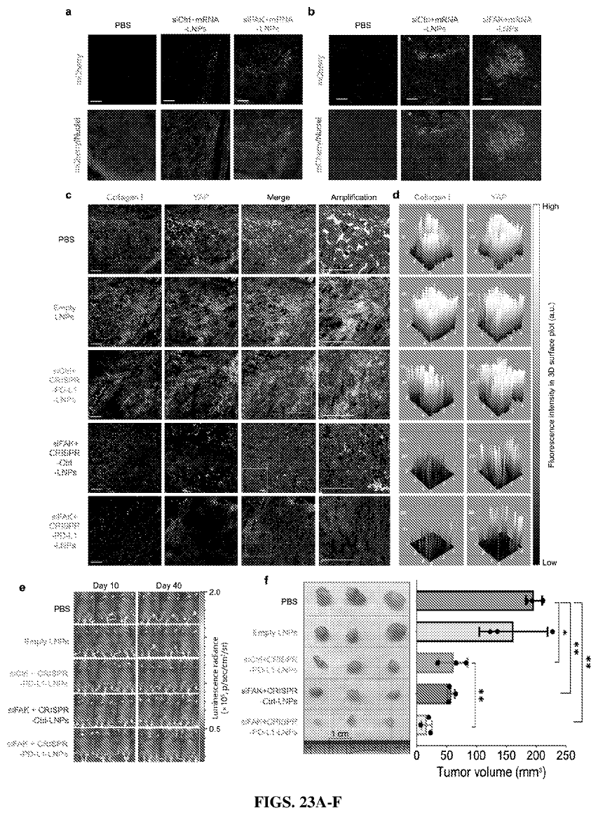

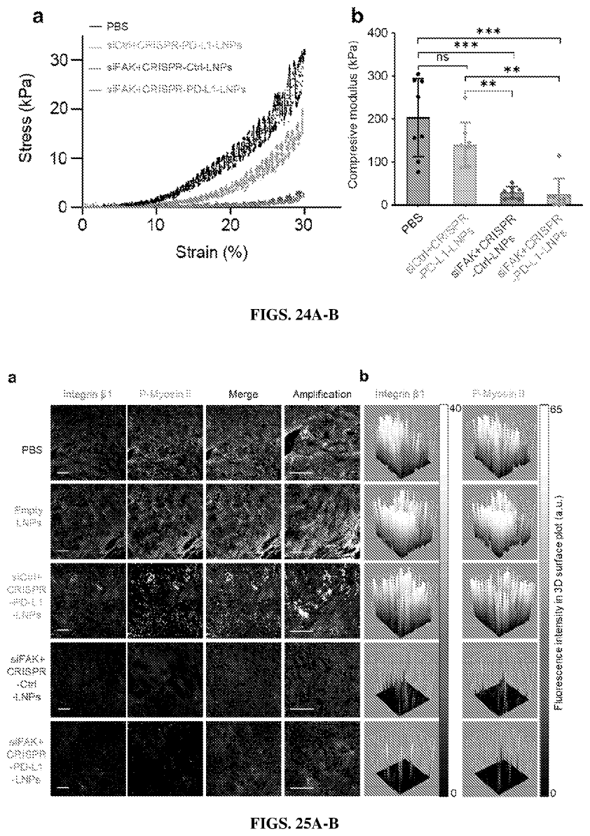

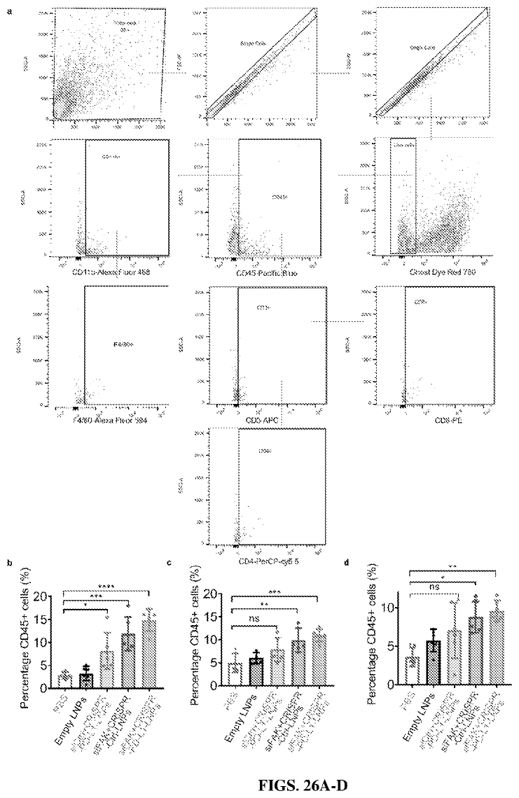

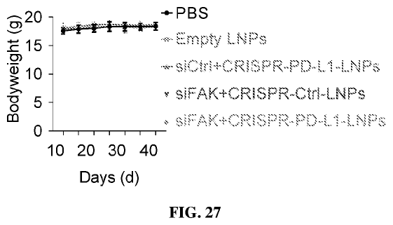

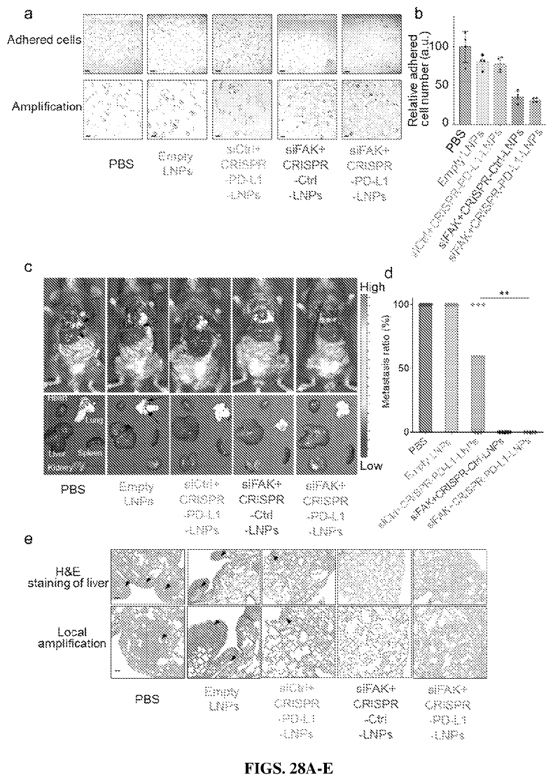

FAK is overexpressed in several advanced-stage solid cancers, especially ovarian cancer (Hoadley et al, 2014), which increases the contraction of tumor cells and stiffness of ECM (Stokes et al, 2011). Therefore, to evaluate gene editing and antitumor efficacy of siFAK+CRISPR-PD-L1-LNPs, C57BL/6 mice bearing ID8-Luc xenograft tumors were used to perform the following experiments through local administration of siFAK+CRISPR-PD-L1-LNPs and other controls (PBS, empty LNPs, siCtrl+CRISPR-PD-L1-LNPs, siFAK+CRISPR-Ctrl-LNPs) (FIG. 22A). Next, (i) LNP distribution and penetration was examined; (ii) protein expression following translation of the delivered mRNA was measured; (iii) the ECM and the infiltration of immune cells in tumor microenvironment was investigated; and (iv) the tumor growth in mice after 30-day treatment was compared. Deep LNP penetration and enhanced mRNA translation to protein expression in tumor tissues were observed for siFAK+Cy5-mRNA-LNPs and siFAK+mCherry mRNA-LNPs (FIG. 23A-23B and FIG. 22B). Changes in tumor stiffness were studied through examining compressive modulus of tumor tissue, deposition and crosslinking of collagen I, and other proteins associated with cell mechanics (YAP, P-myosin II, and integrin β1) (FIG. 23C-23D, and FIG. 24-FIG. 25). Tumor stiffness decreased in the tumor microenvironment of mice treated with siFAK+CRISPR-PD-L1-LNPs and siFAK+CRISPR-Ctrl-LNPs, leading to a less aggressive tumor phenotype evidenced by decreased P-myosin II, integrin β1, and collagen I fibers (FIG. 23C-23D and FIG. 22C, FIG. 24, and FIG. 25). Moreover, the infiltration of immune cells, especially T cells, in the tumor tissue were also increased after administration siFAK+CRISPR-PD-L1-LNPs (FIG. 26). The combination of a reduction in ECM stiffness regulated by gene silencing of FAK, and enhanced gene editing of PD-L1 should inhibit tumor growth (FIG. 23E-23F). The average tumor volume of mice treated with siFAK+CRISPR-PD-L1-LNPs was ˜16 mm³ which was smaller than the tumor volume of mice treated with PBS (˜200 mm³), empty LNPs (˜180 mm³), siCtrl+CRISPR-PD-L1-LNPs (˜90 mm³), and siFAK+CRISPR-Ctrl-LNPs (˜65 mm³) (FIG. 23E-23F). Meanwhile, there was no significant acute toxicity to mice following administration of siFAK+CRISPR-PD-L1-LNPs (FIG. 27). Taken together, regulating the ECM stiffness through FAK knockdown was determined to be beneficial for gene editing of PD-L1, which in turn significantly inhibits tumor growth. Furthermore, using a mouse metastasis model of ovarian cancer, it was demonstrated that siFAK+CRISPR-PD-L1-dLNPs could decrease ovarian cancer metastasis due to the combination of decreasing cellular adhesion by FAK knockdown and improved the gene editing efficacy of PD-L1 (FIG. 28A-28E).

### Example 3: SiFAK+CRISPR-LNPs Enhance Gene Editing in a Genetically Engineered Liver Cancer Model

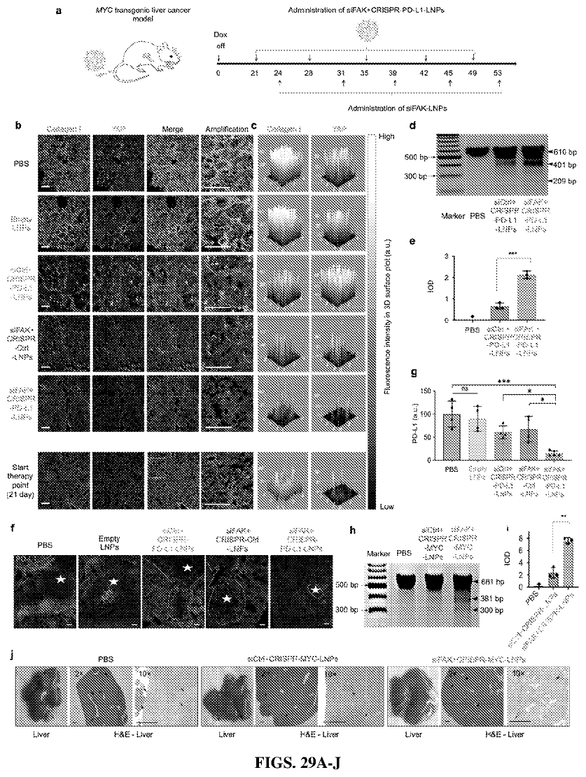

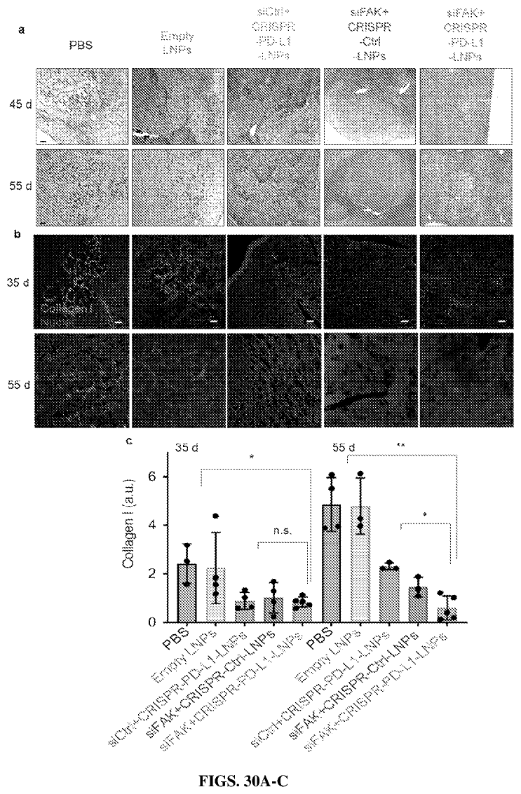

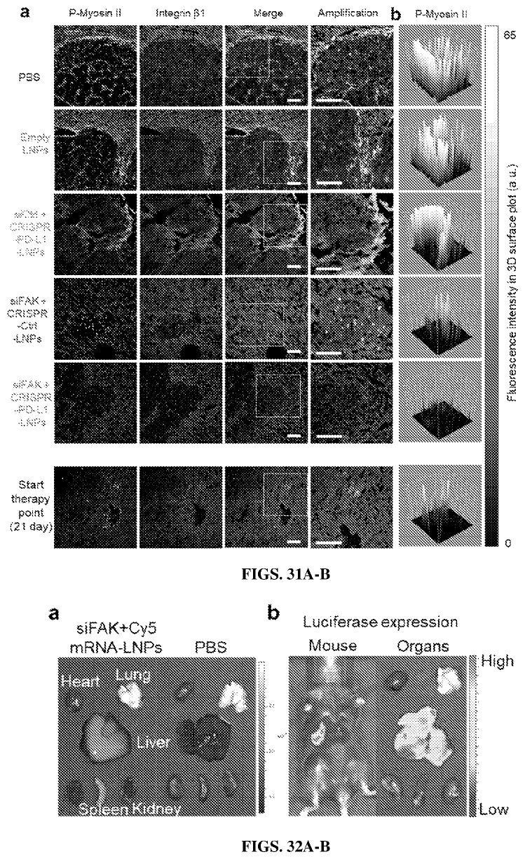

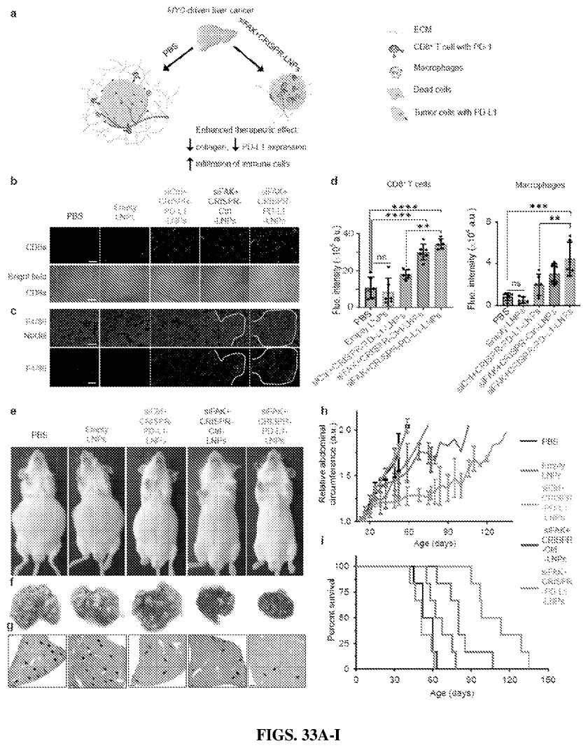

To further evaluate the effects of combined FAK silencing and enhanced gene editing, antitumor efficacy was examined in aggressive, genetically engineered mouse model (GEMM) of liver cancer harboring a tetracycline (tet)-repressible human MYC transgene (tet-MYC) (Shachaf et al, 2004). Upon removal of doxycycline (dox), rapid tumor growth leads to death within 60 days without treatment. Moreover, high levels of fibrosis with collagen deposition have been frequently detected in liver cancer models that involve MYC overexpression (Zheng et al, 2017), leading to stiffer tumors embedded in normal tissue which influences treatment responses and hinders nanoparticle uptake (Egeblad et al, 2010; Paszek et al, 2005). Here, the treatment regimen outlined in FIG. 29A was followed to test whether enhancing the gene editing through reducing the tumor mechanics can improve therapeutic outcomes. Examining the livers at day 35, 45, and 55 (FIG. 29B-29C and FIG. 26A-26C, FIG. 30A-30C, and FIG. 31A-31B), siFAK+CRISPR-PD-L1-LNPs and siFAK+CRISPR-Ctrl-LNPs treated mice had markedly reduced levels of collagen fibrosis (FIG. 29B-29C), indicating that reduction of FAK expression in cancer cells decreased tumor tissue stiffness, which was also confirmed by reduced YAP, P-myosin II, and integrin β1 in tumors (FIG. 29B-29C and FIG. 31A-31B). The high efficacy of mRNA delivery was further demonstrated using Cy5-labeled mRNA and luciferase-mRNA (siFAK+mRNA-LNPs) (FIG. 32A-32B).

Next, CRISPR/Cas-mediated gene editing in MYC mice following i.v. injection of PBS, siFAK+CRISPR-PD-L1-LNPs, and siCtrl+CRISPR-PD-L1-LNPs was investigated. The results showed that DNA cleavage efficacy was significantly enhanced by FAK knockdown, leading to a distinct decrease of PD-L1 expression in the tumor tissue (FIG. 29D-29G).

Significant enhancement of gene editing in vivo was also confirmed through examining gene editing efficacy of siFAK+CRISPR-MYC-LNPs specifically targeting MYC through quantifying indels and tumor growth inhibition in the liver (FIG. 4H-4J).

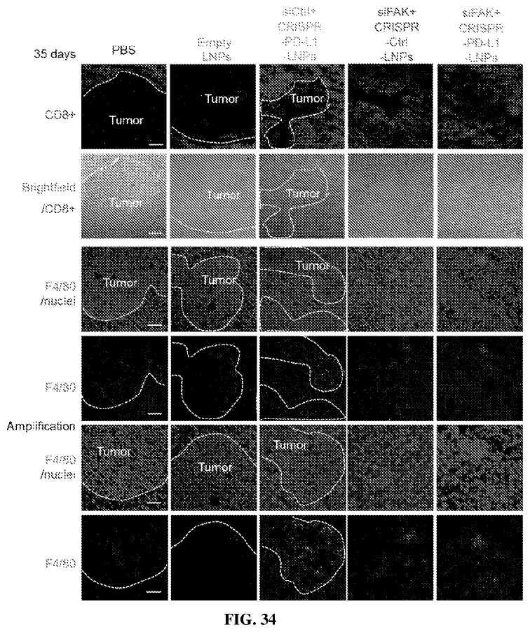

Successful LNP delivery of siFAK, Cas9 mRNA, and sgPD-L1 leads to a reduction of PD-L1 expression and ECM stiffness, which will enhance anti-tumor immune response mediated by increase of immune cell infiltration (FIG. 33A). The infiltration of CD8+ T cells (Fourcade et al, 2012), and macrophages present in the tumor microenvironment (Schreiber et al, 2011; Sica et al, 2012) which are crucial to inhibit tumor growth, were examined. As expected, higher numbers of CD8+ T cells and macrophages infiltrated into the tumors of mice treated with siFAK+CRISPR-PD-L1-LNPs compared with mice treated with siCtrl+CRISPR-PD-L1-LNPs and siFAK+CRISPR-Ctrl-LNPs. There were few CD8+ T cells in the tumors of mice treated with PBS and empty LNPs at days 35 and 55 (FIG. 33B-33D and FIG. 34). This enhancement of CD8+ T cell and macrophage infiltration within the tumor could, without being bound by theory, be representative of host immune reactions against cancer cell growth and were most significantly associated with a better survival (Schreiber et al, 2011; Naito et al, 1998).

### Example 4: SiFAK+CRISPR-PD-L1-LNPs Extend Survival of Mice Bearing MYC-Driven Cancer

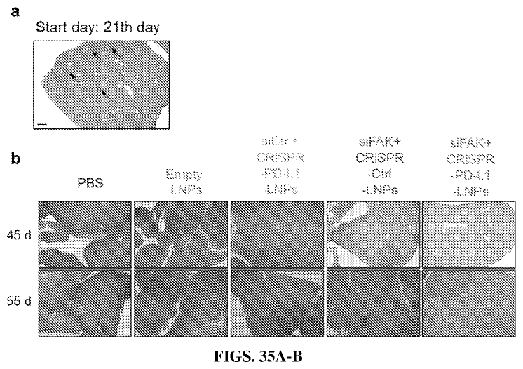

Building on these results tumor growth and survival of MYC mice was further investigated following longer term treatments with PBS, empty LNPs, siCtrl+CRISPR-PD-L1-LNPs, siFAK+CRISPR-Ctrl-LNPs, and siFAK+CRISPR-PD-L1-LNPs with sgRNA specifically targeting PD-L1. A therapeutic regimen by weekly i.v. injection of siFAK+CRISPR-PD-L1-LNPs was initiated. Due to the short duration of siRNA-mediated gene silencing (Zhou et al, 2016; Zhang et al, 2018; Miller et al, 2018; Wu et al, 2014), siFAK-LNPs were administered one more time per week. At day 55, the abdomens of mice that received siFAK+CRISPR-PD-L1-LNPs were similar in circumference to normal, wild type mice and much smaller than abdomens of MYC mice treated with PBS (FIG. 33E), indicating, without being bound by theory, that tumor growth was suppressed. Decreased tumor growth in mice treated with siFAK+CRISPR-PD-L1-LNPs was also confirmed through analysis of liver photographs and H&E staining (FIG. 33F-33G and FIG. 35A-35B). Liver tumor growth in mice treated with siFAK+CRISPR-Ctrl-LNPs and siCtrl+CRISPR-PD-L1-LNPs was also slower than that of mice treated with PBS and empty LNPs at day 35. Among all groups, mice treated with siFAK+CRISPR-PD-L1-LNPs clearly had the smallest tumors at day 55 (FIG. 35B), demonstrating that FAK silencing to improve gene editing enhanced overall cancer therapy.

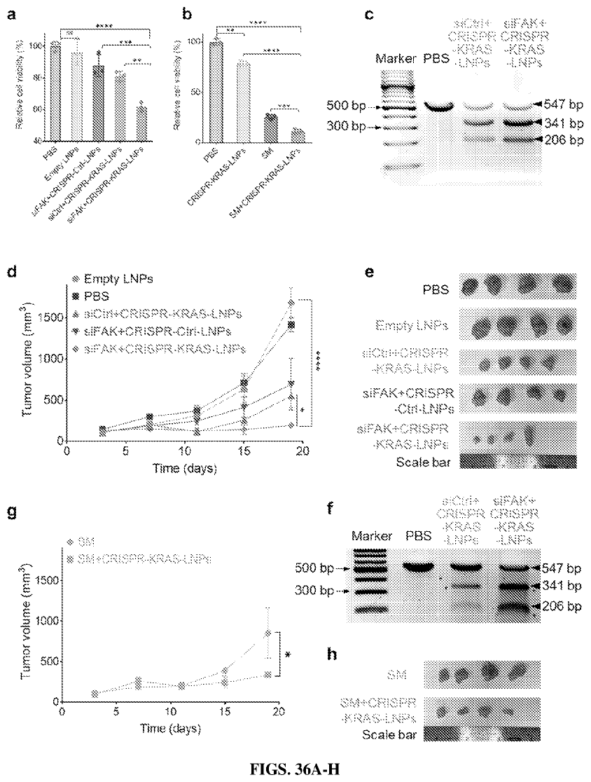

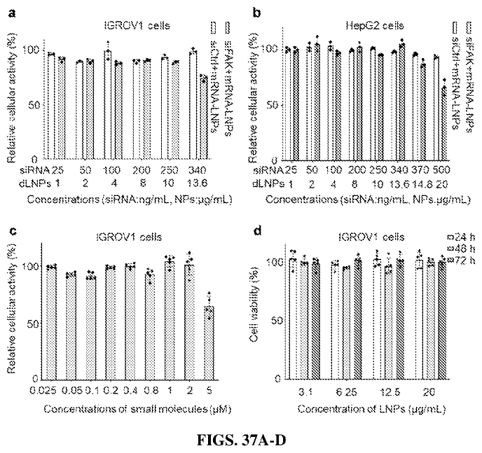

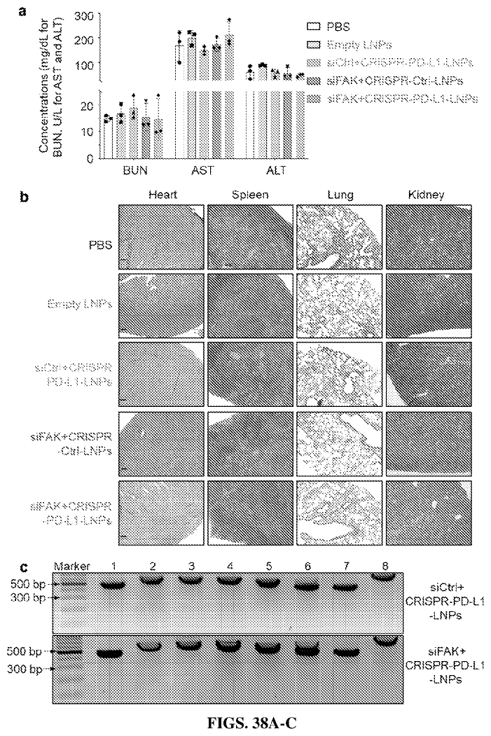

The curve of abdominal circumference of mice treated with only gene editing by siCtrl+CRISPR-PD-L1-LNPs showed a plateau from day 21 to 45 (FIG. 35H), suggesting that gene editing of PD-L1 had therapeutic efficacy at the early time points when tumors were smaller and LNP uptake and penetration were less hindered by ECM (low collagen I deposition at 35 day) (FIG. 30B-30C). After 45 days, the abdominal circumference curve of mice treated with siCtrl+CRISPR-PD-L1-LNPs showed steep increase. Considering that collagen I deposition and crosslinking was significantly increased in tumors at 45 and 55 days (FIG. 29B-29C and FIG. 30A-30C), it was hypothesized, without being bound by theory, that high ECM stiffness in the tumor acted as a barrier to siCtrl+CRISPR-PD-L1-LNP uptake and penetration. After 45 days, gene editing therapeutic efficacy was distinctly decreased. The abdominal circumference curve rose slowly in the groups treated by siFAK+CRISPR-Ctrl-LNPs and siFAK+CRISPR-PD-L1-LNPs, indicating that FAK knockdown inhibited tumor growth. In turn, the enhancement of LNP delivery due to FAK silencing reduced the ECM and thus increasing gene editing of tumor cells mediated by CRISPR further increasing the therapeutic efficacy (FIG. 33E-33G). Therefore, administration of siFAK+CRISPR-PD-L1-LNPs significantly extended the survival of mice (>100 days) in comparison to the PBS treated group (60 days) (FIG. 331). There was a significant increase in survival compared to mice treated with siCtrl+CRISPR-PD-L1-LNPs (˜67 days), which is approximately the effect measured previously for delivery of Let-7g (Zhou et al, 2018) and siRNA against Anilin (Zhang et al, 2018). Enhanced tumor therapy through gene editing increased by FAK knockdown was also demonstrated using small molecule inhibitors of FAK and CRISPR-LNPs targeting KRAS (a RAS human oncogene (Cox et al, 2010) in A549 cells in vitro and in tumor xenografts (FIG. 36A-36H). This general multiplexed approach may therefore be applicable to a variety of oncogenic targets in the future. In addition, in vitro and in vivo safety studies revealed no off-target editing and high tolerability (FIG. 37A-37D and FIG. 38A-38C). Overall, provided herein is evidence that LNPs inhibited the rapid tumor growth and enhanced gene editing of PD-L1 to improve immunotherapeutic efficacy in multiple difficult-to-treat cancer models.

### Example 5: Evaluation of Sequential (Two Separate LNPs) and Simultaneous Delivery of siRNA and mRNA (One LNP Containing Both Nucleic Acids)

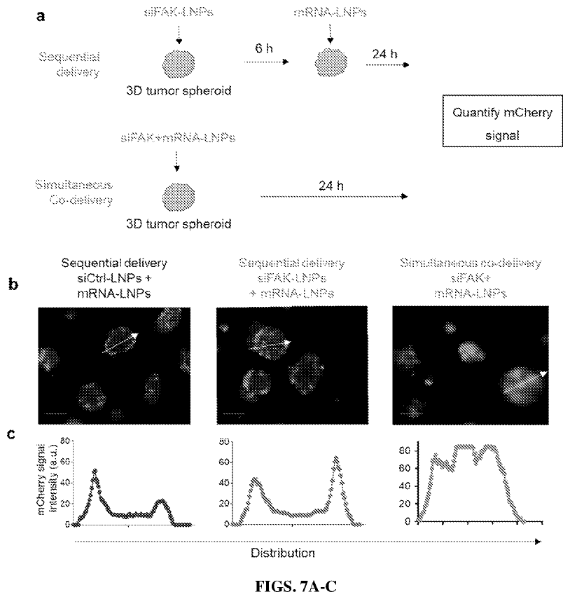

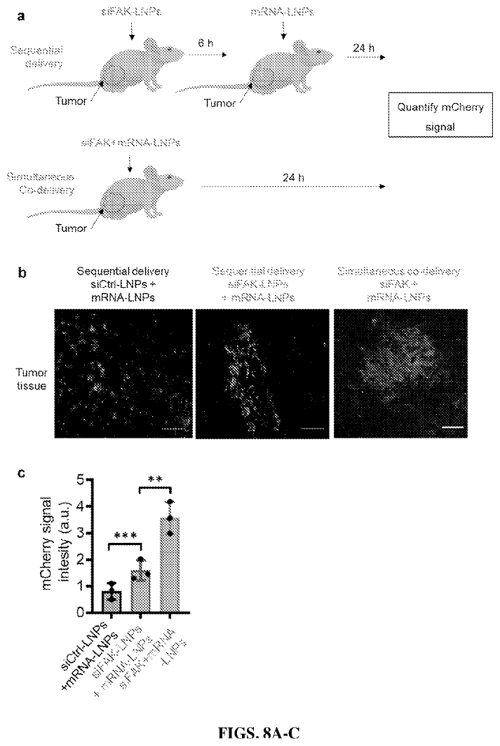

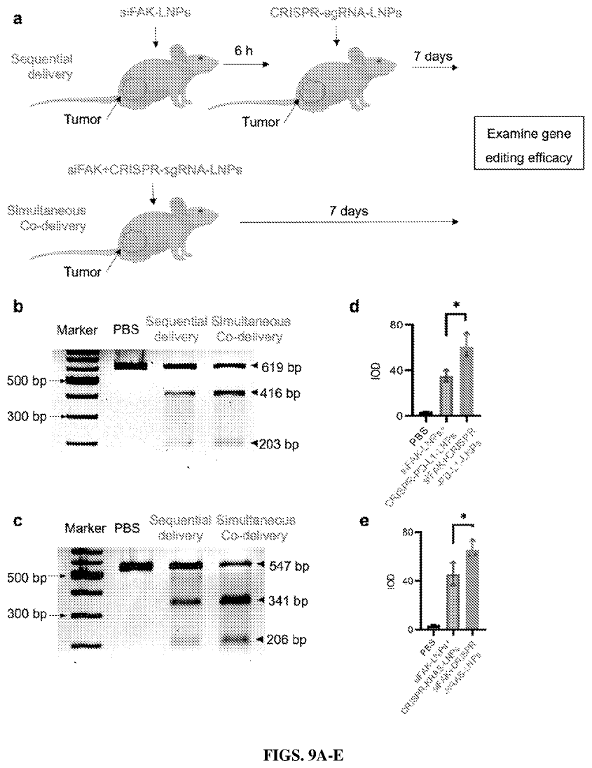

The delivery efficacy of sequential (two separate LNPs) and simultaneous co-delivery of siRNA and mRNA (one LNP containing both nucleic acids) were directly compared. In the sequential delivery approach, siFAK LNPs were administered first, followed by mRNA LNPs 6 hours later. Results indicated that simultaneous co-delivery of mRNA+siRNA in one LNP formulation was more efficacious than sequential delivery of siRNA and mRNA in separate LNPs in three-dimensional (3D) tumor spheroids and in tumor tissues in vivo (FIG. 7 and FIG. 8), capturing important tissue-level effects that were not apparent in static two-dimensional (2D) cell culture (FIG. 5). Moreover, simultaneous delivery was more efficacious than sequential delivery for genome editing (siRNA+Cas9 mRNA+sgRNA) in 3D spheroid cultures and in tumors in vivo possessing physical barriers (FIG. 6 and FIG. 9). Given those empirical results, coupled to the practical application of all-in-one delivery that would ensure all cargoes could be delivered to the same targeted cells, the simultaneous delivery approach was utilized for further studies. It is contemplated that sequential and simultaneous delivery approaches could be useful to control the timing of silencing and editing events.

### Example 6: SiFAK+CRISPR-PD-L1-LNP Treatment can Decrease Metastatic Potential

siFAK+CRISPR-PD-L1-LNPs could potentially inhibit ovarian cancer metastasis due to the combination of decreasing tumor mechanical properties and improving the gene editing efficacy of PD-L1. Metastatic niches depend on the ability of cancer cells to adhere to the vascular endothelium of distant organs through overcoming the effects of fluid shear and immnosurveillance. To study this, the adherent ability of cells was first examined using the centrifuge-induced cell detachment assay. The number of adhered cells was significantly decreased after the cells were treated with siFAK+CRISPR-Ctrl-LNPs and siFAK+CRISPR-PD-L1-LNPs for 48 h (FIG. 28A and FIG. 28B), indicating without being bound by theory that the treated cells had decreased adherence ability, which, again without being bound by theory, should decrease potential metastasis. To further test this in vivo, luciferase expressing mouse ovarian (ID8-Luc) cells were treated with PBS, empty LNPs, siFAK+CRISPR-Ctrl-LNPs, siCtrl+CRISPR-PD-L1-LNPs, and siFAK+CRISPR-PD-L1-LNPs for 48 h and these treated cells were intravenously injected into C57BL/6 mice (2×10⁵ [D8-Luc cells) to examine lung metastasis in this established model 30 days after injection. No metastatic tumors formed in the lungs of mice injected with cells treated with siCtrl+CRISPR-PD-L1-LNPs, siFAK+CRISPR-Ctrl-LNPs, or siFAK+CRISPR-PD-L1-LNPs (FIG. 28C and FIG. 28D). From the H&E staining of lung tissue, it was confirmed that no metastatic niches formed in the lungs, especially in mice injected with cells treated with siFAK+CRISPR-Ctrl-LNPs and siFAK+CRISPR-PD-L1-LNPs. In contrast, lung metastases were obviously formed in mice treated with PBS and empty LNPs (FIG. 28E). This data shows that the combination of FAK silencing and enhanced gene editing of PD-L1 by administration of siFAK+CRISPR-PD-L1-LNPs significantly decreased potential metastasis. The lack of metastasis was likely due to a decrease in cellular adhesion by FAK knockdown.

### Example 7: Materials and Methods

### Materials

The dendrimer 5A2-SC8 was synthesized as described in our previous publication (Zhou et al, 2016). 1,2-Dimyristoyl-sn-glycerol-methoxypolyethylene glycol 2000 (DMG-PEG) was purchased from NOF America Corporation. DSPE-PEG (2000), DSPE-PEG (2000)-Folate, and 1,2-dioleoyl-sn-glycero-3-phosphoethanolamine (DOPE) were purchased from Avanti Polar Lipids. Cholesterol, Pur-A-Lyzer Midi Dialysis Kits (WMCO, 3.5 kDa), 5-(N-ethyl-N-isopropyl)-amiloride, Filipin III from Streptomyces filipinensis, Chlorpromazine hydrochloride, Bafilomycin A1 from Streptomyces griseus, Direct Red 80, and picric acid were purchased from Sigma-Aldrich. RIPA lysis and extraction buffer, DMEM high glucose with L-glutamine and sodium pyruvate, RPMI 1640 medium (ATCC modification), Corning Matrigel Matrix, 4′,6-diamidino-2-phenylindole dihydrochloride (DAPI), DLS Ultramicro cuvettes, Lab-Tek chambered cover glass units, Proteinase K solution, PureLink Genomic DNA Mini Kit, UltraPure DNase/RNase-free distilled water, collagen I, and fetal bovine serum (FBS) (sterile-filtered) were purchased from Thermo Fisher Scientific. D-Luciferin (sodium salt) was purchased from Gold Biotechnology. Passive Lysis 5X Buffer and the ONE-Glo+Tox Luciferase Reporter assay kit were purchased from Promega. All materials for running Western blots were purchased from Bio-rad: nitrocellulose membrane, 10×Tris/Glycine buffer, 10×Tris buffered saline, precision plus protein dual color standards, 10% Mini-protean TGX protein gels, 5×laemmili sample buffer, 10×Tris/glacine/SDS, Goat Anti-Rabbit IgG (H+L)-HRP Conjugate, Goat Anti-Mouse IgG (H+L)-HRP Conjugate). All primers (Table 1) were purchased from Integrated DNA Technologies (IDT). Firefly Luciferase mRNA (FLuc mRNA), Cy5 dye-labeled Luciferase mRNA (Cy5-Luc mRNA), and CleanCap Cas9 mRNA (modified) (L-7206-1000) were purchased from TriLink BioTechnologies. Custom sgRNAs were purchased from Synthego. Mouse and human FAK siRNA were purchased from Sigma-Aldrich. T7 Endonuclease I was purchased from New England Biolabs (NEB).

TABLE 1
Primer, sgRNA, and siRNA sequences.
Primers for PCR

| | | |
|---|---|---|
| hMYC | Forward | ccgctggttcactaagtgcg (SEQ ID NO: 1) |
| | Reverse | ttctctgagacgagcttggcg (SEQ ID NO: 2) |
| mPD-L1 | Forward | caggtaatacagaactaacaggtg (SEQ ID NO: 3) |
| | Reverse | gctgcataatcagctacggt (SEQ ID NO: 4) |
| hPD-L1 | Forward | gtcacatggatatattacatagtg (SEQ ID NO: 5) |
| | Reverse | cagaatattacctgggatgaccaa (SEQ ID NO: 6) |
| KRAS | Forward | cctgactattgatgttgagc (SEQ ID NO: 7) |
| | Reverse | ctgctctaatcccccaagaacttc (SEQ ID NO: 8) |
| GFP | Forward | gtggtgcccatoctggtcgag (SEQ ID NO: 9) |
| | Reverse | cgcttctcgttggggtctttgc (SEQ ID NO: 10) |

Primers for qRT-PCR

| | | |
|---|---|---|
| hFAK | Forward | gcctggtgaaagctgtcatc (SEQ ID NO: 11) |
| | Reverse | gcttctgtgccatctcaatc (SEQ ID NO: 12) |
| GAPDH | Forward | cccctggccaaggtcatcca (SEQ ID NO: 13) |
| | Reverse | acagccttggcagcgccagt (SEQ ID NO: 14) |

sgRNA

| | |
|---|---|
| hPD-L1 | acugcuuguccagaugacuu (SEQ ID NO: 15) |
| mPD-L1 | gguccagcucccguucuaca (SEQ ID NO: 16) |
| hMYC | gtatttctactgcgacgagg (SEQ ID NO: 17) |
| KRAS | ucccuucucaggauuccuac (SEQ ID NO: 18) |
| Control | gcuuucacggagguucgacg (SEQ ID NO: 19) |
| GFP | gaaguucgagggcgacaccc (SEQ ID NO: 20) |

SIRNA

| | |
|---|---|
| h/m FAK | gggcaucauucagaagauadtdt (SEQ ID NO: 21) |

### Nanoparticle Formulations

Ionizable cationic dendrimer lipid nanoparticles (LNPs) were formulated and prepared as described in our previous publications (Zhou et al, 2016; Cheng et al, 2018; Cheng et al, 2020). Briefly, 5A2-SC8, DOPE, cholesterol, DMG-PEG, and DSPE-PEG (2000) were dissolved in ethanol (molar ratio, 15:15:30:2:1) and all RNAs were dissolved in citrate buffer (10 mM, pH 3.8). Under vortex shaking, the lipid solution (40 L) was added into the RNA solution (120 μL). The formulated LNPs were incubated for ˜15 min at room temperature, and then diluted with 1× PBS for use in in vitro experiments. For ID8-Luc xenograft tumor, the formulation was 5A2-SC8, DOPE, cholesterol, DMG-PEG, and DSPE-PEG (2000)-folate (molar ratio, 15:15:30:2:1). LNP characteristics were as follows (Total RNA for siFAK+mRNA-LNPs, 6.25˜25 μg/mL, mRNA: siRNA=from 4:1 to 2:1; Total RNA for siFAK+CRISPR-LNPs, 25 μg/mL, Cas9 mRNA: sgRNA: siRNA=2:1:2). For in vivo experiments, LNPs were dialyzed for at least 3 hours using Pur-A-Lyzer Midi Dialysis Kits, MWCO 3.5 kDa in 1×PBS. The final concentration of RNA was 0.2 μg/μL (Cas9 mRNA: sgRNA: siRNA=2:1:3, w/w). For the MYC-driven liver cancer therapy mouse model experiments, the siFAK+CRISPR-LNPs were assembled from using the formulation engineered to specifically target the liver. The formulation was 5A2-SC8, DOPE, cholesterol and DMG-PEG dissolved in ethanol, molar ratio, 15:15:30:3.

### Characterization of LNPs

The hydrodynamic size and polydispersity index (PDI) of LNPs encapsulating siRNA and mRNA were measured using Dynamic Light Scattering (DLS, Malvern; He—Ne laser, 2. =632 nm; detection angle=173°) with Zetasizer software (Malvern) with fresh LNPs. Then the zeta-potential was tested using diluted solutions. The encapsulation efficiency of total RNA was calculated from quantification of RNA remaining in solution using Quant-iT Ribo Green RNA assay based on the standard protocol (Thermo Fisher). The time-dependent stability of prepared LNPs was also examined through monitoring the sizes of nanoparticles dispersed in bovine serum albumin (BSA, 5% in PBS, w/v) at 2 h, 24 h, 48 h, 72 h, at 37° C.

### Cell Culture

HeLa, A375, HepG2, B16F10, and A549 cell lines were originally obtained from ATCC. Derived HeLa-Luc and HeLa-GFP reporter cells were generated using lentiviruses from HeLa cells originally obtained from ATCC. IGROV1 cells were originally obtained from Millipore-Sigma. ID8-Luc cells were received from the University of Pittsburgh and used with permission under a Materials Transfer Agreement (MTA). The cell lines were not further authenticated after receiving from ATTC, Millipore-Sigma, or the University of Pittsburgh. ID8-Luc, HepG2, A375, B16F10, HeLa-Luc, HeLa-GFP cells, and A549 were cultured in DMEM medium (10% FBS, 1% 100U penicillin and 0.1 mg/mL streptomycin). IGROV1 cells were cultured in RPMI 1640 medium (10% FBS, 1% 100U penicillin and 0.1 mg/ml streptomycin). All cells were cultured at 37° C. and 5% CO₂ in a humidified incubator. For nanoparticle delivery experiments, cells were seeded into new plates and cultured for ˜18 h, then the nanoparticles were added into the fresh medium and cultured for another time point (12 h, 24 h, and 48 h) for monitoring luciferase knockdown, FAK knockdown, mCherry expression, and gene editing of targeting GFP, hPD-L1, hMYC, mPD-L1, and KRAS genes.

For tumor spheroid culture, HeLa-GFP and IGROV1 cells were seeded into 24-well plates over which the substrates were covered with Matrigel matrix. The matrix stiffness was regulated by changing the concentration of the Matrigel matrix (Matrigel HC, Corning) to be 20 mg/mL, 10 mg/mL, and 5 mg/mL for obtaining the substrate stiffness with ˜300 Pa, ˜100 Pa, and ˜20 Pa, according to a previous publication (Chaudhuri et al, 2014). During the culturing process, the medium was exchanged every four days. Fresh medium was added with 2% Matrigel matrix in DMEM (10% FBS, 1% 100U penicillin and 0.1 mg/mL streptomycin) for HeLa-GFP tumor spheroids, and in RPMI 1640 medium (10% FBS, 1% 100U penicillin and 0.1 mg/mL streptomycin) for IGROV1 tumor spheroids. To control for the number of cell binding sites, substrates with different concentrations of Matrigel matrix were modified with the same concentration of collagen I (30 μg/mL). After culturing the cells on the substrates for 21 days, when the tumor spheroids formed, LNPs were added into the medium to examine the parameters of mRNA expression or gene editing in tumor spheroids after 48 h or 72 h.

In Vitro Cas9 mRNA and siRNA Delivery Experiments

For HeLa-GFP tumor spheroid gene editing, when the tumor spheroids formed at the matrigel matrix (20 mg/mL) after 21-day seeding, siFAK+CRISPR-GFP-LNPs were added into the fresh DMEM medium (10% FBS, 1% 100U penicillin, and 0.1 mg/mL streptomycin) and cultured for 48 or 72 h. The final concentration of total RNA was 2.67 μg/mL with Cas 9 mRNA: sgRNA: siRNA=2:0.25:2. For IGROV1 cells, the cells were seeded into the 24-well plates for 18 h, and then changed to the fresh medium with siFAK+CRISPR-PD-L1-LNPs and cultured for 48 h. The final concentration of total RNA was 1.25 μg/mL with Cas 9 mRNA: sgRNA: siRNA=2:1:2. After that time, gene editing was examined using the T7E1 assay. Briefly, the target site was PCR-amplified (all sequence information can be found in Table 1) and previous paper (Wei et al, 2020). The products were purified using PureLink PCR purification Kit (Thermo Fisher) following the manufacturer's protocol. A volume purified PCR products (200 ng), and 2 μL 10× NEBuffer (New England BioLabs) were mixed and then added ultrapure water to a final volume of 19 μL. The mixture was put into thermocycler running with the following hybridation conditions: 95° C. for 5 min, annealing from 95° C. to 85° C. at −2° C. s-1, from 85° C. to 25° C. at −0.1° C. s-1, an holding at 4° C. After re-annealing, 1-μL T7E1 nuclease (New England BioLabs) was added to the mixture (19 μL) and incubated for another 15 min at 37° C. After incubation, digested products were purified with the PureLink PCR Purification Kit and analyzed by electrophoresis in 2.5% agarose gel. Gels were imaged with a Gel Doc gel imaging system (Bio-Rad). Quantification was determined by analyzing integrated optical density of bands.

In Vitro mRNA and siRNA Delivery Experiments

IGROV1, HepG2, A375, B16F10, and HeLa-Luc cells were seeded into white 96-well plates with the density of 1×10⁴ cells per well. After 17 h, the medium was replaced with fresh DMEM (200 μL, 10% FBS). For optimizing the RNA concentrations, different amounts (0.31, 0.63, 1.25, 2.5, 5, 10 μL) of LNPs encapsulating FAK siRNA (siRNA) and mCherry mRNA (siFAK+mRNA-LNPs with total RNA concentration=6.25 g/mL, mRNA: siRNA=4:1) were added into the IGROV1 cell culture medium (200 μL total volume). LNPs encapsulating anti-Luciferase siRNA (siLuc) and mCherry mRNA as control group. For experiments of testing the enhancement of mRNA with knockdown FAK in multiple tumor cells, siFAK+mRNA-Luc-LNPs or siFAK+mRNA-mCherry-LNPs with one RNA concentration (15 μg/mL; mRNA: siRNA=2:1, 10 μL for IGROV1 cells, 20 μL for other cells) were added into the cell culture medium. The luciferase, FAK knockdown, and mCherry expression was monitored at further culture for 24 h, 48 h, and 72 h. At each time point, mCherry expression was measured by fluorescence intensity of mCherry after removing the medium and changing to the PBS with the passive lysis buffer (1×lysis solution). The luciferase expression was measured using ONE-Glo+Tox Luciferase Reporter assay kit according to the manufacturer's protocol. Briefly, ONE-Glo was added into the cell culture following the Promega protocol and luciferase activity was detected while avoiding exposure to light. The same methods for examining luciferase knockdown and mCherry expression were followed under different ratios of RNA from 4:1 to 1:1. mCherry expression was also observed using Confocal imaging together with the immunofluorescence staining of FAK. The mRNA expression and nanoparticle penetration in the tumor spheroids was examined through culturing the IGROV1 tumor spheroids. For observing and analyzing the mRNA expression, the cells were cultured on 6-well plates with Matrigel matrix (20 mg/mL). siFAK+mRNA-LNPs or siFAK+Cy5-mRNA-LNPs were added into the medium with a final concentration of total RNA: 1.125 μg/mL, mRNA: siRNA=2:1, 5A2-SC8: total RNA=13:1) and cultured for another 48 h. mRNA expression was observed directly using Keyence microscopy with a 20× objective. For penetration of nanoparticles in tumor spheroids that was culture on the slide covered with Matrigel matrix, siCtrl+cy5-mRNA-LNPs, and siFAK+cy5-mRNA-LNPs were added into the medium and cultured for 48 h. The tumor spheroids with penetrated siFAK+cy5-mRNA-LNPs were directly scanned using confocal microscopy.

### Real-Time PCR

Total RNA was isolated with RNeasy plus Micro and Mini Kits (QIAGEN) according to the manufacturer's instructions. Reverse transcription of purified RNA was performed using the iScript cDNA synthesis kit (Thermo Scientific). The quantification of gene transcripts was performed by real-time PCR using SYBR green I dye (Invitrogen). Expression values were normalized to control GAPDH. Previous sgRNA (Luc) and PCR primers for T7E1 were used. The primers used are listed in Table 1.

### Immunofluorescence Staining

For in vitro experiments, cells were washed with PBS three times, and then fixed with 4% paraformaldehyde for 15 min. For the staining of FAK, P-myosin II, and F-actin, cells were then treated with 0.2% Triton X-100 in PBS for 5 min and washed three times for 5 min each in PBS and blocked for 1 h in blocking solution (2% BSA in PBS). For staining of cell surface protein (Integrin β1 and PD-L1), the cells were fixed and then directly blocked with 2%-BSA without Triton X-100 treatment. Then the cells were incubated with the following primary antibodies overnight at 4° C.: Anti-FAK antibody (1:150, ab131435), anti-YAP antibody (ab205270, 1:100), anti-integrin beta 1 antibody (1:200, ab24693), Phospho-Myosin Light Chain 2 (Ser19) antibody (1:200, 3675S), F-actin staining with phalloidin-iFluor 555 regent (1:500, ab 176756). Then the cells were washed three times with 5 min each with PBS, followed by incubation with Donkey anti-rabbit IgG H&L (Alexa Fluor 647) (1:500, ab150075) and Goat anti-mouse IgG H&L (Alexa Fluor 488) (1:500, ab150113) for 1 h at room temperature, followed by three times washes with 5 min each. The cells were imaged using confocal microscopy (Zeiss LSM 700 confocal microscope) with ZEN x⁶⁴ software. The images were obtained under 60× oil lens.

For in vivo experiments, mice were anaesthetized by isoflurane. The tissues were taken out, embedded optimal cutting temperature (OCT), and frozen at −80° C. for 3 days. Then, the tissues were sectioned at 10 μm with a Leica VT 1000s vibratome. After drying the slides at room temperature, the sections were washed three times with PBS for immunostaining and fixed with 4% paraformaldehyde for 20 min, followed by adding blocking solution (10% BSA in PBS) and blocked for 1 h. Then, samples were incubated with primary antibodies for 18 h at 4° C. The following primary antibodies were used: Mouse PD-L1 antibody (1:200, AF1019, R&D System), anti-YAP antibody (ab205270, 1:100), Anti-Collagen I antibody (1:500, ab34710), PE anti-mouse CD8a Antibody (Biolegend, Cat #162304) and Alexa Fluor® 594 anti-mouse F4/80 (Biolegend, Cat #123140). After samples were incubated with the primary antibodies, they were washed three times for 5 min each time with PBS. Next, samples were incubated with secondary antibody for 1 h at room temperature. The samples were washed three times for 5 min each time with PBS. The nucleus of some samples was stained with DAPI (1 μg/mL) for 10 min at room temperature. Imaging was performed on a Zeiss LSM 700 confocal microscope with ZEN x⁶⁴ software. The images were obtained under 20× lens.

### Western Blot

IGROV1 cells were cultured in 6-well plates for 17 h, then the medium was changed to fresh medium, and siFAK+CRISPR-PD-L1-LNPs (Total RNA concentration, 1 μg/μL, siRNA: Cas9 mRNA: sgRNA=1:2:1) were added. Cells were cultured for another 12 h and 24 h. Then, Western blot was performed according the general protocol for western blotting from Bio-rad. Briefly, the cells were washed three times at the time point and the cells were collected in a 1.5 mL tube. Then the RIPA lysis, extraction buffer and 1× proteinase inhibitor were added to the samples and maintained constant agitation for 30 min at 4° C. During this time, the samples were sonicated 3 time for 15 sec each. Samples were spun down at 16,000 g for 20 min in a 4° C. precooled centrifuge. The supernatant was transferred to a fresh tube, kept on ice, and the pellet was discarded. 20 μL of lysate was removed to perform a protein assay (BSA assay, Thermo Fisher Scientific) for quantifying total protein concentration. The other samples were mixed with 5× Laemmli sample buffer and boiled each cell lysate in sample buffer at 95° C. for 5 min. The cell debris was removed by centrifugation. The extracted protein samples were separated by 10% SDS-PAGE and transferred onto a membrane. The transferred membrane was blocked with dry milk (5%, in 1X Tris-Buffered Saline and 0.1% Tween® 20, TBST) for 1 h and then incubated overnight at 4° C. with FAK primary antibody (1:800, ab131435), anti-YAP antibody (ab205270, 1:500), anti-integrin beta 1 antibody (1:500, ab24693). Then the blotted membrane was washed three times for 5 min each and then cultured with Goat anti-Rabbit IgG (H+L)-HRP conjugate (Bio-rad, 1706515) for 1 h. The protein was finally detected using the chemiluminescent method.

### Cell Viability Analysis

Cells were seeded at a density of 1×10⁴ cells per well into a 96-well plate and allowed to attach for 17 h. Then the cells were treated with PBS, empty LNPs, siFAK+mRNA-LNPs, small molecules (FAK inhibitor, PF-573228, Sigma), and siFAK+CRISPR-KRAS-LNPs. The medium was changed to fresh medium without FBS (50 μL) for examining the cell viability using the ONE-Glo+Tox Luciferase Reporter assay kit according to the manufacturer's protocol.

### Examination of Cellular Uptake

Cells were plated into 35-mm confocal plates with an initial density 5× 10⁴ cells per mL of medium and incubated for 18 h at 37° C. in 5% CO₂. Then the cells were pre-treated with serum-free medium containing inhibitors for 30 min. The inhibitor concentrations were Filipin III (1 μg mL⁻¹), chlorpromazine (Chl., 5 μg mL⁻¹), 5-(N-ethyl-N-isopropyl)-amiloride (EIPA, μg mL⁻¹) and Bafilomycin A1 (Baf A1, 200 nM). The cells were then treated with siFAK+Cy5-mRNA-LNPs with final concentration of total RNA: 0.75 pg/mL, mRNA: siRNA 10=2:1, Dendrimer: total RNA=13:1). For investigation of the cellular uptake under FAK knockdown, the cells were pre-treated with siFAK+mRNA-LNPs for 4 h and then exchanged the cell medium to serum-free medium containing inhibitors for another 30 min. These cells were finally treated with siFAK+Cy5-mRNA-LNPs (concentration of total RNA: 0.75 μg/mL, mRNA: siRNA=2:1, Dendrimer: total RNA=13:1). Internalized LNPs were analyzed through statistically analyzing the fluorescence intensity of Cy5 inside of cells using the Image J software.

### Bio-Distribution In Vivo

Female C57BL/6 mice received i.v. injections with siFAK+Cy5-mRNA-LNPs or siFAK+mRNA-LNPs (Luciferase mRNA) (Total RNA, 0.75 mg/kg, Cy5-mRNA: 0.5 mg/kg, mRNA: siRNA=2:1) in 50 μL. For examining biodistribution of LNPs, mice were euthanized at 6 h post injection and organs were removed. The biodistribution was assessed by imaging whole organs with IVIS Lumina System (Caliper Life Sciences) with the Cy5 filter setting. Data was analyzed using Living image software.

mRNA Delivery in Tumors

After 10 days following xenograft tumor implantation when tumors reached the size of ˜50 mm³, animals bearing xenograft ID8-Luc tumors were administrated with local injection of PBS, siCtrl+mRNA-LNPs and siFAK+mRNA-LNPs (50 μL, total RNA, 5 μg, mRNA: siRNA=2:1). After 7 days, the tumor tissue was excised and cryo-sectioned (10 μm) and fixed using 4% paraformaldehyde at room temperature for 10 min. The tissue was stained with DIPA for 10 min and rinsed for 3 times and then the sections were imaged using an LSM 700 point scanning confocal microscope (Zeiss) equipped with a 20X lens.

### Animal Experiments

All experiments were approved by the Institution Animal Care and Use Committees of The University of Texas Southwestern Medical Center and were consistent with local, state and federal regulations as applicable. Normal wild-type C57BL/6 female mice (6-8 weeks old) were purchased from Charles River. Athymic nude Foxn Inu mice (C₆-8 weeks old) were purchased from Envigo. The mouse experiments were done in the UTSW animal facility. In the xenograft tumor models, six-week-old mice were subcutaneous xenograft using ID8-Luc or A549 (5×106 tumor cells in 0.2 mL PBS were injected s.c.) to C57BL/6 mice and Athymic nude Foxn Inu mice, respectively. For xenograft ID8-Luc tumors, when tumors formed after 10-day injection, mice were randomly allocated into each treatment group and locally injected with siFAK+CRISPR-LNPs and other control groups (PBS, empty LNPs, siCtrl+CRISPR-LNPs, siFAK+CRISPR-Ctrl-LNPs (50 μL, total RNA, 10 μg, Cas9 mRNA: sgRNA: siRNA=2:1:3) specifically targeting PD-L1 to ID8-Luc tumor (3 mice/group). During the process of treatment, the tumor growth was monitored through luciferase bioluminescence of tumor cells (ID8-Luc) using IVIS Lumina In Vivo Imaging System. Each time, the mice were injected 150-mg/kg D-Luciferin in PBS. For xenograft human A549 tumors in Athymic nude Foxn Inu mice, when tumors formed after 3-day injection, the mice were locally administrated with siFAK+CRISPR-KRAS-LNPs or SM+CRISPR-KRAS-LNPs with other control groups (50 μL, 10 μg, Cas9 mRNA: sgRNA: siRNA=2:1:3, 4 mice/group, PBS, Empty LNPs, siFAK+CRISPR-Ctrl-LNPs, siCtrl+CRISPR-KRAS-LNPs, or SM (5 mg/kg)). The mice were injected 3 times at days 3, 5, and 7. After administration, the tumor tissues were excised and the tumor size were measured using digital caliper.

Systematic Administration of siFAK+CRISPR-PD-L1-LNPs for Evaluating Therapeutic Efficacy in MYC-Driven Aggressive Cancer Mouse Models

Transgenic mice bearing MYC-driven liver tumors were generated by crossing the TRE-MYC strain with LAP-tTA strain. Mice bearing the LAP-tTA and TRE-MYC genotype were maintained on 1 mg/mL of dox, and MYC was induced by withdrawing dox. For therapy, dox was removed when the mice were born (Day 0). Starting when the mice were 21 days old, which is after tumorigenesis, mice were randomly allocated into each treatment group and weekly injected with siFAK+CRISPR-PD-L1-LNPs. Injection total RNA of siFAK+CRISPR-PD-L1: 3 mg/kg (Cas9 mRNA (1.0 mg/kg): sgRNA (PD-L1, 0.5 mg/kg); siRNA (1.5 mg/kg)═2:1:3). Dendrimer: RNA=10:1. siFAK-LNPs were injected in the middle of each week (1.5 mg/kg).

For gene editing of MYC, the mice were randomly allocated into each treatment group injected PBS, siCtrl+CRISPR-MYC-LNPs. and siFAK+CRISPR-MYC-LNPs with sgRNA specifically targeting MYC (Injection total RNA, 3 mg/kg with Cas9 mRNA (1.0 mg/kg): sgRNA (MYC, 0.5 mg/kg); siRNA (1.5 mg/kg)═2:1:3. Dendrimer: RNA=10:1. 300 μL). After 10-day administration, the gene editing in liver tissue was examined using the T7E1 assay. The Pure Link Genomic DNA mini kit (Thermo Fisher) was used to extract genomic DNA.

### Toxicity and Liver Function Testing

At each end point, whole blood was collected into BD Microtainer tubes. Serum was separated by centrifuging at the speed of 5000 rpm for 10 min, then blood urea nitrogen (BUN), Alanine Aminotransferase (ALT) and Aspartate Aminotransferase (AST) were tested by the UTSW Molecular Genetics Core.

### H&E Staining of Tissues

Tissues were excised and then fixed with 10% formalin (Sigma). After 72 h fixation, the tissues were sent to the UTSW tissue management shared resource core to perform Haemotoxylin and Eosin (H&E) staining.

### Sirius Red Staining

Tissue sections were deparaffinazed and rehydrated by the following steps: First, the slide was placed in a rack and gently put into staining jars with 100% xylene and washed twice (10 min each), and then placed in a 50% xylene (in ethanol, v/v) a staining jar with 2 distinct washes (10 min each). The slides were then washed using ethanol with different concentrations (95%, 75%, 50%, 2 washes each, 5 min each). The slices were then washed twice using distilled water and submerged 10 min each, following with dropping the agents of Weigert's haematoxylin on the samples and culturing for 8 minutes to stain the nuclei. The slides were washed for 10 minutes in running tap water, placed in picro-sirius red staining solution (0.5 g sirius red to 500 mL picric acid (1.3% in water, Sigma-Aldrich)) for one hour and then washed with acidified water (5 mL acetic acid in 1 L of water) with two changes of the washing solution. Finally, the slides were dehydrated in three changes of 100% ethanol and cleared in xylene. The section was scanned under microscopy with a 10X lens.

### Flow-Cytometric Analysis

Optimized flow cytometry protocols were based on published methods (Cheng et al, 2018; Cheng et al, 2020; Zhu et al, 2014). The fresh tumor tissues were dissected into 10 cm tissue culture dishes and cut into small pieces with a sterile razor blade. The tissues were transferred into 50 mL tubes containing a 100 μm cell strainer and washed with PBS (20 mL) followed by centrifuging at 2000 rpm for 3 min. Then, tumor digestion buffer (5 mL RPMI with 1% FBS and 0.25 mL 10× digestion buffer (2 mg/mL Collagenase D, 250 units/μL DNAse I, Sigma Aldrich)) was added into the tube with pelleted tissue after removing the supernatant. The tubes were put onto a shaker at 37° C. and shaken for 1 hour. The samples were filtered using 100 μm cell strainer and washed by adding 35 mL of PBS and spinning at 2000 rpm for 3 min. The pellets were re-suspended with 2 mL ACK lysis buffer to lyse the red blood cells by incubating for 5 min on ice and washed again through centrifugation at 2000 rpm for 3 min after adding 30 mL PBS. Next, the cells (5×10⁶ cells/mL) were incubated with an antibody cocktail solution (1 μL each antibody to 100 μL cell staining buffer) with 0.5 μL Ghost Dye Red 780 (Tonbo Bioscience) at 2-8° C. for 40 min and protected from light. The labeled samples were washed 2 times with 1.5 mL cell staining buffer (BioLegend). The samples were re-suspended into 500 μL cell staining buffer. Data acquisition was performed on an LSRFortessa (BD Biosciences). In addition, single color compensation controls were run on the LSRFortessa prior to the sample data collection. FlowJo was used for data analysis. All the fluorophore-conjugated anti-mouse antibodies are used for flow cytometry were purchased from BioLegend: Pacific Blue anti-mouse CD45 (Biolegend, Cat #1031266), APC anti-mouse CD3 Antibody (Biolegend, Cat #100236), PE anti-mouse CD8a (Biolegend, Cat #162304), PerCP/Cyanine5.5 anti-mouse CD4 (Biolegend, Cat #116012), Alexa Fluor 488 anti-mouse/human CD11b (Biolegend, Cat #101217), Alexa Fluor® 594 anti-mouse F4/80 (Biolegend, Cat #123140).

### Compressive Modulus Measurement

The unconfined compression experimental protocol was employed to measure the compressive modulus of tumor tissue according to published work (Voutouri et al. 2018) (Rashid et al, 2012). Fresh tumors were collected from mice within each group after therapy and mechanical testing was performed within 4 h. Each tumor tissue was preserved in PBS and all samples remained on the ice during transportation and measurement. All samples were cut into small sizes to allow for testing (The size of each sample is detailed in Table 2) and measured at a room temperature ˜22° C. The tumor specimens (n=8) were loaded on a mechanical testing system with a 5.6 lbf load cell (TestResources, MN, USA, 250 lbf actuator). The compression measurement of the tumor tissues was performed to a final strain of 30% with a compression rate of 0.1 mm/min. The compressive modulus was calculated from the slope of the stress-strain curve in the range of 25-30% strain (Voutouri et al, 2018). Representative compressive stress-strain curves and compressive moduli for each group are shown in FIG. 24A and FIG. 24B.

TABLE 2
The size of tumor tissue for measuring the compressive modulus.

| | Length (mm) | Width (mm) | Thickness (mm) |
|---|---|---|---|
| PBS | 6.3 | 4.56 | 3.56 |
| | 5.39 | 5.01 | 3.89 |
| | 5.21 | 4.58 | 3.78 |
| | 4.1 | 3.73 | 3.52 |
| | 2.69 | 4.07 | 2 |
| | 4.29 | 4.09 | 3.03 |
| | 4.6 | 4.05 | 2.66 |
| | 4.95 | 4.58 | 2.3 |
| siCtrl + CRISPR-PD- | 5.64 | 4.71 | 5.04 |
| L1-LNPs | 5.5 | 4.5 | 3.82 |
| | 5.47 | 5.16 | 3.38 |
| | 4.67 | 4.99 | 3.5 |
| | 3.68 | 4.49 | 3.35 |
| | 4.97 | 3.54 | 3.1 |
| | 3.46 | 4.17 | 3.07 |
| | 3.29 | 3.83 | 2.78 |
| siFAK + CRISPR-Ctrl- | 4.7 | 3.52 | 2.43 |
| LNPs | 5.9 | 3.2 | 2.8 |
| | 4.21 | 5.12 | 2.82 |
| | 4.34 | 4.06 | 3.28 |
| | 3.79 | 4.62 | 3.18 |
| | 4.23 | 3.13 | 1.92 |
| | 4.02 | 3.94 | 2.61 |
| | 5.17 | 3.94 | 2.39 |
| siFAK + CRISPR-PD- | 5.08 | 4.03 | 3.26 |
| L1-LNPs | 3.61 | 3.71 | 3.07 |
| | 5.07 | 3.74 | 2.75 |
| | 5.51 | 3.74 | 2.33 |
| | 4.27 | 4.7 | 3.08 |
| | 4.81 | 4.27 | 2.85 |
| | 4.15 | 4.46 | 2.61 |
| | 2.71 | 3.56 | 2.81 |

### Statistics and Reproducibility

Data are reported as mean±s.d. Statistical analysis was performed using the two-tailed t-test, or one-way ANOVA with multiple comparison test. * P<0.05, ** P<0.01, *** P<0.001, **** P<0.0001, no significant (ns) difference using GraphPad Prism software (GraphPad Software, USA). Exact P values which <0.0001 obtained from Excel with the same statistical analysis. The data obtained from micrograph, T7E1, and Western blot are representative images of 3 biologically independent samples (n=3).

All of the compounds, material, compositions, and methods disclosed and claimed herein can be made and executed without undue experimentation in light of the present disclosure. While the disclosure may have focused on several embodiments or may have been described in terms of preferred embodiments, it will be apparent to those of skill in the art that variations and modifications may be applied to the compounds, compositions, and methods without departing from the spirit, scope, and concept of the invention. All variations and modifications apparent to those skilled in the art are deemed to be within the spirit, scope, and concept of the invention as defined by the appended claims.

### REFERENCES

The following references, to the extent that they provide exemplary procedural or other details supplementary to those set forth herein, are specifically incorporated herein by reference.

### WO 2019/183635

Abumanhal-Masarweh, H. et al. Tailoring the lipid composition of nanoparticles modulates their cellular uptake and affects the viability of triple negative breast cancer cells. J. Control. Release 307, 331-341 (2019).
Ball, R. L., Hajj, K. A., Vizelman, J., Bajaj, P. & Whitehead, K.A. Lipid nanoparticle formulations for enhanced co-delivery of siRNA and mRNA. Nano Lett. 18, 3814-3822 (2018).
Casey, S.C. et al. MYC regulates the antitumor immune response through CD47 and PD-L1. Science 352, 227-231 (2016).
Chaudhuri, O. et al. Extracellular matrix stiffness and composition jointly regulate the induction of malignant phenotypes in mammary epithelium. Nat. Mater. 13, q₇₀-978 (2014).
Cheng, Q. et al. Dendrimer-based lipid nanoparticles deliver therapeutic FAH mRNA to normalize liver function and extend survival in a mouse model of hepatorenal tyrosinemia type I. Adv. Mater. 30, e1805308 (2018).
Cheng, Q. et al. Selective ORgan Targeting (SORT) nanoparticles for tissue specific mRNA delivery and CRISPR/Cas gene editing. Nat. Nanotechnol. 15, 313-320 (2020).
Cong, L. et al. Multiplex genome engineering using CRISPR/Cas systems. Science 339, 819-823 (2013).
Cox, A.D. & Der, C. J. Ras history: The saga continues. Small GTPases 1, 2-27 (2010).
Cox, D. B., Platt, R. J. & Zhang, F. Therapeutic genome editing: prospects and challenges. Nat. Med. 21, 121-131 (2015).
Dupont, S. et al. Role of YAP/TAZ in mechanotransduction. Nature 474, 179-183 (2011).
Echarri, A. & Del Pozo, M. A. Caveolae-mechanosensitive membrane invaginations linked to actin filaments. J. Cell Sci. 128, 2747-2758 (2015).
Egeblad, M., Rasch, M. G. & Weaver, V. M. Dynamic interplay between the collagen scaffold and tumor evolution. Curr. Opin. Cell Biol. 22, 697-706 (2010).
Fourcade, J. et al. CD8+ T cells specific for tumor antigens can be rendered dysfunctional by the tumor microenvironment through upregulation of the inhibitory receptors BTLA and PD-1. Cancer Res. 72, 887-896 (2012).
Hanahan, D. & Weinberg, R.A. Hallmarks of cancer: The next generation. Cell 144, 646-674 (2011).
Hoadley, K. A. et al. Multiplatform analysis of 12 cancer types reveals molecular classification within and across tissues of origin. Cell 158, q₂₉-944 (2014).
Huang, C. H., Lee, K. C. & Doudna, J. A. Applications of CRISPR-Cas enzymes in cancer therapeutics and detection. Trends Cancer 4, 499-512 (2018).
Humphrey, J. D., Dufresne, E. R. & Schwartz, M. A. Mechanotransduction and extracellular matrix homeostasis. Nat. Rev. Mol. Cell Biol. 15, 802-812 (2014).
Jiang, H. et al. Targeting focal adhesion kinase renders pancreatic cancers responsive to checkpoint immunotherapy. Nat. Med. 22, 851-860 (2016).
Jinek, M. et al. A programmable dual-RNA-guided DNA endonuclease in adaptive bacterial immunity. Science 337, 816-821 (2012).
Kaksonen, M. & Roux, A. Mechanisms of clathrin-mediated endocytosis. Nat. Rev. Mol. Cell Biol. 19, 313-326 (2018).
Kraning-Rush, C. M., Califano, J. P. & Reinhart-King, C. A. Cellular traction stresses increase with increasing metastatic potential. Plos One 7, e32572 (2012).
Laklai, H. et al. Genotype tunes pancreatic ductal adenocarcinoma tissue tension to induce matricellular fibrosis and tumor progression. Nat. Med. 22, 497-505 (2016).
Lampi, M.C. & Reinhart-King, C. A. Targeting extracellular matrix stiffness to attenuate disease: From molecular mechanisms to clinical trials. Sci. Trans. Med. 10, eaao0475 (2018).
Lee, S. M. et al. A systematic study of unsaturation in lipid nanoparticles leads to improved mRNA transfection in vivo. Angew. Chem. Int. Ed. 60, 5848-5853 (2021).
Liu, S. et al. Membrane-destabilizing ionizable phospholipids for organ-selective mRNA delivery and CRISPR-Cas gene editing. Nat. Mater. 20, 701-710 (2021).
Long, C. Z. et al. Postnatal genome editing partially restores dystrophin expression in a mouse model of muscular dystrophy. Science 351, 400-403 (2016).
Maddalo, D. et al. In vivo engineering of oncogenic chromosomal rearrangements with the CRISPR/Cas9 system. Nature 516, 423 (2014).
Mali, P. et al. RNA-guided human genome engineering via Cas9. Science 339, 823-826 (2013).
Mehta, G., Hsiao, A. Y., Ingram, M., Luker, G. D. & Takayama, S. Opportunities and challenges for use of tumor spheroids as models to test drug delivery and efficacy. J. Control. Release 164, 192-204 (2012).
Miller, J. B. & Siegwart, D. J. Design of synthetic materials for intracellular delivery of RNAs: From siRNA-mediated gene silencing to CRISPR/Cas gene editing. Nano Res. 11, 5310-5337 (2018).
Mohammadi, H. & Sahai, E. Mechanisms and impact of altered tumour mechanics. Nat. Cell Biol. 20, 766-774 (2018).
Naito, Y. et al. CD8+ T cells infiltrated within cancer cell nests as a prognostic factor in human colorectal cancer. Cancer Res. 58, 3491-3494 (1998).
Paszek, M. J. et al. Tensional homeostasis and the malignant phenotype. Cancer Cell 8, 241-254 (2005).
Patel, S. et al. Naturally-occurring cholesterol analogues in lipid nanoparticles induce polymorphic shape and enhance intracellular delivery of mRNA. Nat. Commun. 11, q83 (2020).
Rashid, B., Destrade, M. & Gilchrist, M. D. Mechanical characterization of brain tissue in compression at dynamic strain rates. J. Mech. Behav. Biomed. 10, 23-38 (2012).
Schreiber, R. D., Old, L. J. & Smyth, M. J. Cancer immunoediting: integrating immunity's roles in cancer suppression and promotion. Science 331, 1565-1570 (2011).
Seong, J., Wang, N. & Wang, Y. Mechanotransduction at focal adhesions: from physiology to cancer development. J. Cell. Mol. Med. 17, 597-604 (2013).
Serrels, A. et al. Nuclear FAK controls chemokine transcription, Tregs, and evasion of anti-tumor immunity. Cell 163, 160-173 (2015).
Shachaf, C. M. et al. MYC inactivation uncovers pluripotent differentiation and tumour dormancy in hepatocellular cancer. Nature 431, 1112-1117 (2004).
Sica, A. & Mantovani, A. Macrophage plasticity and polarization: in vivo veritas. J. Clin. Invest. 122, 787-795 (2012).
Stokes, J. B. et al. Inhibition of focal adhesion kinase by PF-562,271 inhibits the growth and metastasis of pancreatic cancer concomitant with altering the tumor microenvironment. Mol. Cancer Ther. 10, 2135-2145 (2011).
Topalian, S. L., Drake, C. G. & Pardoll, D. M. Targeting the PD-1/B7-H₁ (PD-L1) pathway to activate anti-tumor immunity. Curr. Opin. Immunol. 24, 207-212 (2012).
Voutouri, C. & Stylianopoulos, T. Accumulation of mechanical forces in tumors is related to hyaluronan content and tissue stiffness. Plos One 13, e0193801 (2018).
Wang, H. X. et al. CRISPR/Cas9-based genome editing for disease modeling and therapy: Challenges and opportunities for nonviral delivery. Chem. Rev. 117, q₈₇₄-9906 (2017).
Wei, T. et al. Delivery of tissue-targeted scalpels: Opportunities and challenges for in vivo CRISPR/Cas -based genome editing. ACS Nuno 14, q₂₄₃-9262 (2020).
Wu, S. Y., Lopez-Berestein, G., Calin, G. A. & Sood, A. K. RNAi therapies: Drugging the undruggable. Sci. Trans. Med. 6, 240-247 (2014).
Xue, W. et al. CRISPR-mediated direct mutation of cancer genes in the mouse liver. Nature 514, 380-384 (2014).
Yin, H. et al. Genome editing with Cas9 in adult mice corrects a disease mutation and phenotype. Nat. Biotechnol. 32, 551-553 (2014).
Zhang, S. et al. Knockdown of anillin actin binding protein blocks cytokinesis in hepatocytes and reduces liver tumor development in mice without affecting regeneration. Gastroenterology 154, 1421-1434 (2018).
Zheng, K., Cubero, F. J. & Nevzorova, Y. A. c-MYC making liver sick: Role of c-MYC in hepatic cell function, homeostasis and disease. Genes 8, 123 (2017).
Zhou, K. et al. Modular degradable dendrimers enable small RNAs to extend survival in an aggressive liver cancer model. Proc. Natl. Acad. Sci. USA 113, 520-525 (2016).
Zhu, Y. et al. CSF1/CSF1R blockade reprograms tumor-infiltrating macrophages and improves response to T-cell checkpoint immunotherapy in pancreatic cancer models. Cancer Res. 74, 5057-5069 (2014).

What is claimed is:

1. A composition comprising:
(A) an interfering polynucleotide;
(B) a second polynucleotide, wherein the second polynucleotide is either:
(1) a polynucleotide comprising a sequence encoding for a polynucleotide guided nuclease; or
(2) a guide polynucleotide;
(C) a lipid nanoparticle comprising at least one ionizable lipid.

2. The composition of claim 1, wherein the composition comprises a polynucleotide comprising a sequence encoding for a polynucleotide guided nuclease and a guide polynucleotide.

3. The composition of either claim 1 or claim 2, wherein the interfering polynucleotide is an siRNA.

4. The composition according to any one of claims 1-3, wherein the polynucleotide comprising a sequence encoding for a polynucleotide guided nuclease is a mRNA.

5. The composition of claim 4, wherein the mRNA encodes for a Cas protein.

6. The composition of claim 5, wherein the Cas protein is a Cas9 protein.

7. The composition according to any one of claims 1-6, wherein the guide polynucleotide is a polynucleotide configured to complex with at least a portion of a target gene or transcript.

8. The composition according to any one of claims 1-6, wherein the guide polynucleotide is a polynucleotide that encodes for a polynucleotide that is configured to complex with at least a portion of a target gene or transcript.

9. The composition according to any one of claims 1-8, wherein the guide polynucleotide comprises from about 10 nucleotides to about 50 nucleotides.

10. The composition of claim 9, wherein the guide polynucleotide comprises from about 12 nucleotides to about 40 nucleotides.

11. The composition of either claim 9 or claim 10, wherein the guide polynucleotide comprises from about 15 nucleotides to about 30 nucleotides.

12. The composition according to any one of claims 1-11, wherein the interfering polynucleotide comprises from about 10 nucleotides to about 50 nucleotides.

13. The composition of claim 12, wherein the interfering polynucleotide comprises from about 15 nucleotides to about 40 nucleotides.

14. The composition of either claim 12 or claim 13, wherein the interfering polynucleotide comprises from about 18 nucleotides to about 30 nucleotides.

15. The composition according to any one of claims 12-14, wherein the interfering polynucleotide comprise about 20 nucleotides to about 24 nucleotides.

16. The composition according to any one of claims 1-15, wherein the polynucleotide comprising a sequence encoding for a polynucleotide guided nuclease comprises from about comprises from about 250 nucleotides to about 15,000 nucleotides.

17. The composition of claim 16, wherein the polynucleotide comprising a sequence encoding for a polynucleotide guided nuclease comprises from about 500 nucleotides to about 5,000 nucleotides.

18. The composition of either claim 16 or claim 17, wherein the polynucleotide comprising a sequence encoding for a polynucleotide guided nuclease comprises from about 800 nucleotides to about 2,500 nucleotides.

19. The composition according to any one of claims 1-18, wherein the composition comprises a weight ratio of the polynucleotide comprising a sequence encoding for a polynucleotide guided nuclease to the guide polynucleotide from about 10:1 to about 1:5.

20. The composition according to claim 19, wherein the weight ratio of a polynucleotide comprising a sequence encoding for a polynucleotide guided nuclease to the guide polynucleotide is from about 8:1 to about 1:2.

21. The composition according to claim 20, wherein the weight ratio of the polynucleotide comprising a sequence encoding for a polynucleotide guided nuclease to the guide polynucleotide is from about 5:1 to about 1:1.

22. The composition according to claim 21, wherein the weight ratio of the polynucleotide comprising a sequence encoding for a polynucleotide guided nuclease to the guide polynucleotide is 2:1.

23. The composition according to any one of claims 1-22, wherein the composition comprises a weight ratio of the polynucleotide comprising a sequence encoding for a polynucleotide guided nuclease to the inhibitory polynucleotide from about 10:1 to about 1:10.

24. The composition according to claim 23, wherein the weight ratio of the polynucleotide comprising a sequence encoding for a polynucleotide guided nuclease to the inhibitory polynucleotide is from about 5:1 to about 1:5.

25. The composition according to claim 24, wherein the weight ratio of the polynucleotide comprising a sequence encoding for a polynucleotide guided nuclease to the inhibitory polynucleotide is from about 2:1 to about 1:2.

26. The composition according to claim 25, wherein the weight ratio of the polynucleotide comprising a sequence encoding for a polynucleotide guided nuclease to the inhibitory polynucleotide is 1:1 or 2:3.

27. The composition according to any one of claims 1-26, wherein the composition comprises a weight ratio of the guide polynucleotide to the inhibitory polynucleotide from about 4:1 to about 1:10.

28. The composition according to claim 27, wherein the weight ratio of the guide polynucleotide to the inhibitory polynucleotide is from about 2:1 to about 1:8.

29. The composition according to claim 28, wherein the weight ratio of the guide polynucleotide to the inhibitory polynucleotide is from about 1:1 to about 1:4.

30. The composition according to claim 29, wherein the weight ratio of the guide polynucleotide to the inhibitory polynucleotide is 1:2 or 1:3.

31. The composition according to any one of claims 1-30, wherein the ionizable lipid is a cationic lipid.

32. The composition according to any one of claims 1-31, wherein the ionizable lipid is a dendron or dendrimer.

33. The composition according to any one of claims 1-32, wherein the ionizable lipid is a compound of the formula:
Core-Repeating Unit-Terminating Group (D-I)
wherein the core is linked to the repeating unit by removing one or more hydrogen atoms from the core and replacing the atom with the repeating unit and wherein:
the core has the formula: 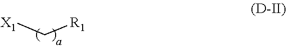
wherein:
X₁ is amino or alkylamino_{(C≤12)}, dialkylamino_{(C≤12)}, heterocycloalkyl_{(C≤12)}, heteroaryl_{(C≤12)}, or a substituted version thereof;
R₁ is amino, hydroxy, or mercapto, or alkylamino_{(C≤12)}, dialkylamino (<12), or a substituted version of either of these groups; and
a is 1, 2, 3, 4, 5, or 6; or
the core has the formula: 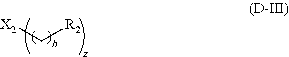
wherein:
X₂ is N(R₅)_{y};
R₅ is hydrogen, alkyl_{(C≤18)}, or substituted alkyl_{(C≤18)}; and
y is 0, 1, or 2, provided that the sum of y and z is 3;
R₂ is amino, hydroxy, or mercapto, or alkylamino_{(C≤12)}, dialkylamino_{(C≤12)}, or a substituted version of either of these groups;
b is 1, 2, 3, 4, 5, or 6; and
z is 1, 2, 3; provided that the sum of z and y is 3; or
the core has the formula: 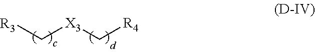
wherein:
X₃ is —NR₆—, wherein R₆ is hydrogen, alkyl_{(C≤8)}, or substituted alkyl_{(C≤8)}, —O—, or alkylaminodiyl_{(C≤8)}, alkoxydiyl_{(C≤8)}, arenediyl_{(C≤8)}, heteroarenediyl_{(C≤8)}, heterocycloalkanediyl_{(C≤8)}, or a substituted version of any of these groups;
R₃ and R₄ are each independently amino, hydroxy, or mercapto, or
alkylamino_{(C≤12)}, dialkylamino (<12), or a substituted version of either of these groups; or a group of the formula: —N(R_{f})_{f}(CH₂CH₂N(R_{c}))ₑR_{d}, 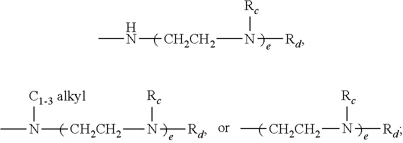
wherein:
e and f are each independently 1, 2, or 3; provided that the sum of e and f is 3;
R_{c}, R_{d}, and R_{f} are each independently hydrogen, alkyl_{(C≤6)}, or substituted alkyl_{(C≤6)};
c and d are each independently 1, 2, 3, 4, 5, or 6; or
the core is alkylamine_{(C≤18)}, dialkylamine_{(C≤36)}, heterocycloalkane_{(C≤12)}, or a substituted version of any of these groups;
wherein the repeating unit comprises a degradable diacyl and a linker;
the degradable diacyl group has the formula: 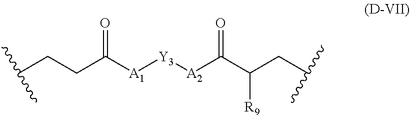
wherein:
A₁ and A₂ are each independently —O—, —S—, or —NRₐ—, wherein:
Rₐ is hydrogen, alkyl_{(C≤6)}, or substituted alkyl_{(C≤6)};
Y₃ is alkanediyl_{(C≤12)}, alkenediyl_{(C≤12)}, arenediyl_{(C≤12)}, or a substituted version of any of these groups; or a group of the formula: 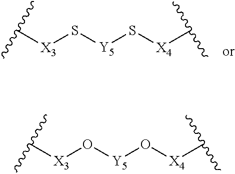
wherein:
X₃ and X₄ are alkanediyl_{(C≤12)}, alkenediyl_{(C≤12)}, arenediyl_{(C≤12)}, or a substituted version of any of these groups;
Y₅ is a covalent bond, alkanediyl_{(C≤12)}, alkenediyl_{(C≤12)}, arenediyl_{(C≤12)}, or a substituted version of any of these groups; and
R₉ is alkyl_{(C≤8)} or substituted alkyl_{(C≤8)};
the linker group has the formula: 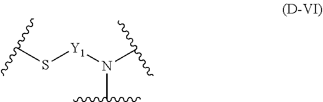
wherein:
Y₁ is alkanediyl_{(C≤12)}, alkenediyl_{(C≤12)}, arenediyl_{(C≤12)}, or a substituted version of any of these groups; and
wherein when the repeating unit comprises a linker group, then the linker group comprises an independent degradable diacyl group attached to both the nitrogen and the sulfur atoms of the linker group if n is greater than 1, wherein the first group in the repeating unit is a degradable diacyl group, wherein for each linker group, the next repeating unit comprises two degradable diacyl groups attached to the nitrogen atom of the linker group; and wherein n is the number of linker groups present in the repeating unit; and
the terminating group has the formula: 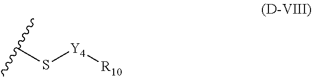
wherein:
Y₄ is alkanediyl_{(C≤24)}, alkenediyl_{(C≤24)}, or a substituted version thereof;
R₁₀ is hydrogen, amino, carboxy, hydroxy, or
aryl_{(C≤12)}, alkylamino_{(C≤12)}, dialkylamino_{(C≤12)}, N-heterocycloalkyl_{(C≤12)}, —C(O)N(R₁₁)-alkanediyl(C≤6)-heterocycloalkyl_{(C≤12)}, —C(O)-alkyl-amino_{(C≤12)}, —C(O)-dialkylamino_{(C≤12)}, —C(O)—N-heterocyclo-alkyl_{(C≤12)}, wherein:
R₁₁ is hydrogen, alkyl_{(C≤6)}, or substituted alkyl_{(C≤6)};
wherein the final degradable diacyl in the chain is attached to a terminating group;
n is 0, 1, 2, 3, 4, 5, or 6;
or a pharmaceutically acceptable salt thereof.

34. The composition according to claim 33, wherein, in Formula (D-I), the core is further defined by the formula: 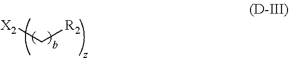
wherein:
X₂ is N(R₅)_{y};
R₅ is hydrogen or alkyl_{(C≤8)}, or substituted alkyl_{(C≤18)}; and
y is 0, 1, or 2, provided that the sum of y and z is 3;
R₂ is amino, hydroxy, or mercapto, or alkylamino_{(C≤12)}, dialkylamino_{(C≤12)}, or a substituted version of either of these groups;
b is 1, 2, 3, 4, 5, or 6; and
z is 1, 2, 3; provided that the sum of z and y is 3.

35. The composition according to claim 33 or 34, wherein, in Formula (D-I), the core is further defined as: 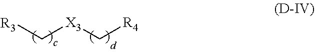
wherein:
X₃ is —NR₆—, wherein R₆ is hydrogen, alkyl_{(C≤8)}, or substituted alkyl_{(C≤8)}, —O—, or alkylaminodiyl(C≤), alkoxydiyl (≤8), heteroarenediyl_{(C≤8)}, heterocycloalkanediyl(C≤), or a substituted version of any of these groups;
R₃ and Rₐ are each independently amino, hydroxy, or mercapto, or alkylamino_{(C≤12)}, dialkylamino_{(C≤12)}, or a substituted version of either of these groups; or a group of the formula: —N(R_{f})_{f}(CH₂CH₂N(Rₑ))ₑR_{d}, 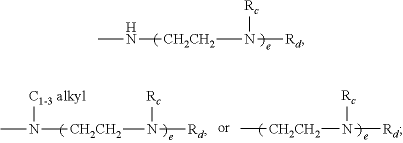
wherein:
e and f are each independently 1, 2, or 3; provided that the sum of e and f is 3;
R_{c}, R_{d}, and R_{f} are each independently hydrogen, alkyl_{(C≤6)}, or substituted alkyl_{(C≤6)};
c and d are each independently 1, 2, 3, 4, 5, or 6.

36. The composition according to any one of claims 33-35, wherein, in Formula (D-I), the core is further defined as:

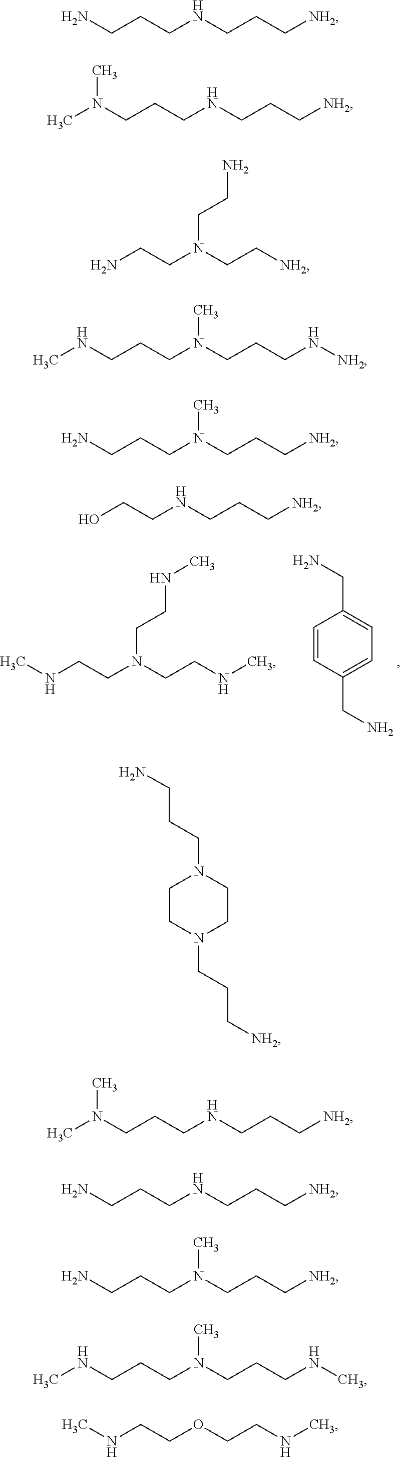

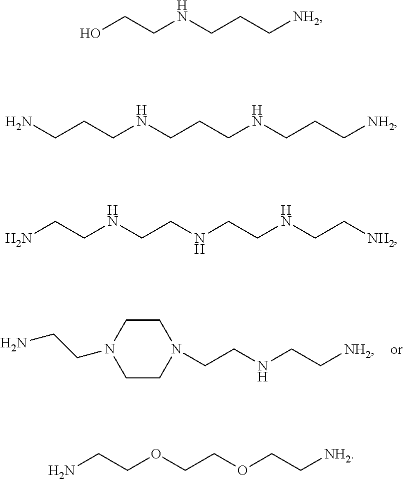 [SMILES: NCCCNCCO ; NCCCNCCCNCCCN ; NCCNCCNCCNCCN ; NCCNCCN1CCN(CCN)CC1 ; NCCOCCOCCN]

37. The composition according to any one of claims 33-36, wherein, in Formula (D-I), the core is further defined as:

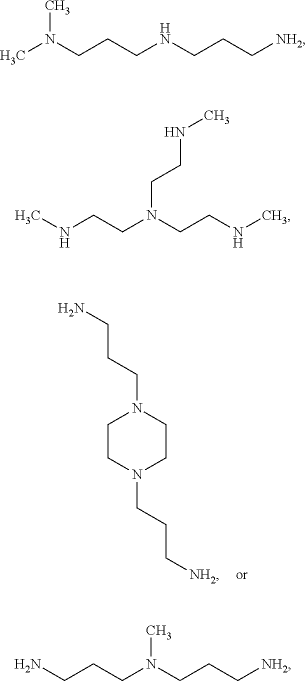

38. The composition according to any one of claims 33-37, wherein, in Formula (D-I), the core is further defined as:

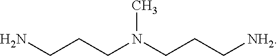

39. The composition according to any one of claims 33-38, wherein A₁ and A₂ are O.

40. The composition according to any one of claims 33-39, wherein Y₃ is alkanediyl_{(C≤12)} or substituted alkanediyl_{(C≤12)}.

41. The composition according to any one of claims 33-40, wherein Y₁ is alkanediyl_{(C≤}12) or substituted alkanediyl_{(C≤12)}.

42. The composition according to any one of claims 33-41, wherein the terminating group is further defined as: 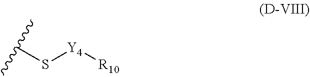
wherein:
Y₄ is alkanediyl_{(C≤18)} or alkenediyl_{(C≤18)}; and
R₁₀ is hydrogen.

43. The composition according to any one of claims 33-42, wherein the terminating group is further defined as: 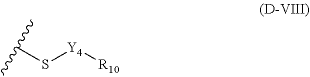
wherein:
Y₄ is alkanediyl_{(C≤18)}; and
R₁₀ is hydrogen.

44. The composition according to any one of claims 33-43, wherein the dendrimer or dendron is further defined as: 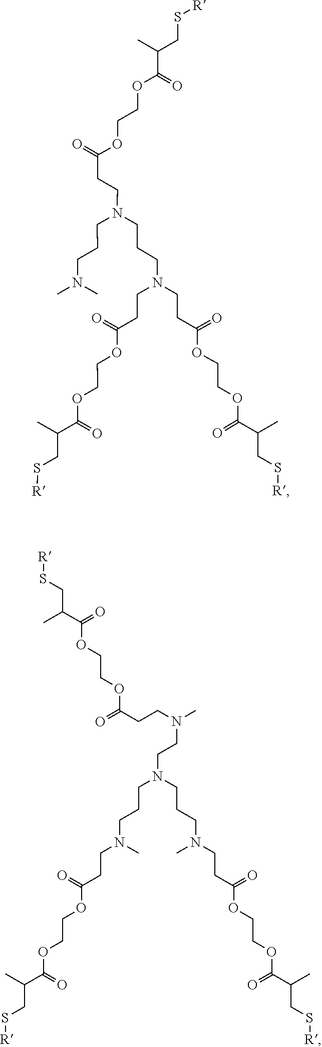 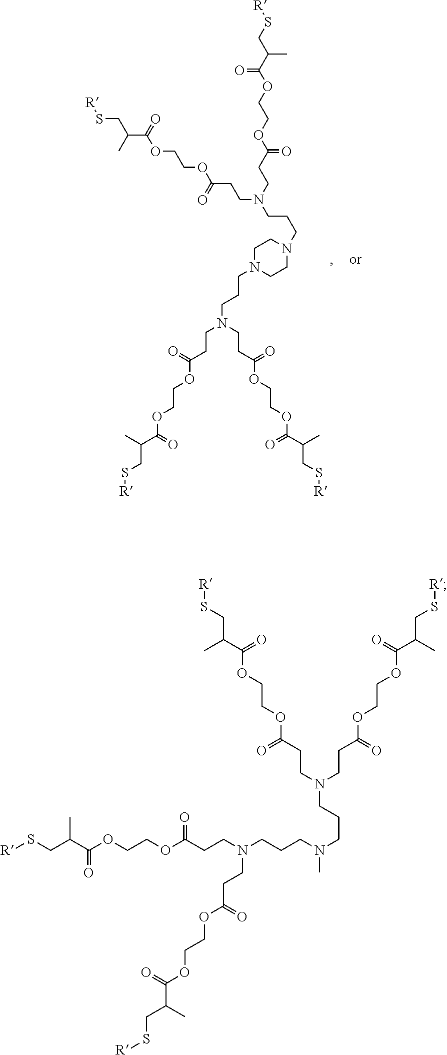
wherein:
R′ is alkyl_{(C≤18)}, alkenyl_{(C≤18)}, or a substituted version thereof.

45. The composition according to any one of claims 33-44, wherein the dendrimer or dendron is further defined as: 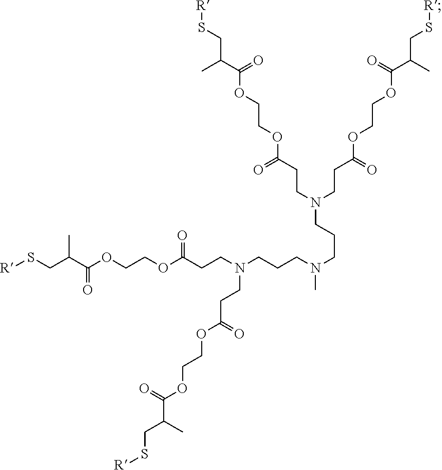
wherein:
R′ is alkyl_{(C≤18)}, alkenyl_{(C≤18)}, or a substituted version thereof.

46. The composition according to claim 45, wherein the dendrimer or dendron is further defined as: 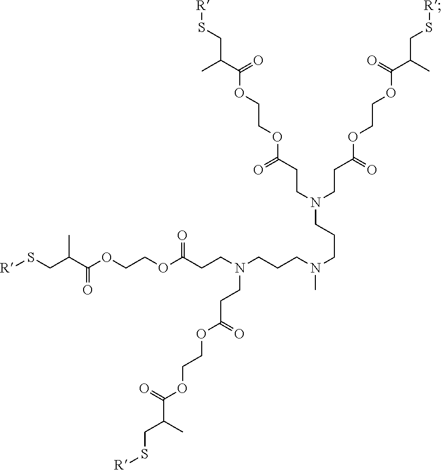
wherein:
R′is alkyl_{(C6-18)}.

47. The composition according to any one of claims 1-32, wherein the ionizable cationic lipid is a dendrimer or dendron of a generation (g) having a structural formula: 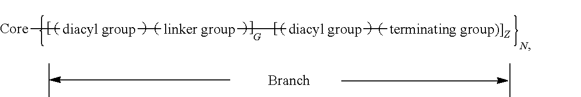
or a pharmaceutically acceptable salt thereof, wherein:
(a) the core comprises a structural formula (X_{Core}): 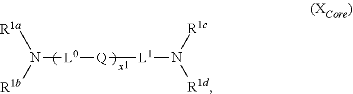
wherein:
Q is independently at each occurrence a covalent bond, —O—, —S—, —NR₂—, or —CR^{3a}R^{3b}—;
R² is independently at each occurrence R^{1g} or -L²—NR^{1e}R^{1f};
R^{3a} and R^{3b} are each independently at each occurrence hydrogen or an optionally substituted alkyl;
R^{1a}, R^{1b}, R^{1c}, R^{1d}, R^{1e}, R^{1f}, and R^{1g} (if present) are each independently at each occurrence a point of connection to a branch, hydrogen, or an optionally substituted alkyl;
L⁰, L¹, and L² are each independently at each occurrence selected from a covalent bond, alkanediyl, heteroalkanediyl, [alkanediyl]-[heterocycloalkanediyl]-[alkanediyl], [alkanediyl]-(arenediyl)-[alkanediyl], heterocycloalkyl, and arenediyl; or,
alternatively, part of L¹ form a heterocycloalkyl with one of R^{1c} and R^{1d}, and x¹ is 0, 1, 2, 3, 4, 5, or 6; and
(b) each branch of the plurality (N) of branches independently comprises a structural formula (X_{Branch}): 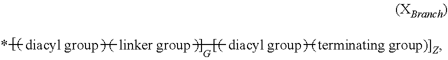
wherein:
* indicates a point of attachment of the branch to the core;
g is 1, 2, 3, or 4;
Z=2^{(g-1)};
G=0, when g=1; or G=Σᵢ₌₀^{i=g-2}2ⁱ, when g≠1;
(c) each diacyl group independently comprises a structural formula 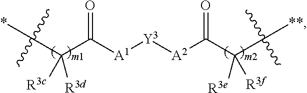
wherein:
* indicates a point of attachment of the diacyl group at the proximal end thereof;
** indicates a point of attachment of the diacyl group at the distal end thereof;
Y₃ is independently at each occurrence an optionally substituted alkanediyl, an optionally substituted alkenediyl, or an optionally substituted arenediyl;
A₁ and A₂ are each independently at each occurrence —O—, —S—, or —NR⁴—, wherein:
R⁴ is hydrogen or optionally substituted alkyl;
m¹ and m² are each independently at each occurrence 1, 2, or 3; and
R^{3c}, R^{3d}, R^{3e}, and R^{3f} are each independently at each occurrence hydrogen or an optionally substituted (e.g., C₁-C₈) alkyl; and
(d) each linker group independently comprises a structural formula 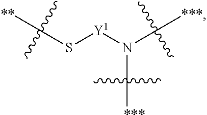
wherein:
** indicates a point of attachment of the linker to a proximal diacyl group;
*** indicates a point of attachment of the linker to a distal diacyl group; and
Y₁ is independently at each occurrence an optionally substituted alkanediyl, an optionally substituted alkenediyl, or an optionally substituted arenediyl; and
(e) each terminating group is independently selected from optionally substituted alkylthiol and optionally substituted alkenylthiol.

48. The composition of claim 47, wherein x¹ is 0, 1, 2, or 3.

49. The composition of claim 47 or 48, wherein R^{1a}, R^{1b}, R^{1c}, R^{1d}, R^{1e}, R^{1f}, and R^{1g} (if present) are each independently at each occurrence a point of connection to a branch as indicated by *, hydrogen, or C₁-C₁₂ alkyl, wherein the alkyl moiety is optionally substituted with one or more substituents each independently selected from —OH, C₄-C₈ heterocycloalkyl, N-(alkyl)-piperidinyl, piperazinyl, N-(alkyl)-piperadizinyl, morpholinyl, N-pyrrolidinyl, pyrrolidinyl, or N-(alkyl)-pyrrolidinyl, aryl, and heteroaryl, or pyridinyl.

50. The composition of claim 49, wherein R^{1a}, R^{1b}, R^{1c}, R^{1d}, R^{1e}, R^{1f}, and R^{1g} (if present) are each independently at each occurrence a point of connection to a branch as indicated by *, hydrogen, or alkyl, wherein the alkyl moiety is optionally substituted with one substituent-OH.

51. The composition of any one of claims 47-50, wherein R^{3a} and R^{3b} are each independently at each occurrence hydrogen.

52. The composition of any one of claims 47-51, wherein the plurality (N) of branches comprises at least 3 branches.

53. The composition of any one of claims 47-52, wherein the plurality (N) of branches comprises at least 4 branches.

54. The composition of any one of claims 47-53, wherein the plurality (N) of branches comprises at least 5 branches.

55. The composition of any one of claims 47-54, wherein g=1; G=0; and Z=1.

56. The composition of claim 55, wherein each branch of the plurality of branches comprises a structural formula

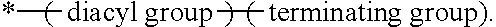

57. The composition of any one of claims 47-54, wherein g=2; G=1; and Z=2.

58. The composition of claim 57, wherein each branch of the plurality of branches comprises a structural formula

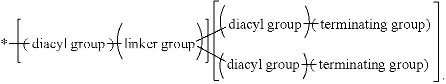

59. The composition of any one of claims 47-58, wherein the core comprises a structural formula:

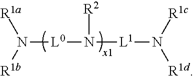

60. The composition of claim 59, wherein the core comprises a structural formula:

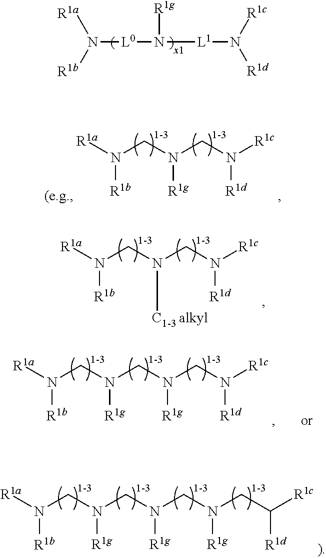

61. The composition of claim 60, wherein the core comprises a structure selected from:

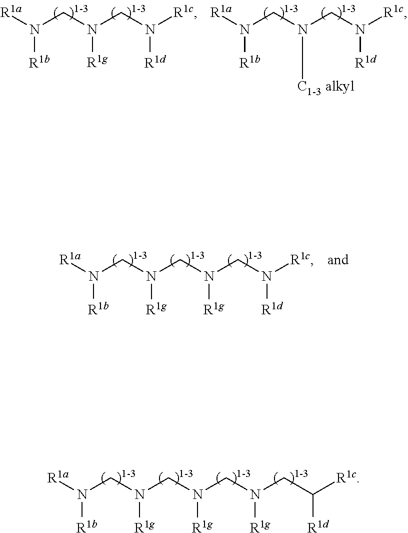

62. The composition of any one of claims 47-61, wherein the core comprises a structural formula selected from the group consisting of: 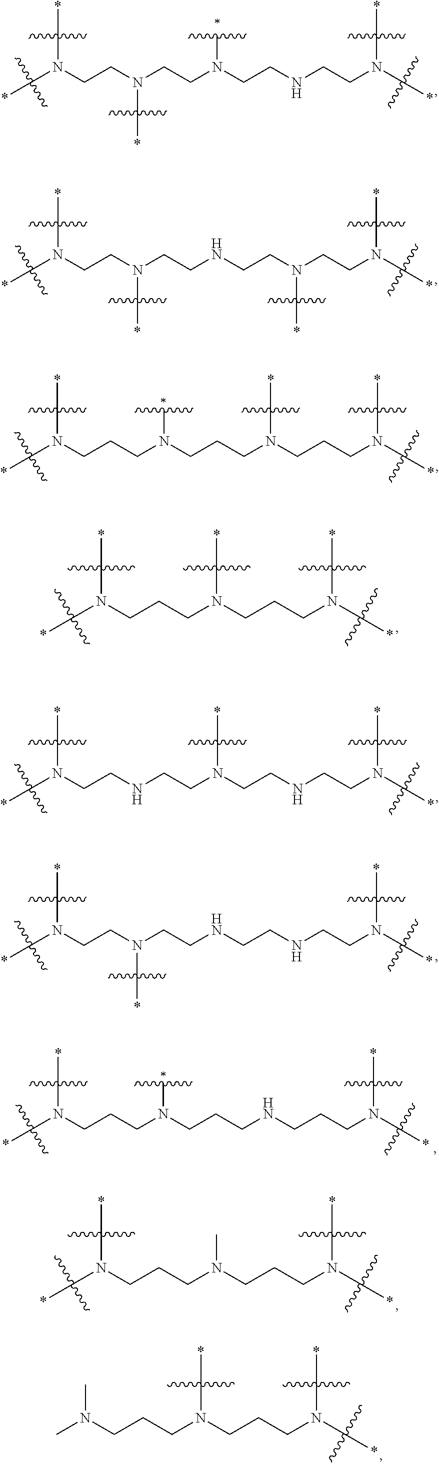
and pharmaceutically acceptable salts thereof, wherein * indicates a point of attachment of the core to a branch of the plurality of branches.

63. The composition of any one of claims 49-62, wherein the core has the structure 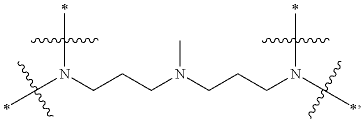
wherein * indicates a point of attachment of the core to a branch of the plurality of branches or H, wherein at least 2 (e.g., at least 3, or at least 4) branches are attached to the core.

64. The composition of claim 63, wherein the core further comprises at least 3 branches attached to the core.

65. The composition of either claim 63 or claim 64, wherein the core further comprises at least 4 branches attached to the core.

66. The composition of any one of claims 49-65, wherein the core has the structure 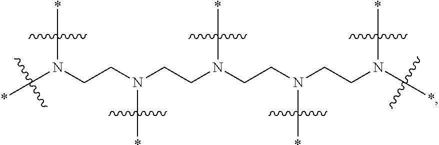
wherein * indicates a point of attachment of the core to a branch of the plurality of branches or H, wherein at least 4 branches are attached to the core.

67. The composition of claim 66, wherein the core comprises at least 5 branches attached to the core.

68. The composition of either claim 66 or claim 67, wherein the core comprises at least 6 branches attached to the core.

69. The composition of any one of claims 49-68, wherein A₁ is —O—or —NH—.

70. The composition of any one of claims 49-69, wherein A₂ is —O—or —NH—.

71. The composition of any one of claims 49-70, wherein Y₃ is C₁-C₁₂ alkanediyl.

72. The composition of any one of claims 49-71, wherein the diacyl group independently at each occurrence comprises a structural formula 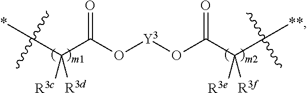
optionally wherein R^{3c}, R^{3d}, R^{3e}, and R^{3f} are each independently at each occurrence hydrogen or C₁-C₃ alkyl.

73. The composition of claim 72, wherein the diacyl group is further defined as:

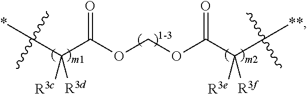

74. The composition of claim 73, wherein the diacyl group is further defined as:

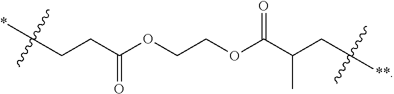

75. The composition of any one of claims 49-74, wherein each terminating group is independently C₁-C₁₈ alkylthiol.

76. The composition of any one of claims 49-74, wherein each terminating group is independently C₁-C₁₈ alkenylthiol.

77. The composition according to any one of claims 1-76, wherein the composition comprises a molar percentage of the ionizable lipid from about 5 to about 50 of the ionizable lipid relative to the total lipid composition.

78. The composition according to claim 77, wherein the molar percentage of the ionizable lipid is from about 15 to about 40 of the ionizable lipid relative to the total lipid composition.

79. The composition according to claim 78, wherein the molar percentage of the ionizable lipid is from about 20 to about 30 of the ionizable lipid relative to the total lipid composition.

80. The composition according to any one of claims 1-79, wherein the composition further comprises a phospholipid.

81. The composition according to claim 80, wherein the phospholipid comprises one or two long chain alkyl or alkenyl groups, a glycerol or a sphingosine, one or two phosphate groups, and a small organic molecule, wherein the small organic molecule is an amino acid, a sugar, or an amino substituted alkoxy group.

82. The composition according to claim 80 or 81, wherein the phospholipid is 1,2-distearoyl-sn-glycero-3-phosphocholine (DSPC) or 1,2-dioleoyl-sn-glycero-3-phosphoethanolamine (DOPE).

83. The composition according to any one of claims 80-82, wherein the phospholipid is DOPE.

84. The composition according to any one of claims 1-83, wherein the composition comprises a molar percentage of the phospholipid from about 5 to about 50 of the phospholipid relative to the total lipid composition.

85. The composition according to claim 84, wherein the molar percentage of the phospholipid is from about 10 to about 40 of the phospholipid relative to the total lipid composition.

86. The composition according to claim 85, wherein the molar percentage of the phospholipid is from about 20 to about 30 of the phospholipid relative to the total lipid composition.

87. The composition according to any one of claims 1-86, wherein the composition further comprises a steroid.

88. The composition according to claim 87, wherein the steroid is cholesterol.

89. The composition according to claim 87 or 88, wherein the composition comprises a molar percentage of the steroid from about 10 to about 65 of the steroid relative to the total lipid composition.

90. The composition of claim 89, wherein the molar percentage of the steroid is from about to about 55 of the steroid relative to the total lipid composition.

91. The composition of claim 90, wherein the molar percentage of the steroid is from about to about 50 of the steroid relative to the total lipid composition.

92. The composition according to any one of claims 1-91, wherein the composition further comprises a polymer-conjugated lipid.

93. The composition of claim 92, wherein the polymer-conjugated lipid is a PEGylated lipid.

94. The composition of either claim 92 or claim 93, wherein the polymer-conjugated lipid comprises a polyethylene glycol (PEG) component from about 1000 to about 10,000 daltons.

95. The composition according to any one of claims 92-94, wherein the polymer-conjugated lipid is a PEGylated diacylglycerol.

96. The composition according to claim 95, wherein the polymer-conjugated lipid is further defined by the formula: 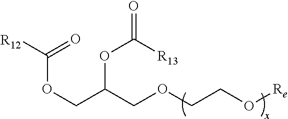
wherein:
R₁₂ and R₁₃ are each independently alkyl_{(C≤24)}, alkenyl_{(C≤24)}, or a substituted version of either of these groups;
Rₑ is hydrogen, alkyl_{(C≤8)}, or substituted alkyl_{(C≤8)}; and
x is 1-250.

97. The composition according to any one of claims 1-94, wherein the polymer-conjugated lipid is a PEGylated dimyristoyl-sn-glycerol or a compound of the formula: 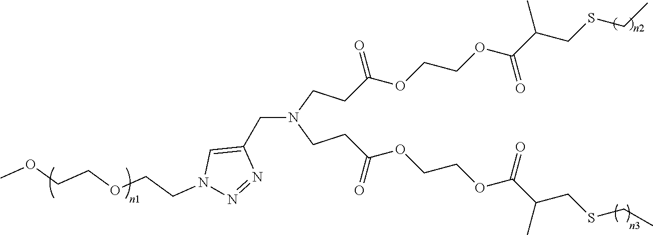
wherein:
n₁ is 5-250; and
n₂ and n₃ are each independently 2-25.

98. The composition according to any one of claims 1-97, wherein the composition comprises a molar percentage of the polymer-conjugated lipid from about 0.1 to about of the polymer-conjugated lipid relative to the total lipid composition.

99. The composition according to claim 98, wherein the molar percentage of the polymer-conjugated lipid is from about 0.5 to about 10 of the polymer-conjugated lipid relative to the total lipid composition.

100. The composition according to claim 99, wherein the molar percentage of the polymer-conjugated lipid is from about 1 to about 6 of the polymer-conjugated lipid relative to the total lipid composition.

101. The composition according to any one of claims 1-100, wherein the composition further comprises a second polymer-conjugated lipid.

102. The composition of claim 101, wherein the second polymer-conjugate lipid is a PEGylated lipid.

103. The composition of either claim 101 or claim 102, wherein the polymer-conjugated lipid comprises a polyethylene glycol (PEG) component from about 1000 to about 10,000 daltons.

104. The composition according to any one of claims 101-103, wherein the second polymer-conjugated lipid is a PEGylated diacylglycerol.

105. The composition of claim 104, wherein the PEGylated diacylglycerol further comprises one or more phosphate groups.

106. The composition of either claim 104 or claim 105, wherein the PEGylated diacylglycerol further comprises one or more cell targeting moieties.

107. The composition of claim 106, wherein the cell targeting moiety is an antibody, a nucleic acid, a protein or peptide, or a small molecule.

108. The composition of either claim 106 or claim 107, wherein the cell targeting moiety is a small molecule.

109. The composition of claim 108, wherein the small molecule is a vitamin or cofactor.

110. The composition of claim 109, wherein the small molecule is folate.

111. The composition according to any one of claims 101-110, wherein the second polymer-conjugated lipid is DSPE-PEG2000-Folate.

112. The composition according to any one of claims 101-110, wherein the second polymer-conjugated lipid is DSPE-PEG2000.

113. The composition according to any one of claims 1-112, wherein the composition comprises a molar ratio of the ionizable lipid to total polynucleotide components of from about 1:10 to about 100:1.

114. The composition according to claim 113, wherein the composition comprises a molar ratio of the ionizable lipid to total polynucleotide components of from about 1:1 to about 50:1.

115. The composition according to claim 114, wherein the composition comprises a molar ratio the ionizable lipid to total polynucleotide components from about 5:1 to about 15:1

116. The composition according to any one of claims 1-115, wherein the composition comprises 5A2-SC8, cholesterol, DOPE, and DMG-PEG2000.

117. The composition according to claim 116, wherein the composition comprises a molar ratio of 5A2-SC8: DOPE: cholesterol: DMG-PEG2000 of from about 15:15:30:2.

118. The composition according to any one of claims 1-117, wherein the composition further comprises a pharmaceutically excipient or carrier.

119. The composition according to any one of claims 1-118, wherein the composition is formulated as a solution.

120. The composition according to any one of claims 1-119, wherein the composition is formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctivally, subcutaneously, sublingually, topically, transbuccally, transdermally, vaginally, in crèmes, in lipid compositions, via a catheter, via a lavage, via continuous infusion, via infusion, via inhalation, via injection, via local delivery, or via localized perfusion.

121. The composition of claim 120, wherein the composition is formulated for administration via injection or via inhalation.

122. The composition of claim 121, wherein the composition is formulated for administration via injection.

123. The composition of claim 121, wherein the composition is formulated for administration via inhalation.

124. The composition according to any one of claims 1-123, wherein the composition is formulated as a unit dose.

125. A method of treating a disease or disorder comprising administering to a patient a therapeutically effective amount of a composition according to any one of claims 1-124.

126. A composition according to any one of claims 1-124 for use in the treatment of a disease or disorder.

127. Use of a composition according to any one of claims 1-124 in the treatment of a disease or disorder.

128. The method according to any one of claims 125-127, wherein the disease or disorder is cancer.

129. The method according to any one of claims 125-128, wherein the cancer is a carcinoma, sarcoma, lymphoma, leukemia, melanoma, mesothelioma, multiple myeloma, or seminoma.

130. The method according to any one of claims 125-128, wherein the cancer is of the bladder, blood, bone, brain, breast, central nervous system, cervix, colon, endometrium, esophagus, gall bladder, gastrointestinal tract, genitalia, genitourinary tract, head, kidney, larynx, liver, lung, muscle tissue, neck, oral or nasal mucosa, ovary, pancreas, prostate, skin, spleen, small intestine, large intestine, stomach, testicle, or thyroid.

131. The method according to any one of claims 125-130, wherein the cancer is a solid tumor.

132. The method according to any one of claims 125-131, wherein the cancer is ovarian cancer or liver cancer.

133. The method according to any one of claims 125-132, wherein the inhibitory polynucleotide inhibits a protein overexpressed in the disease or disorder.

134. The method of claim 133, wherein the protein is a focal adhesion kinase.

135. The method according to any one of claims 125-134, wherein the guide polynucleotide targets PD-L1.

136. The method according to any one of claims 125-135, wherein the guide polynucleotide results in reduced expression of PD-L1.

137. The method according to any one of claims 125-136, wherein the method further comprises one or more additional therapeutic modalities.

138. The method of claim 137, wherein the additional therapeutic modality is radiation therapy, an additional chemotherapy, surgery, or immunotherapy.

139. The method according to any one of claims 125-138, wherein the method comprises administering the composition: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctivally, subcutaneously, sublingually, topically, transbuccally, transdermally, vaginally, in crèmes, in lipid compositions, via a catheter, via a lavage, via continuous infusion, via infusion, via inhalation, via injection, via local delivery, or via localized perfusion.

140. The method of claim 139, wherein the composition is administered via inhalation or via injection.

141. The method according to any one of claims 125-140, wherein the composition is administered locally.

142. The method according to any one of claims 125-140, wherein the composition is administered systemically.

143. The method according to any one of claims 125-142, wherein the method comprises administering the composition once.

144. The method according to any one of claims 125-142, wherein the method comprises administering the composition two or more times.

145. A method of inhibiting a focal adhesion kinase (FAK) in a patient comprising administering a composition according to any one of claims 1-124, wherein the inhibitory polynucleotide inhibits FAK.

146. A composition according to any one of claims 1-124 for use in inhibiting a focal adhesion kinase (FAK) in a patient, wherein the composition comprises an inhibitory polynucleotide that inhibits FAK.

147. Use of a composition according to any one of claims 1-124 to inhibit a focal adhesion kinase (FAK), wherein the composition comprises an inhibitory polynucleotide that inhibits FAK.

148. The method according to any one of claims 145-147, wherein the method results in increased delivery of the nuclease or polynucleotide encoding for the nuclease.

149. The method of claim 148, wherein the increased delivery results in improved cell editing compared to a composition without a FAK inhibitory polynucleotide.

150. The method of claim 149, wherein the cell editing is gene editing.

151. The method of claim 150, wherein the cell editing is gene silencing.

152. A method of delivering of a polynucleotide that encodes for a protein comprising contacting the cell with a composition according to any one of claims 1-124, wherein the inhibitory polynucleotide is a focal adhesion kinase (FAK) inhibitory polynucleotide.

153. A composition according to any one of claims 1-124 for use in delivering a polynucleotide that encodes for a protein to a cell, wherein the composition comprises a focal adhesion kinase (FAK) inhibitory polynucleotide.

154. Use of a composition according to any one of claims 1-124 to deliver a polynucleotide that encodes for a protein, wherein the composition comprises a focal adhesion kinase (FAK) inhibitory polynucleotide.

155. The method according to any one of claims 152-154, wherein the method results in improved delivery of the polynucleotide compared to a method without a non-FAK inhibitory polynucleotide.

156. A method of editing the genome of a cell that encodes for a protein comprising contacting the cell with a composition according to any one of claims 1-124, wherein the inhibitory polynucleotide is a focal adhesion kinase (FAK) inhibitory polynucleotide.

157. A composition according to any one of claims 1-124 for use in editing the genome of a cell that encodes for a protein, wherein the composition comprises a focal adhesion kinase (FAK) inhibitory polynucleotide.

158. Use of a composition according to any one of claims 1-124 for editing the genome of a cell that encodes for a protein, wherein the composition comprises a focal adhesion kinase (FAK) inhibitory polynucleotide.

159. The method according to any one of claims 156-158, wherein the method results in improved genome editing of the polynucleotide compared to a method without a non-FAK inhibitory polynucleotide.

160. A method of silencing the genome of a cell that encodes for a protein comprising contacting the cell with a composition according to any one of claims 1-124, wherein the inhibitory polynucleotide is a focal adhesion kinase (FAK) inhibitory polynucleotide.

161. A composition according to any one of claims 1-124 for use in silencing the genome of a cell that encodes for a protein, wherein the composition comprises a focal adhesion kinase (FAK) inhibitory polynucleotide.

162. Use of a composition according to any one of claims 1-124 for silencing the genome of a cell that encodes for a protein, wherein the composition comprises a focal adhesion kinase (FAK) inhibitory polynucleotide.

163. The method according to any one of claims 160-162, wherein the method results in improved silencing of the genome compared to a method without a non-FAK inhibitory polynucleotide.

164. The method according to any one of claims 152-163, wherein the method is carried out in vitro.

165. The method according to any one of claims 152-163, wherein the method is carried out in vivo.

166. The method of claim 165, wherein the in vivo method comprises administering to the cell in a patient.

167. The method of claim 166, wherein the patient is a mammal.

168. The method of claim 167, wherein the mammal is a human.

169. The method according to any one of claims 145-168, wherein the method is sufficient to treat a disease or disorder.


SEQUENCE LISTING (21 sequences)

SEQ ID NO: 1 - DNA, 20 bp, synthetic construct
  misc_feature 1..20: note = Synthetic primer
  source 1..20: mol_type = other DNA
ccgctggttcactaagtgcg

SEQ ID NO: 2 - DNA, 21 bp, synthetic construct
  misc_feature 1..21: note = Synthetic primer
  source 1..21: mol_type = other DNA
ttctctgagacgagcttggcg

SEQ ID NO: 3 - DNA, 24 bp, synthetic construct
  misc_feature 1..24: note = Synthetic primer
  source 1..24: mol_type = other DNA
caggtaatacagaactaacaggtg

SEQ ID NO: 4 - DNA, 20 bp, synthetic construct
  misc_feature 1..20: note = Synthetic primer
  source 1..20: mol_type = other DNA
gctgcataatcagctacggt

SEQ ID NO: 5 - DNA, 24 bp, synthetic construct
  misc_feature 1..24: note = Synthetic primer
  source 1..24: mol_type = other DNA
gtcacatggatatattacatagtg

SEQ ID NO: 6 - DNA, 24 bp, synthetic construct
  misc_feature 1..24: note = Synthetic primer
  source 1..24: mol_type = other DNA
cagaatattacctgggatgaccaa

SEQ ID NO: 7 - DNA, 20 bp, synthetic construct
  misc_feature 1..20: note = Synthetic primer
  source 1..20: mol_type = other DNA
cctgactattgatgttgagc

SEQ ID NO: 8 - DNA, 24 bp, synthetic construct
  misc_feature 1..24: note = Synthetic primer
  source 1..24: mol_type = other DNA
ctgctctaatcccccaagaacttc

SEQ ID NO: 9 - DNA, 21 bp, synthetic construct
  misc_feature 1..21: note = Synthetic primer
  source 1..21: mol_type = other DNA
gtggtgcccatcctggtcgag

SEQ ID NO: 10 - DNA, 22 bp, synthetic construct
  misc_feature 1..22: note = Synthetic primer
  source 1..22: mol_type = other DNA
cgcttctcgttggggtctttgc

SEQ ID NO: 11 - DNA, 20 bp, synthetic construct
  misc_feature 1..20: note = Synthetic primer
  source 1..20: mol_type = other DNA
gcctggtgaaagctgtcatc

SEQ ID NO: 12 - DNA, 20 bp, synthetic construct
  misc_feature 1..20: note = Synthetic primer
  source 1..20: mol_type = other DNA
gcttctgtgccatctcaatc

SEQ ID NO: 13 - DNA, 20 bp, synthetic construct
  misc_feature 1..20: note = Synthetic primer
  source 1..20: mol_type = other DNA
cccctggccaaggtcatcca

SEQ ID NO: 14 - DNA, 20 bp, synthetic construct
  misc_feature 1..20: note = Synthetic primer
  source 1..20: mol_type = other DNA
acagccttggcagcgccagt

SEQ ID NO: 15 - RNA, 20 nt, synthetic construct
  source 1..20: mol_type = other RNA
actgcttgtccagatgactt

SEQ ID NO: 16 - RNA, 20 nt, synthetic construct
  source 1..20: mol_type = other RNA
ggtccagctcccgttctaca

SEQ ID NO: 17 - RNA, 20 nt, synthetic construct
  source 1..20: mol_type = other RNA
  modified_base 1..20: mod_base = OTHER; note = t is thymine
gtatttctactgcgacgagg

SEQ ID NO: 18 - RNA, 20 nt, synthetic construct
  source 1..20: mol_type = other RNA
tcccttctcaggattcctac

SEQ ID NO: 19 - RNA, 20 nt, synthetic construct
  source 1..20: mol_type = other RNA
gctttcacggaggttcgacg

SEQ ID NO: 20 - RNA, 20 nt, synthetic construct
  source 1..20: mol_type = other RNA
gaagttcgagggcgacaccc

SEQ ID NO: 21 - RNA, 23 nt, synthetic construct
  source 1..23: mol_type = other RNA
  modified_base 20..23: mod_base = OTHER; note = t is thymine
gggcatcattcagaagatadtdt